(19) United States
(12) Patent Application Publication
(10) Pub. No.: US 2023/0292600 A1
(43) Pub. Date: Sep. 14, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Changwoo KIM, Suwon-si (KR); Seungjae LEE, Suwon-si (KR); Chang Ju SHIN, Suwon-si (KR); Jongwoo WON, Suwon-si (KR); Youngkyoung JO, Suwon-si (KR); Hyung Sun KIM, Suwon-si (KR); Hyungyu LEE, Suwon-si (KR); Sung-Hyun JUNG, Suwon-si (KR)

(21) Appl. No.: 17/899,037

(22) Filed: Aug. 30, 2022

(30) Foreign Application Priority Data
Sep. 9, 2021 (KR) 10-2021-0120580
Aug. 29, 2022 (KR) 10-2022-0108579

(51) Int. Cl. H01L 51/00; C07D 405/14; C07D 403/14

(52) U.S. Cl. CPC H01L 51/0067; C07D 403/14; C07D 405/14; H01L 51/0072; H01L 51/0073; H01L 51/5016

(57) ABSTRACT

A compound for an organic optoelectronic device, a composition for an organic optoelectronic device, an organic optoelectronic device including the same, and a display device, the compound being represented by Chemical Formula 1:

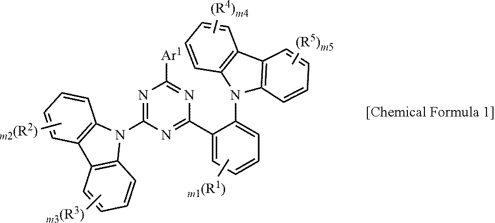

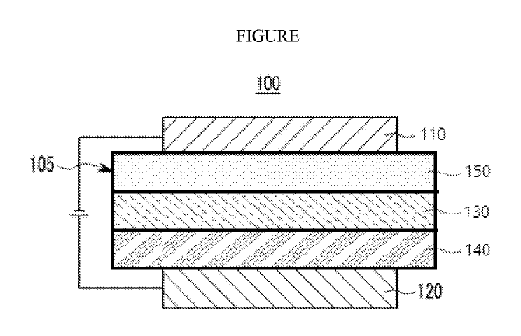

### CROSS-REFERENCE TO RELATED APPLICATION

This application claims priority to and the benefit of Korean Patent Application No. 10-2021-0120580 filed in the Korean Intellectual Property Office on Sep. 9, 2021, and Korean Patent Application No. 10-2022-0108579 filed in the Korean Intellectual Property Office on Aug. 29, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photo conductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

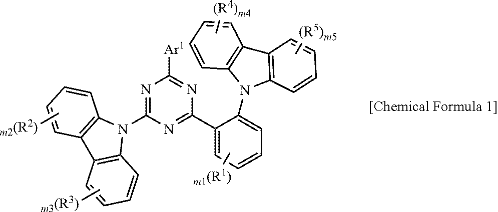

wherein, in Chemical Formula 1, Ar¹ is a substituted or unsubstituted dibenzofuranyl group, a substituted or unsubstituted dibenzothiophenyl group, or a substituted or unsubstituted carbazolyl group, R¹ is hydrogen, deuterium, or an unsubstituted C6 to C12 aryl group, R² to R⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and m1 to m5 are each independently an integer 1 to 4.

The embodiments may be realized by providing a composition for an organic optoelectronic device, the composition including a first compound; and a second compound, wherein the first compound is the compound according to an embodiment, and the second compound is represented by Chemical Formula 2 or is represented by a combination ofChemical Formulae 3 and 4:

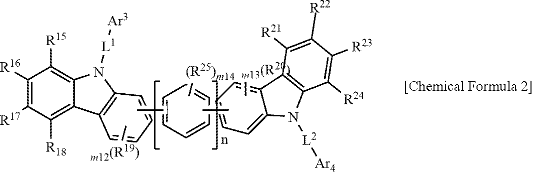

in Chemical Formula 2, Ar³ and Ar⁴ are each independently a substituted or unsubstituted C6 to C20 aryl group or a substituted or unsubstituted C2 to C30 heterocyclic group, L¹ and L² are each independently a single bond or a substituted or unsubstituted C6 to C20 arylene group, R¹⁵ to R²⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, m12 and m13 are each independently an integer of 1 to 3, m14 is an integer of 1 to 4, and n is an integer of 0 to 2;

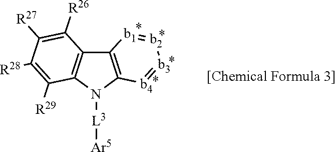

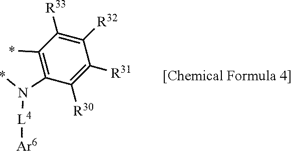

in Chemical Formulas 3 and 4, Ar⁵ and Ar⁶ are each independently a substituted or unsubstituted C6 to C20 aryl group or a substituted or unsubstituted C2 to C30 heterocyclic group, two adjacent ones of b₁* to b₄* of Chemical Formula 3 are linking carbons linked at * of Chemical Formula 4, the remaining two of b₁* to b₄* of Chemical Formula 3, not linked at * of Chemical Formula 4, are each independently C—L^{a}—R^{a}, L^{a}, L³, and L⁴ are each independently a single bond or a substituted or unsubstituted C6 to C20 arylene group, and R^{a} and R²⁶ to R³³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, and at least one organic layer between the anode and the cathode, wherein the at least one organic layer includes the composition for an organic optoelectronic device according to an embodiment.

The embodiments may be realized by providing a display device comprising the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (-H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an embodiment is described.

The compound for the organic optoelectronic device according to an embodiment may be represented by, e.g., Chemical Formula 1.

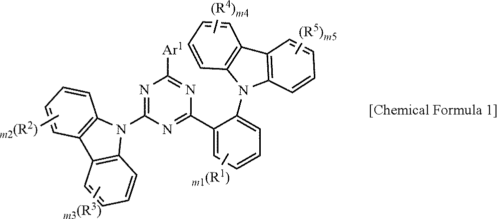

In Chemical Formula 1, Ar¹ may be or may include, e.g., a substituted or unsubstituted dibenzofuranyl group, a substituted or unsubstituted dibenzothiophenyl group, or a substituted or unsubstituted carbazolyl group.

R¹ may be or may include, e.g., hydrogen, deuterium, or an unsubstituted C6 to C12 aryl group.

R² to R⁵ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof.

m1 to m5 may each independently be, e.g., an integer of 1 to 4.

The compound represented by Chemical Formula 1 may have a structure in which one carbazole group is directly linked to the triazine without a linking group in an N-direction centering on the triazine, another carbazole group is linked to the triazine in an N-direction through ortho-phenylene, and the triazine is substituted with a substituted or unsubstituted dibenzofuranyl group, a substituted or unsubstituted dibenzothiophenyl group, or a substituted or unsubstituted carbazolyl group.

One carbazole group may be directly linked to the triazine without a linking group in the N-direction, e.g., the 9th position, so that it has a relatively deep LUMO energy level, which is advantageous for electron injection and movement.

In addition, the other carbazole group may be linked to the triazine in the N-direction, e.g., 9th position, so that a π-bonding through the C—N bond is broken, and the electron cloud between HOMO-LUMO may be clearly localized into a hole transport moiety and an electron transport moiety.

In an implementation, the HOMO-LUMO band gap may be widened due to the ortho-phenylene, an efficiency improvement effect may be maximized, and a steric hindrance of molecules may be increased, so that a deposition temperature is not relatively high, which is advantageous in the process.

In an implementation, as the triazine is substituted with the substituted or unsubstituted dibenzofuranyl group, the substituted or unsubstituted dibenzothiophenyl group, or the substituted or unsubstituted carbazolyl group, the electron transport capability may be adjusted by the appropriate mobility and charge balance in the light emitting layer to manufacture a low-driving, high-efficiency, long life-span organic electroluminescent device.

In an implementation, Ar¹ of Chemical Formula 1 may be, e.g., a substituted or unsubstituted dibenzofuranyl group, and the compound may be represented by, e.g., Chemical Formula 1A.

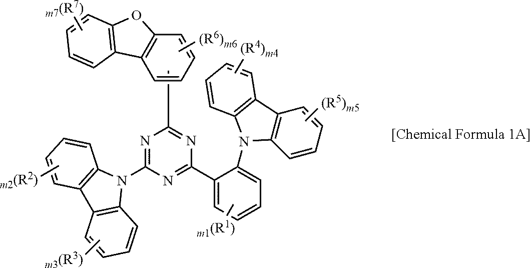

In an implementation, Ar¹ of Chemical Formula 1 may be, e.g., a substituted or unsubstituted dibenzothiophenyl group, and the compound may be represented by, e.g., Chemical Formula 1B.

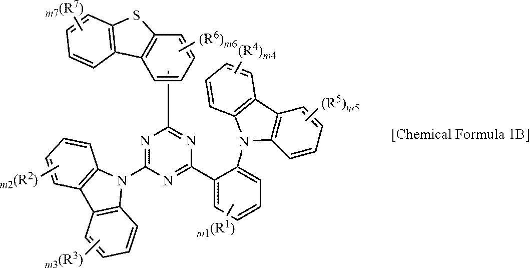

In an implementation, Ar¹ of Chemical Formula 1 may be, e.g., a substituted or unsubstituted carbazolyl group, and the compound may be represented by, e.g., Chemical Formula 1C or Chemical Formula 1D.

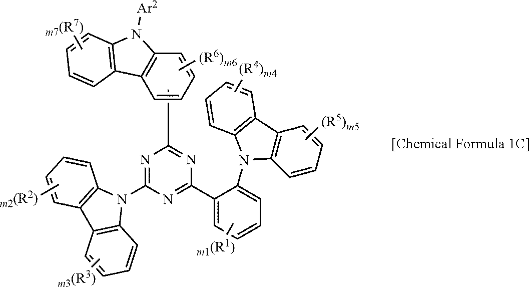

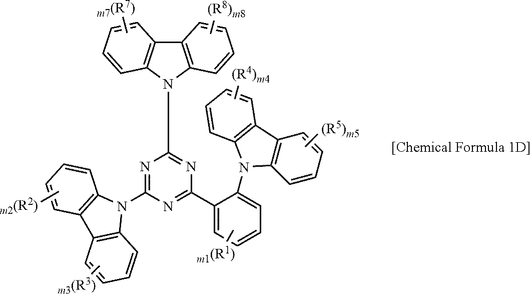

In Chemical Formula 1A to Chemical Formula 1D, R¹ to R⁵ and m1 to m5 may be defined the same as those described above.

R⁶ to R⁸ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof.

m6 may be, e.g., an integer 1 to 3.

m7 and m8 may each independently be, e.g., an integer of 1 to 4.

Ar² may be, e.g., a substituted or unsubstituted C6 to C12 aryl group.

In an implementation, Chemical Formula 1A may be represented by, e.g., one of Chemical Formula 1A-1 to Chemical Formula 1A-4. [0060]

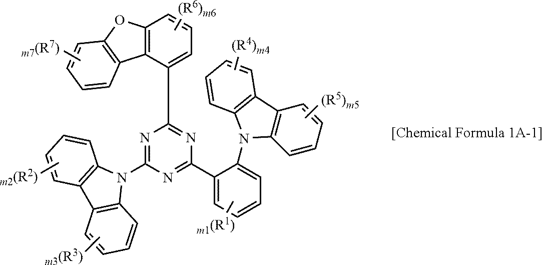

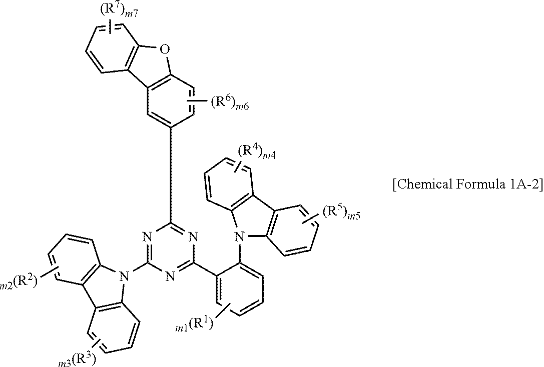

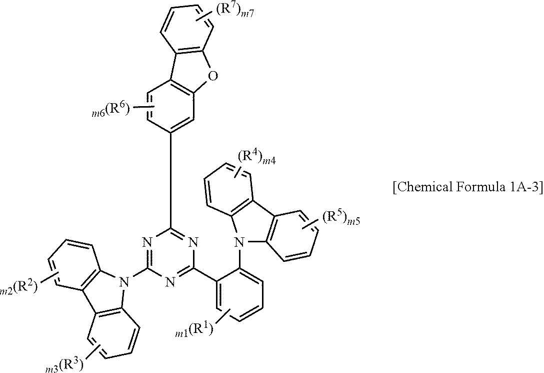

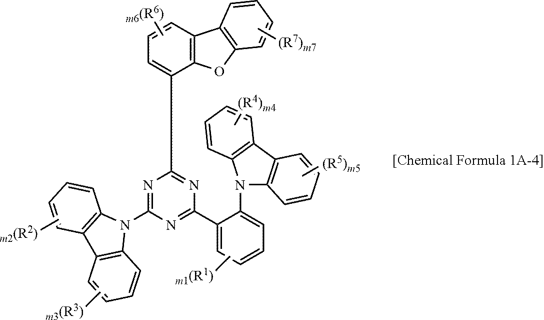

In Chemical Formula 1A-1 to Chemical Formula 1A-4, R¹ to R⁷ and m1 to m7 may be defined the same as those described above.

In an implementation, Chemical Formula 1B may be represented by, e.g., one of Chemical Formula 1B-1 to Chemical Formula 1B-4.

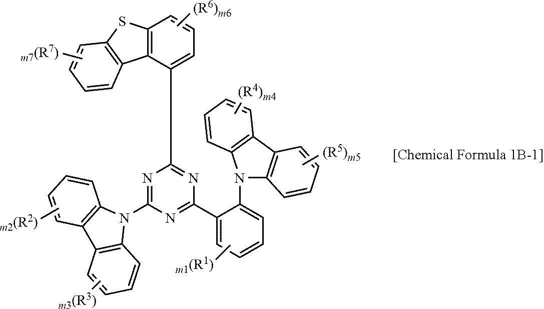

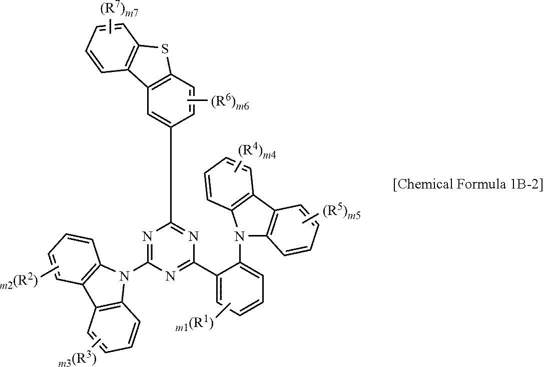

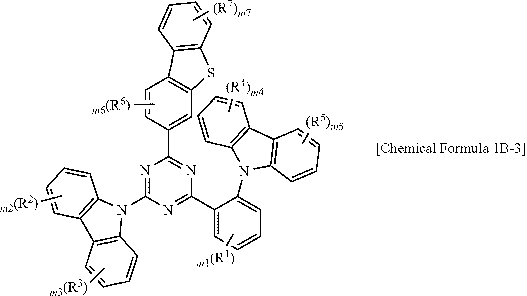

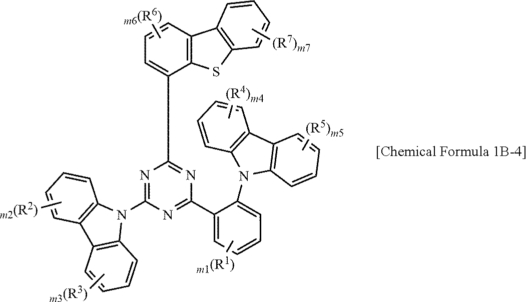

In Chemical Formula 1B-1 to Chemical Formula 1B-4, R¹ to R⁷ and m1 to m7 may be defined the same as those described above.

In an implementation, Chemical Formula 1C may be represented by, e.g., one of Chemical Formula 1C-1 to Chemical Formula 1C-4.

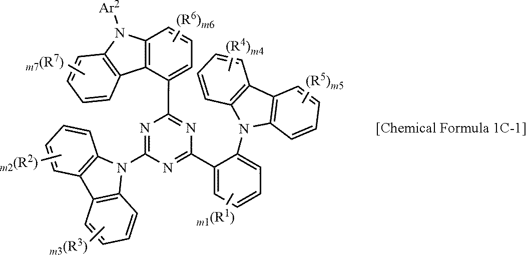

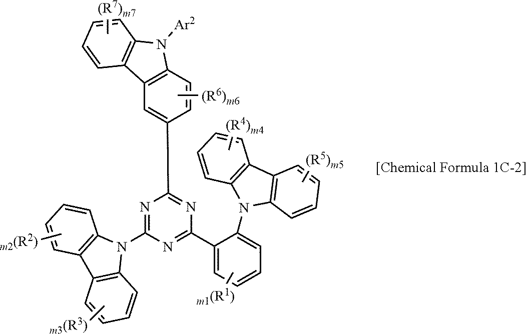

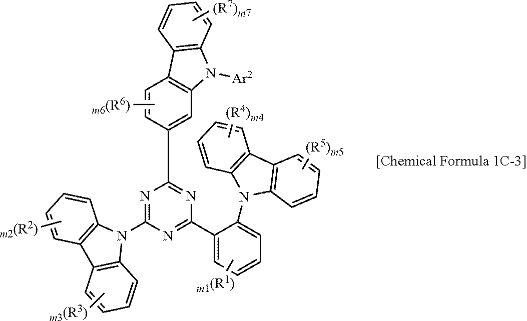

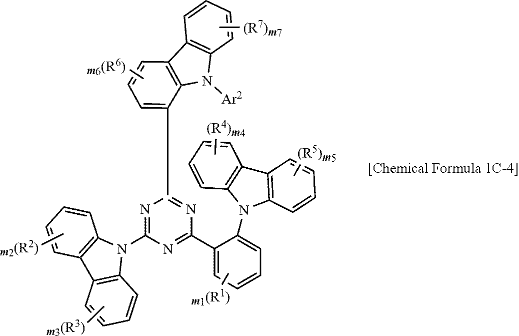

In Chemical Formula 1C-1 to Chemical Formula 1C-4, R¹ to R⁷, m1 to m7, and Ar² may be defined the same as those described above.

In an implementation, R⁶ and R⁷ may each independently be, e.g., hydrogen, deuterium, a substituted or unsubstituted C1 to C10 alkyl group, a substituted or unsubstituted C6 to C12 aryl group, or a combination thereof.

In an implementation, R⁶ and R⁷ may each independently be, e.g., hydrogen, deuterium, a substituted or unsubstituted C1 to C5 alkyl group, a substituted or unsubstituted phenyl group, a substituted or unsubstituted biphenyl group, or a substituted or unsubstituted naphthyl group.

In an implementation, Ar¹ may be, e.g., a group of Group I.

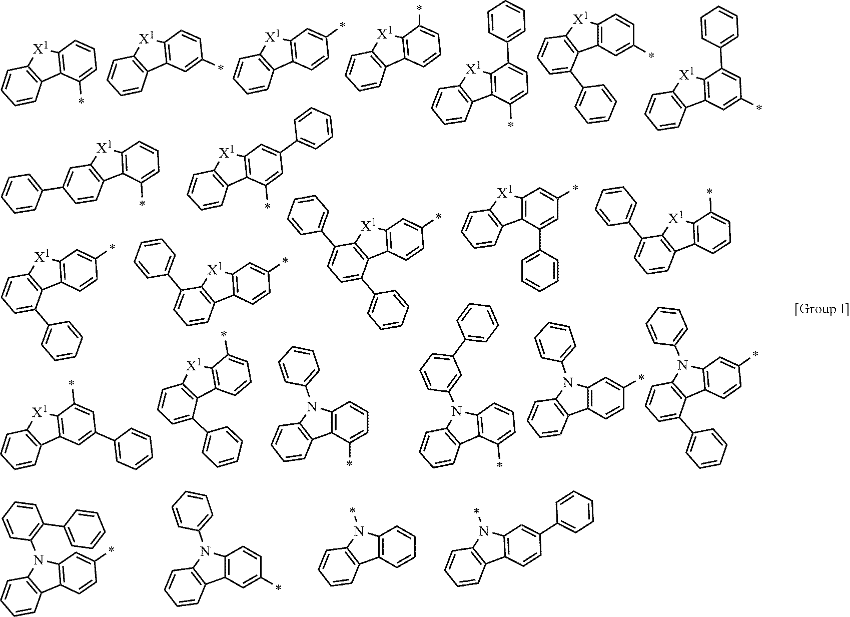

In Group I, X¹ may be, e.g., O or S, and * is a linking point.

In an implementation, groups of Group I may be unsubstituted or further substituted with additional substituents.

The additional substituents may include, e.g., deuterium, a cyano group, a C1 to C10 alkyl group, or a C6 to C12 aryl group.

In an implementation, the additional substituents may include, e.g., deuterium, a C1 to C5 alkyl group, a phenyl group, a biphenyl group, or a naphthyl group.

In an implementation, R¹ to R⁸ may each independently be, e.g., hydrogen, deuterium, or a substituted or unsubstituted phenyl group.

In an implementation, Ar² may be, e.g., a substituted or unsubstituted phenyl group or a substituted or unsubstituted biphenyl group.

In an implementation, Chemical Formula 1 may be represented by, e.g., Chemical Formula 1A-3, Chemical Formula 1B-3, Chemical Formula 1C-3, or Chemical Formula 1D.

In an implementation, Chemical Formula 1 may be represented by, e.g., Chemical Formula 1A-3, Chemical Formula 1B-3, or Chemical Formula 1C-3.

In an implementation, the compound for the organic optoelectronic device represented by Chemical Formula 1 may be, e.g., a compound of Group 1.

### Group 1

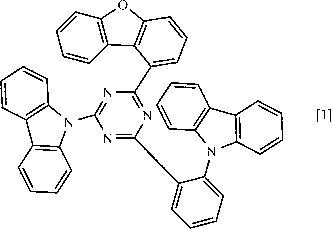 [SMILES: c1ccc(-n2c3ccccc3c3ccccc32)c(-c2nc(-c3cccc4oc5ccccc5c34)nc(-n3c4ccccc4c4ccccc43)n2)c1]

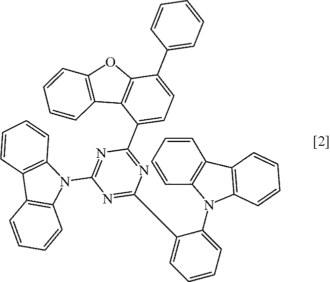 [SMILES: c1ccc(-c2ccc(-c3nc(-c4ccccc4-n4c5ccccc5c5ccccc54)nc(-n4c5ccccc5c5ccccc54)n3)c3c2oc2ccccc23)cc1]

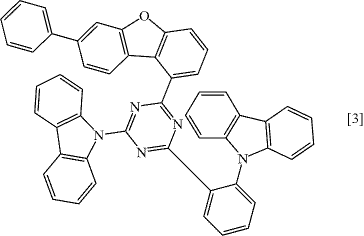 [SMILES: c1ccc(-c2ccc3c(c2)oc2cccc(-c4nc(-c5ccccc5-n5c6ccccc6c6ccccc65)nc(-n5c6ccccc6c6ccccc65)n4)c23)cc1]

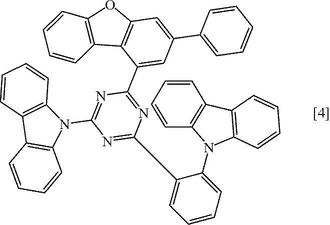 [SMILES: c1ccc(-c2cc(-c3nc(-c4ccccc4-n4c5ccccc5c5ccccc54)nc(-n4c5ccccc5c5ccccc54)n3)c3c(c2)oc2ccccc23)cc1]

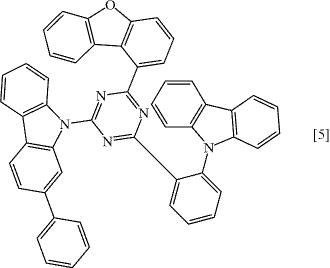 [SMILES: c1ccc(-c2ccc3c4ccccc4n(-c4nc(-c5ccccc5-n5c6ccccc6c6ccccc65)nc(-c5cccc6oc7ccccc7c56)n4)c3c2)cc1]

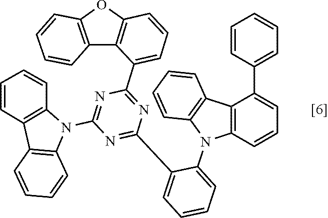 [SMILES: c1ccc(-c2cccc3c2c2ccccc2n3-c2ccccc2-c2nc(-c3cccc4oc5ccccc5c34)nc(-n3c4ccccc4c4ccccc43)n2)cc1]

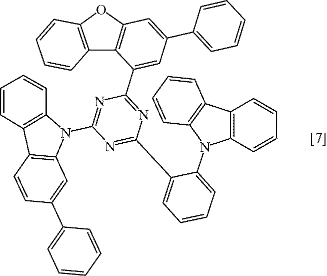 [SMILES: c1ccc(-c2cc(-c3nc(-c4ccccc4-n4c5ccccc5c5ccccc54)nc(-n4c5ccccc5c5ccc(-c6ccccc6)cc54)n3)c3c(c2)oc2ccccc23)cc1]

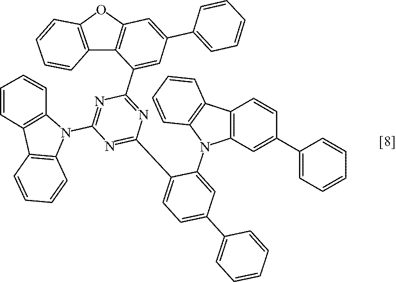 [SMILES: c1ccc(-c2ccc(-c3nc(-c4cc(-c5ccccc5)cc5oc6ccccc6c45)nc(-n4c5ccccc5c5ccccc54)n3)c(-n3c4ccccc4c4ccc(-c5ccccc5)cc43)c2)cc1]

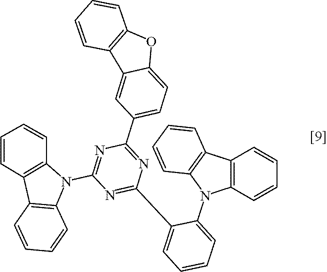 [SMILES: c1ccc(-n2c3ccccc3c3ccccc32)c(-c2nc(-c3ccc4oc5ccccc5c4c3)nc(-n3c4ccccc4c4ccccc43)n2)c1]

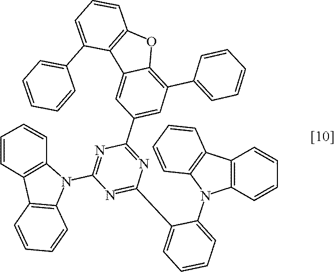 [SMILES: c1ccc(-c2cc(-c3nc(-c4ccccc4-n4c5ccccc5c5ccccc54)nc(-n4c5ccccc5c5ccccc54)n3)cc3c2oc2cccc(-c4ccccc4)c23)cc1]

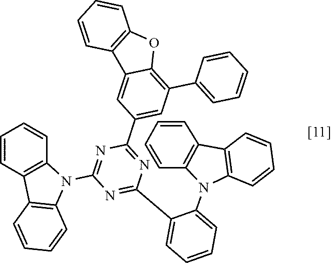 [SMILES: c1ccc(-c2cc(-c3nc(-c4ccccc4-n4c5ccccc5c5ccccc54)nc(-n4c5ccccc5c5ccccc54)n3)cc3c2oc2ccccc23)cc1]

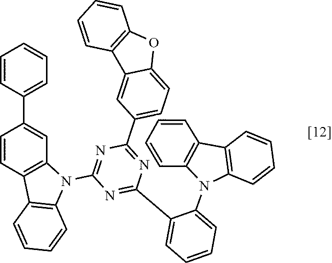 [SMILES: c1ccc(-c2ccc3c4ccccc4n(-c4nc(-c5ccc6oc7ccccc7c6c5)nc(-c5ccccc5-n5c6ccccc6c6ccccc65)n4)c3c2)cc1]

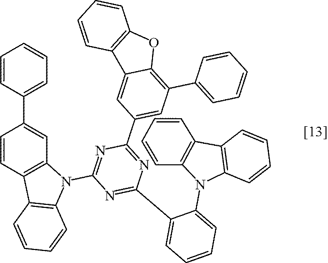 [SMILES: c1ccc(-c2ccc3c4ccccc4n(-c4nc(-c5cc(-c6ccccc6)c6oc7ccccc7c6c5)nc(-c5ccccc5-n5c6ccccc6c6ccccc65)n4)c3c2)cc1]

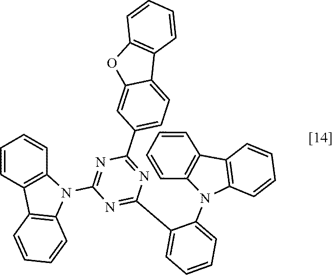 [SMILES: c1ccc(-n2c3ccccc3c3ccccc32)c(-c2nc(-c3ccc4c(c3)oc3ccccc34)nc(-n3c4ccccc4c4ccccc43)n2)c1]

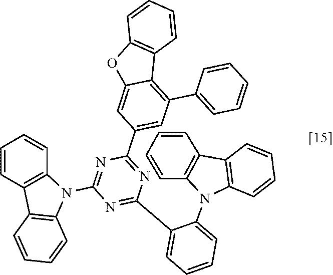 [SMILES: c1ccc(-c2cc(-c3nc(-c4ccccc4-n4c5ccccc5c5ccccc54)nc(-n4c5ccccc5c5ccccc54)n3)cc3oc4ccccc4c23)cc1]

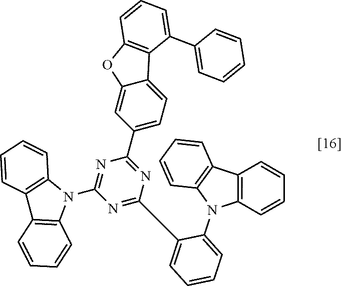 [SMILES: c1ccc(-c2cccc3oc4cc(-c5nc(-c6ccccc6-n6c7ccccc7c7ccccc76)nc(-n6c7ccccc7c7ccccc76)n5)ccc4c23)cc1]

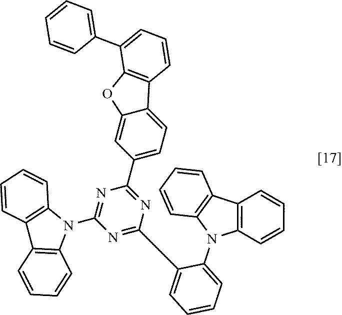 [SMILES: c1ccc(-c2cccc3c2oc2cc(-c4nc(-c5ccccc5-n5c6ccccc6c6ccccc65)nc(-n5c6ccccc6c6ccccc65)n4)ccc23)cc1]

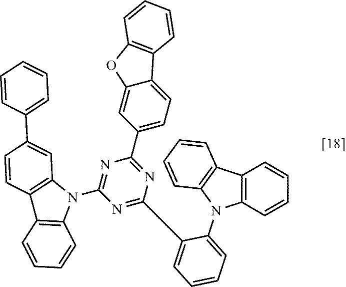 [SMILES: c1ccc(-c2ccc3c4ccccc4n(-c4nc(-c5ccc6c(c5)oc5ccccc56)nc(-c5ccccc5-n5c6ccccc6c6ccccc65)n4)c3c2)cc1]

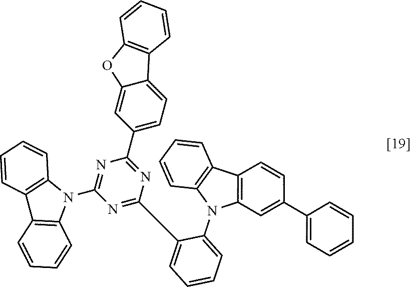 [SMILES: c1ccc(-c2ccc3c4ccccc4n(-c4ccccc4-c4nc(-c5ccc6c(c5)oc5ccccc56)nc(-n5c6ccccc6c6ccccc65)n4)c3c2)cc1]

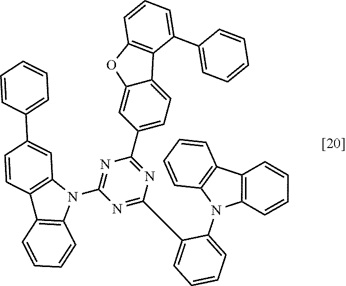 [SMILES: c1ccc(-c2ccc3c4ccccc4n(-c4nc(-c5ccc6c(c5)oc5cccc(-c7ccccc7)c56)nc(-c5ccccc5-n5c6ccccc6c6ccccc65)n4)c3c2)cc1]

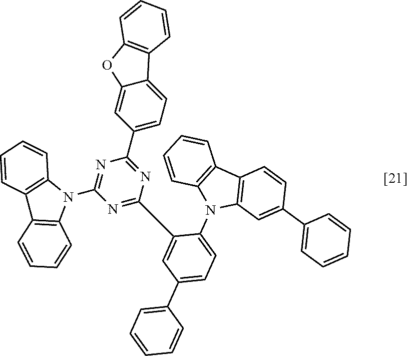 [SMILES: c1ccc(-c2ccc(-n3c4ccccc4c4ccc(-c5ccccc5)cc43)c(-c3nc(-c4ccc5c(c4)oc4ccccc45)nc(-n4c5ccccc5c5ccccc54)n3)c2)cc1]

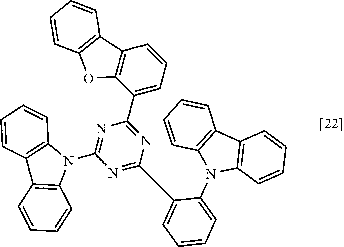 [SMILES: c1ccc(-n2c3ccccc3c3ccccc32)c(-c2nc(-c3cccc4c3oc3ccccc34)nc(-n3c4ccccc4c4ccccc43)n2)c1]

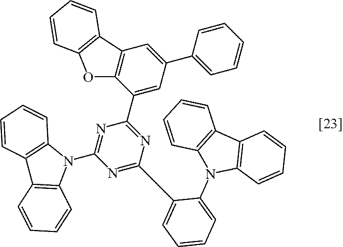 [SMILES: c1ccc(-c2cc(-c3nc(-c4ccccc4-n4c5ccccc5c5ccccc54)nc(-n4c5ccccc5c5ccccc54)n3)c3oc4ccccc4c3c2)cc1]

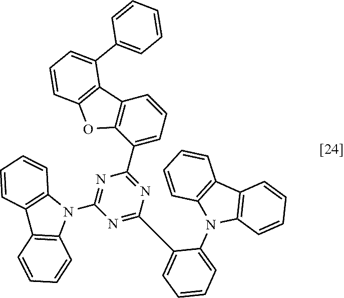 [SMILES: c1ccc(-c2cccc3oc4c(-c5nc(-c6ccccc6-n6c7ccccc7c7ccccc76)nc(-n6c7ccccc7c7ccccc76)n5)cccc4c23)cc1]

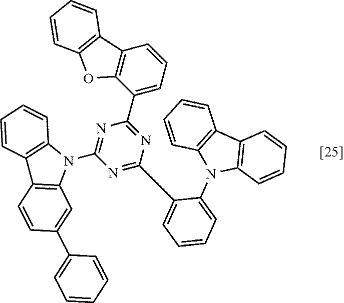 [SMILES: c1ccc(-c2ccc3c4ccccc4n(-c4nc(-c5ccccc5-n5c6ccccc6c6ccccc65)nc(-c5cccc6c5oc5ccccc56)n4)c3c2)cc1]

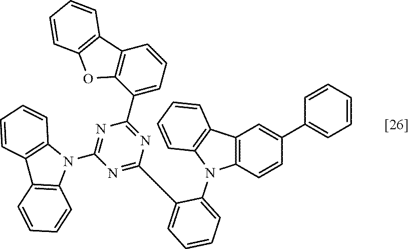 [SMILES: c1ccc(-c2ccc3c(c2)c2ccccc2n3-c2ccccc2-c2nc(-c3cccc4c3oc3ccccc34)nc(-n3c4ccccc4c4ccccc43)n2)cc1]

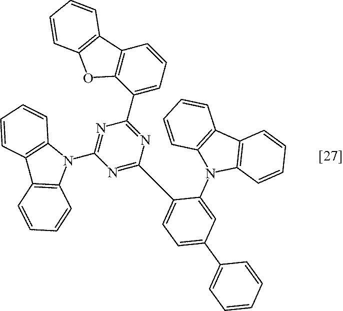 [SMILES: c1ccc(-c2ccc(-c3nc(-c4cccc5c4oc4ccccc45)nc(-n4c5ccccc5c5ccccc54)n3)c(-n3c4ccccc4c4ccccc43)c2)cc1]

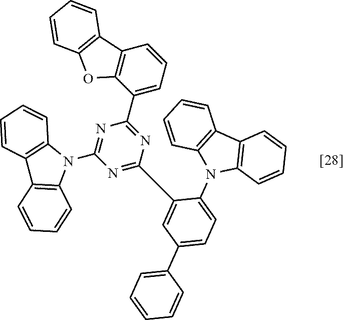 [SMILES: c1ccc(-c2ccc(-n3c4ccccc4c4ccccc43)c(-c3nc(-c4cccc5c4oc4ccccc45)nc(-n4c5ccccc5c5ccccc54)n3)c2)cc1]

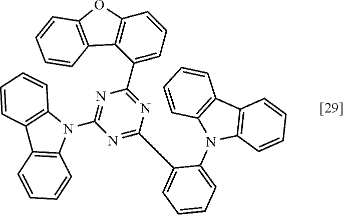 [SMILES: c1ccc(-n2c3ccccc3c3ccccc32)c(-c2nc(-c3cccc4oc5ccccc5c34)nc(-n3c4ccccc4c4ccccc43)n2)c1]

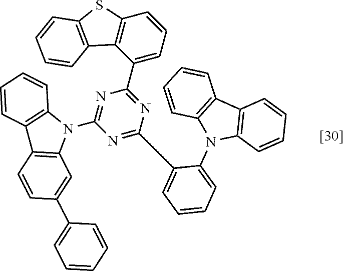 [SMILES: c1ccc(-c2ccc3c4ccccc4n(-c4nc(-c5ccccc5-n5c6ccccc6c6ccccc65)nc(-c5cccc6sc7ccccc7c56)n4)c3c2)cc1]

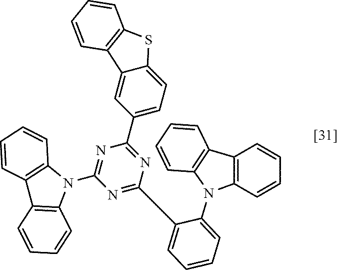 [SMILES: c1ccc(-n2c3ccccc3c3ccccc32)c(-c2nc(-c3ccc4sc5ccccc5c4c3)nc(-n3c4ccccc4c4ccccc43)n2)c1]

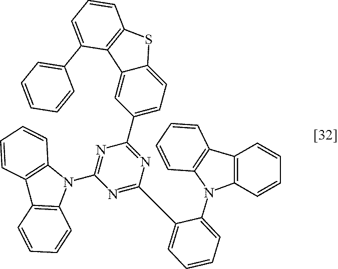 [SMILES: c1ccc(-c2cccc3sc4ccc(-c5nc(-c6ccccc6-n6c7ccccc7c7ccccc76)nc(-n6c7ccccc7c7ccccc76)n5)cc4c23)cc1]

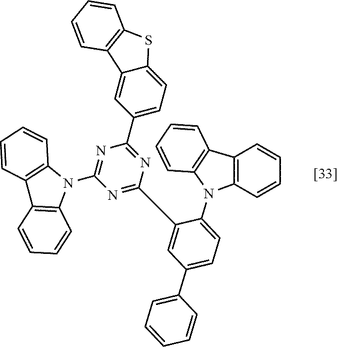 [SMILES: c1ccc(-c2ccc(-n3c4ccccc4c4ccccc43)c(-c3nc(-c4ccc5sc6ccccc6c5c4)nc(-n4c5ccccc5c5ccccc54)n3)c2)cc1]

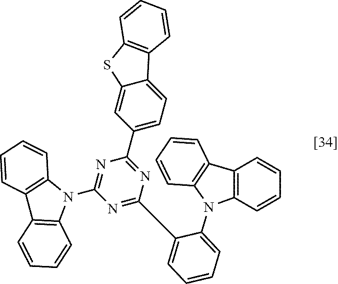 [SMILES: c1ccc(-n2c3ccccc3c3ccccc32)c(-c2nc(-c3ccc4c(c3)sc3ccccc34)nc(-n3c4ccccc4c4ccccc43)n2)c1]

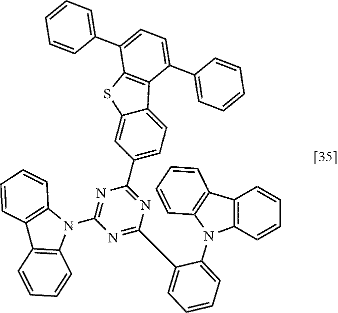 [SMILES: c1ccc(-c2ccc(-c3ccccc3)c3c2sc2cc(-c4nc(-c5ccccc5-n5c6ccccc6c6ccccc65)nc(-n5c6ccccc6c6ccccc65)n4)ccc23)cc1]

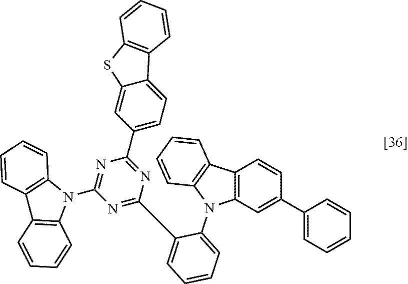 [SMILES: c1ccc(-c2ccc3c4ccccc4n(-c4ccccc4-c4nc(-c5ccc6c(c5)sc5ccccc56)nc(-n5c6ccccc6c6ccccc65)n4)c3c2)cc1]

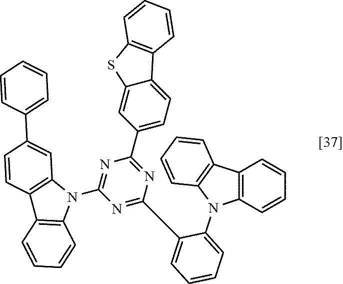 [SMILES: c1ccc(-c2ccc3c4ccccc4n(-c4nc(-c5ccc6c(c5)sc5ccccc56)nc(-c5ccccc5-n5c6ccccc6c6ccccc65)n4)c3c2)cc1]

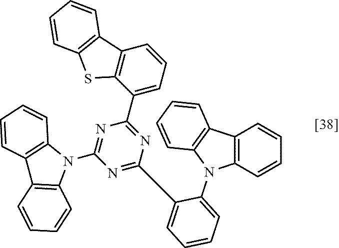 [SMILES: c1ccc(-n2c3ccccc3c3ccccc32)c(-c2nc(-c3cccc4c3sc3ccccc34)nc(-n3c4ccccc4c4ccccc43)n2)c1]

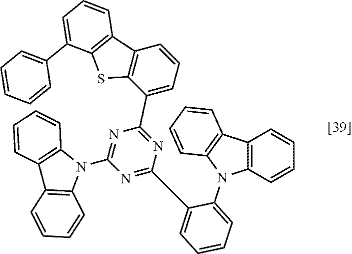 [SMILES: c1ccc(-c2cccc3c2sc2c(-c4nc(-c5ccccc5-n5c6ccccc6c6ccccc65)nc(-n5c6ccccc6c6ccccc65)n4)cccc23)cc1]

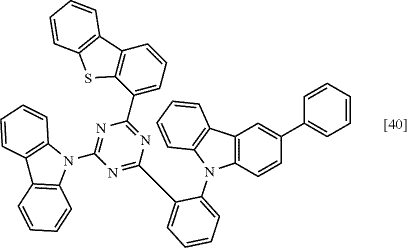 [SMILES: c1ccc(-c2ccc3c(c2)c2ccccc2n3-c2ccccc2-c2nc(-c3cccc4c3sc3ccccc34)nc(-n3c4ccccc4c4ccccc43)n2)cc1]

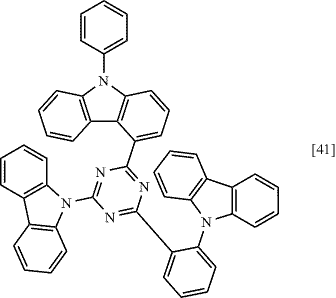 [SMILES: c1ccc(-n2c3ccccc3c3c(-c4nc(-c5ccccc5-n5c6ccccc6c6ccccc65)nc(-n5c6ccccc6c6ccccc65)n4)cccc32)cc1]

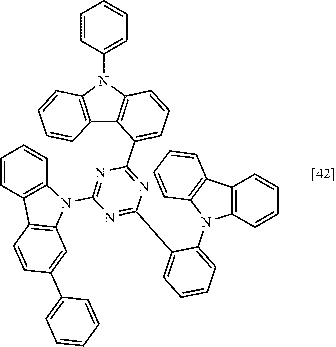 [SMILES: c1ccc(-c2ccc3c4ccccc4n(-c4nc(-c5ccccc5-n5c6ccccc6c6ccccc65)nc(-c5cccc6c5c5ccccc5n6-c5ccccc5)n4)c3c2)cc1]

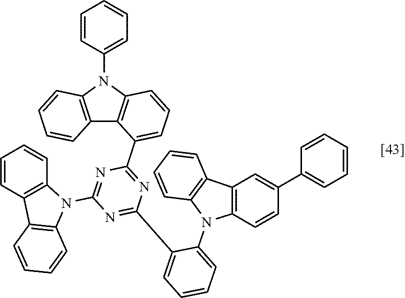 [SMILES: c1ccc(-c2ccc3c(c2)c2ccccc2n3-c2ccccc2-c2nc(-c3cccc4c3c3ccccc3n4-c3ccccc3)nc(-n3c4ccccc4c4ccccc43)n2)cc1]

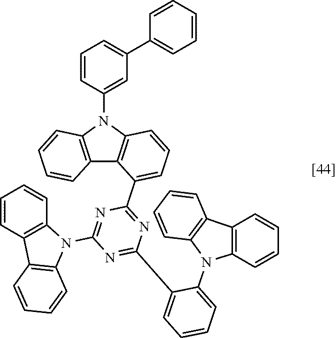 [SMILES: c1ccc(-c2cccc(-n3c4ccccc4c4c(-c5nc(-c6ccccc6-n6c7ccccc7c7ccccc76)nc(-n6c7ccccc7c7ccccc76)n5)cccc43)c2)cc1]

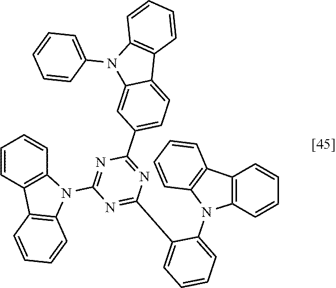 [SMILES: c1ccc(-n2c3ccccc3c3ccc(-c4nc(-c5ccccc5-n5c6ccccc6c6ccccc65)nc(-n5c6ccccc6c6ccccc65)n4)cc32)cc1]

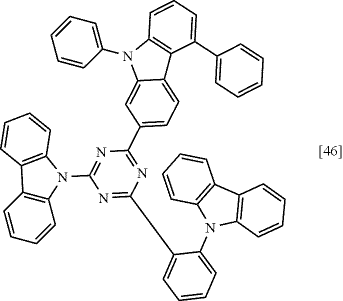 [SMILES: c1ccc(-c2cccc3c2c2ccc(-c4nc(-c5ccccc5-n5c6ccccc6c6ccccc65)nc(-n5c6ccccc6c6ccccc65)n4)cc2n3-c2ccccc2)cc1]

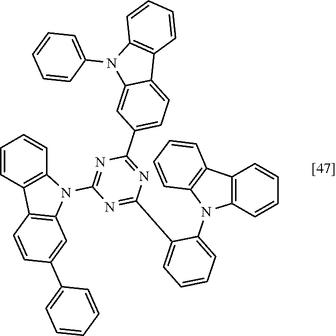 [SMILES: c1ccc(-c2ccc3c4ccccc4n(-c4nc(-c5ccc6c7ccccc7n(-c7ccccc7)c6c5)nc(-c5ccccc5-n5c6ccccc6c6ccccc65)n4)c3c2)cc1]

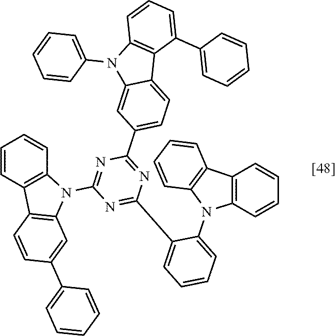 [SMILES: c1ccc(-c2ccc3c4ccccc4n(-c4nc(-c5ccc6c7c(-c8ccccc8)cccc7n(-c7ccccc7)c6c5)nc(-c5ccccc5-n5c6ccccc6c6ccccc65)n4)c3c2)cc1]

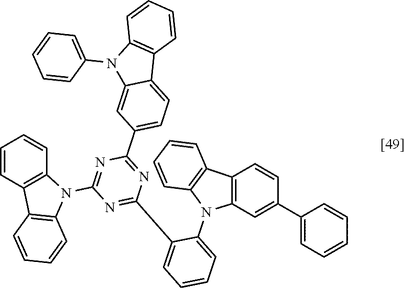 [SMILES: c1ccc(-c2ccc3c4ccccc4n(-c4ccccc4-c4nc(-c5ccc6c7ccccc7n(-c7ccccc7)c6c5)nc(-n5c6ccccc6c6ccccc65)n4)c3c2)cc1]

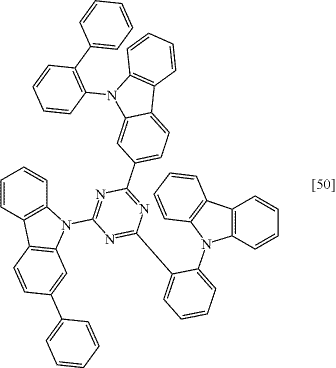 [SMILES: c1ccc(-c2ccc3c4ccccc4n(-c4nc(-c5ccc6c7ccccc7n(-c7ccccc7-c7ccccc7)c6c5)nc(-c5ccccc5-n5c6ccccc6c6ccccc65)n4)c3c2)cc1]

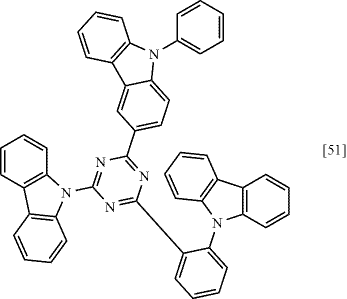 [SMILES: c1ccc(-n2c3ccccc3c3cc(-c4nc(-c5ccccc5-n5c6ccccc6c6ccccc65)nc(-n5c6ccccc6c6ccccc65)n4)ccc32)cc1]

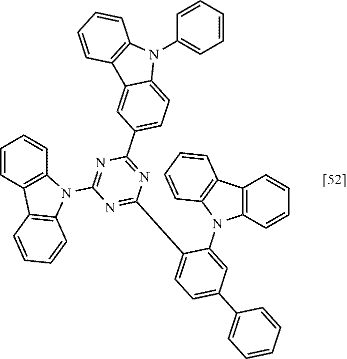 [SMILES: c1ccc(-c2ccc(-c3nc(-c4ccc5c(c4)c4ccccc4n5-c4ccccc4)nc(-n4c5ccccc5c5ccccc54)n3)c(-n3c4ccccc4c4ccccc43)c2)cc1]

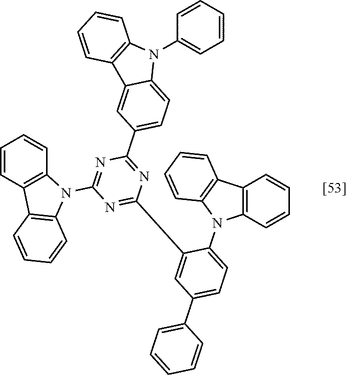 [SMILES: c1ccc(-c2ccc(-n3c4ccccc4c4ccccc43)c(-c3nc(-c4ccc5c(c4)c4ccccc4n5-c4ccccc4)nc(-n4c5ccccc5c5ccccc54)n3)c2)cc1]

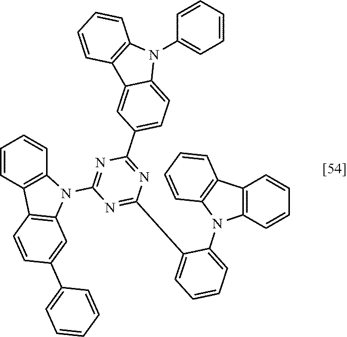 [SMILES: c1ccc(-c2ccc3c4ccccc4n(-c4nc(-c5ccc6c(c5)c5ccccc5n6-c5ccccc5)nc(-c5ccccc5-n5c6ccccc6c6ccccc65)n4)c3c2)cc1]

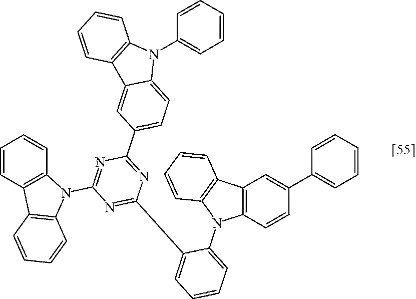 [SMILES: c1ccc(-c2ccc3c(c2)c2ccccc2n3-c2ccccc2-c2nc(-c3ccc4c(c3)c3ccccc3n4-c3ccccc3)nc(-n3c4ccccc4c4ccccc43)n2)cc1]

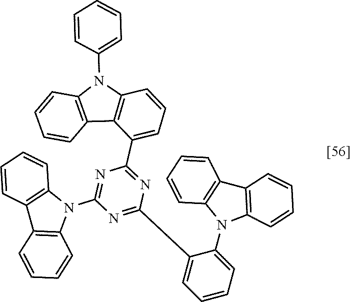 [SMILES: c1ccc(-n2c3ccccc3c3c(-c4nc(-c5ccccc5-n5c6ccccc6c6ccccc65)nc(-n5c6ccccc6c6ccccc65)n4)cccc32)cc1]

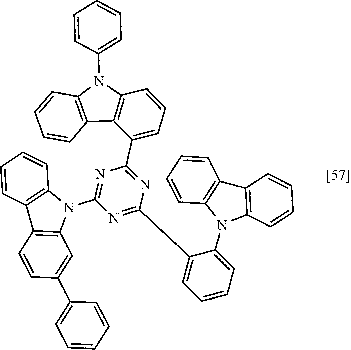 [SMILES: c1ccc(-c2ccc3c4ccccc4n(-c4nc(-c5ccccc5-n5c6ccccc6c6ccccc65)nc(-c5cccc6c5c5ccccc5n6-c5ccccc5)n4)c3c2)cc1]

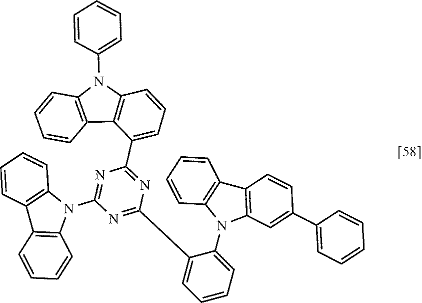 [SMILES: c1ccc(-c2ccc3c4ccccc4n(-c4ccccc4-c4nc(-c5cccc6c5c5ccccc5n6-c5ccccc5)nc(-n5c6ccccc6c6ccccc65)n4)c3c2)cc1]

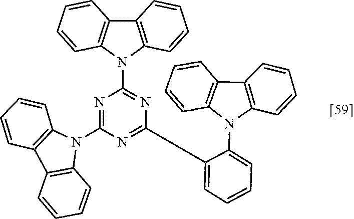 [SMILES: c1ccc(-n2c3ccccc3c3ccccc32)c(-c2nc(-n3c4ccccc4c4ccccc43)nc(-n3c4ccccc4c4ccccc43)n2)c1]

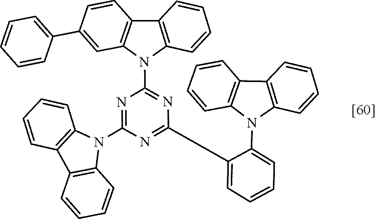 [SMILES: c1ccc(-c2ccc3c4ccccc4n(-c4nc(-c5ccccc5-n5c6ccccc6c6ccccc65)nc(-n5c6ccccc6c6ccccc65)n4)c3c2)cc1]

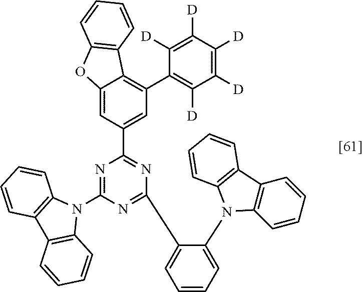 [SMILES: [2H]c1c([2H])c([2H])c(-c2cc(-c3nc(-c4ccccc4-n4c5ccccc5c5ccccc54)nc(-n4c5ccccc5c5ccccc54)n3)cc3oc4ccccc4c23)c([2H])c1[2H]]

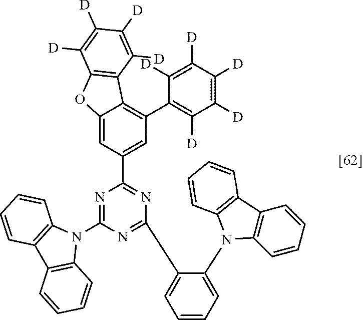 [SMILES: [2H]c1c([2H])c([2H])c(-c2cc(-c3nc(-c4ccccc4-n4c5ccccc5c5ccccc54)nc(-n4c5ccccc5c5ccccc54)n3)cc3oc4c([2H])c([2H])c([2H])c([2H])c4c23)c([2H])c1[2H]]

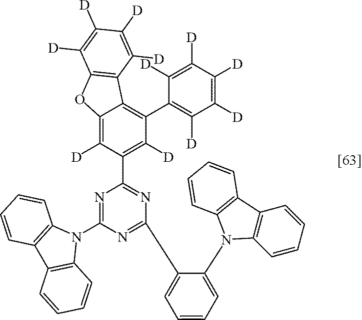 [SMILES: [2H]c1c([2H])c([2H])c(-c2c([2H])c(-c3nc(-c4ccccc4-n4c5ccccc5c5ccccc54)nc(-n4c5ccccc5c5ccccc54)n3)c([2H])c3oc4c([2H])c([2H])c([2H])c([2H])c4c23)c([2H])c1[2H]]

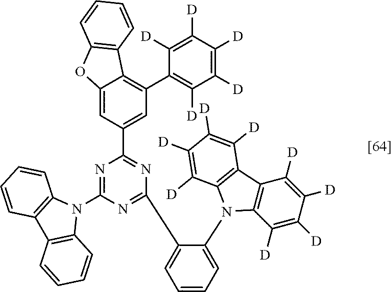 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5ccccc5c5ccccc54)n3)cc3oc4ccccc4c23)c([2H])c1[2H]]

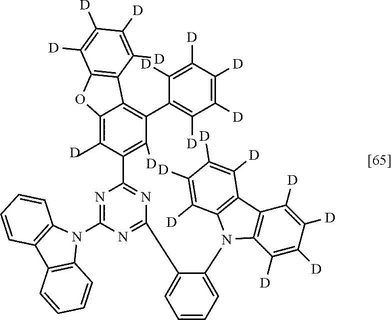

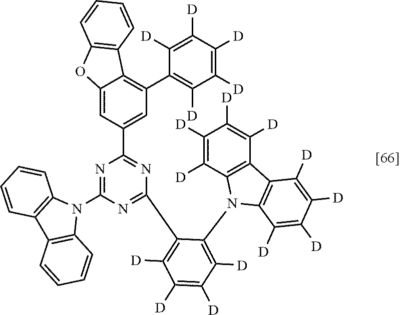 [SMILES: [2H]c1c([2H])c([2H])c(-c2cc(-c3nc(-c4c([2H])c([2H])c([2H])c([2H])c4-n4c5c([2H])c([2H])c([2H])c([2H])c5c5c([2H])c([2H])c([2H])c([2H])c54)nc(-n4c5ccccc5c5ccccc54)n3)cc3oc4ccccc4c23)c([2H])c1[2H]]

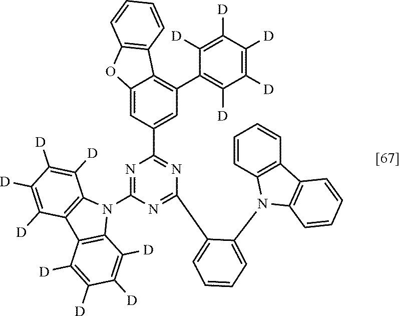 [SMILES: [2H]c1c([2H])c([2H])c(-c2cc(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)cc3oc4ccccc4c23)c([2H])c1[2H]]

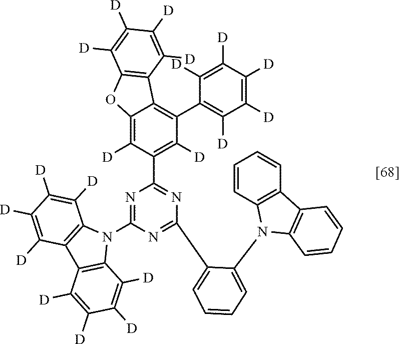 [SMILES: [2H]c1c([2H])c([2H])c(-c2c([2H])c(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]]

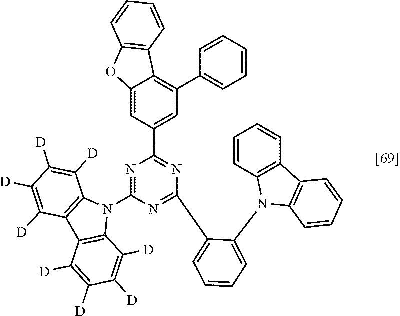 [SMILES: [2H]c1c([2H])c([2H])c2c(c1[2H])c1c([2H])c([2H])c([2H])c([2H])c1n2-c1nc(-c2cc(-c3ccccc3)c3c(c2)oc2ccccc23)nc(-c2ccccc2-n2c3ccccc3c3ccccc32)n1]

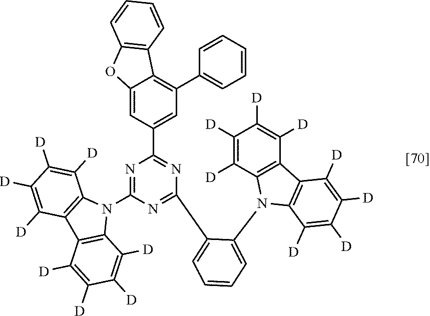 [SMILES: [2H]c1c([2H])c([2H])c2c(c1[2H])c1c([2H])c([2H])c([2H])c([2H])c1n2-c1nc(-c2cc(-c3ccccc3)c3c(c2)oc2ccccc23)nc(-c2ccccc2-n2c3c([2H])c([2H])c([2H])c([2H])c3c3c([2H])c([2H])c([2H])c([2H])c32)n1]

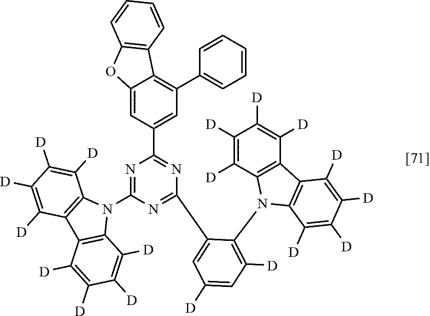 [SMILES: [2H]c1cc([2H])c(-n2c3c([2H])c([2H])c([2H])c([2H])c3c3c([2H])c([2H])c([2H])c([2H])c32)c(-c2nc(-c3cc(-c4ccccc4)c4c(c3)oc3ccccc34)nc(-n3c4c([2H])c([2H])c([2H])c([2H])c4c4c([2H])c([2H])c([2H])c([2H])c43)n2)c1]

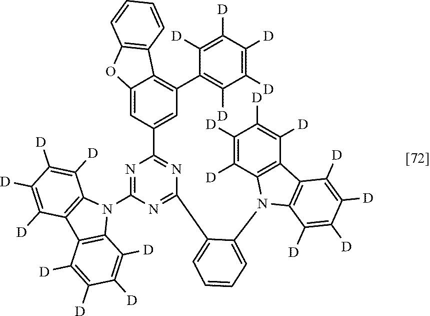 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)cc3oc4ccccc4c23)c([2H])c1[2H]]

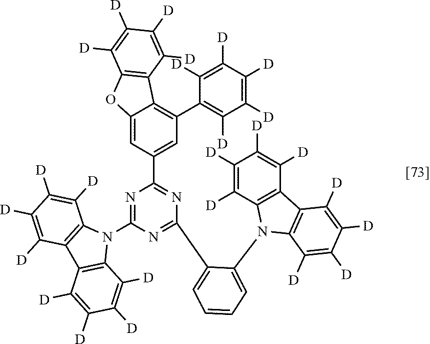 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)cc3oc4c([2H])c([2H])c([2H])c([2H])c4c23)c([2H])c1[2H]]

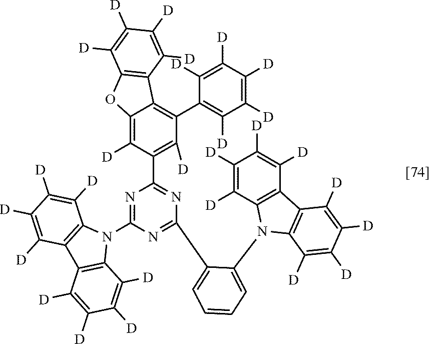 [SMILES: [2H]c1c([2H])c([2H])c(-c2c([2H])c(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]]

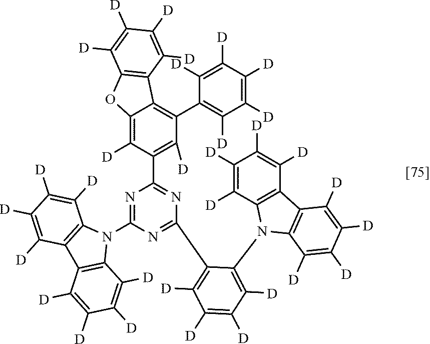 [SMILES: [2H]c1c([2H])c([2H])c(-c2c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]]

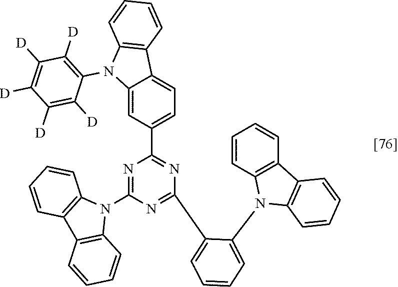 [SMILES: [2H]c1c([2H])c([2H])c(-n2c3ccccc3c3ccc(-c4nc(-c5ccccc5-n5c6ccccc6c6ccccc65)nc(-n5c6ccccc6c6ccccc65)n4)cc32)c([2H])c1[2H]]

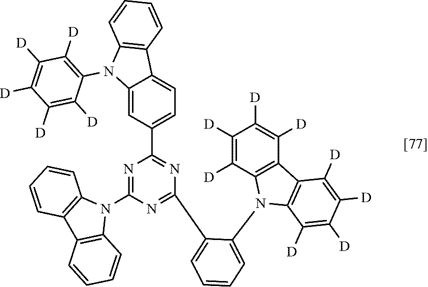 [SMILES: [2H]c1c([2H])c([2H])c(-n2c3ccccc3c3ccc(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)cc32)c([2H])c1[2H]]

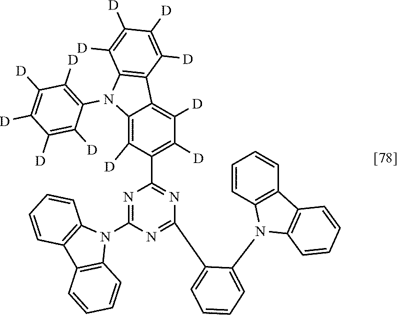 [SMILES: [2H]c1c([2H])c([2H])c(-n2c3c([2H])c([2H])c([2H])c([2H])c3c3c([2H])c([2H])c(-c4nc(-c5ccccc5-n5c6ccccc6c6ccccc65)nc(-n5c6ccccc6c6ccccc65)n4)c([2H])c32)c([2H])c1[2H]]

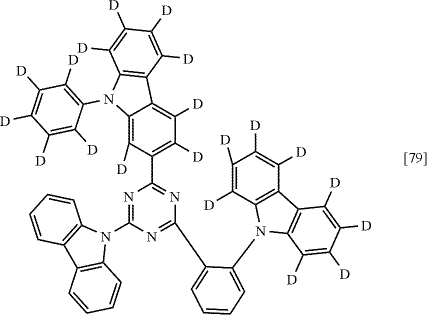 [SMILES: [2H]c1c([2H])c([2H])c(-n2c3c([2H])c([2H])c([2H])c([2H])c3c3c([2H])c([2H])c(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)c([2H])c32)c([2H])c1[2H]]

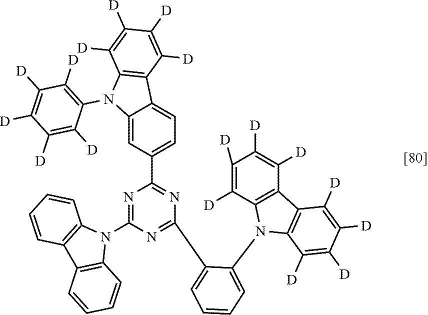 [SMILES: [2H]c1c([2H])c([2H])c(-n2c3cc(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)ccc3c3c([2H])c([2H])c([2H])c([2H])c32)c([2H])c1[2H]]

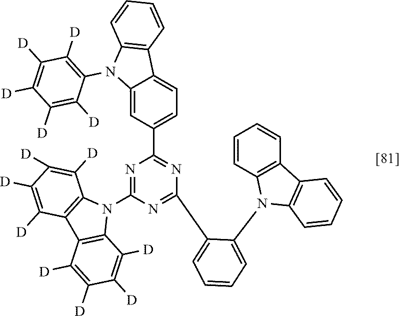 [SMILES: [2H]c1c([2H])c([2H])c(-n2c3ccccc3c3ccc(-c4nc(-c5ccccc5-n5c6ccccc6c6ccccc65)nc(-n5c6c([2H])c([2H])c([2H])c([2H])c6c6c([2H])c([2H])c([2H])c([2H])c65)n4)cc32)c([2H])c1[2H]]

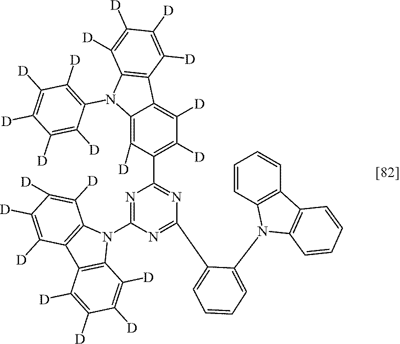 [SMILES: [2H]c1c([2H])c([2H])c(-n2c3c([2H])c([2H])c([2H])c([2H])c3c3c([2H])c([2H])c(-c4nc(-c5ccccc5-n5c6ccccc6c6ccccc65)nc(-n5c6c([2H])c([2H])c([2H])c([2H])c6c6c([2H])c([2H])c([2H])c([2H])c65)n4)c([2H])c32)c([2H])c1[2H]]

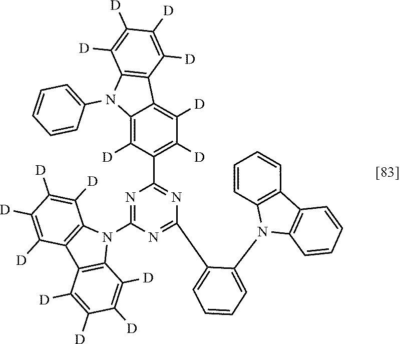 [SMILES: [2H]c1c([2H])c([2H])c2c(c1[2H])c1c([2H])c([2H])c(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)c([2H])c1n2-c1ccccc1]

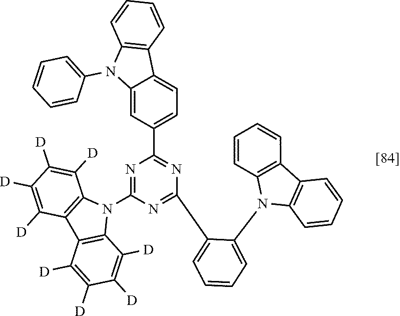 [SMILES: [2H]c1c([2H])c([2H])c2c(c1[2H])c1c([2H])c([2H])c([2H])c([2H])c1n2-c1nc(-c2ccc3c4ccccc4n(-c4ccccc4)c3c2)nc(-c2ccccc2-n2c3ccccc3c3ccccc32)n1]

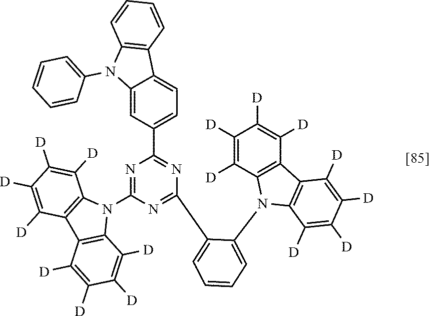 [SMILES: [2H]c1c([2H])c([2H])c2c(c1[2H])c1c([2H])c([2H])c([2H])c([2H])c1n2-c1nc(-c2ccc3c4ccccc4n(-c4ccccc4)c3c2)nc(-c2ccccc2-n2c3c([2H])c([2H])c([2H])c([2H])c3c3c([2H])c([2H])c([2H])c([2H])c32)n1]

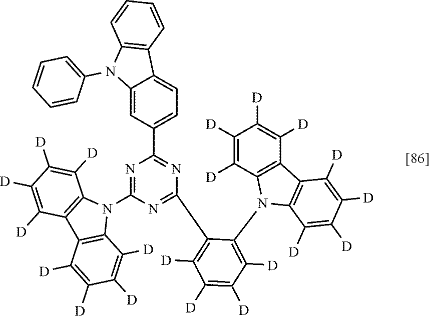 [SMILES: [2H]c1c([2H])c([2H])c(-n2c3c([2H])c([2H])c([2H])c([2H])c3c3c([2H])c([2H])c([2H])c([2H])c32)c(-c2nc(-c3ccc4c5ccccc5n(-c5ccccc5)c4c3)nc(-n3c4c([2H])c([2H])c([2H])c([2H])c4c4c([2H])c([2H])c([2H])c([2H])c43)n2)c1[2H]]

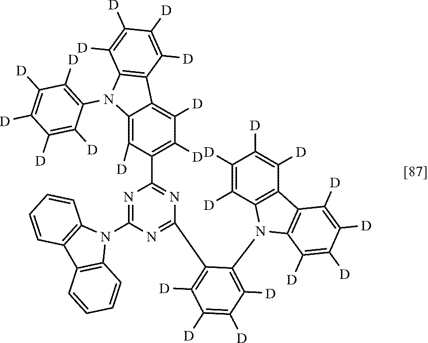 [SMILES: [2H]c1c([2H])c([2H])c(-n2c3c([2H])c([2H])c([2H])c([2H])c3c3c([2H])c([2H])c(-c4nc(-c5c([2H])c([2H])c([2H])c([2H])c5-n5c6c([2H])c([2H])c([2H])c([2H])c6c6c([2H])c([2H])c([2H])c([2H])c65)nc(-n5c6ccccc6c6ccccc65)n4)c([2H])c32)c([2H])c1[2H]]

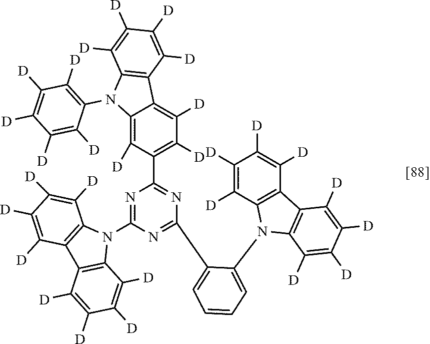 [SMILES: [2H]c1c([2H])c([2H])c(-n2c3c([2H])c([2H])c([2H])c([2H])c3c3c([2H])c([2H])c(-c4nc(-c5ccccc5-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c([2H])c32)c([2H])c1[2H]]

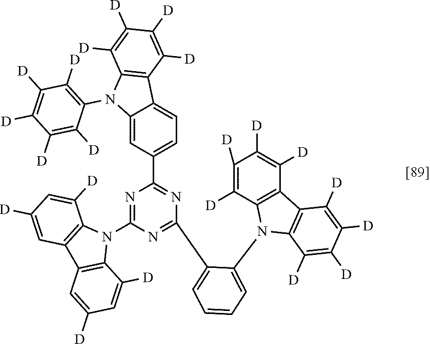 [SMILES: [2H]c1cc([2H])c2c(c1)c1cc([2H])cc([2H])c1n2-c1nc(-c2ccc3c4c([2H])c([2H])c([2H])c([2H])c4n(-c4c([2H])c([2H])c([2H])c([2H])c4[2H])c3c2)nc(-c2ccccc2-n2c3c([2H])c([2H])c([2H])c([2H])c3c3c([2H])c([2H])c([2H])c([2H])c32)n1]

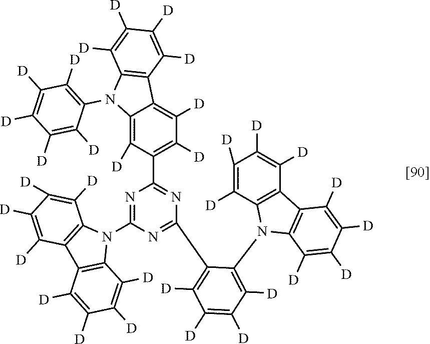 [SMILES: [2H]c1c([2H])c([2H])c(-n2c3c([2H])c([2H])c([2H])c([2H])c3c3c([2H])c([2H])c(-c4nc(-c5c([2H])c([2H])c([2H])c([2H])c5-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c([2H])c32)c([2H])c1[2H]]

A composition for an organic optoelectronic device according to another embodiment may include, e.g., a first compound and a second compound. In an implementation, the first compound may be, e.g., the aforementioned compound for the organic optoelectronic device and the second compound may be, e.g., a compound represented by Chemical Formula 2; or a compound represented by a combination of Chemical Formula 3 and Chemical Formula 4.

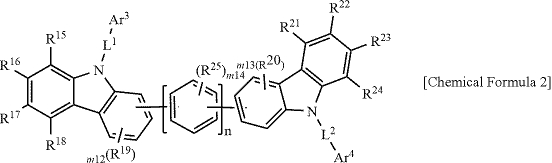

In Chemical Formula 2, Ar³ and Ar⁴ may each independently be or include, e.g., a substituted or unsubstituted C6 to C20 aryl group or a substituted or unsubstituted C2 to C30 heterocyclic group.

L¹ and L² may each independently be or include, e.g., a single bond or a substituted or unsubstituted C6 to C20 arylene group.

R¹⁵ to R²⁵ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

m12 and m13 may each independently be, e.g., an integer of 1 to 3.

m14 may be, e.g., an integer of 1 to 4.

n may be, e.g., an integer of 0 to 2. [Chemical Formula 3]

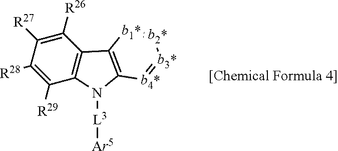

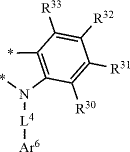

In Chemical Formulae 3 and 4, Ar⁵ and Ar⁶ may each independently be or include, e.g., a substituted or unsubstituted C6 to C20 aryl group or a substituted or unsubstituted C2 to C30 heterocyclic group.

Two adjacent ones of b₁* to b₄* of Chemical Formula 3 are linking carbons linked at * of Chemical Formula 4. The remaining two of b₁* to b₄* of Chemical Formula 3, not linked to * of Chemical Formula 4, may each independently be, e.g., C—L^{a}—R^{a}. As used herein, the term “linking carbon” refers to a shared carbon at which fused rings are linked.

L^{a}, L³, and L⁴ may each independently be or include, e.g., a single bond or a substituted or unsubstituted C6 to C20 arylene group.

R^{a} and R²⁶ to R³³ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

The second compound may be used in the light emitting layer together with the first compound to help improve luminous efficiency and life-span characteristics by increasing charge mobility and increasing stability.

In an implementation, Ar³ and Ar⁴ in Chemical Formula 2 may each independently be, e.g., a substituted or unsubstituted C6 to C20 aryl group or a substituted or unsubstituted C2 to C30 heterocyclic group.

In an implementation, at least one of Ar³ and Ar⁴ may be, e.g., a C6 to C20 aryl group substituted with deuterium or a C2 to C30 heterocyclic group substituted with deuterium.

In an implementation, R¹⁵ to R²⁵ in Chemical Formula 2 may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

In an implementation, at least one of R¹⁵ to R²⁵ may be, e.g., deuterium, a C1 to C30 alkyl group substituted with deuterium, a C6 to C30 aryl group substituted with deuterium, or a C2 to C30 heterocyclic group substituted with deuterium.

In an implementation, at least one of Ar³ and Ar⁴ in Chemical Formula 2 may be, e.g., a C6 to C20 aryl group substituted with deuterium, or a C2 to C30 heterocyclic group substituted with deuterium.

In an implementation, at least one of R¹⁵ to R²⁵ in Chemical Formula 2 may be, e.g., deuterium, a C1 to C30 alkyl group substituted with deuterium, a C6 to C30 aryl group substituted with deuterium, or a C2 to C30 heterocyclic group substituted with deuterium.

In an implementation, Ar⁵ and Ar⁶ in Chemical Formulas 3 and 4 may each independently be, e.g., a substituted or unsubstituted C6 to C20 aryl group or a substituted or unsubstituted C2 to C30 heterocyclic group.

In an implementation, at least one of Ar⁵ and Ar⁶ may be, e.g., a C6 to C20 aryl group substituted with deuterium or a C2 to C30 heterocyclic group substituted with deuterium.

In an implementation, in Chemical Formulae 3 and 4, R^{a} and R²⁶ to R³³ may each independently be, e.g., hydrogen, deuterium, a cyano group, a halogen group, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

In an implementation, at least one of R^{a} and R²⁶ to R³³ may be, e.g., deuterium, a C1 to C30 alkyl group substituted with deuterium, a C6 to C30 aryl group substituted with deuterium, or a C2 to C30 heterocyclic group substituted with deuterium.

In an implementation, at least one of Ar⁵ and Ar⁶ in Chemical Formulas 3 and 4 may be, e.g., a C6 to C20 aryl group substituted with deuterium or a C2 to C30 heterocyclic group substituted with deuterium.

In an implementation, at least one of R^{a} and R²⁶ to R³³ may be, e.g., deuterium, a C1 to C30 alkyl group substituted with deuterium, a C6 to C30 aryl group substituted with deuterium, or a C2 to C30 heterocyclic group substituted with deuterium.

In an implementation example, Ar³ and Ar⁴ of Chemical Formula 2 may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted substituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, L¹ and L² of Chemical Formula 2 may each independently be, e.g., a single bond, a substituted or unsubstituted phenylene group, or a substituted or unsubstituted biphenylene group.

In an implementation, R¹⁵ to R²⁵ of Chemical Formula 2 may each independently be, e.g., hydrogen, deuterium, or a substituted or unsubstituted C6 to C12 aryl group.

n may be, e.g., 0 or 1.

In an implementation, Ar³ and Ar⁴ of Chemical Formula 2 may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, at least one of Ar³ and Ar⁴ may be, e.g., a phenyl group substituted with deuterium, a biphenyl group substituted with deuterium, a terphenyl group substituted with deuterium, a naphthyl group substituted with deuterium, an anthracenyl group substituted with deuterium, a triphenylenyl group substituted with deuterium, a carbazolyl group substituted with deuterium, a dibenzothiophenyl group substituted with deuterium, a dibenzofuranyl group substituted with deuterium, or a fluorenyl group substituted with deuterium.

In an implementation, R¹⁵ to R²⁵ in Chemical Formula 2 may each independently be, e.g., hydrogen, deuterium, or a substituted or unsubstituted C6 to C12 aryl group.

In an implementation at least one of R¹⁵ to R²⁵ may be, e.g., deuterium or a C6 to C12 aryl group substituted with deuterium.

In an implementation, at least one of Ar³ and Ar⁴ in Chemical Formula 2 may be, e.g., a phenyl group substituted with deuterium, a biphenyl group substituted with deuterium, a terphenyl group substituted with deuterium, a naphthyl group substituted with deuterium, an anthracenyl group substituted with deuterium, a triphenylenyl group substituted with deuterium, a carbazolyl group substituted with deuterium, a dibenzothiophenyl group substituted with deuterium, a dibenzofuranyl group substituted with deuterium, or a fluorenyl group substituted with deuterium.

In an implementation, at least one of R¹⁵ to R²⁵ in Chemical Formula 2 may be, e.g., deuterium or a C6 to C12 aryl group substituted with deuterium.

For example, “substituted” in Chemical Formula 2 means that at least one hydrogen is replaced by deuterium, a C1 to C4 alkyl group, a C6 to C18 aryl group, or a C2 to C30 heteroaryl group.

In an implementation, Chemical Formula 2 may be represented by, e.g., one of Chemical Formula 2-1 to Chemical Formula 2-15.

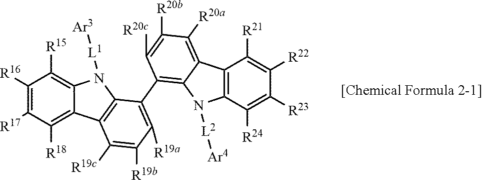

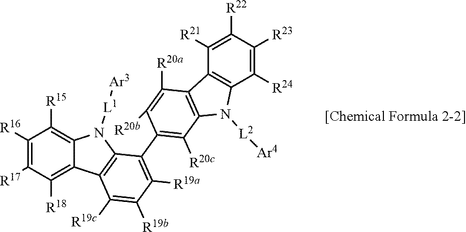

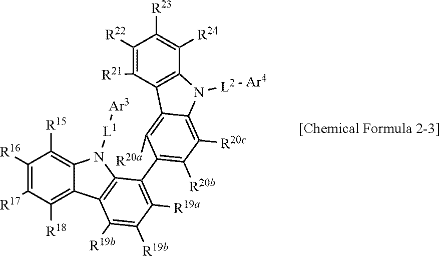

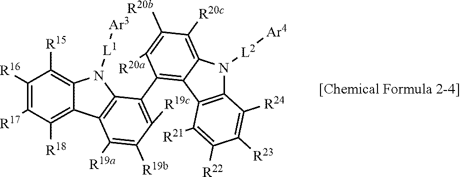

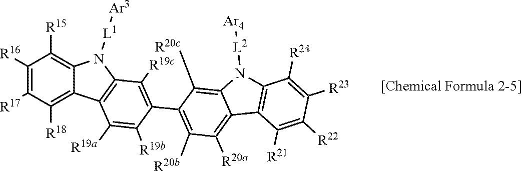

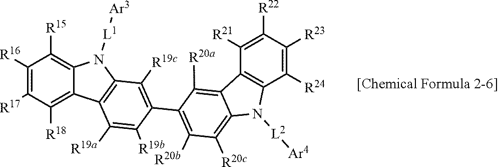

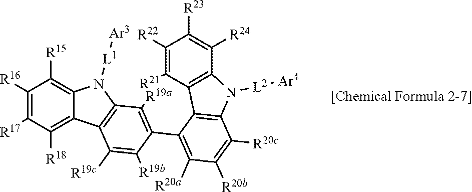

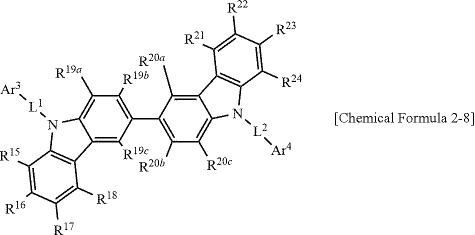

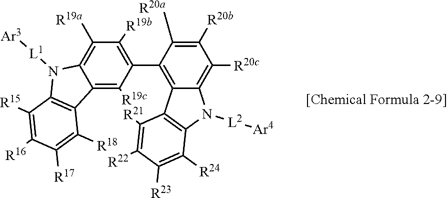

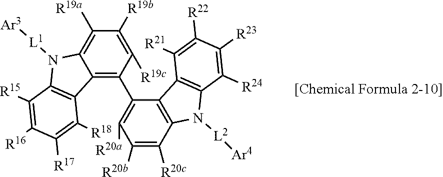

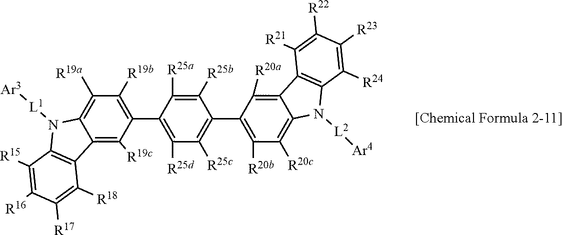

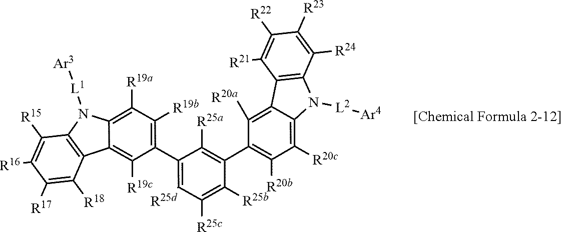

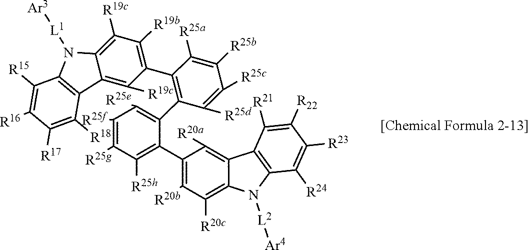

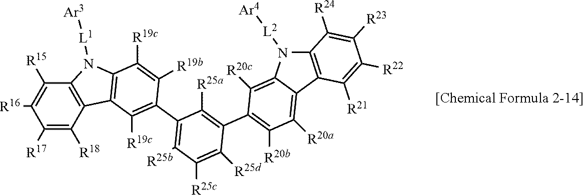

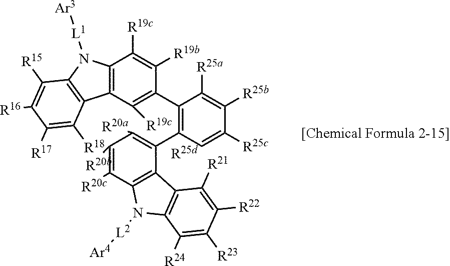

In Chemical Formula 2-1 to Chemical Formula 2-15, R¹⁵ to R¹⁸, R^{19a}, R^{19b}, R^{19c}, R^{20a}, R^{20b}, R^{20c}, R²¹ to R²⁴, R^{25a}, R^{25b}, R^{25c}, R^{25d}, R^{25e}, R^{25f}, R^{25g}, and R^{25h} may each independently be, e.g., hydrogen, deuterium, or a substituted or unsubstituted C6 to C12 aryl group, and moieties *—L¹—Ar³ and *—L²—Ar⁴ may each independently be, e.g., a moiety of Group II.

### Group II

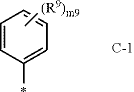

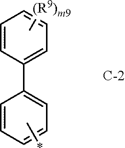

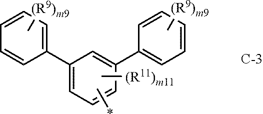

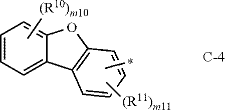

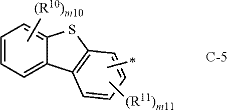

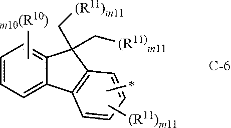

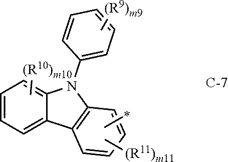

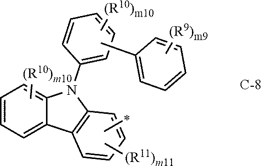

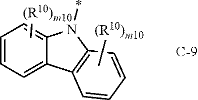

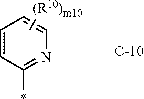

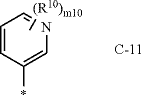

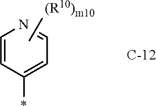

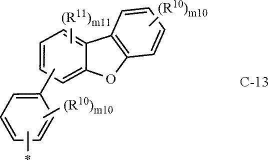

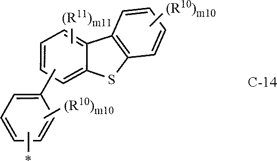

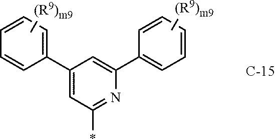

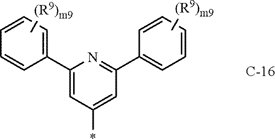

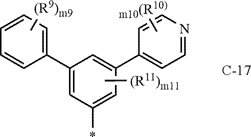

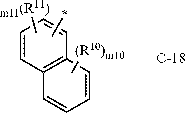

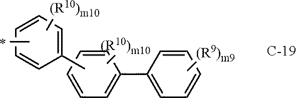

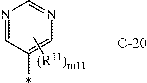

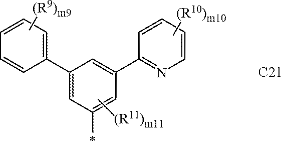

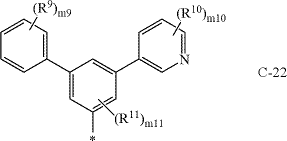

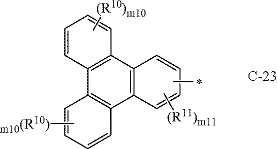

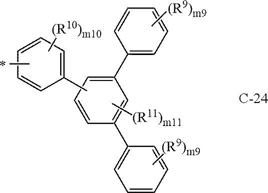

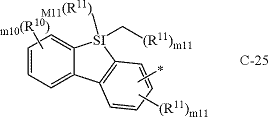

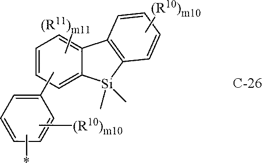

In Group II, R⁹ to R¹¹ may each independently be, e.g., hydrogen, deuterium, a substituted or unsubstituted C1 to C4 alkyl group, a substituted or unsubstituted C6 to C18 aryl group, or a substituted or unsubstituted C2 to C30 heteroaryl group.

m9 may be, e.g., an integer of 1 to 5.

m10 may be, e.g., an integer of 1 to 4.

m11 may be, e.g., an integer of 1 to 3.

* is a linking point.

In an implementation, Chemical Formula 2 may be represented by, e.g., Chemical Formula 2-8.

In an implementation, moieties *—L¹—Ar³ and *—L²—Ar⁴ of Chemical Formula 2-8 may each independently be, e.g., one of C-1, C-2, C-3, C-4, C-7, C-8, and C-9.

In an implementation, the second compound represented by the combination of Chemical Formulae 3 and 4 may be represented by, e.g., Chemical Formula 3A, Chemical Formula 3B, Chemical Formula 3C, Chemical Formula 3D, or Chemical Formula 3E.

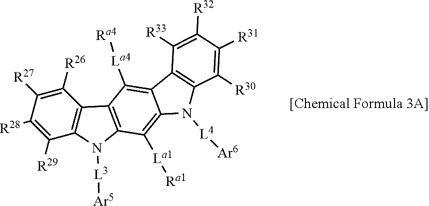

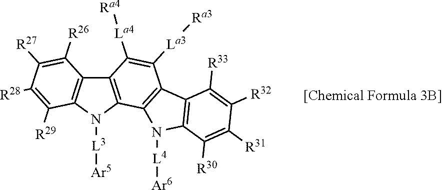

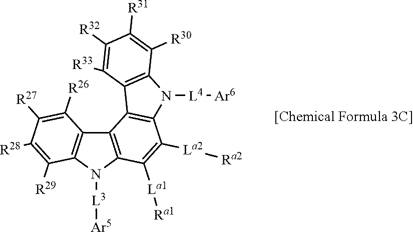

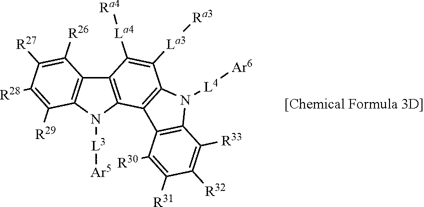

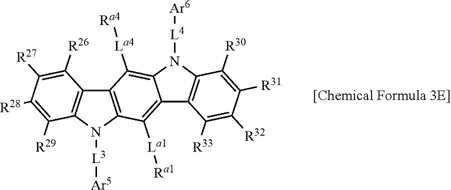

In Chemical Formula 3A to Chemical Formula 3E, Ar⁵, Ar⁶, L³, L⁴, and R²⁶ to R³³ may be defined the same as those described above.

L^{a1} to L^{a4} may be defined the same as L³ and L⁴.

R^{a1} to R^{a4} may be defined the same as R²⁶ to R³³.

In an implementation, Ar⁵ and Ar⁶ in Chemical Formulae 3 and 4 may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4} and R²⁶ to R³³ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, moieties *—L³—Ar⁵ and *—L⁴—Ar⁶ of Chemical Formulae 3 and 4 may each independently be, e.g., a moiety of Group II.

In an implementation, R^{a1} to R^{a4} and R²⁶ to R³³ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4} and R²⁶ to R³³ may each independently be, e.g., hydrogen, deuterium, a cyano group, or a substituted or unsubstituted phenyl group.

In an implementation, R^{a1} to R^{a4}, and R²⁶ to R³³ may each independently be, e.g., hydrogen, deuterium, or a phenyl group.

In an implementation, Ar⁵ and Ar⁶ in Chemical Formulae 3 and 4 may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, at least one of Ar⁵ and Ar⁶ may be, e.g., a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an implementation, in Chemical Formulae 3 and 4, R^{a1} to R^{a4} and R²⁶ to R³³ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, at least one of R^{a1} to R^{a4} and R²⁶ to R³³ may be, e.g., deuterium, a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an implementation, at least one of Ar⁵ and Ar⁶ in Chemical Formulae 3 and 4 may be, e.g., a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an implementation, at least one of R^{a1} to R^{a4} and R²⁶ to R³³ may be, e.g., deuterium, a phenyl group substituted with deuterium, a biphenyl group substituted with deuterium, a pyridinyl group substituted with deuterium, a carbazolyl group substituted with deuterium, a dibenzofuranyl group substituted with deuterium, or a dibenzothiophenyl group substituted with deuterium.

In an implementation, the second compound may be represented by, e.g., Chemical Formula 2-8. In an implementation, in Chemical Formula 2-8, Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L¹ and L² may each independently be, e.g., a single bond, or a substituted or unsubstituted C6 to C20 arylene group, and R¹⁵ to R¹⁸, R^{19a}, R^{19b}, R^{19c}, R^{20a}, R^{20b}, R^{20c}, and R²¹ to R²⁴ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, moieties ^{∗}—L¹—Ar³ and ^{∗}—L²—Ar⁴ of Chemical Formula 2-8 may each independently be, e.g., a moiety of Group II.

In an implementation, the second compound may be represented by, e.g., Chemical Formula 3C. In an implementation, in Chemical Formula 3C, L^{a1} and L^{a2} may be, e.g., a single bond, and L³ and L⁴ may each independently be, e.g., a single bond or a substituted or unsubstituted C6 to C12 arylene group, R²⁶ to R³³, R^{a1} and R^{a2} may each independently be, e.g., hydrogen, deuterium, or a phenyl group, and Ar⁵ and Ar⁶ may each independently be, e.g., a substituted or unsubstituted phenyl group, a substituted or unsubstituted biphenyl group, or a substituted or unsubstituted terphenyl group.

In an implementation, moieties *—L³—Ar⁵ and *—L⁴—Ar⁶ of Chemical Formula 3C may each independently be, e.g., a moiety of Group II.

In an implementation, the second compound may be, e.g., a compound of Group 2.

### Group 2

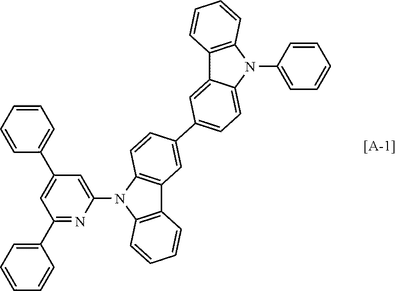 [SMILES: c1ccc(-c2cc(-c3ccccc3)nc(-n3c4ccccc4c4cc(-c5ccc6c(c5)c5ccccc5n6-c5ccccc5)ccc43)c2)cc1]

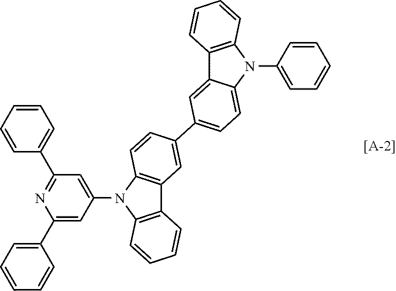 [SMILES: c1ccc(-c2cc(-n3c4ccccc4c4cc(-c5ccc6c(c5)c5ccccc5n6-c5ccccc5)ccc43)cc(-c3ccccc3)n2)cc1]

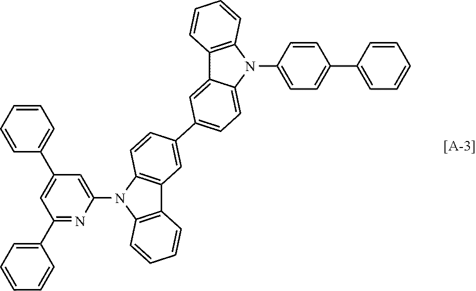 [SMILES: c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

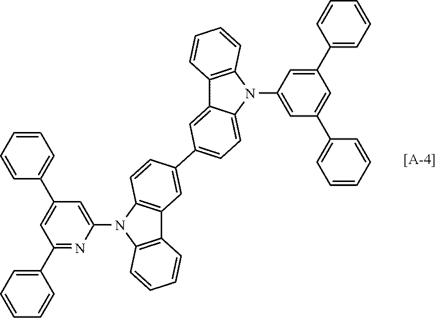 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

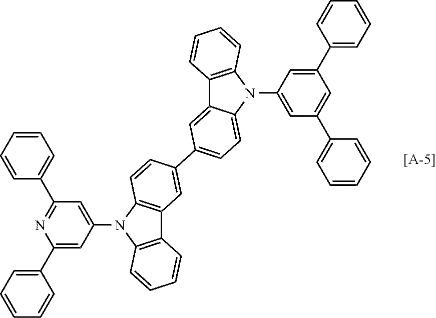 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]

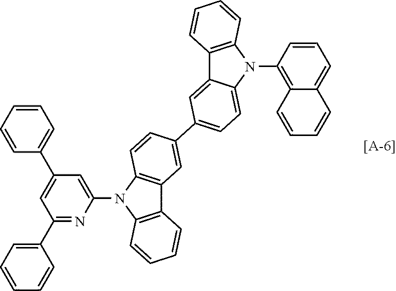 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1]

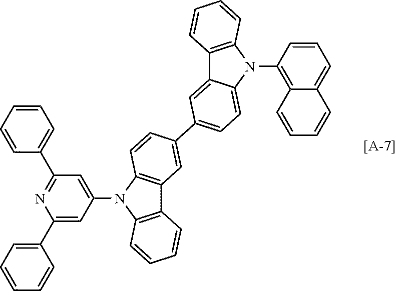 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1]

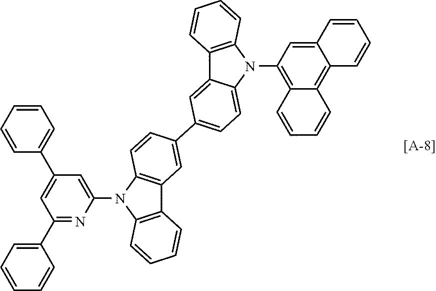 [SMILES: c1ccc(-c2cc(-c3ccccc3)nc(-n3c4ccccc4c4cc(-c5ccc6c(c5)c5ccccc5n6-c5cc6ccccc6c6ccccc56)ccc43)c2)cc1]

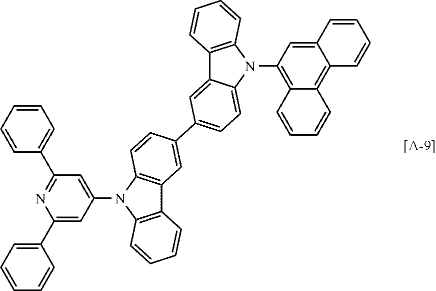 [SMILES: c1ccc(-c2cc(-n3c4ccccc4c4cc(-c5ccc6c(c5)c5ccccc5n6-c5cc6ccccc6c6ccccc56)ccc43)cc(-c3ccccc3)n2)cc1]

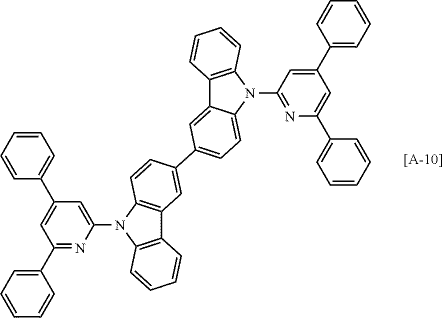 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1]

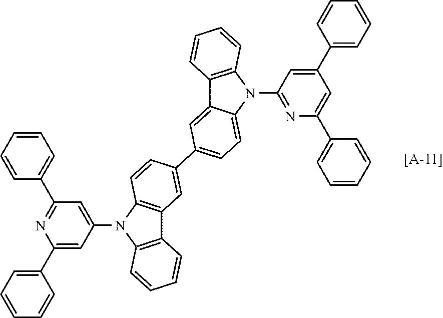 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1]

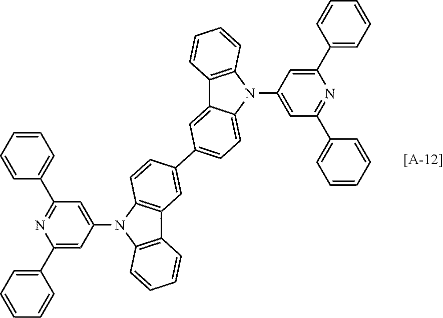 [SMILES: c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

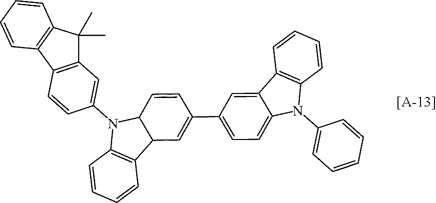 [SMILES: CC1(C)c2ccccc2-c2ccc(N3c4ccccc4C4C=C(c5ccc6c(c5)c5ccccc5n6-c5ccccc5)C=CC43)cc21]

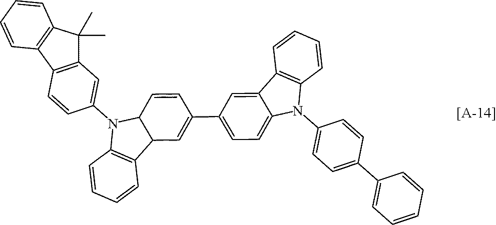 [SMILES: CC1(C)c2ccccc2-c2ccc(N3c4ccccc4C4C=C(c5ccc6c(c5)c5ccccc5n6-c5ccc(-c6ccccc6)cc5)C=CC43)cc21]

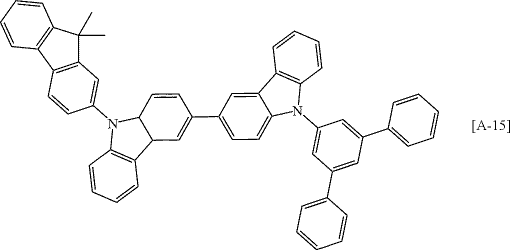 [SMILES: CC1(C)c2ccccc2-c2ccc(N3c4ccccc4C4C=C(c5ccc6c(c5)c5ccccc5n6-c5cc(-c6ccccc6)cc(-c6ccccc6)c5)C=CC43)cc21]

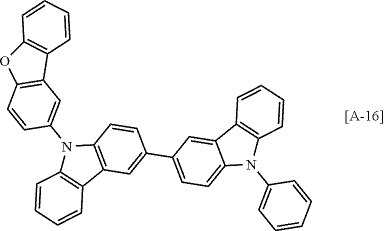 [SMILES: c1ccc(-n2c3ccccc3c3cc(-c4ccc5c(c4)c4ccccc4n5-c4ccc5oc6ccccc6c5c4)ccc32)cc1]

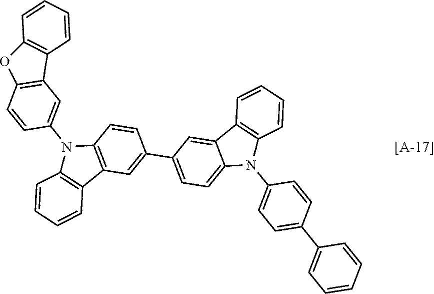 [SMILES: c1ccc(-c2ccc(-n3c4ccccc4c4cc(-c5ccc6c(c5)c5ccccc5n6-c5ccc6oc7ccccc7c6c5)ccc43)cc2)cc1]

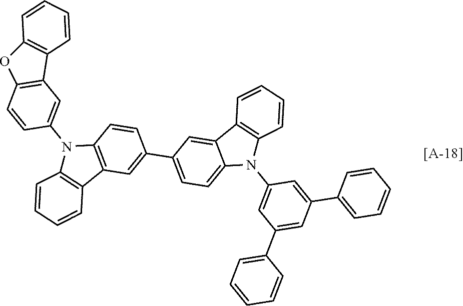 [SMILES: c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

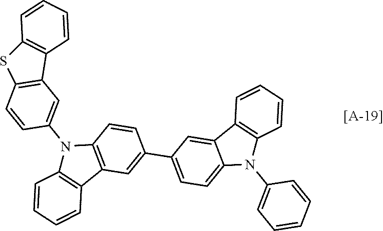 [SMILES: c1ccc(-n2c3ccccc3c3cc(-c4ccc5c(c4)c4ccccc4n5-c4ccc5sc6ccccc6c5c4)ccc32)cc1]

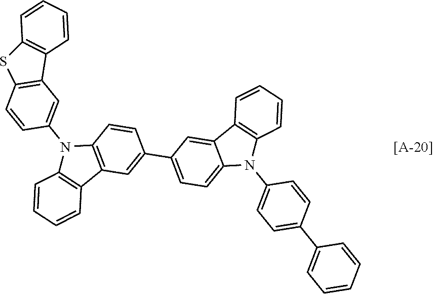 [SMILES: c1ccc(-c2ccc(-n3c4ccccc4c4cc(-c5ccc6c(c5)c5ccccc5n6-c5ccc6sc7ccccc7c6c5)ccc43)cc2)cc1]

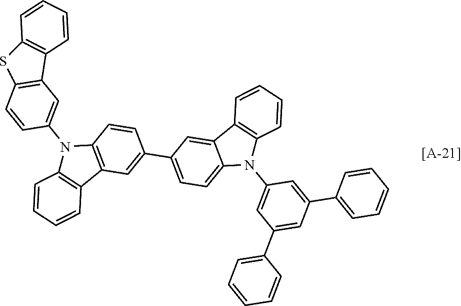 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

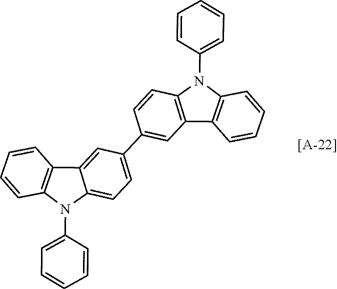 [SMILES: c1ccc(-n2c3ccccc3c3cc(-c4ccc5c(c4)c4ccccc4n5-c4ccccc4)ccc32)cc1]

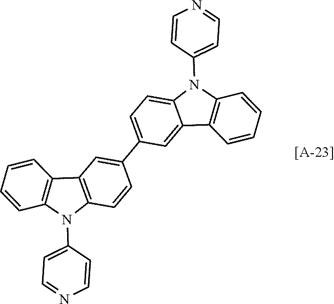 [SMILES: c1ccc2c(c1)c1cc(-c3ccc4c(c3)c3ccccc3n4-c3ccncc3)ccc1n2-c1ccncc1]

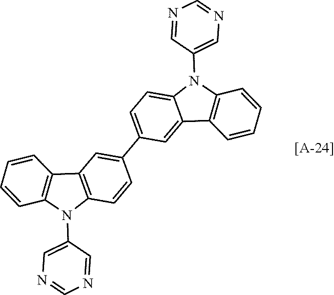 [SMILES: c1ccc2c(c1)c1cc(-c3ccc4c(c3)c3ccccc3n4-c3cncnc3)ccc1n2-c1cncnc1]

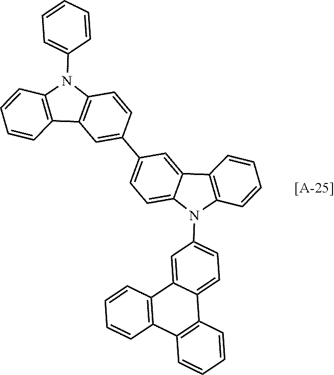 [SMILES: c1ccc(-n2c3ccccc3c3cc(-c4ccc5c(c4)c4ccccc4n5-c4ccc5c6ccccc6c6ccccc6c5c4)ccc32)cc1]

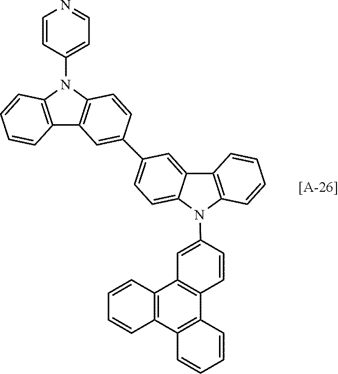 [SMILES: c1ccc2c(c1)c1ccccc1c1cc(-n3c4ccccc4c4cc(-c5ccc6c(c5)c5ccccc5n6-c5ccncc5)ccc43)ccc21]

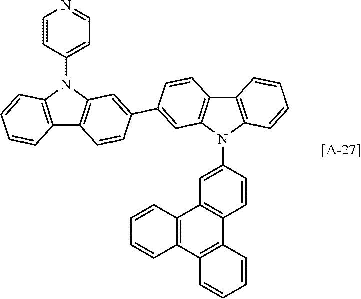 [SMILES: c1ccc2c(c1)c1ccccc1c1cc(-n3c4ccccc4c4ccc(-c5ccc6c7ccccc7n(-c7ccncc7)c6c5)cc43)ccc21]

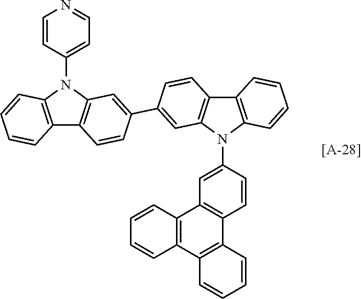 [SMILES: c1ccc2c(c1)c1ccccc1c1cc(-n3c4ccccc4c4ccc(-c5ccc6c7ccccc7n(-c7ccncc7)c6c5)cc43)ccc21]

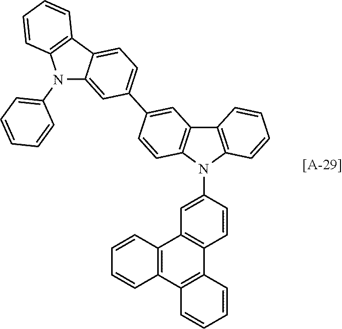 [SMILES: c1ccc(-n2c3ccccc3c3ccc(-c4ccc5c(c4)c4ccccc4n5-c4ccc5c6ccccc6c6ccccc6c5c4)cc32)cc1]

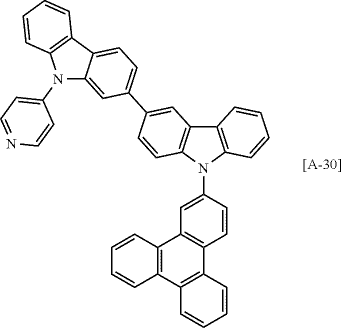 [SMILES: c1ccc2c(c1)c1ccccc1c1cc(-n3c4ccccc4c4cc(-c5ccc6c7ccccc7n(-c7ccncc7)c6c5)ccc43)ccc21]

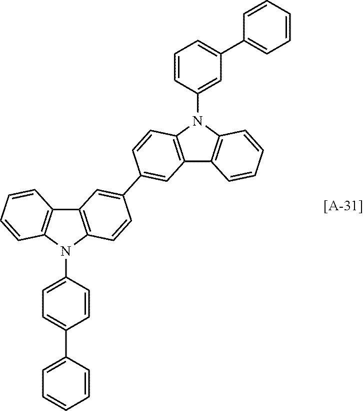 [SMILES: c1ccc(-c2ccc(-n3c4ccccc4c4cc(-c5ccc6c(c5)c5ccccc5n6-c5cccc(-c6ccccc6)c5)ccc43)cc2)cc1]

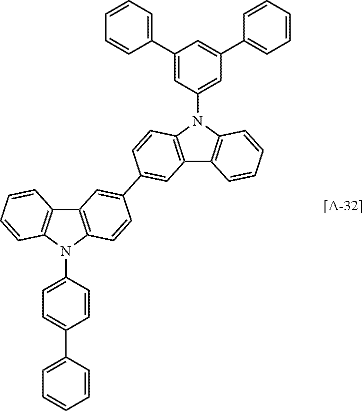 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

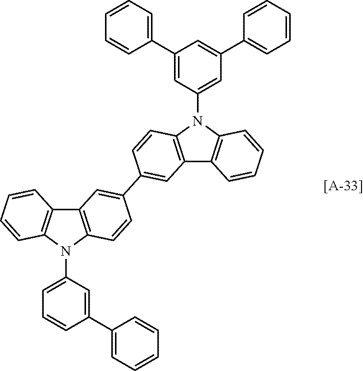 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

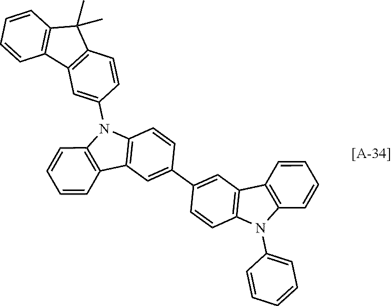 [SMILES: CC1(C)c2ccccc2-c2cc(-n3c4ccccc4c4cc(-c5ccc6c(c5)c5ccccc5n6-c5ccccc5)ccc43)ccc21]

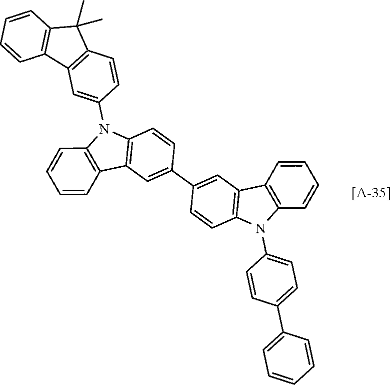 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21]

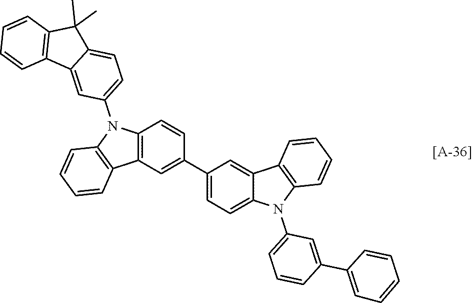 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21]

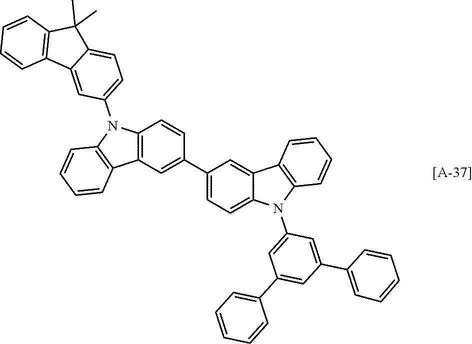 [SMILES: CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21]

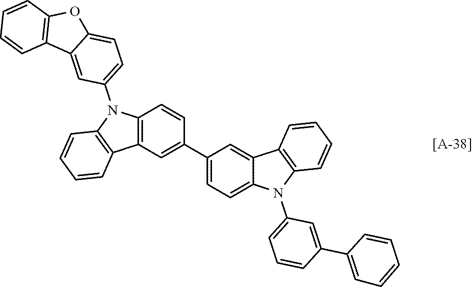 [SMILES: c1ccc(-c2cccc(-n3c4ccccc4c4cc(-c5ccc6c(c5)c5ccccc5n6-c5ccc6oc7ccccc7c6c5)ccc43)c2)cc1]

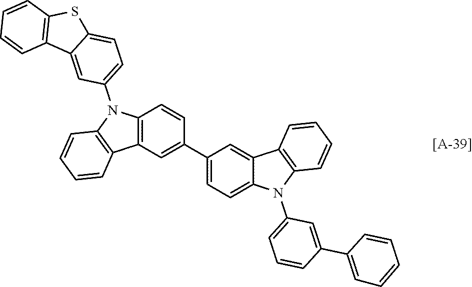 [SMILES: c1ccc(-c2cccc(-n3c4ccccc4c4cc(-c5ccc6c(c5)c5ccccc5n6-c5ccc6sc7ccccc7c6c5)ccc43)c2)cc1]

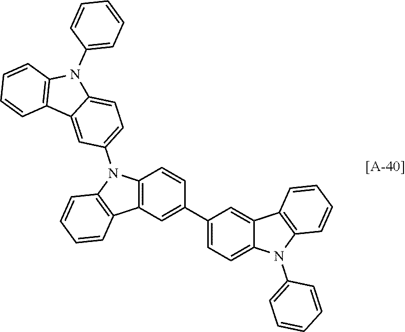 [SMILES: c1ccc(-n2c3ccccc3c3cc(-c4ccc5c(c4)c4ccccc4n5-c4ccc5c(c4)c4ccccc4n5-c4ccccc4)ccc32)cc1]

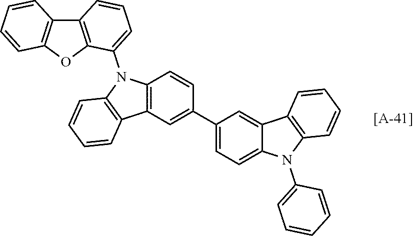 [SMILES: c1ccc(-n2c3ccccc3c3cc(-c4ccc5c(c4)c4ccccc4n5-c4cccc5c4oc4ccccc45)ccc32)cc1]

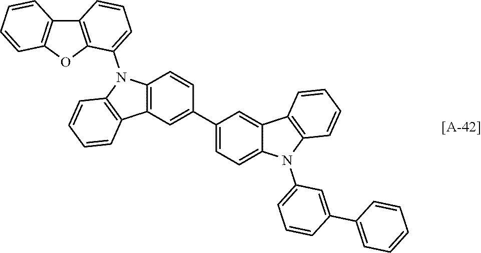 [SMILES: c1ccc(-c2cccc(-n3c4ccccc4c4cc(-c5ccc6c(c5)c5ccccc5n6-c5cccc6c5oc5ccccc56)ccc43)c2)cc1]

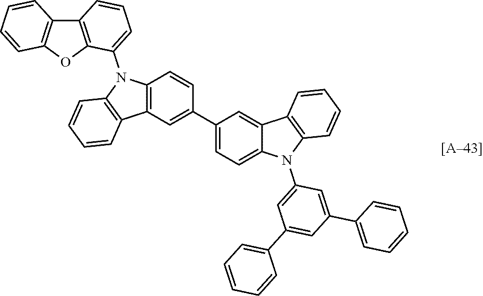 [SMILES: c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

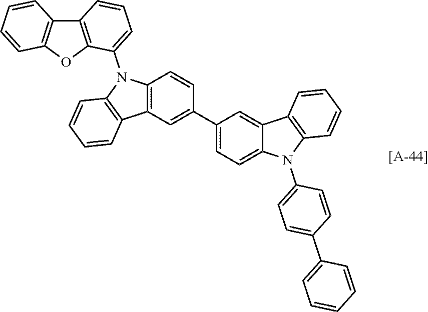 [SMILES: c1ccc(-c2ccc(-n3c4ccccc4c4cc(-c5ccc6c(c5)c5ccccc5n6-c5cccc6c5oc5ccccc56)ccc43)cc2)cc1]

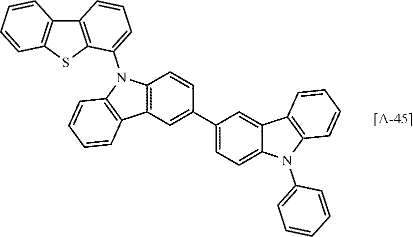 [SMILES: c1ccc(-n2c3ccccc3c3cc(-c4ccc5c(c4)c4ccccc4n5-c4cccc5c4sc4ccccc45)ccc32)cc1]

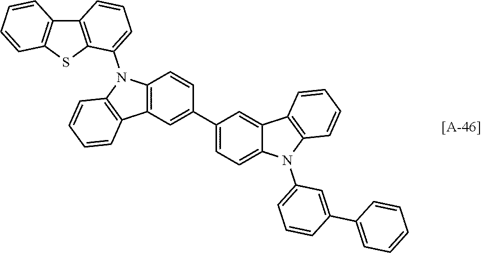 [SMILES: c1ccc(-c2cccc(-n3c4ccccc4c4cc(-c5ccc6c(c5)c5ccccc5n6-c5cccc6c5sc5ccccc56)ccc43)c2)cc1]

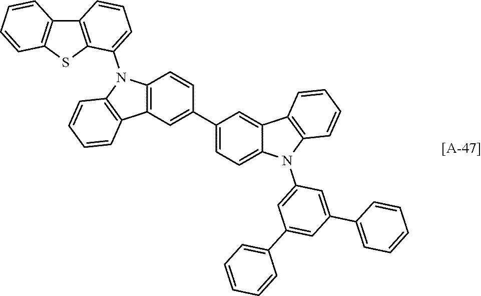 [SMILES: c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

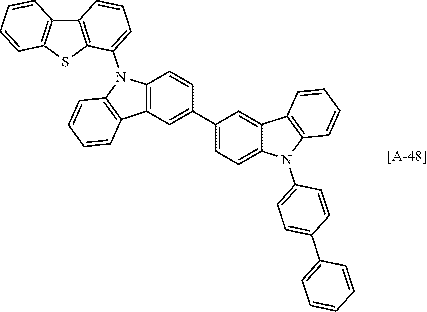 [SMILES: c1ccc(-c2ccc(-n3c4ccccc4c4cc(-c5ccc6c(c5)c5ccccc5n6-c5cccc6c5sc5ccccc56)ccc43)cc2)cc1]

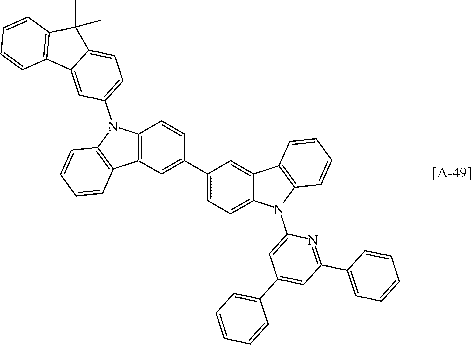 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21]

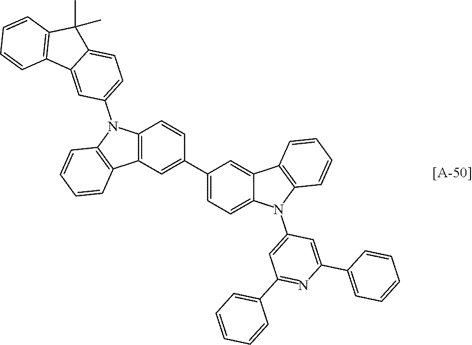 [SMILES: CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

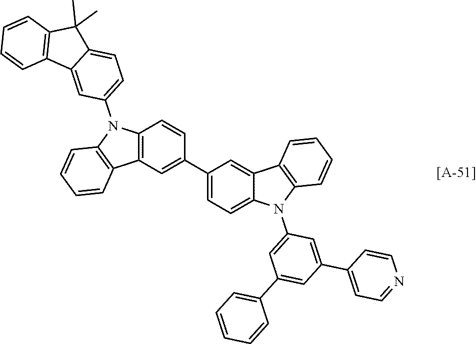 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

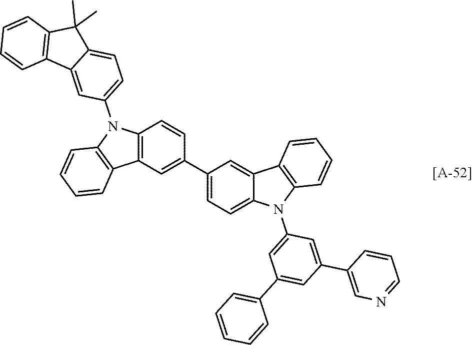 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21]

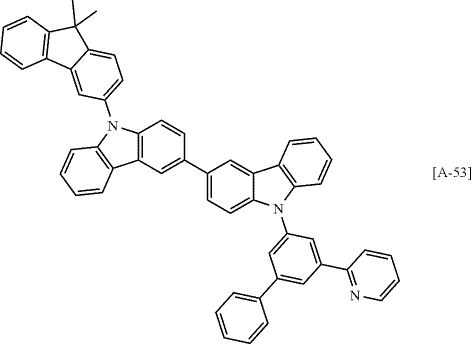 [SMILES: CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

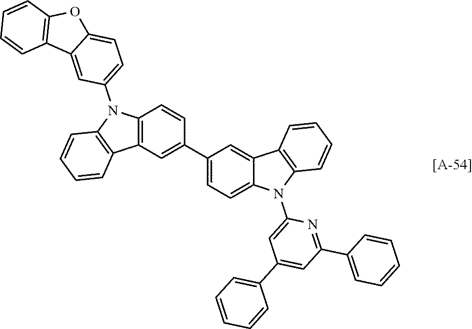 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

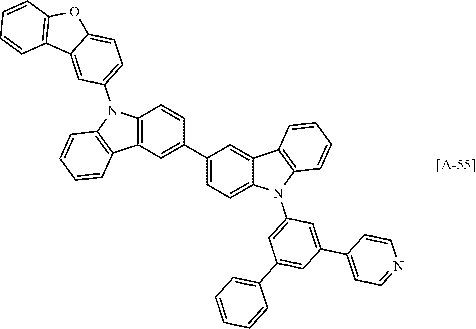 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1]

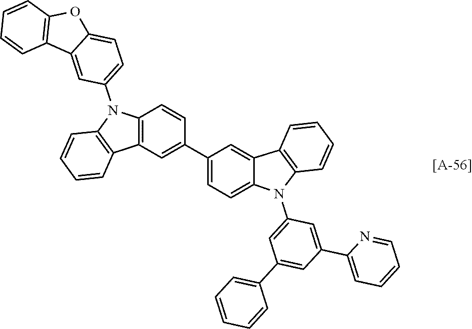 [SMILES: c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

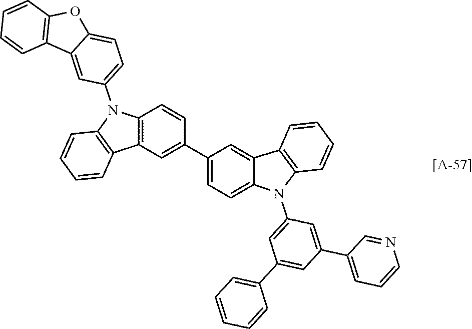 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

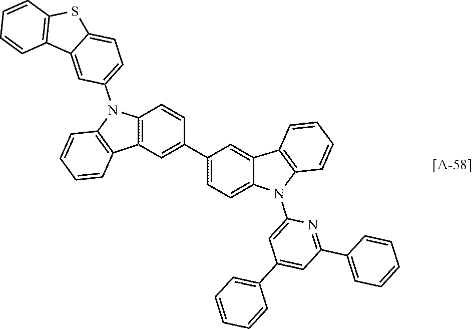 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1]

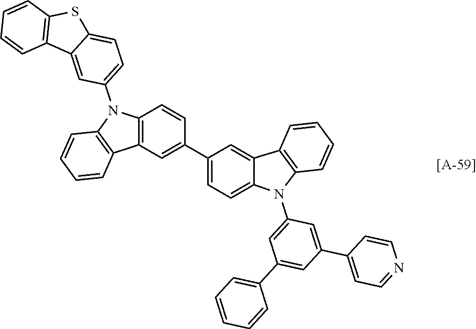 [SMILES: c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

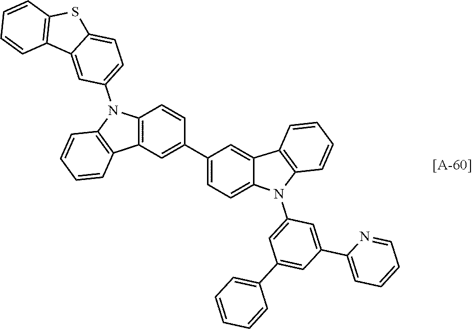 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

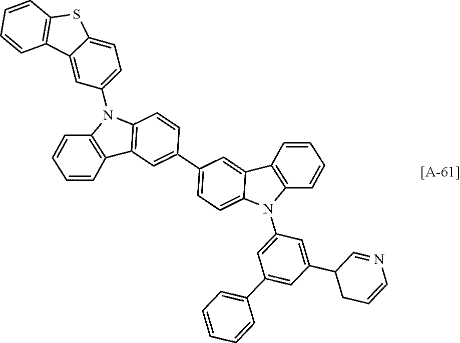 [SMILES: C1=CN=CC(c2cc(-c3ccccc3)cc(-n3c4ccccc4c4cc(-c5ccc6c(c5)c5ccccc5n6-c5ccc6sc7ccccc7c6c5)ccc43)c2)C1]

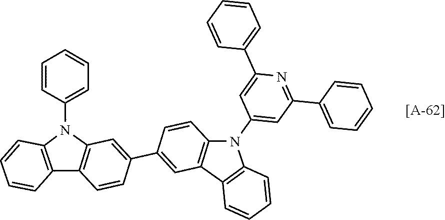 [SMILES: c1ccc(-c2cc(-n3c4ccccc4c4cc(-c5ccc6c7ccccc7n(-c7ccccc7)c6c5)ccc43)cc(-c3ccccc3)n2)cc1]

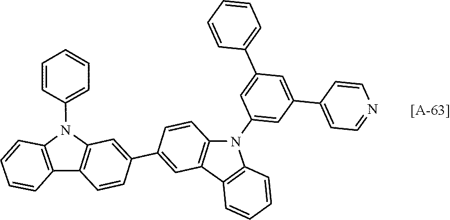 [SMILES: c1ccc(-c2cc(-c3ccncc3)cc(-n3c4ccccc4c4cc(-c5ccc6c7ccccc7n(-c7ccccc7)c6c5)ccc43)c2)cc1]

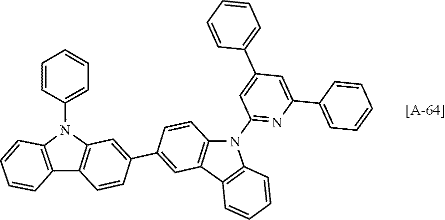 [SMILES: c1ccc(-c2cc(-c3ccccc3)nc(-n3c4ccccc4c4cc(-c5ccc6c7ccccc7n(-c7ccccc7)c6c5)ccc43)c2)cc1]

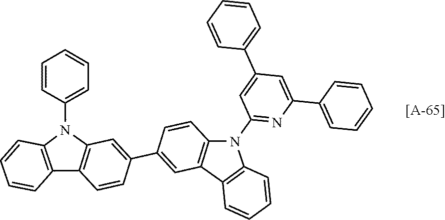 [SMILES: c1ccc(-c2cc(-c3ccccc3)nc(-n3c4ccccc4c4cc(-c5ccc6c7ccccc7n(-c7ccccc7)c6c5)ccc43)c2)cc1]

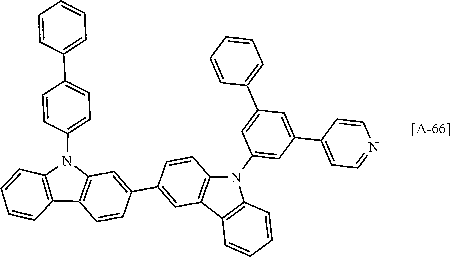 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1]

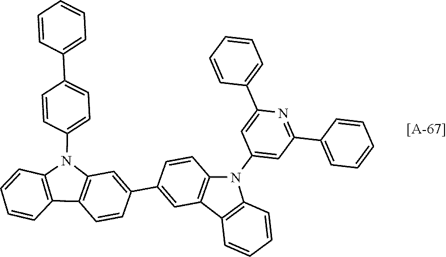 [SMILES: c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1]

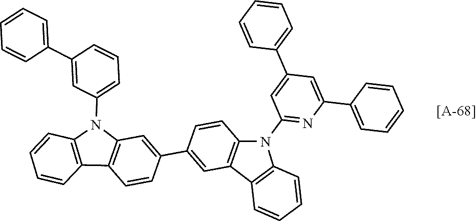 [SMILES: c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

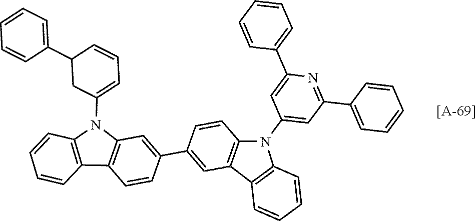 [SMILES: C1=CC(c2ccccc2)CC(n2c3ccccc3c3ccc(-c4ccc5c(c4)c4ccccc4n5-c4cc(-c5ccccc5)nc(-c5ccccc5)c4)cc32)=C1]

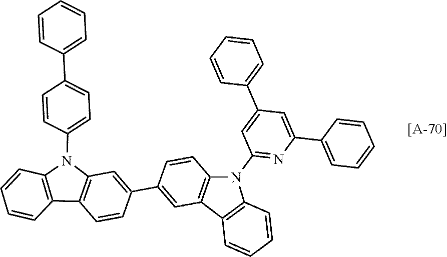 [SMILES: c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1]

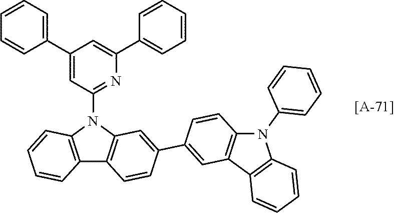 [SMILES: c1ccc(-c2cc(-c3ccccc3)nc(-n3c4ccccc4c4ccc(-c5ccc6c(c5)c5ccccc5n6-c5ccccc5)cc43)c2)cc1]

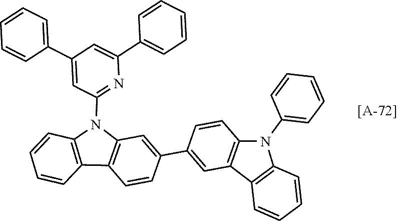 [SMILES: c1ccc(-c2cc(-c3ccccc3)nc(-n3c4ccccc4c4ccc(-c5ccc6c(c5)c5ccccc5n6-c5ccccc5)cc43)c2)cc1]

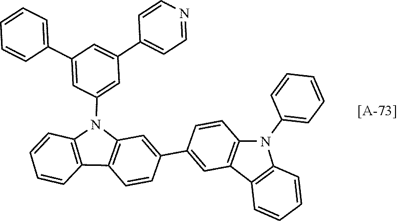 [SMILES: c1ccc(-c2cc(-c3ccncc3)cc(-n3c4ccccc4c4ccc(-c5ccc6c(c5)c5ccccc5n6-c5ccccc5)cc43)c2)cc1]

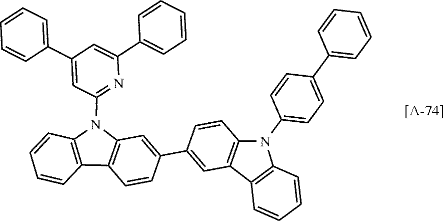 [SMILES: c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1]

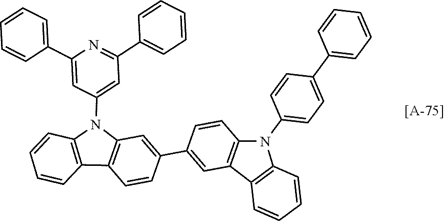 [SMILES: c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

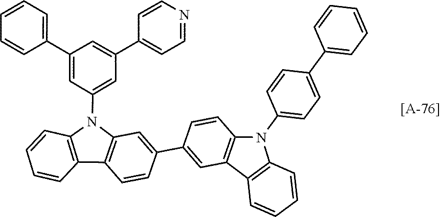 [SMILES: c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1]

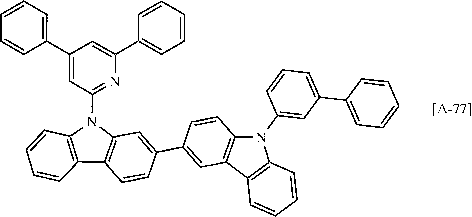 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1]

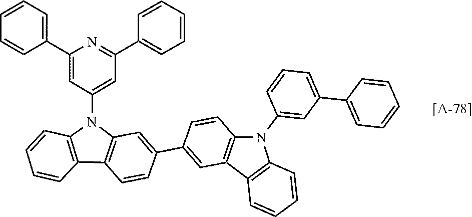 [SMILES: c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1]

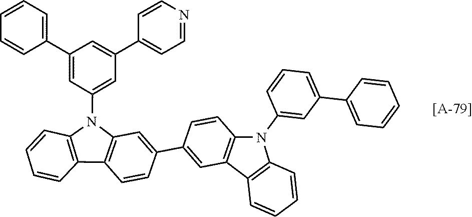 [SMILES: c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1]

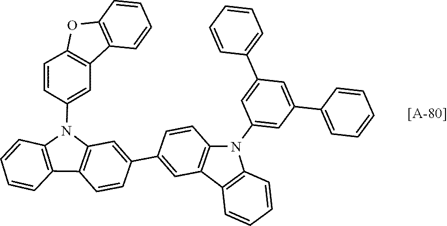 [SMILES: c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

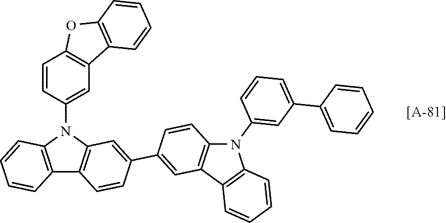 [SMILES: c1ccc(-c2cccc(-n3c4ccccc4c4cc(-c5ccc6c7ccccc7n(-c7ccc8oc9ccccc9c8c7)c6c5)ccc43)c2)cc1]

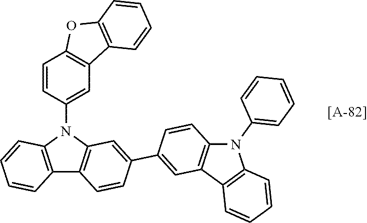 [SMILES: c1ccc(-n2c3ccccc3c3cc(-c4ccc5c6ccccc6n(-c6ccc7oc8ccccc8c7c6)c5c4)ccc32)cc1]

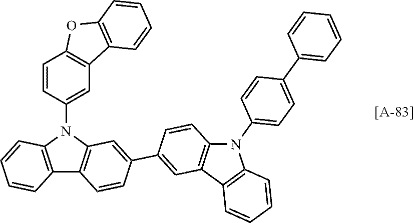 [SMILES: c1ccc(-c2ccc(-n3c4ccccc4c4cc(-c5ccc6c7ccccc7n(-c7ccc8oc9ccccc9c8c7)c6c5)ccc43)cc2)cc1]

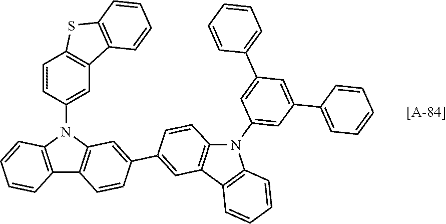 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1]

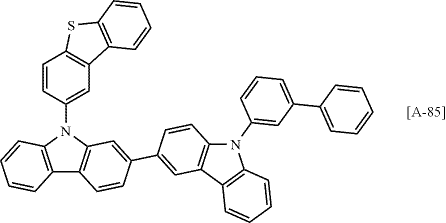 [SMILES: c1ccc(-c2cccc(-n3c4ccccc4c4cc(-c5ccc6c7ccccc7n(-c7ccc8sc9ccccc9c8c7)c6c5)ccc43)c2)cc1]

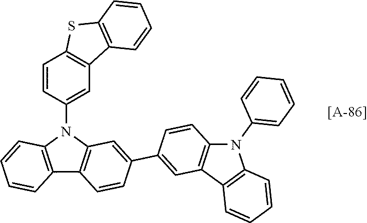 [SMILES: c1ccc(-n2c3ccccc3c3cc(-c4ccc5c6ccccc6n(-c6ccc7sc8ccccc8c7c6)c5c4)ccc32)cc1]

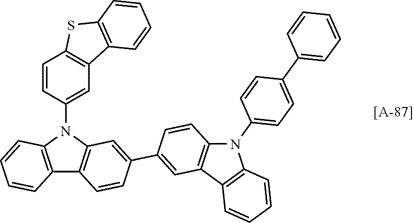 [SMILES: c1ccc(-c2ccc(-n3c4ccccc4c4cc(-c5ccc6c7ccccc7n(-c7ccc8sc9ccccc9c8c7)c6c5)ccc43)cc2)cc1]

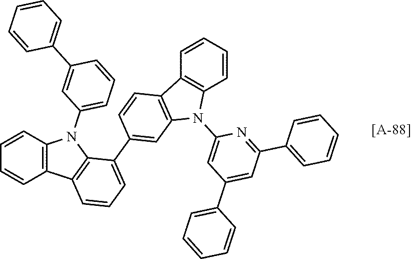 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1]

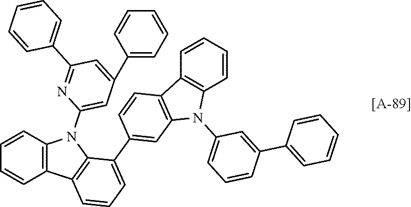 [SMILES: c1ccc(-c2cccc(-n3c4ccccc4c4ccc(-c5cccc6c7ccccc7n(-c7cc(-c8ccccc8)cc(-c8ccccc8)n7)c56)cc43)c2)cc1]

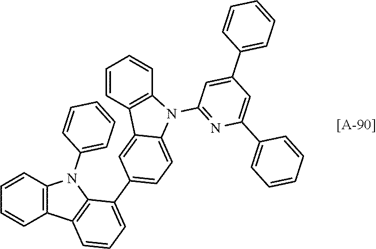 [SMILES: c1ccc(-c2cc(-c3ccccc3)nc(-n3c4ccccc4c4cc(-c5cccc6c7ccccc7n(-c7ccccc7)c56)ccc43)c2)cc1]

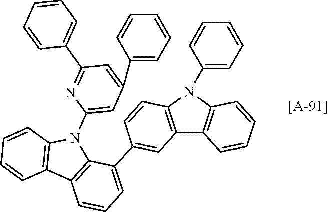 [SMILES: c1ccc(-c2cc(-c3ccccc3)nc(-n3c4ccccc4c4cccc(-c5ccc6c(c5)c5ccccc5n6-c5ccccc5)c43)c2)cc1]

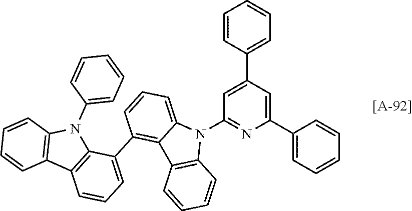 [SMILES: c1ccc(-c2cc(-c3ccccc3)nc(-n3c4ccccc4c4c(-c5cccc6c7ccccc7n(-c7ccccc7)c56)cccc43)c2)cc1]

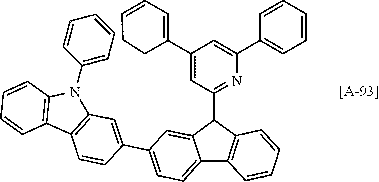 [SMILES: C1=CCCC(c2cc(-c3ccccc3)nc(C3c4ccccc4-c4ccc(-c5ccc6c7ccccc7n(-c7ccccc7)c6c5)cc43)c2)=C1]

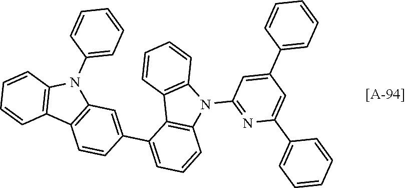 [SMILES: c1ccc(-c2cc(-c3ccccc3)nc(-n3c4ccccc4c4c(-c5ccc6c7ccccc7n(-c7ccccc7)c6c5)cccc43)c2)cc1]

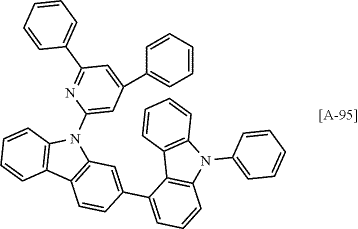 [SMILES: c1ccc(-c2cc(-c3ccccc3)nc(-n3c4ccccc4c4ccc(-c5cccc6c5c5ccccc5n6-c5ccccc5)cc43)c2)cc1]

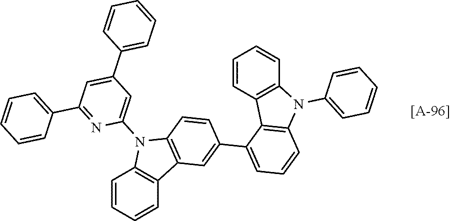 [SMILES: c1ccc(-c2cc(-c3ccccc3)nc(-n3c4ccccc4c4cc(-c5cccc6c5c5ccccc5n6-c5ccccc5)ccc43)c2)cc1]

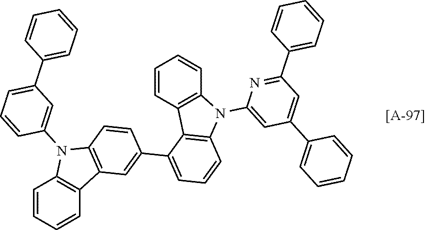 [SMILES: c1ccc(-c2cccc(-n3c4ccccc4c4cc(-c5cccc6c5c5ccccc5n6-c5cc(-c6ccccc6)cc(-c6ccccc6)n5)ccc43)c2)cc1]

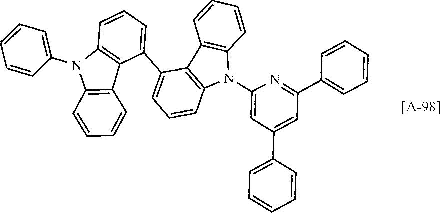 [SMILES: c1ccc(-c2cc(-c3ccccc3)nc(-n3c4ccccc4c4c(-c5cccc6c5c5ccccc5n6-c5ccccc5)cccc43)c2)cc1]

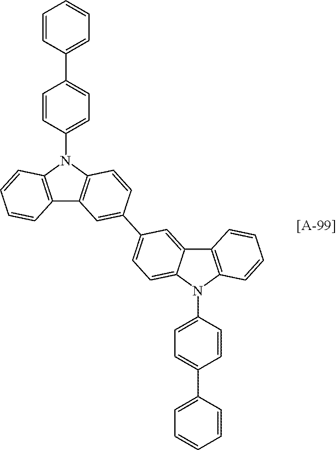 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

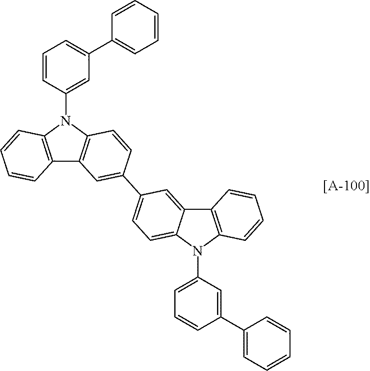 [SMILES: c1ccc(-c2cccc(-n3c4ccccc4c4cc(-c5ccc6c(c5)c5ccccc5n6-c5cccc(-c6ccccc6)c5)ccc43)c2)cc1]

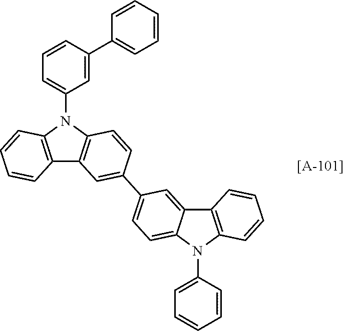 [SMILES: c1ccc(-c2cccc(-n3c4ccccc4c4cc(-c5ccc6c(c5)c5ccccc5n6-c5ccccc5)ccc43)c2)cc1]

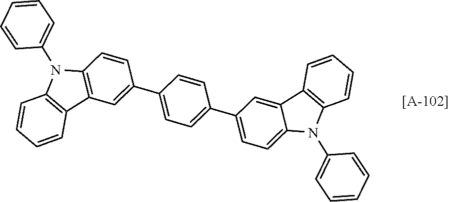 [SMILES: c1ccc(-n2c3ccccc3c3cc(-c4ccc(-c5ccc6c(c5)c5ccccc5n6-c5ccccc5)cc4)ccc32)cc1]

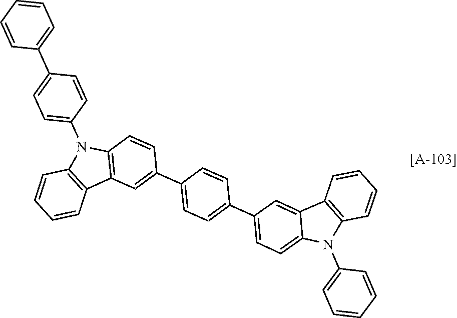 [SMILES: c1ccc(-c2ccc(-n3c4ccccc4c4cc(-c5ccc(-c6ccc7c(c6)c6ccccc6n7-c6ccccc6)cc5)ccc43)cc2)cc1]

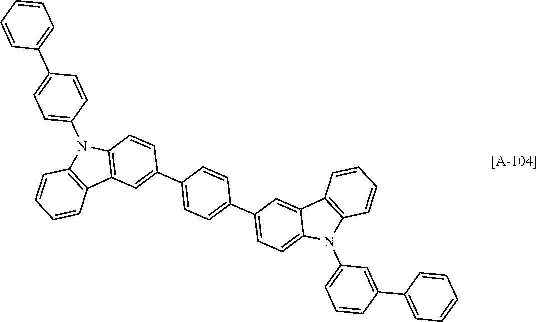 [SMILES: c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

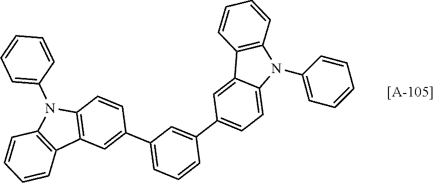 [SMILES: c1ccc(-n2c3ccccc3c3cc(-c4cccc(-c5ccc6c(c5)c5ccccc5n6-c5ccccc5)c4)ccc32)cc1]

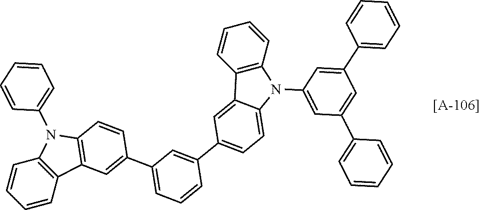 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

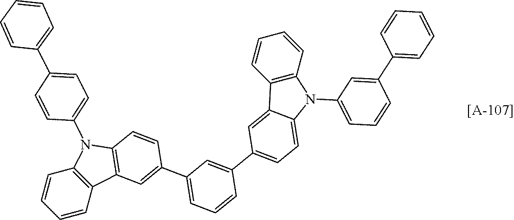 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

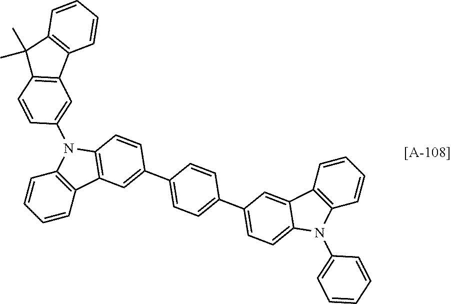 [SMILES: CC1(C)c2ccccc2-c2cc(-n3c4ccccc4c4cc(-c5ccc(-c6ccc7c(c6)c6ccccc6n7-c6ccccc6)cc5)ccc43)ccc21]

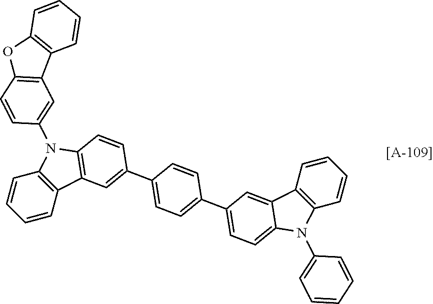 [SMILES: c1ccc(-n2c3ccccc3c3cc(-c4ccc(-c5ccc6c(c5)c5ccccc5n6-c5ccc6oc7ccccc7c6c5)cc4)ccc32)cc1]

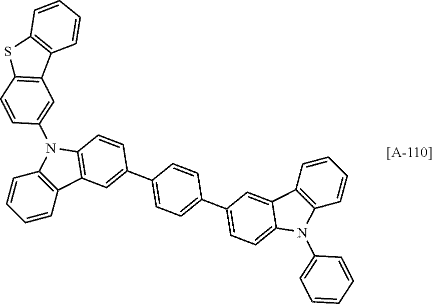 [SMILES: c1ccc(-n2c3ccccc3c3cc(-c4ccc(-c5ccc6c(c5)c5ccccc5n6-c5ccc6sc7ccccc7c6c5)cc4)ccc32)cc1]

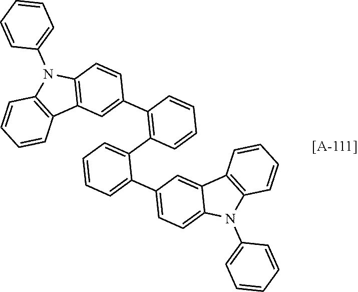 [SMILES: c1ccc(-n2c3ccccc3c3cc(-c4ccccc4-c4ccccc4-c4ccc5c(c4)c4ccccc4n5-c4ccccc4)ccc32)cc1]

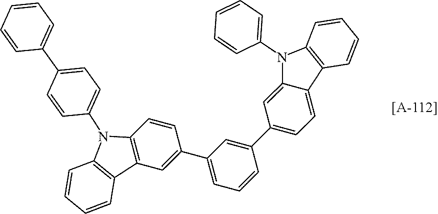 [SMILES: c1ccc(-c2ccc(-n3c4ccccc4c4cc(-c5cccc(-c6ccc7c8ccccc8n(-c8ccccc8)c7c6)c5)ccc43)cc2)cc1]

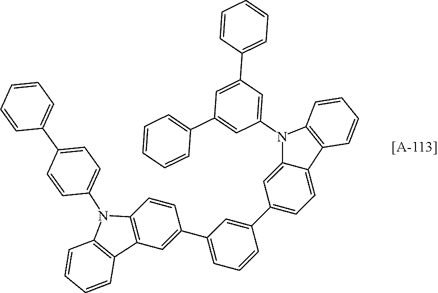 [SMILES: c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

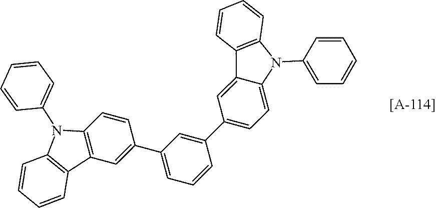 [SMILES: c1ccc(-n2c3ccccc3c3cc(-c4cccc(-c5ccc6c(c5)c5ccccc5n6-c5ccccc5)c4)ccc32)cc1]

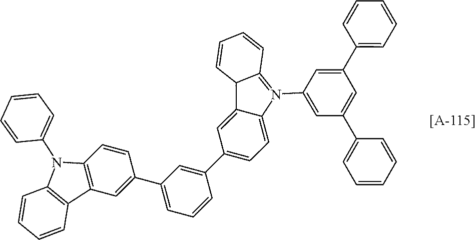

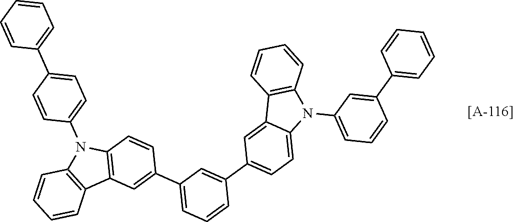 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

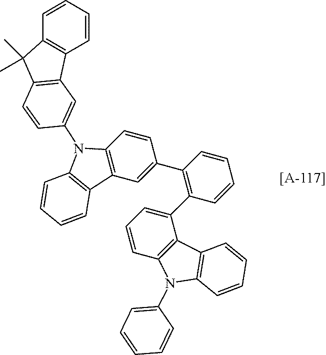 [SMILES: CC1(C)c2ccccc2-c2cc(-n3c4ccccc4c4cc(-c5ccccc5-c5cccc6c5c5ccccc5n6-c5ccccc5)ccc43)ccc21]

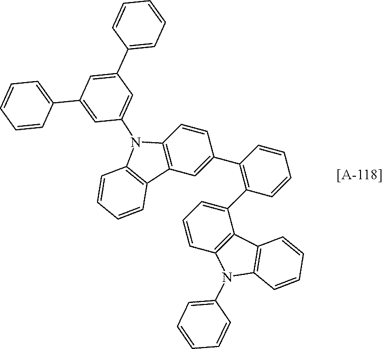 [SMILES: c1ccc(-c2cc(-c3ccccc3)cc(-n3c4ccccc4c4cc(-c5ccccc5-c5cccc6c5c5ccccc5n6-c5ccccc5)ccc43)c2)cc1]

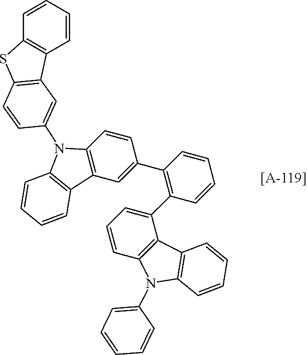 [SMILES: c1ccc(-n2c3ccccc3c3c(-c4ccccc4-c4ccc5c(c4)c4ccccc4n5-c4ccc5sc6ccccc6c5c4)cccc32)cc1]

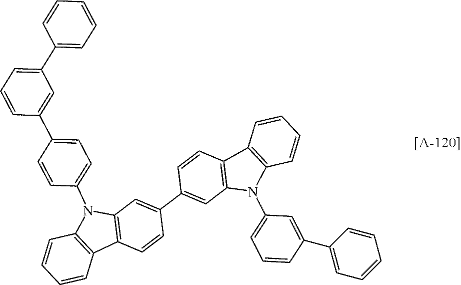 [SMILES: c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

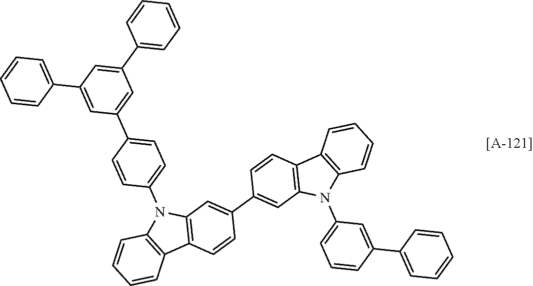 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

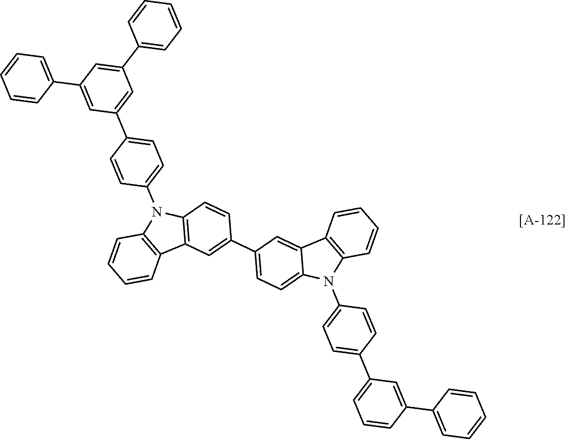 [SMILES: c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

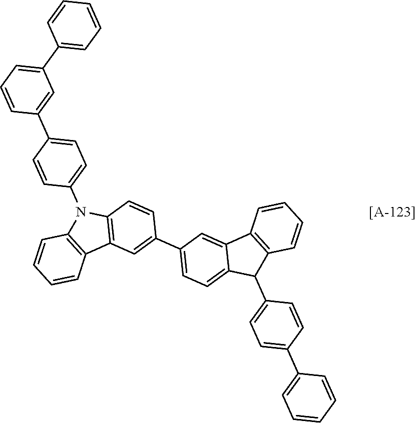 [SMILES: c1ccc(-c2ccc(C3c4ccccc4-c4cc(-c5ccc6c(c5)c5ccccc5n6-c5ccc(-c6cccc(-c7ccccc7)c6)cc5)ccc43)cc2)cc1]

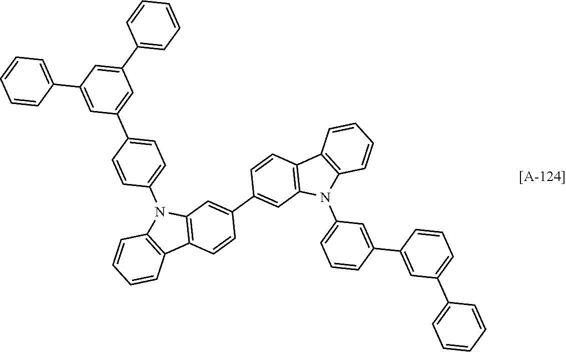 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

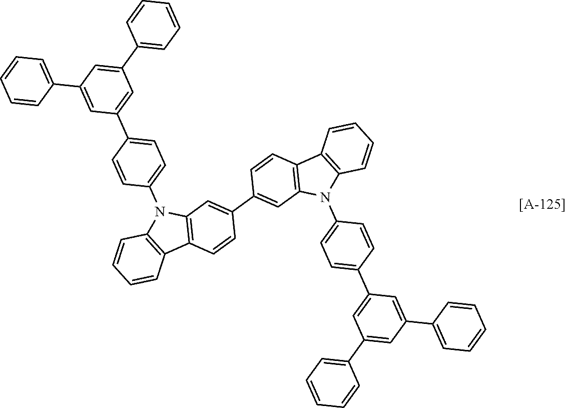 [SMILES: c1ccc(-c2cc(-c3ccccc3)cc(-c3ccc(-n4c5ccccc5c5ccc(-c6ccc7c8ccccc8n(-c8ccc(-c9cc(-c%10ccccc%10)cc(-c%10ccccc%10)c9)cc8)c7c6)cc54)cc3)c2)cc1]

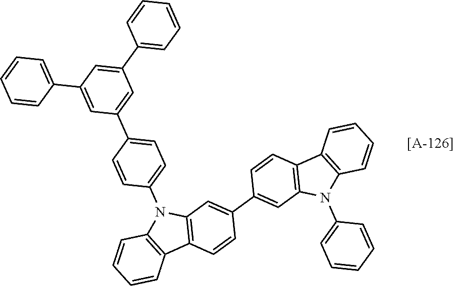 [SMILES: c1ccc(-c2cc(-c3ccccc3)cc(-c3ccc(-n4c5ccccc5c5ccc(-c6ccc7c8ccccc8n(-c8ccccc8)c7c6)cc54)cc3)c2)cc1]

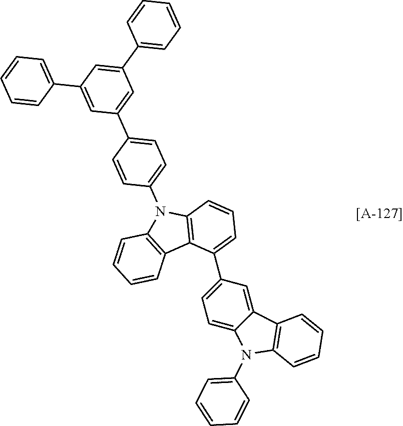 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

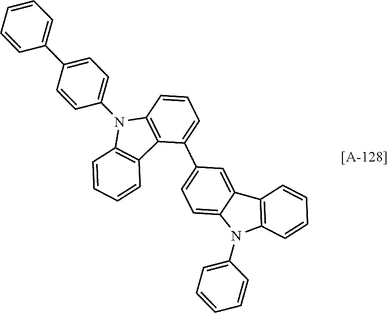 [SMILES: c1ccc(-c2ccc(-n3c4ccccc4c4c(-c5ccc6c(c5)c5ccccc5n6-c5ccccc5)cccc43)cc2)cc1]

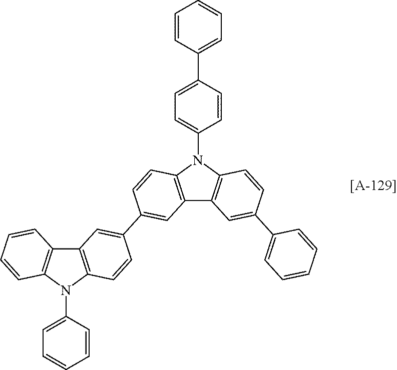 [SMILES: c1ccc(-c2ccc(-n3c4ccc(-c5ccccc5)cc4c4cc(-c5ccc6c(c5)c5ccccc5n6-c5ccccc5)ccc43)cc2)cc1]

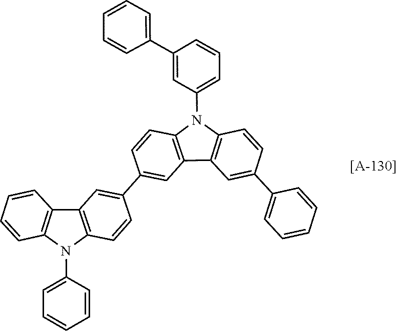 [SMILES: c1ccc(-c2cccc(-n3c4ccc(-c5ccccc5)cc4c4cc(-c5ccc6c(c5)c5ccccc5n6-c5ccccc5)ccc43)c2)cc1]

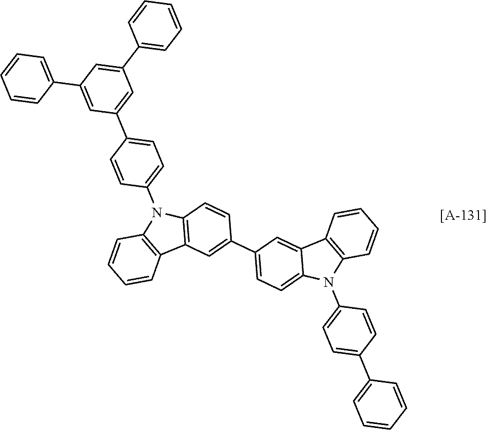 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

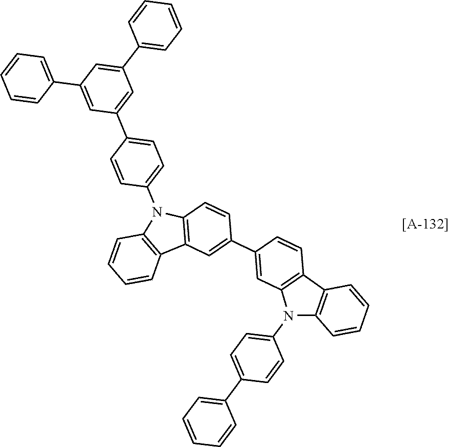 [SMILES: c1ccc(-c2ccc(-n3c4ccccc4c4ccc(-c5ccc6c(c5)c5ccccc5n6-c5ccc(-c6cc(-c7ccccc7)cc(-c7ccccc7)c6)cc5)cc43)cc2)cc1]

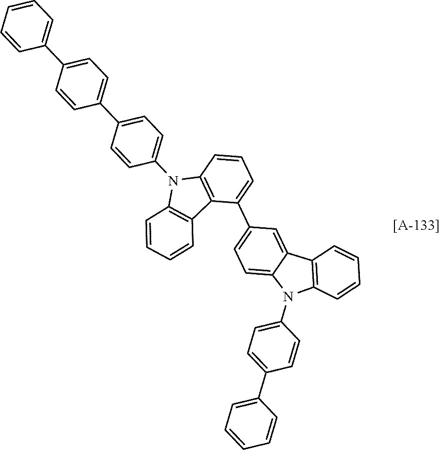 [SMILES: c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1]

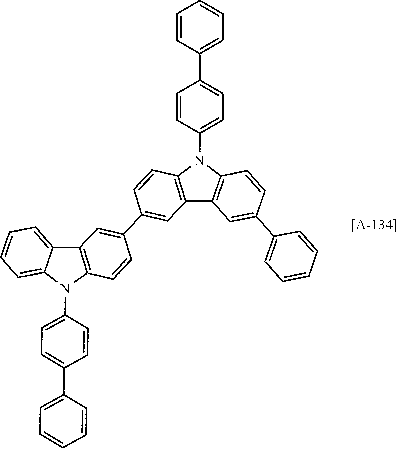 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

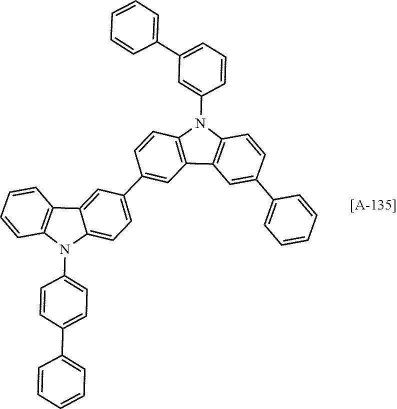 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1]

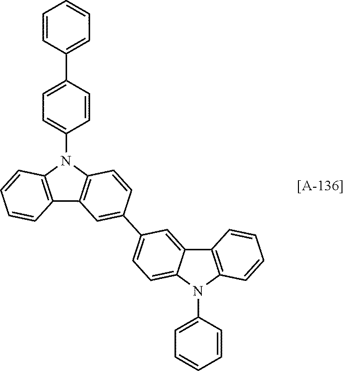 [SMILES: c1ccc(-c2ccc(-n3c4ccccc4c4cc(-c5ccc6c(c5)c5ccccc5n6-c5ccccc5)ccc43)cc2)cc1]

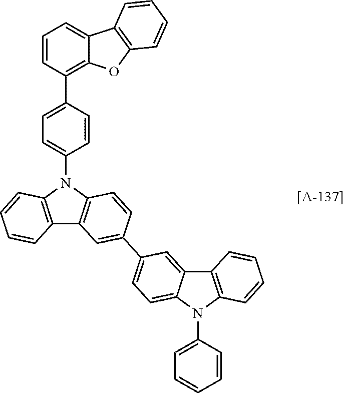 [SMILES: c1ccc(-n2c3ccccc3c3cc(-c4ccc5c(c4)c4ccccc4n5-c4ccc(-c5cccc6c5oc5ccccc56)cc4)ccc32)cc1]

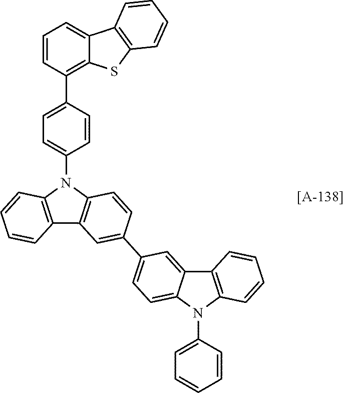 [SMILES: c1ccc(-n2c3ccccc3c3cc(-c4ccc5c(c4)c4ccccc4n5-c4ccc(-c5cccc6c5sc5ccccc56)cc4)ccc32)cc1]

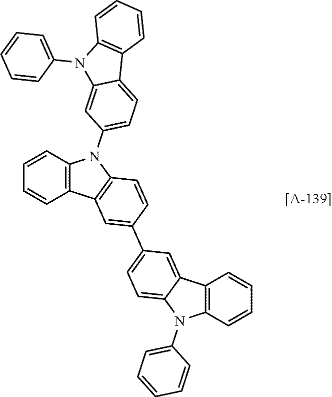 [SMILES: c1ccc(-n2c3ccccc3c3cc(-c4ccc5c(c4)c4ccccc4n5-c4ccc5c6ccccc6n(-c6ccccc6)c5c4)ccc32)cc1]

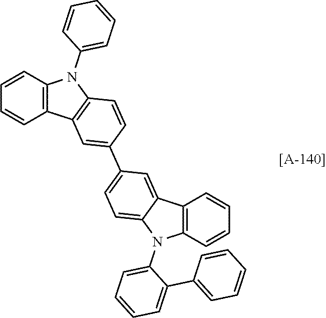 [SMILES: c1ccc(-c2ccccc2-n2c3ccccc3c3cc(-c4ccc5c(c4)c4ccccc4n5-c4ccccc4)ccc32)cc1]

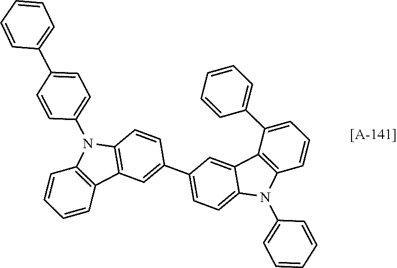 [SMILES: c1ccc(-c2ccc(-n3c4ccccc4c4cc(-c5ccc6c(c5)c5c(-c7ccccc7)cccc5n6-c5ccccc5)ccc43)cc2)cc1]

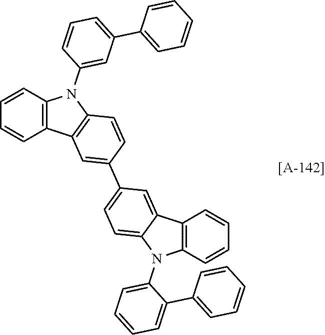 [SMILES: c1ccc(-c2cccc(-n3c4ccccc4c4cc(-c5ccc6c(c5)c5ccccc5n6-c5ccccc5-c5ccccc5)ccc43)c2)cc1]

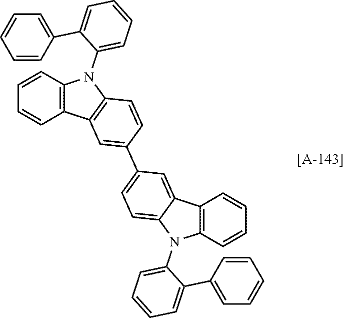 [SMILES: c1ccc(-c2ccccc2-n2c3ccccc3c3cc(-c4ccc5c(c4)c4ccccc4n5-c4ccccc4-c4ccccc4)ccc32)cc1]

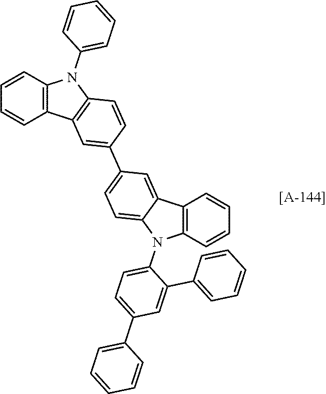 [SMILES: c1ccc(-c2ccc(-n3c4ccccc4c4cc(-c5ccc6c(c5)c5ccccc5n6-c5ccccc5)ccc43)c(-c3ccccc3)c2)cc1]

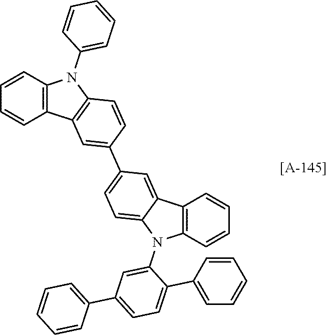 [SMILES: c1ccc(-c2ccc(-c3ccccc3)c(-n3c4ccccc4c4cc(-c5ccc6c(c5)c5ccccc5n6-c5ccccc5)ccc43)c2)cc1]

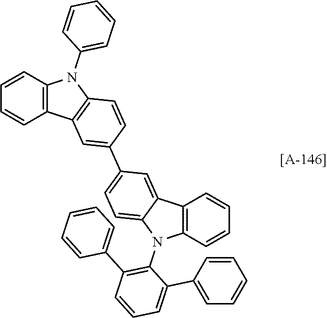 [SMILES: c1ccc(-c2cccc(-c3ccccc3)c2-n2c3ccccc3c3cc(-c4ccc5c(c4)c4ccccc4n5-c4ccccc4)ccc32)cc1]

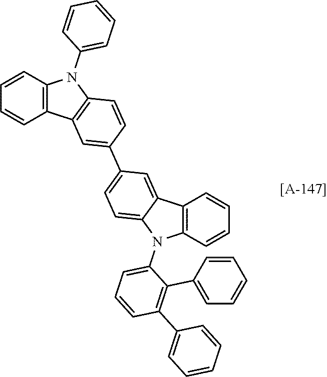 [SMILES: c1ccc(-c2cccc(-n3c4ccccc4c4cc(-c5ccc6c(c5)c5ccccc5n6-c5ccccc5)ccc43)c2-c2ccccc2)cc1]

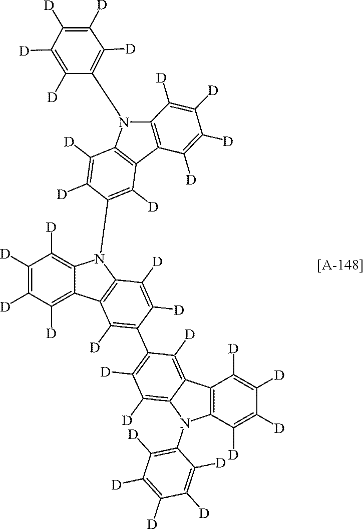 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

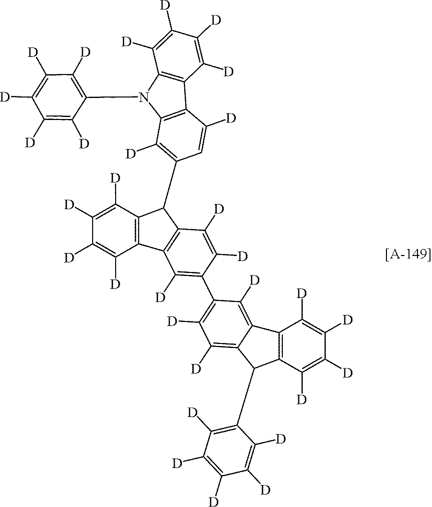 [SMILES: [2H]c1c([2H])c([2H])c(C2c3c([2H])c([2H])c([2H])c([2H])c3-c3c([2H])c(-c4c([2H])c([2H])c5c(c4[2H])-c4c([2H])c([2H])c([2H])c([2H])c4C5c4cc([2H])c5c6c([2H])c([2H])c([2H])c([2H])c6n(-c6c([2H])c([2H])c([2H])c([2H])c6[2H])c5c4[2H])c([2H])c([2H])c32)c([2H])c1[2H]]

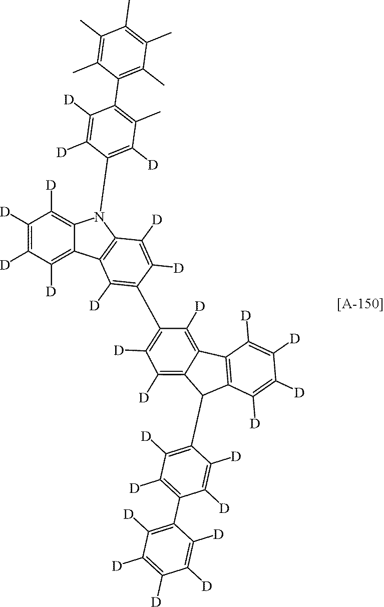 [SMILES: [2H]c1c([2H])c([2H])c(-c2c([2H])c([2H])c(C3c4c([2H])c([2H])c([2H])c([2H])c4-c4c([2H])c(-c5c([2H])c([2H])c6c(c5[2H])c5c([2H])c([2H])c([2H])c([2H])c5n6-c5c([2H])c([2H])c(-c6c(C)c(C)c(C)c(C)c6C)c(C)c5[2H])c([2H])c([2H])c43)c([2H])c2[2H])c([2H])c1[2H]]

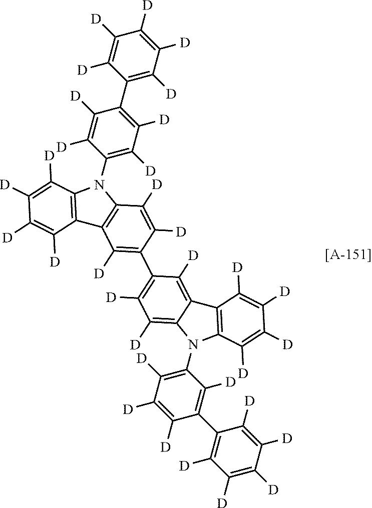 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]]

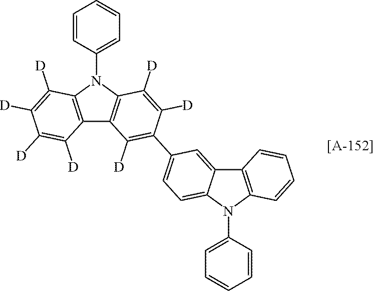 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1]

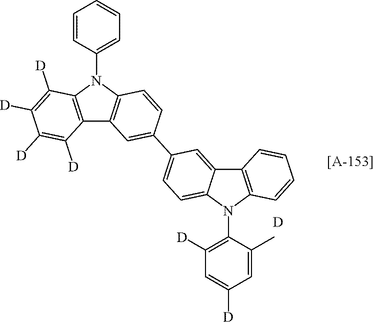

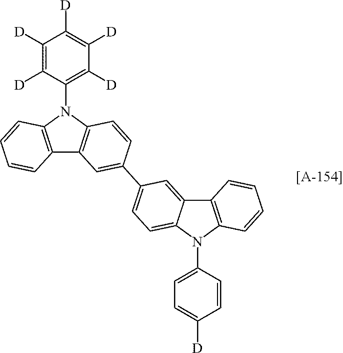 [SMILES: [2H]c1ccc(-n2c3ccccc3c3cc(-c4ccc5c(c4)c4ccccc4n5-c4c([2H])c([2H])c([2H])c([2H])c4[2H])ccc32)cc1]

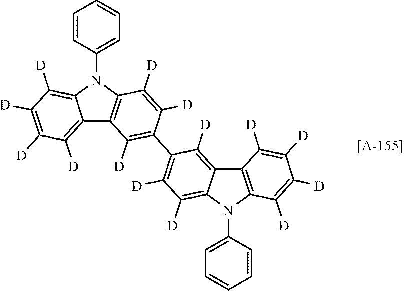 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

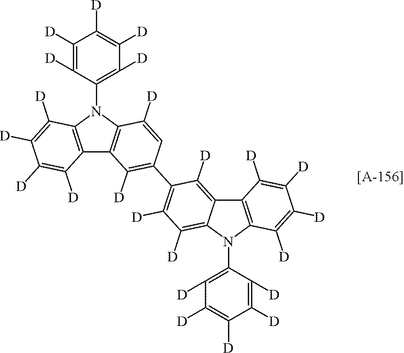 [SMILES: [2H]c1c([2H])c([2H])c(-n2c3c([2H])cc(-c4c([2H])c([2H])c5c(c4[2H])c4c([2H])c([2H])c([2H])c([2H])c4n5-c4c([2H])c([2H])c([2H])c([2H])c4[2H])c([2H])c3c3c([2H])c([2H])c([2H])c([2H])c32)c([2H])c1[2H]]

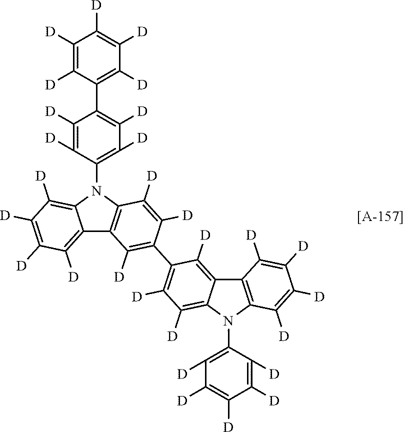 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

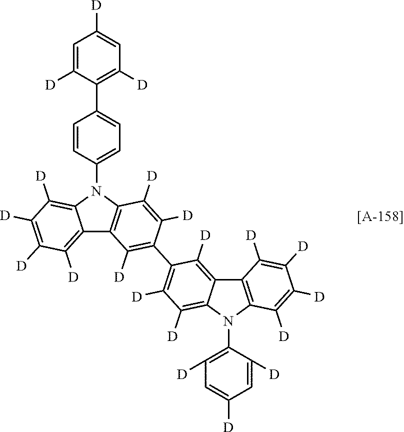 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1]

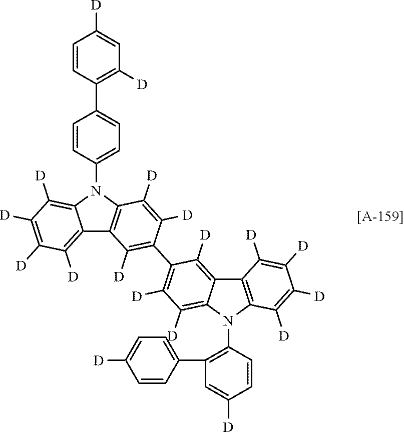 [SMILES: [2H]c1ccc(-c2cc([2H])ccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

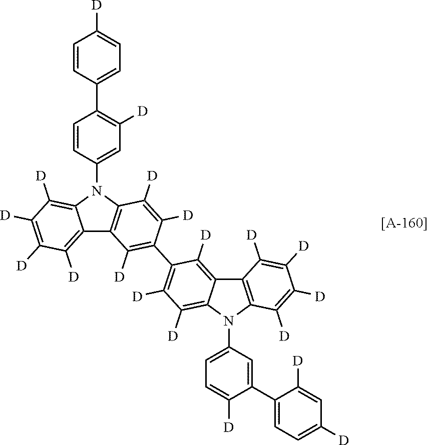 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1]

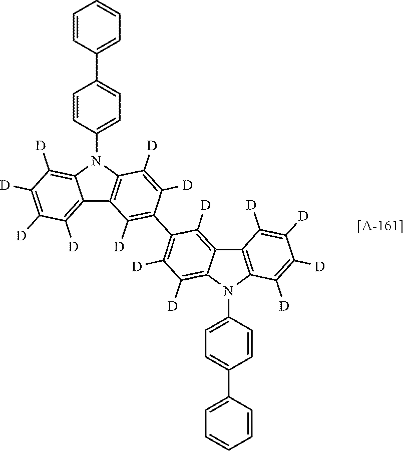 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

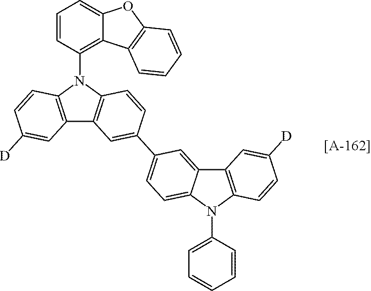 [SMILES: [2H]c1ccc2c(c1)c1cc(-c3ccc4c(c3)c3cc([2H])ccc3n4-c3cccc4oc5ccccc5c34)ccc1n2-c1ccccc1]

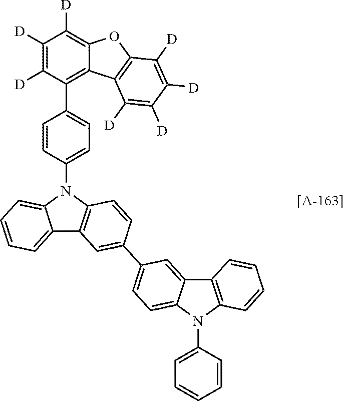 [SMILES: [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H]]

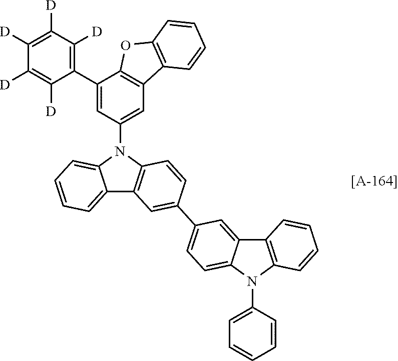 [SMILES: [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

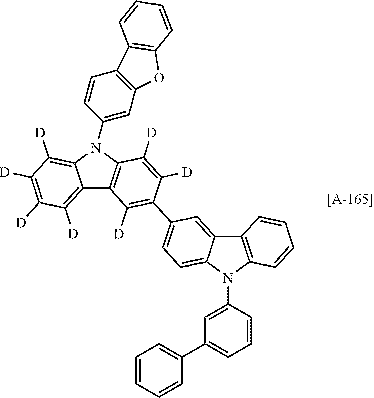 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1ccc2c(c1)oc1ccccc12]

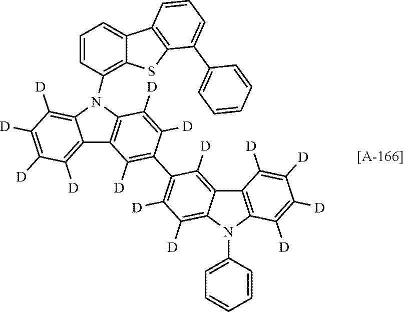 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

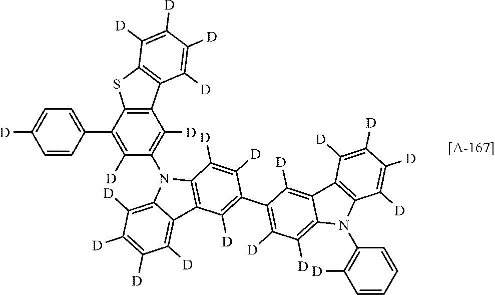 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1]

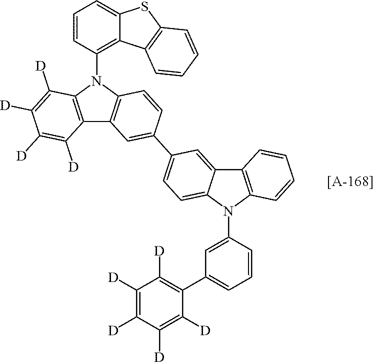 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H]]

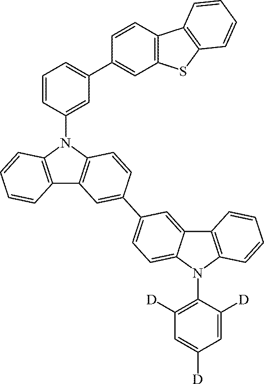 [SMILES: [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

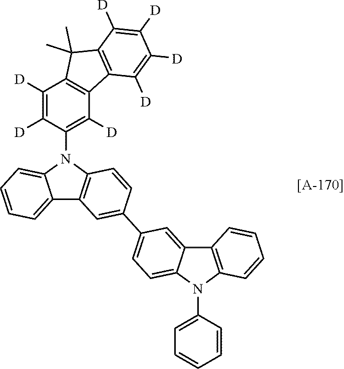 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C]

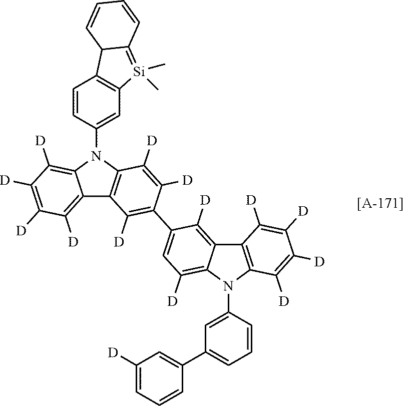

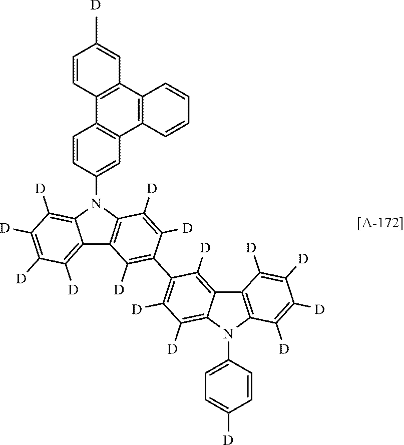

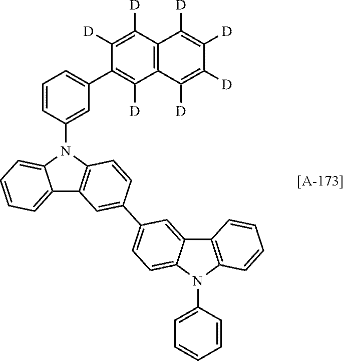 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

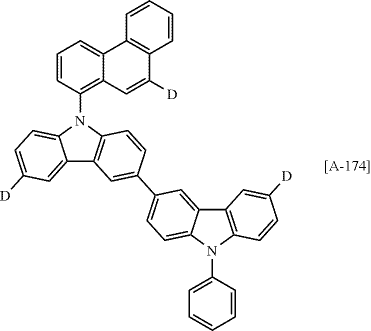 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1]

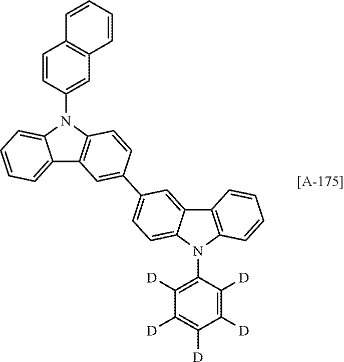 [SMILES: [2H]c1c([2H])c([2H])c(-n2c3ccccc3c3cc(-c4ccc5c(c4)c4ccccc4n5-c4ccc5ccccc5c4)ccc32)c([2H])c1[2H]]

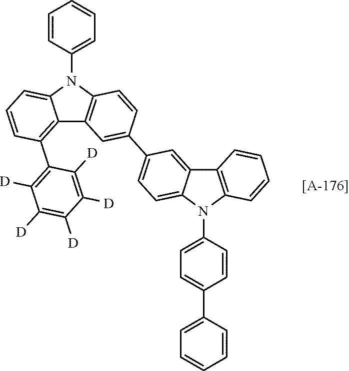 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H]]

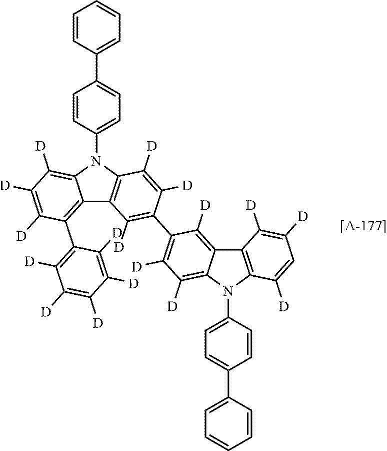 [SMILES: [2H]c1cc([2H])c2c(c1[2H])c1c([2H])c(-c3c([2H])c([2H])c4c(c3[2H])c3c(-c5c([2H])c([2H])c([2H])c([2H])c5[2H])c([2H])c([2H])c([2H])c3n4-c3ccc(-c4ccccc4)cc3)c([2H])c([2H])c1n2-c1ccc(-c2ccccc2)cc1]

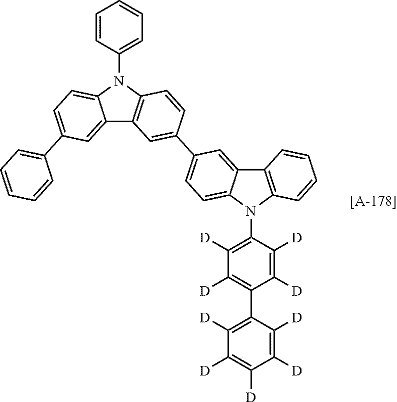 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

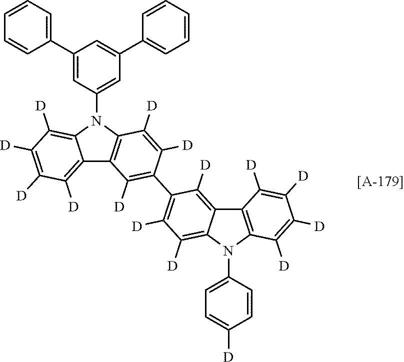 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

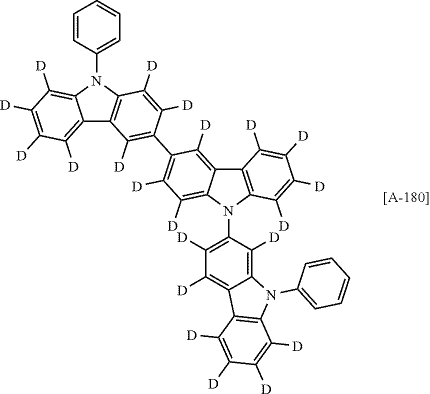 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

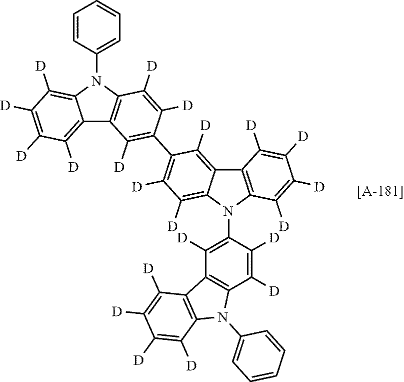 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

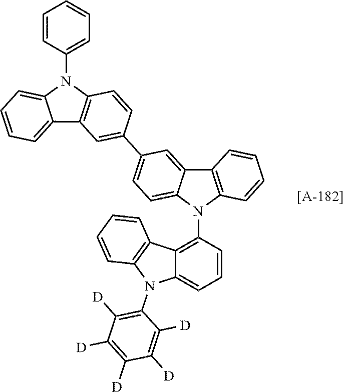 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H]]

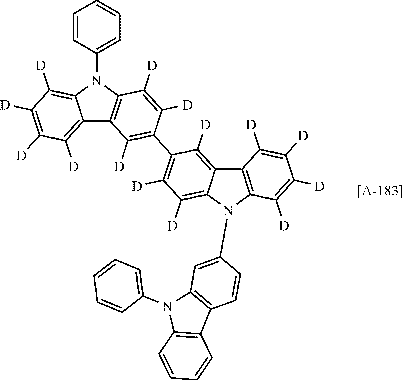 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5n(-c5ccccc5)c4c3)c([2H])c([2H])c1n2-c1ccccc1]

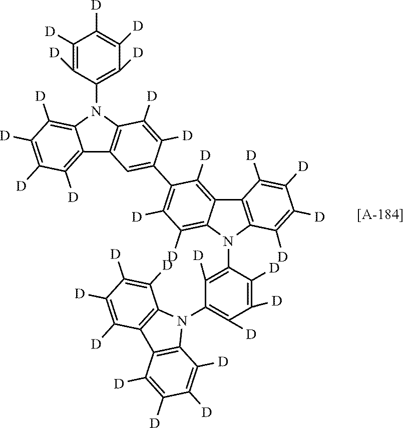 [SMILES: [2H]c1c([2H])c([2H])c(-n2c3c([2H])c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])cc3c3c([2H])c([2H])c([2H])c([2H])c32)c([2H])c1[2H]]

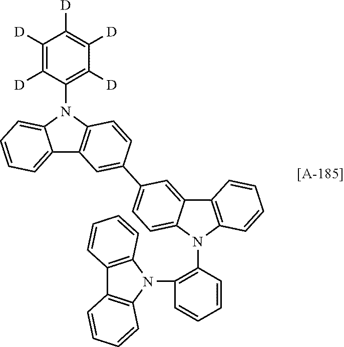 [SMILES: [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

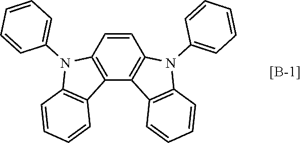 [SMILES: c1ccc(-n2c3ccccc3c3c4c5ccccc5n(-c5ccccc5)c4ccc32)cc1]

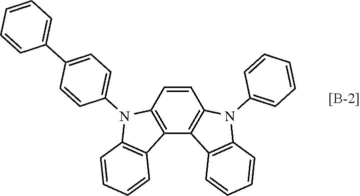 [SMILES: c1ccc(-c2ccc(-n3c4ccccc4c4c5c6ccccc6n(-c6ccccc6)c5ccc43)cc2)cc1]

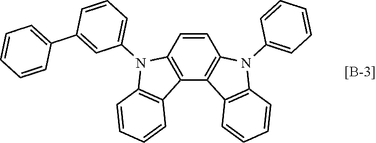 [SMILES: c1ccc(-c2cccc(-n3c4ccccc4c4c5c6ccccc6n(-c6ccccc6)c5ccc43)c2)cc1]

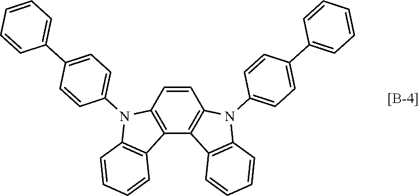 [SMILES: c1ccc(-c2ccc(-n3c4ccccc4c4c5c6ccccc6n(-c6ccc(-c7ccccc7)cc6)c5ccc43)cc2)cc1]

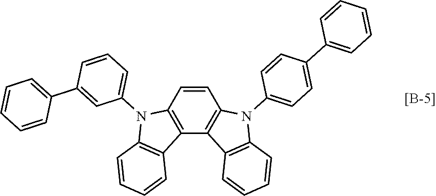 [SMILES: c1ccc(-c2ccc(-n3c4ccccc4c4c5c6ccccc6n(-c6cccc(-c7ccccc7)c6)c5ccc43)cc2)cc1]

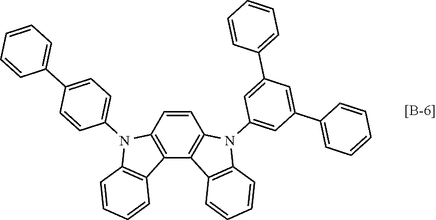 [SMILES: c1ccc(-c2ccc(-n3c4ccccc4c4c5c6ccccc6n(-c6cc(-c7ccccc7)cc(-c7ccccc7)c6)c5ccc43)cc2)cc1]

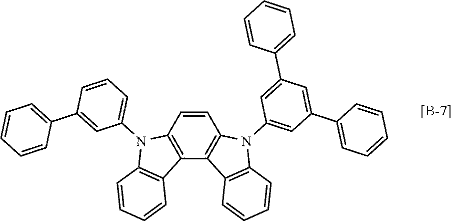 [SMILES: c1ccc(-c2cccc(-n3c4ccccc4c4c5c6ccccc6n(-c6cc(-c7ccccc7)cc(-c7ccccc7)c6)c5ccc43)c2)cc1]

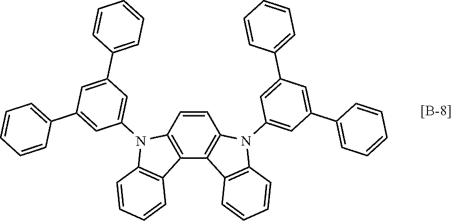 [SMILES: c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

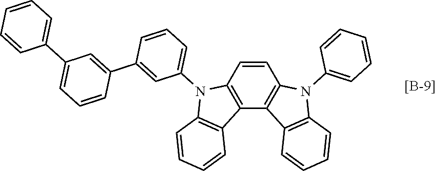 [SMILES: c1ccc(-c2cccc(-c3cccc(-n4c5ccccc5c5c6c7ccccc7n(-c7ccccc7)c6ccc54)c3)c2)cc1]

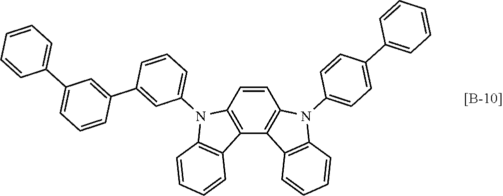 [SMILES: c1ccc(-c2ccc(-n3c4ccccc4c4c5c6ccccc6n(-c6cccc(-c7cccc(-c8ccccc8)c7)c6)c5ccc43)cc2)cc1]

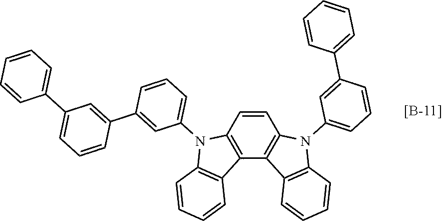 [SMILES: c1ccc(-c2cccc(-c3cccc(-n4c5ccccc5c5c6c7ccccc7n(-c7cccc(-c8ccccc8)c7)c6ccc54)c3)c2)cc1]

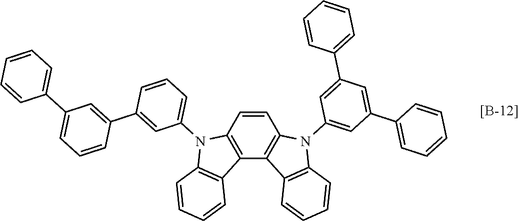 [SMILES: c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

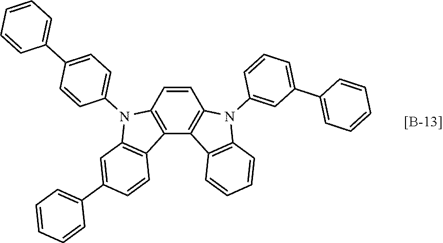 [SMILES: c1ccc(-c2ccc(-n3c4cc(-c5ccccc5)ccc4c4c5c6ccccc6n(-c6cccc(-c7ccccc7)c6)c5ccc43)cc2)cc1]

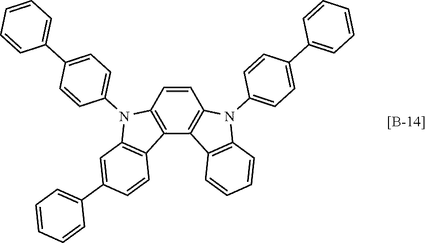 [SMILES: c1ccc(-c2ccc(-n3c4ccccc4c4c5c6ccc(-c7ccccc7)cc6n(-c6ccc(-c7ccccc7)cc6)c5ccc43)cc2)cc1]

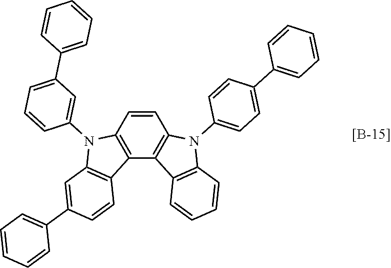 [SMILES: c1ccc(-c2ccc(-n3c4ccccc4c4c5c6ccc(-c7ccccc7)cc6n(-c6cccc(-c7ccccc7)c6)c5ccc43)cc2)cc1]

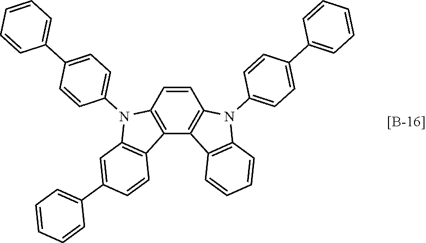 [SMILES: c1ccc(-c2ccc(-n3c4ccccc4c4c5c6ccc(-c7ccccc7)cc6n(-c6ccc(-c7ccccc7)cc6)c5ccc43)cc2)cc1]

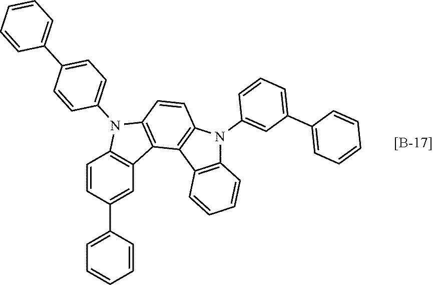 [SMILES: c1ccc(-c2ccc(-n3c4ccc(-c5ccccc5)cc4c4c5c6ccccc6n(-c6cccc(-c7ccccc7)c6)c5ccc43)cc2)cc1]

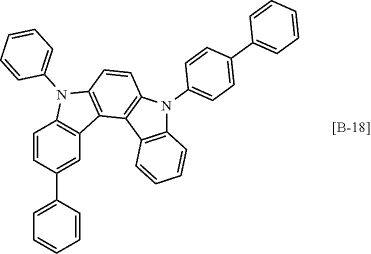 [SMILES: c1ccc(-c2ccc(-n3c4ccccc4c4c5c6cc(-c7ccccc7)ccc6n(-c6ccccc6)c5ccc43)cc2)cc1]

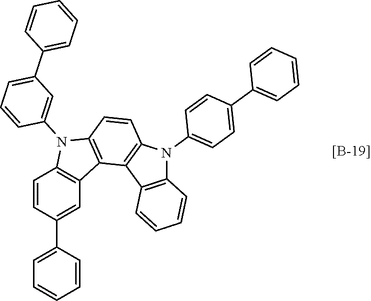 [SMILES: c1ccc(-c2ccc(-n3c4ccccc4c4c5c6cc(-c7ccccc7)ccc6n(-c6cccc(-c7ccccc7)c6)c5ccc43)cc2)cc1]

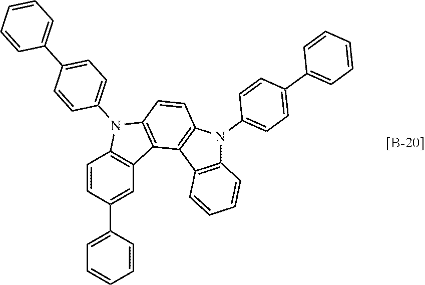 [SMILES: c1ccc(-c2ccc(-n3c4ccccc4c4c5c6cc(-c7ccccc7)ccc6n(-c6ccc(-c7ccccc7)cc6)c5ccc43)cc2)cc1]

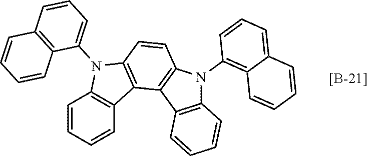 [SMILES: c1ccc2c(-n3c4ccccc4c4c5c6ccccc6n(-c6cccc7ccccc67)c5ccc43)cccc2c1]

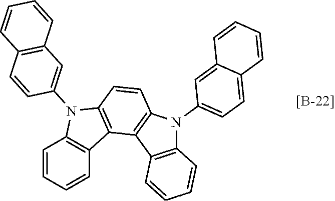 [SMILES: c1ccc2cc(-n3c4ccccc4c4c5c6ccccc6n(-c6ccc7ccccc7c6)c5ccc43)ccc2c1]

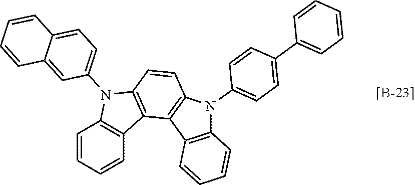 [SMILES: c1ccc(-c2ccc(-n3c4ccccc4c4c5c6ccccc6n(-c6ccc7ccccc7c6)c5ccc43)cc2)cc1]

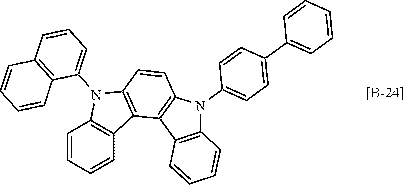 [SMILES: c1ccc(-c2ccc(-n3c4ccccc4c4c5c6ccccc6n(-c6cccc7ccccc67)c5ccc43)cc2)cc1]

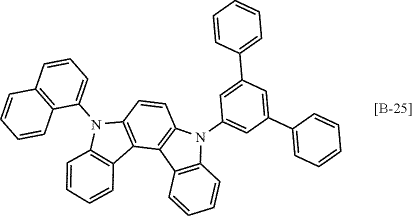 [SMILES: c1ccc(-c2cc(-c3ccccc3)cc(-n3c4ccccc4c4c5c6ccccc6n(-c6cccc7ccccc67)c5ccc43)c2)cc1]

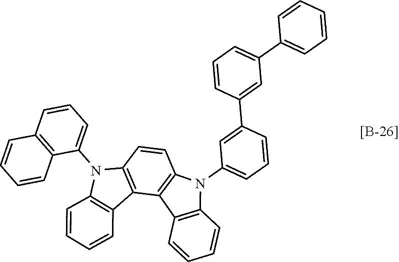 [SMILES: c1ccc(-c2cccc(-c3cccc(-n4c5ccccc5c5c6c7ccccc7n(-c7cccc8ccccc78)c6ccc54)c3)c2)cc1]

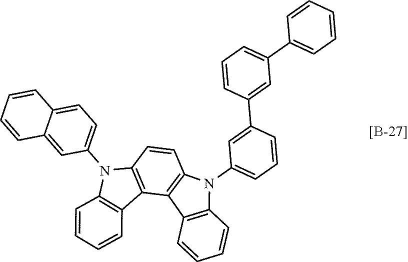 [SMILES: c1ccc(-c2cccc(-c3cccc(-n4c5ccccc5c5c6c7ccccc7n(-c7ccc8ccccc8c7)c6ccc54)c3)c2)cc1]

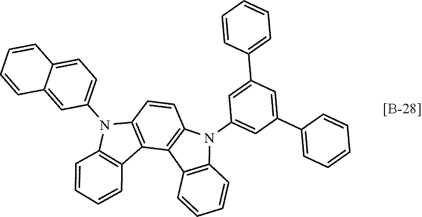 [SMILES: c1ccc(-c2cc(-c3ccccc3)cc(-n3c4ccccc4c4c5c6ccccc6n(-c6ccc7ccccc7c6)c5ccc43)c2)cc1]

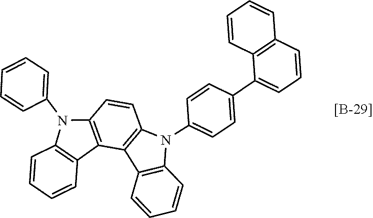 [SMILES: c1ccc(-n2c3ccccc3c3c4c5ccccc5n(-c5ccc(-c6cccc7ccccc67)cc5)c4ccc32)cc1]

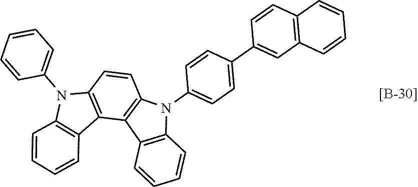 [SMILES: c1ccc(-n2c3ccccc3c3c4c5ccccc5n(-c5ccc(-c6ccc7ccccc7c6)cc5)c4ccc32)cc1]

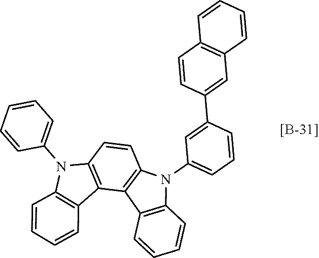 [SMILES: c1ccc(-n2c3ccccc3c3c4c5ccccc5n(-c5cccc(-c6ccc7ccccc7c6)c5)c4ccc32)cc1]

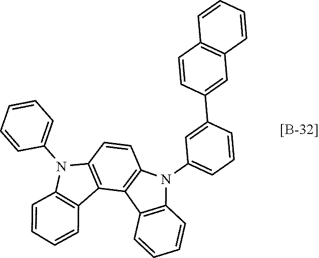 [SMILES: c1ccc(-n2c3ccccc3c3c4c5ccccc5n(-c5cccc(-c6ccc7ccccc7c6)c5)c4ccc32)cc1]

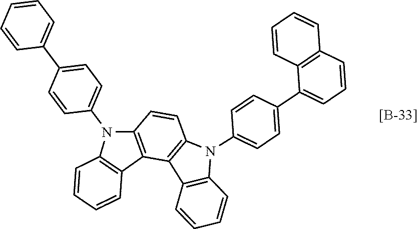 [SMILES: c1ccc(-c2ccc(-n3c4ccccc4c4c5c6ccccc6n(-c6ccc(-c7cccc8ccccc78)cc6)c5ccc43)cc2)cc1]

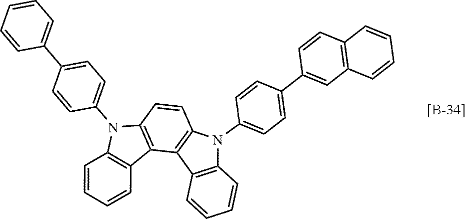 [SMILES: c1ccc(-c2ccc(-n3c4ccccc4c4c5c6ccccc6n(-c6ccc(-c7ccc8ccccc8c7)cc6)c5ccc43)cc2)cc1]

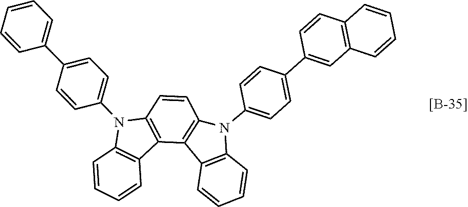 [SMILES: c1ccc(-c2ccc(-n3c4ccccc4c4c5c6ccccc6n(-c6ccc(-c7ccc8ccccc8c7)cc6)c5ccc43)cc2)cc1]

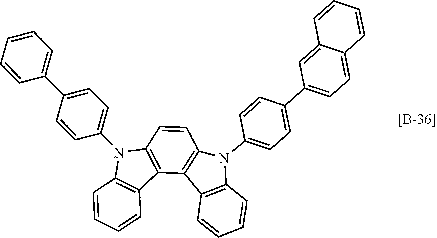 [SMILES: c1ccc(-c2ccc(-n3c4ccccc4c4c5c6ccccc6n(-c6ccc(-c7ccc8ccccc8c7)cc6)c5ccc43)cc2)cc1]

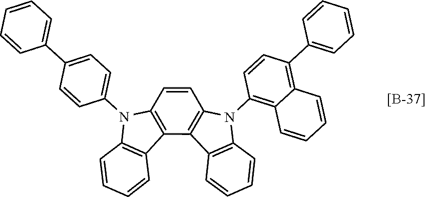 [SMILES: c1ccc(-c2ccc(-n3c4ccccc4c4c5c6ccccc6n(-c6ccc(-c7ccccc7)c7ccccc67)c5ccc43)cc2)cc1]

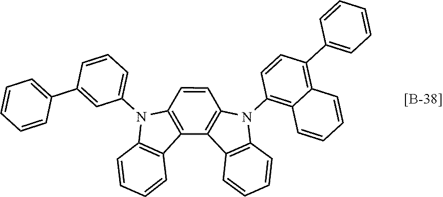 [SMILES: c1ccc(-c2cccc(-n3c4ccccc4c4c5c6ccccc6n(-c6ccc(-c7ccccc7)c7ccccc67)c5ccc43)c2)cc1]

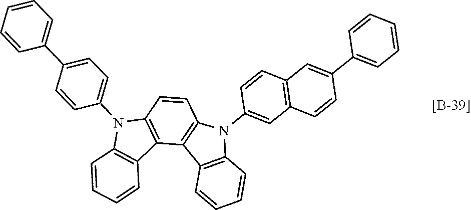 [SMILES: c1ccc(-c2ccc(-n3c4ccccc4c4c5c6ccccc6n(-c6ccc7cc(-c8ccccc8)ccc7c6)c5ccc43)cc2)cc1]

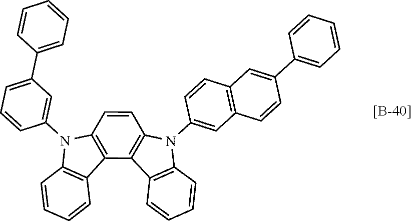 [SMILES: c1ccc(-c2cccc(-n3c4ccccc4c4c5c6ccccc6n(-c6ccc7cc(-c8ccccc8)ccc7c6)c5ccc43)c2)cc1]

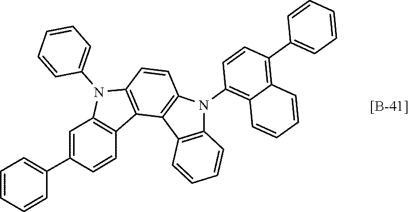 [SMILES: c1ccc(-c2ccc3c4c5c6ccccc6n(-c6ccc(-c7ccccc7)c7ccccc67)c5ccc4n(-c4ccccc4)c3c2)cc1]

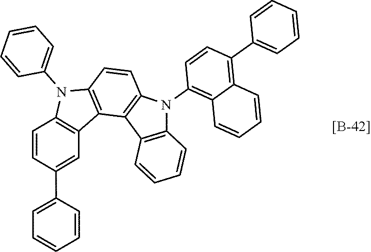 [SMILES: c1ccc(-c2ccc3c(c2)c2c4c5ccccc5n(-c5ccc(-c6ccccc6)c6ccccc56)c4ccc2n3-c2ccccc2)cc1]

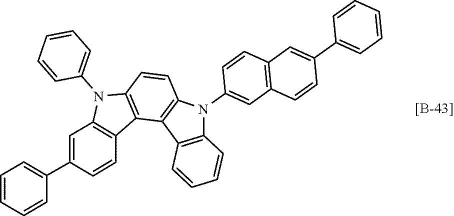 [SMILES: c1ccc(-c2ccc3cc(-n4c5ccccc5c5c6c7ccc(-c8ccccc8)cc7n(-c7ccccc7)c6ccc54)ccc3c2)cc1]

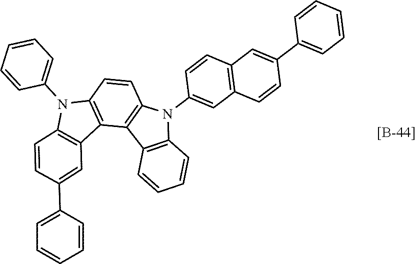 [SMILES: c1ccc(-c2ccc3cc(-n4c5ccccc5c5c6c7cc(-c8ccccc8)ccc7n(-c7ccccc7)c6ccc54)ccc3c2)cc1]

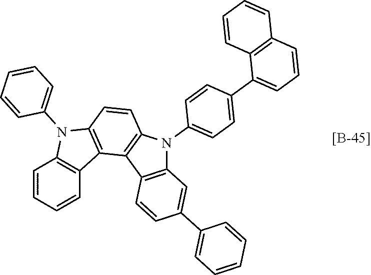 [SMILES: c1ccc(-c2ccc3c4c5c6ccccc6n(-c6ccccc6)c5ccc4n(-c4ccc(-c5cccc6ccccc56)cc4)c3c2)cc1]

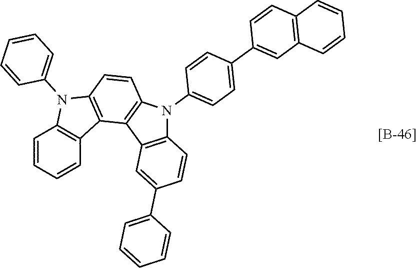 [SMILES: c1ccc(-c2ccc3c(c2)c2c4c5ccccc5n(-c5ccccc5)c4ccc2n3-c2ccc(-c3ccc4ccccc4c3)cc2)cc1]

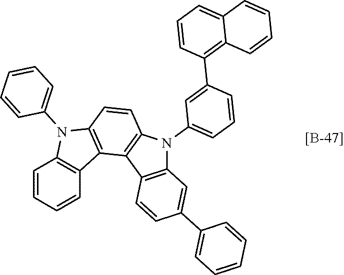 [SMILES: c1ccc(-c2ccc3c4c5c6ccccc6n(-c6ccccc6)c5ccc4n(-c4cccc(-c5cccc6ccccc56)c4)c3c2)cc1]

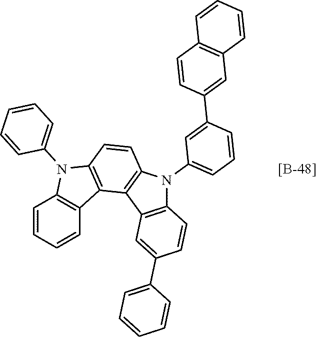 [SMILES: c1ccc(-c2ccc3c(c2)c2c4c5ccccc5n(-c5ccccc5)c4ccc2n3-c2cccc(-c3ccc4ccccc4c3)c2)cc1]

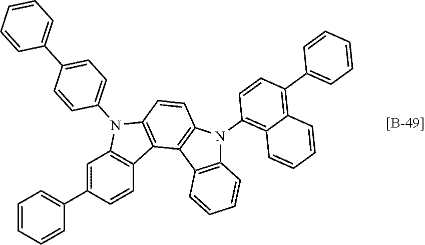 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1]

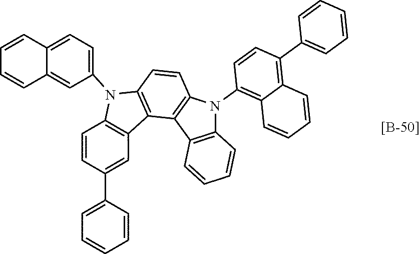 [SMILES: c1ccc(-c2ccc3c(c2)c2c4c5ccccc5n(-c5ccc(-c6ccccc6)c6ccccc56)c4ccc2n3-c2ccc3ccccc3c2)cc1]

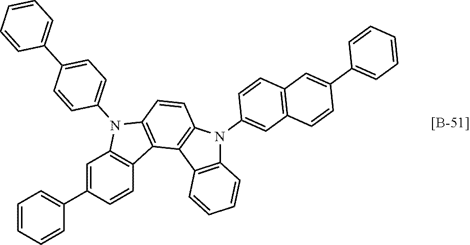 [SMILES: c1ccc(-c2ccc(-n3c4cc(-c5ccccc5)ccc4c4c5c6ccccc6n(-c6ccc7cc(-c8ccccc8)ccc7c6)c5ccc43)cc2)cc1]

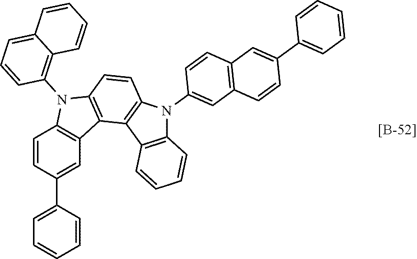 [SMILES: c1ccc(-c2ccc3cc(-n4c5ccccc5c5c6c7cc(-c8ccccc8)ccc7n(-c7cccc8ccccc78)c6ccc54)ccc3c2)cc1]

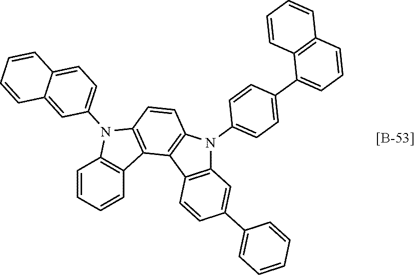 [SMILES: c1ccc(-c2ccc3c4c5c6ccccc6n(-c6ccc7ccccc7c6)c5ccc4n(-c4ccc(-c5cccc6ccccc56)cc4)c3c2)cc1]

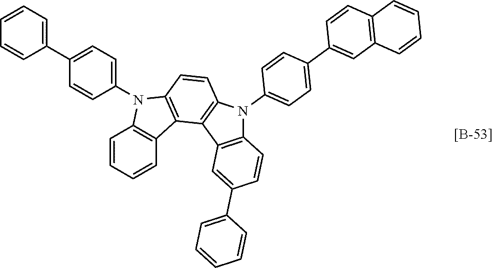 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1]

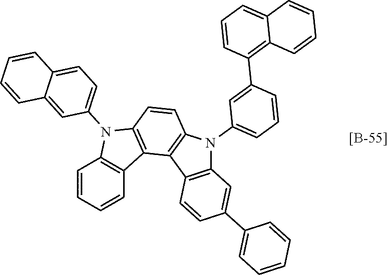 [SMILES: c1ccc(-c2ccc3c4c5c6ccccc6n(-c6ccc7ccccc7c6)c5ccc4n(-c4cccc(-c5cccc6ccccc56)c4)c3c2)cc1]

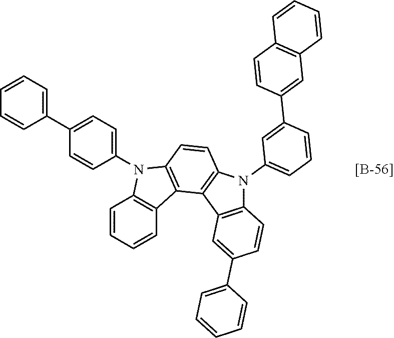 [SMILES: c1ccc(-c2ccc(-n3c4ccccc4c4c5c6cc(-c7ccccc7)ccc6n(-c6cccc(-c7ccc8ccccc8c7)c6)c5ccc43)cc2)cc1]

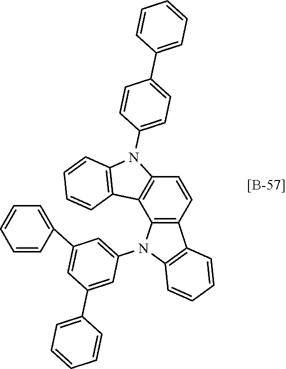 [SMILES: c1ccc(-c2ccc(-n3c4ccccc4c4c3ccc3c5ccccc5n(-c5cc(-c6ccccc6)cc(-c6ccccc6)c5)c34)cc2)cc1]

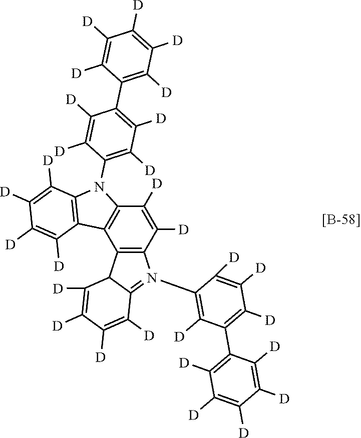

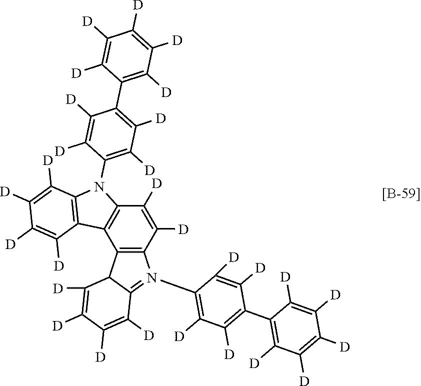

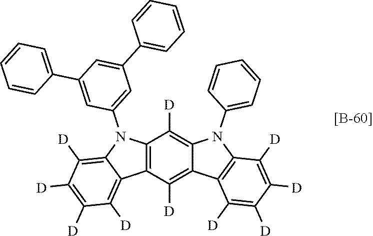 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1]

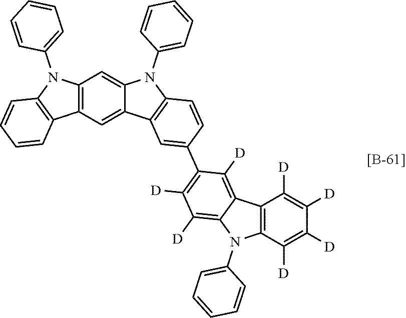 [SMILES: [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1]

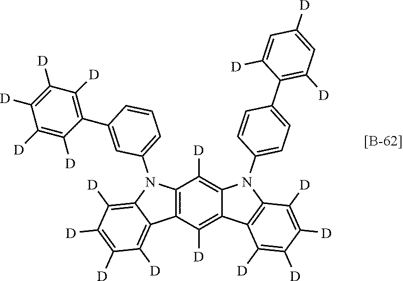 [SMILES: [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

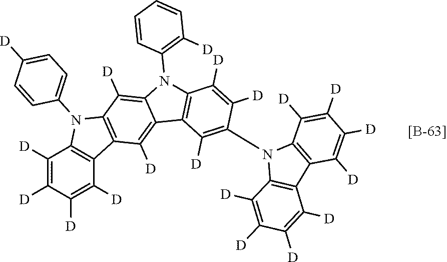 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1]

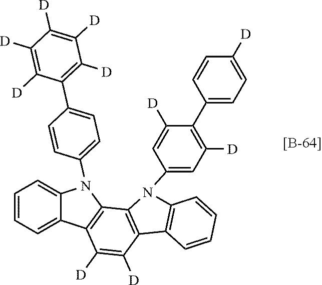 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1]

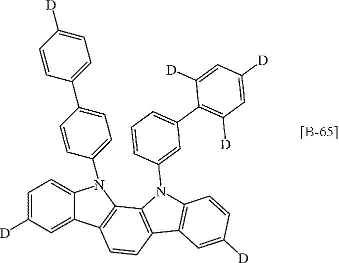 [SMILES: [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

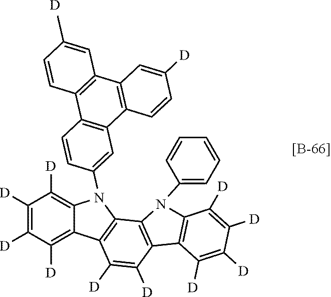

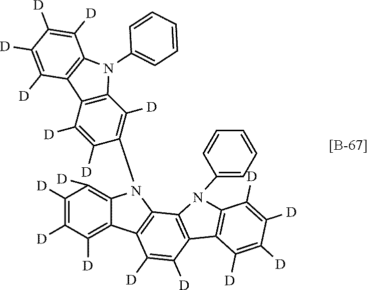 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1]

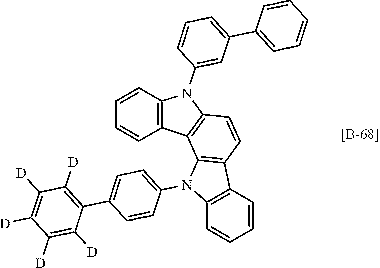 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H]]

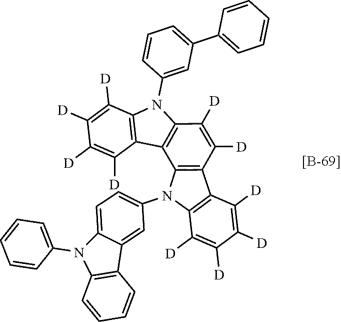 [SMILES: [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1]

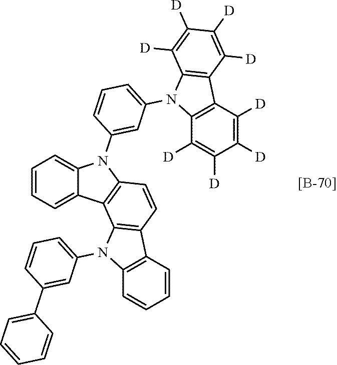 [SMILES: [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

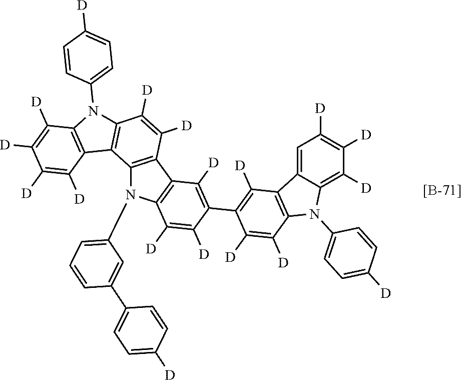 [SMILES: [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1]

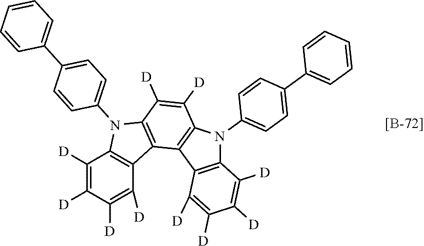 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

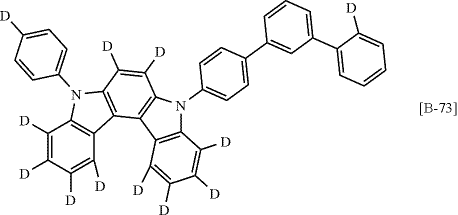 [SMILES: [2H]c1ccc(-n2c3c([2H])c([2H])c([2H])c([2H])c3c3c4c5c([2H])c([2H])c([2H])c([2H])c5n(-c5ccc(-c6cccc(-c7ccccc7[2H])c6)cc5)c4c([2H])c([2H])c32)cc1]

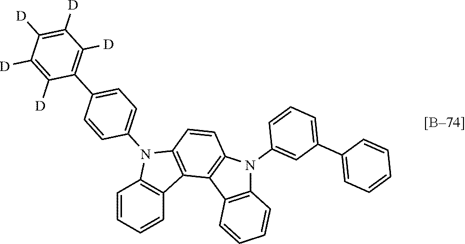 [SMILES: [2H]c1c([2H])c([2H])c(-c2ccc(-n3c4ccccc4c4c5c6ccccc6n(-c6cccc(-c7ccccc7)c6)c5ccc43)cc2)c([2H])c1[2H]]

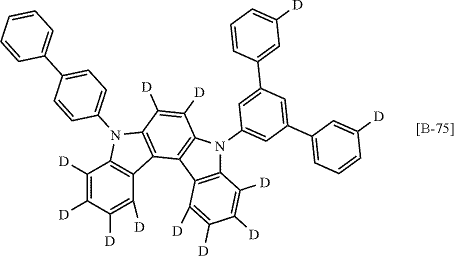 [SMILES: [2H]c1cccc(-c2cc(-c3cccc([2H])c3)cc(-n3c4c([2H])c([2H])c([2H])c([2H])c4c4c5c6c([2H])c([2H])c([2H])c([2H])c6n(-c6ccc(-c7ccccc7)cc6)c5c([2H])c([2H])c43)c2)c1]

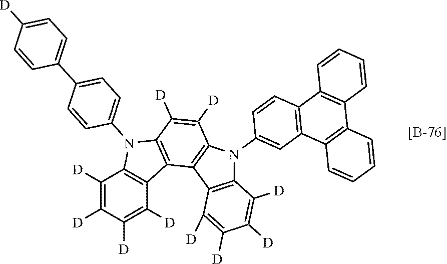 [SMILES: [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1]

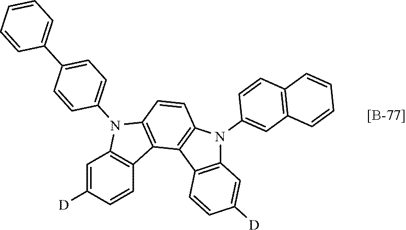 [SMILES: [2H]c1ccc2c3c4c5ccc([2H])cc5n(-c5ccc6ccccc6c5)c4ccc3n(-c3ccc(-c4ccccc4)cc3)c2c1]

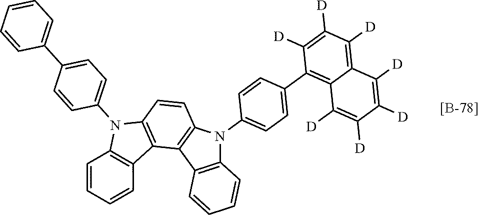 [SMILES: [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H]]

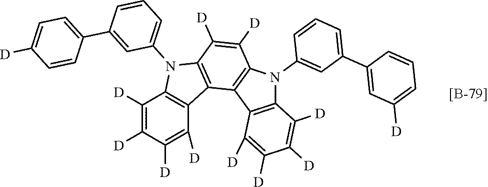 [SMILES: [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1]

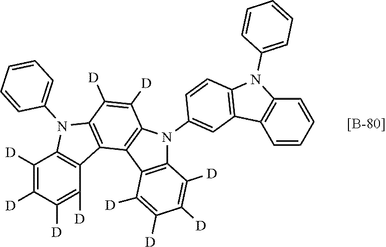 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1]

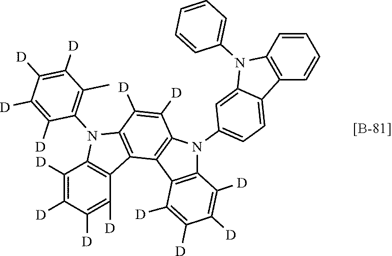 [SMILES: [2H]c1c([2H])c([2H])c(-n2c3c([2H])c([2H])c([2H])c([2H])c3c3c4c5c([2H])c([2H])c([2H])c([2H])c5n(-c5ccc6c7ccccc7n(-c7ccccc7)c6c5)c4c([2H])c([2H])c32)c(C)c1[2H]]

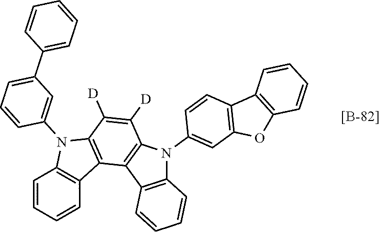 [SMILES: [2H]c1c([2H])c2c(c3ccccc3n2-c2ccc3c(c2)oc2ccccc23)c2c3ccccc3n(-c3cccc(-c4ccccc4)c3)c12]

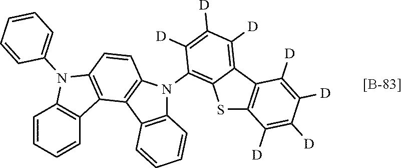 [SMILES: [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

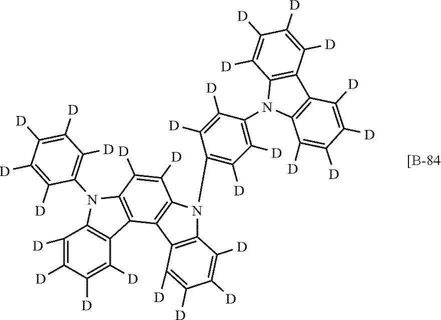 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H]]

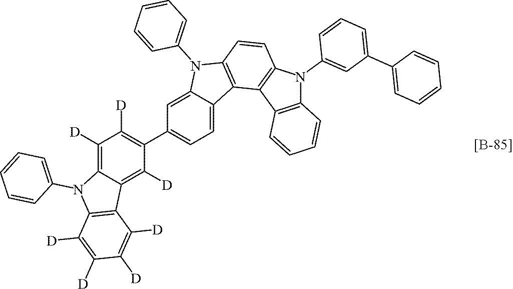 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1]

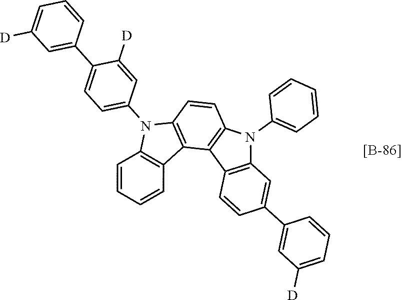 [SMILES: [2H]c1cccc(-c2ccc3c4c5c6ccccc6n(-c6ccc(-c7cccc([2H])c7)c([2H])c6)c5ccc4n(-c4ccccc4)c3c2)c1]

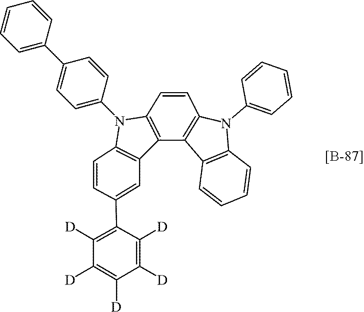 [SMILES: [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

The first compound and the second compound may be, e.g., included (e.g., mixed) in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. Within the range, they may be, e.g., included in a weight ratio of about 10:90 to about 90:10, about 20:80 to about 80:20, about 20:80 to about 70: 30, about 20:80 to about 60:40, or about 20:80 to about 50:50. In an implementation, they may be included in a weight ratio of, e.g., about 20:80, about 30:70, or about 40:60.

One or more compounds may be further included in addition to the aforementioned first and second compounds.

In an implementation, the aforementioned compound for the organic optoelectronic device or composition for the organic optoelectronic device may further include a dopant.

The dopant may be, e.g., a phosphorescent dopant, such as a red, green, or blue phosphorescent dopant, and may be, e.g., a red or green phosphorescent dopant.

The dopant is a material mixed with the compound in a small amount to cause light emission and may be generally a material such as a metal complex that emits light by multiple excitation into a triplet or more. The dopant may be, e.g., an inorganic, organic, or organic-inorganic compound, and one or more types thereof may be used.

The dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

[Chemical Formula Z]

L⁵MX²

In Chemical Formula Z, M may be, e.g., a metal, and L⁵ and X² are the same as or different from each other, and may be, e.g., ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁵ and X² may be, e.g., a bidentate ligand.

The ligands represented by L⁵ and X² may include, e.g., ligands of Group A.

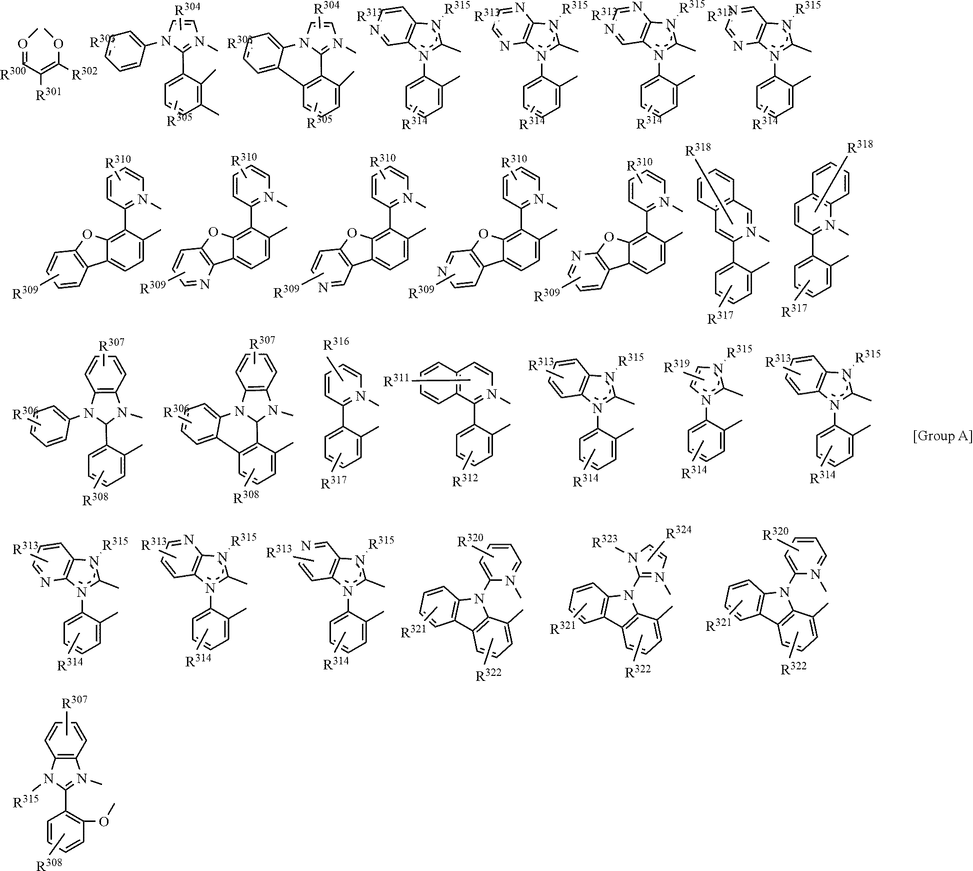

In Group A,

R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, the dopant may be, e.g., represented by Chemical Formula V.

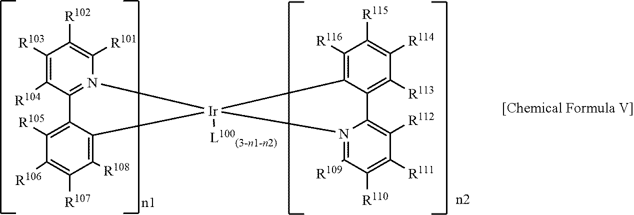

In Chemical Formula V,
R¹⁰¹ to R¹¹⁶ may each independently be, e.g., hydrogen, deuterium, a substituted or unsubstituted C1 to C10 alkyl group, a substituted or unsubstituted C6 to C20 aryl group, or -SiR¹³²R¹³³R¹³⁴,
R¹³² to R¹³⁴ may each independently be, e.g., a substituted or unsubstituted C1 to C6 alkyl group,
at least one of R¹⁰¹ to R¹¹⁶ may be, e.g., a functional group represented by Chemical Formula V-1,
L¹⁰⁰ may be, e.g., a bidentate ligand of a monovalent anion, and is a ligand that coordinates to iridium through a lone pair of electrons of carbon or heteroatom,
n1 and n2 may each independently be, e.g., an integer of 0 to 3, and
n1 + n2 may be, e.g., an integer of 1 to 3.

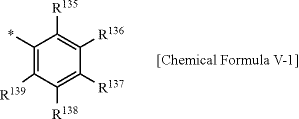

In Chemical Formula V-1,
R¹³⁵ to R¹³⁹ may each independently be, e.g., hydrogen, deuterium, a substituted or unsubstituted C1 to C10 alkyl group, a substituted or unsubstituted C6 to C20 aryl group, or -SiR¹³²R¹³³R¹³⁴, and
* means a portion linked to a carbon atom.
In an implementation, a dopant represented by Chemical Formula Z-1 may be included. [00196]

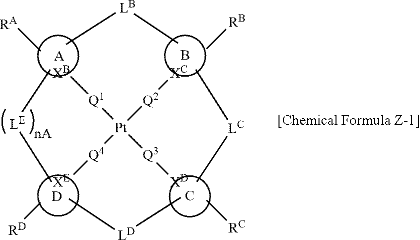

In Chemical Formula Z-1, rings A, B, C, and D may each independently be, e.g., a 5-membered or 6-membered carbocyclic or heterocyclic ring;
R^{A}, R^{B}, R^{C}, and R^{D} may each independently be, e.g., mono-, di-, tri-, or tetra-substitution, or unsubstitution;
L^{B}, L^{C}, and L^{D} may each independently be, e.g., a direct bond, BR, NR, PR, O, S, Se, C═O, S═O, SO₂, CRR′, SiRR′, GeRR′, or a combination thereof;
when nA is 1, L^{E} may be, e.g., a direct bond, BR, NR, PR, O, S, Se, C═O, S═O, SO₂, CRR′, SiRR′, GeRR′, or a combination thereof; when nA is 0, L^{E} may not exist; and
R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} may each independently be selected from carbon and nitrogen; and Q¹, Q², Q³, and Q⁴ may each independently be oxygen or a direct bond.

The dopant according to an embodiment may be a platinum complex, and may be, e.g., represented by Chemical Formula VI.

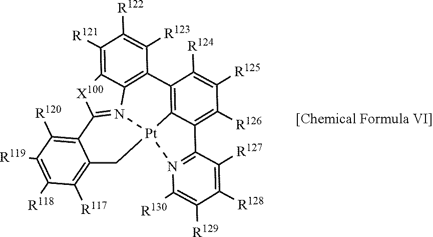

In Chemical Formula VI,
X¹⁰⁰ may be, e.g., O, S, or NR¹³¹,
R¹¹⁷ to R¹³¹ may each independently be, e.g., hydrogen, deuterium, a substituted or unsubstituted C1 to C10 alkyl group, a substituted or unsubstituted C6 to C20 aryl group, or -SiR¹³²R¹³³R¹³⁴,
R¹³² to R¹³⁴ may each independently be, e.g., a C1 to C6 alkyl group, and
at least one of R¹¹⁷ to R¹³¹ may be, e.g., -SiR¹³²R¹³³R¹³⁴ or a tert-butyl group.

Hereinafter, an organic optoelectronic device including the aforementioned compound for the organic optoelectronic device or composition for the organic optoelectronic device is described.

The organic optoelectronic device may be a suitable device to convert electrical energy into photoenergy and vice versa, and may be, e.g., an organic photoelectric device, an organic light emitting diode, an organic solar cell, or an organic photo-conductor drum.

Herein, an organic light emitting diode as one example of an organic optoelectronic device is described referring to the drawing.

The FIGURE is a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for the organic optoelectronic device or composition for the organic optoelectronic device.

The organic layer 105 may include a light emitting layer 130 and the light emitting layer 130 may include the aforementioned compound for the organic optoelectronic device or composition for the organic optoelectronic device.

The composition for the organic optoelectronic device further including the dopant may be, e.g., a green light emitting composition.

The light emitting layer 130 may include, e.g., the aforementioned compound for the organic optoelectronic device or composition for the organic optoelectronic device as a phosphorescent host.

The organic layer may further include a charge transport region in addition to the light emitting layer.

The charge transport region may be, e.g., a hole transport region 140.

The hole transport region 140 may help further increase hole injection and/or hole mobility between the anode 120 and the light emitting layer 130 and block electrons. In an implementation, the hole transport region 140 may include a hole transport layer between the anode 120 and the light emitting layer 130, and a hole transport auxiliary layer between the light emitting layer 130 and the hole transport layer and a compound of Group B may be included in at least one of the hole transport layer and the hole transport auxiliary layer.

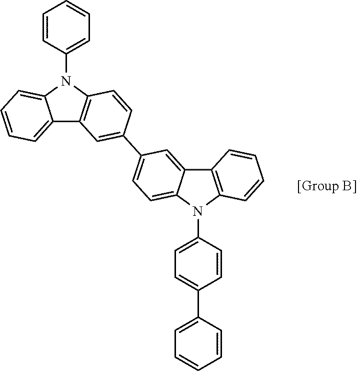 [SMILES: c1ccc(-c2ccc(-n3c4ccccc4c4cc(-c5ccc6c(c5)c5ccccc5n6-c5ccccc5)ccc43)cc2)cc1]

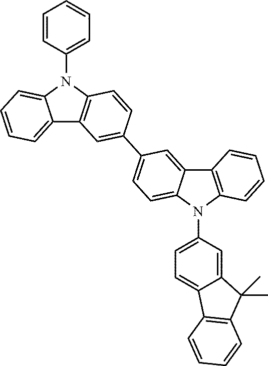 [SMILES: CC1(C)c2ccccc2-c2ccc(-n3c4ccccc4c4cc(-c5ccc6c(c5)c5ccccc5n6-c5ccccc5)ccc43)cc21]

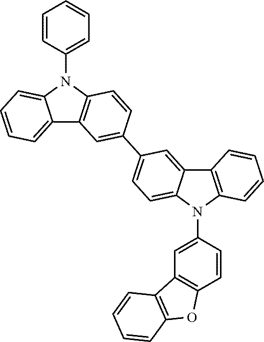 [SMILES: c1ccc(-n2c3ccccc3c3cc(-c4ccc5c(c4)c4ccccc4n5-c4ccc5oc6ccccc6c5c4)ccc32)cc1]

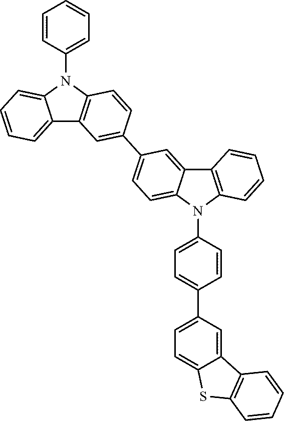 [SMILES: c1ccc(-n2c3ccccc3c3cc(-c4ccc5c(c4)c4ccccc4n5-c4ccc(-c5ccc6sc7ccccc7c6c5)cc4)ccc32)cc1]

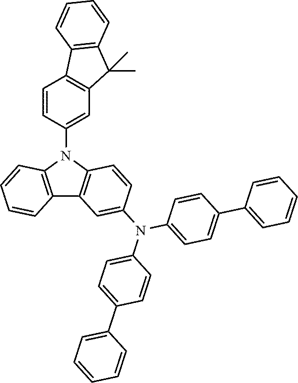 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21]

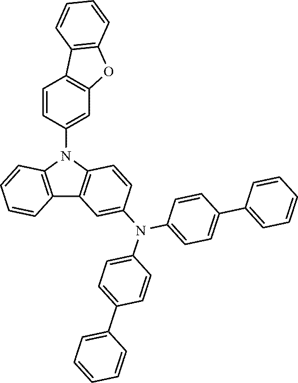 [SMILES: c1ccc(-c2ccc(N(c3ccc(-c4ccccc4)cc3)c3ccc4c(c3)c3ccccc3n4-c3ccc4c(c3)oc3ccccc34)cc2)cc1]

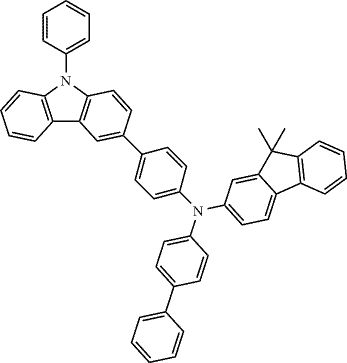 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

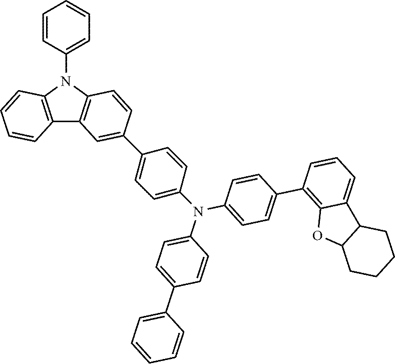 [SMILES: c1ccc(-c2ccc(N(c3ccc(-c4ccc5c(c4)c4ccccc4n5-c4ccccc4)cc3)c3ccc(-c4cccc5c4OC4CCCCC54)cc3)cc2)cc1]

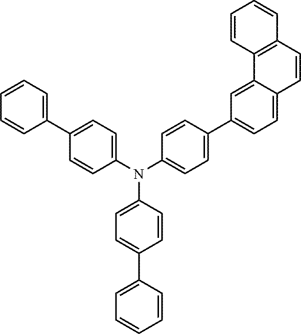 [SMILES: c1ccc(-c2ccc(N(c3ccc(-c4ccccc4)cc3)c3ccc(-c4ccc5ccc6ccccc6c5c4)cc3)cc2)cc1]

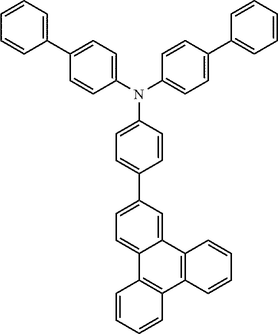 [SMILES: c1ccc(-c2ccc(N(c3ccc(-c4ccccc4)cc3)c3ccc(-c4ccc5c6ccccc6c6ccccc6c5c4)cc3)cc2)cc1]

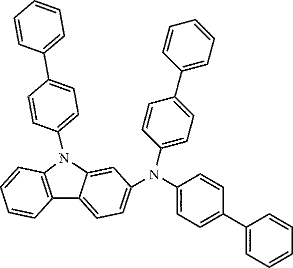 [SMILES: c1ccc(-c2ccc(N(c3ccc(-c4ccccc4)cc3)c3ccc4c5ccccc5n(-c5ccc(-c6ccccc6)cc5)c4c3)cc2)cc1]

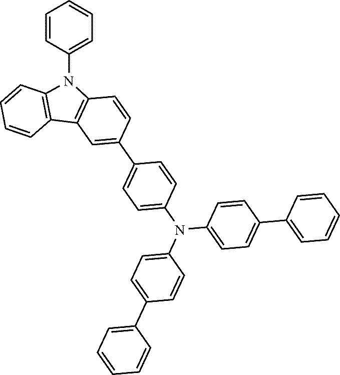 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

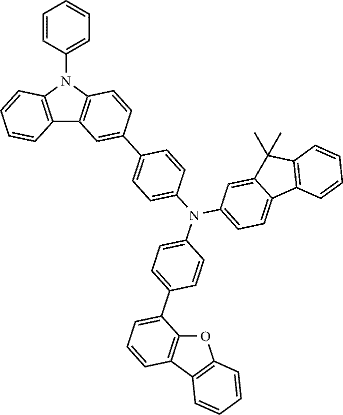 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

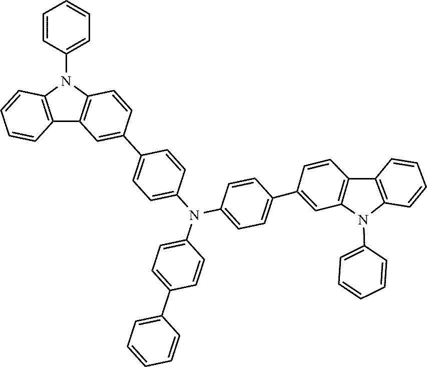 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

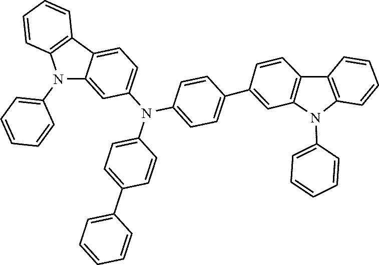 [SMILES: c1ccc(-c2ccc(N(c3ccc(-c4ccc5c6ccccc6n(-c6ccccc6)c5c4)cc3)c3ccc4c5ccccc5n(-c5ccccc5)c4c3)cc2)cc1]

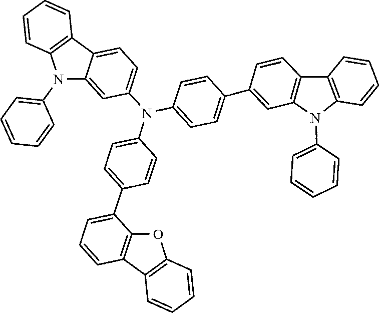 [SMILES: c1ccc(-n2c3ccccc3c3ccc(-c4ccc(N(c5ccc(-c6cccc7c6oc6ccccc67)cc5)c5ccc6c7ccccc7n(-c7ccccc7)c6c5)cc4)cc32)cc1]

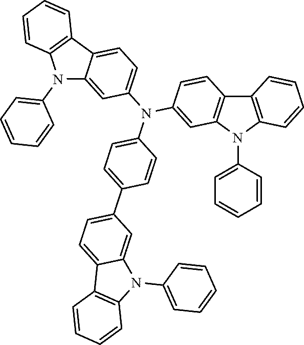 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

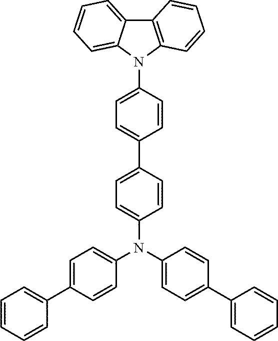 [SMILES: c1ccc(-c2ccc(N(c3ccc(-c4ccccc4)cc3)c3ccc(-c4ccc(-n5c6ccccc6c6ccccc65)cc4)cc3)cc2)cc1]

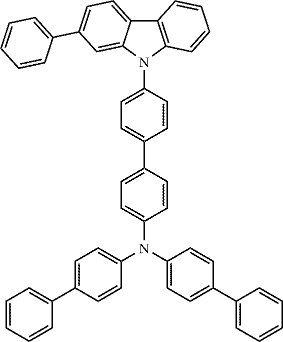 [SMILES: c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

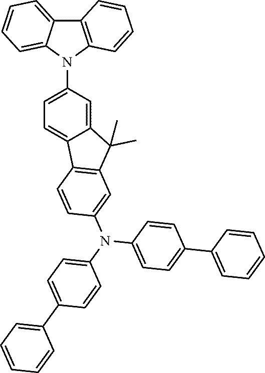 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21]

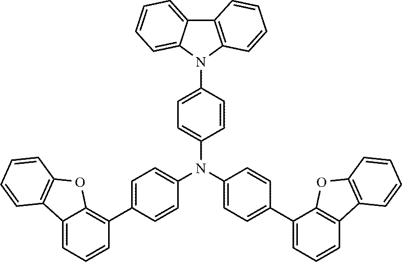 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12]

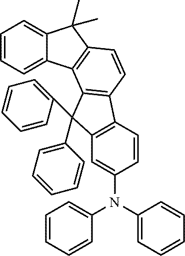 [SMILES: CC1(C)c2ccccc2-c2c1ccc1c2C(c2ccccc2)(c2ccccc2)c2cc(N(c3ccccc3)c3ccccc3)ccc2-1]

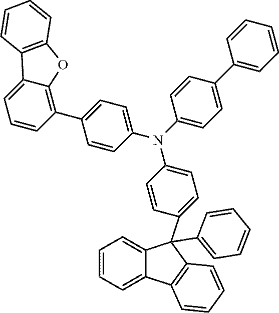 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1]

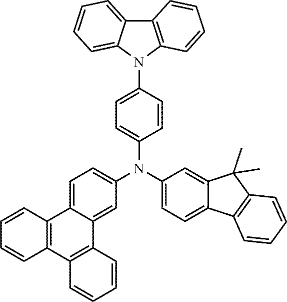 [SMILES: CC1(C)c2ccccc2-c2ccc(N(c3ccc(-n4c5ccccc5c5ccccc54)cc3)c3ccc4c5ccccc5c5ccccc5c4c3)cc21]

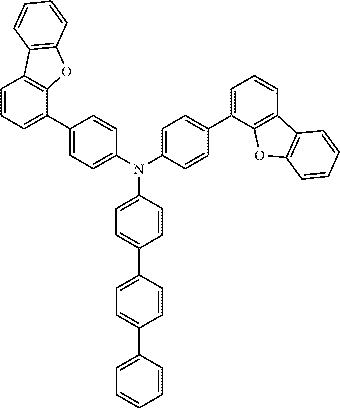 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

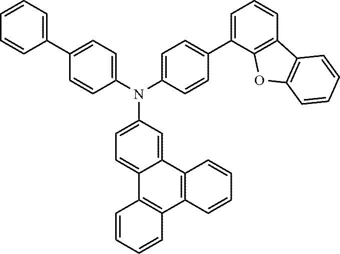 [SMILES: c1ccc(-c2ccc(N(c3ccc(-c4cccc5c4oc4ccccc45)cc3)c3ccc4c5ccccc5c5ccccc5c4c3)cc2)cc1]

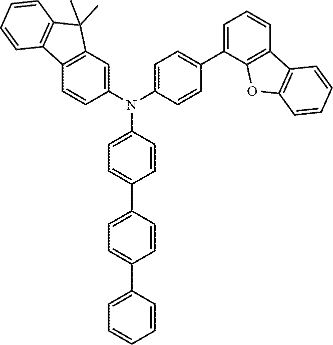 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21]

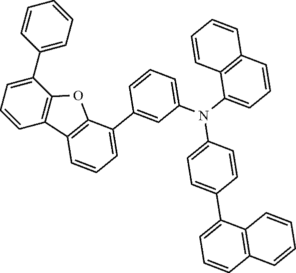 [SMILES: c1ccc(-c2cccc3c2oc2c(-c4cccc(N(c5ccc(-c6cccc7ccccc67)cc5)c5cccc6ccccc56)c4)cccc23)cc1]

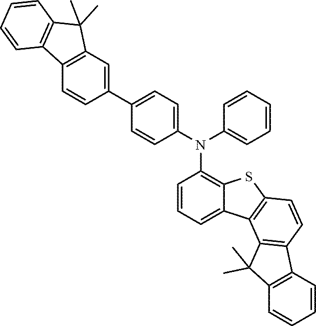 [SMILES: CC1(C)c2ccccc2-c2ccc(-c3ccc(N(c4ccccc4)c4cccc5c4sc4ccc6c(c45)C(C)(C)c4ccccc4-6)cc3)cc21]

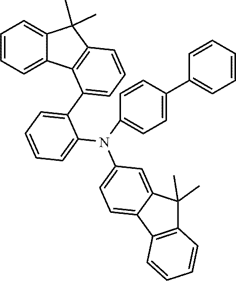 [SMILES: CC1(C)c2ccccc2-c2ccc(N(c3ccc(-c4ccccc4)cc3)c3ccccc3-c3cccc4c3-c3ccccc3C4(C)C)cc21]

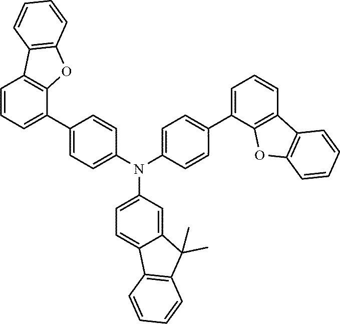 [SMILES: CC1(C)c2ccccc2-c2ccc(N(c3ccc(-c4cccc5c4oc4ccccc45)cc3)c3ccc(-c4cccc5c4oc4ccccc45)cc3)cc21]

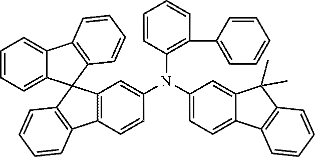 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

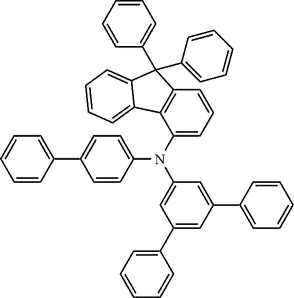 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1]

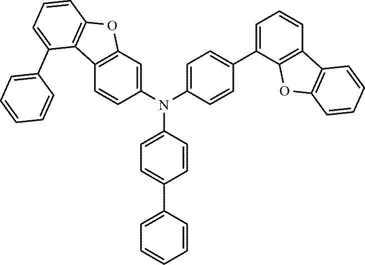 [SMILES: c1ccc(-c2ccc(N(c3ccc(-c4cccc5c4oc4ccccc45)cc3)c3ccc4c(c3)oc3cccc(-c5ccccc5)c34)cc2)cc1]

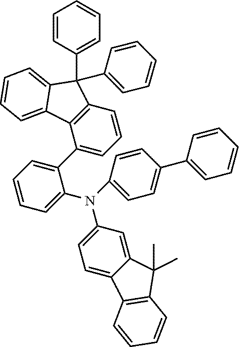 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21]

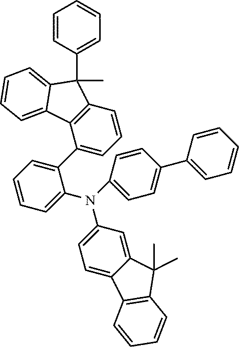 [SMILES: CC1(C)c2ccccc2-c2ccc(N(c3ccc(-c4ccccc4)cc3)c3ccccc3-c3cccc4c3-c3ccccc3C4(C)c3ccccc3)cc21]

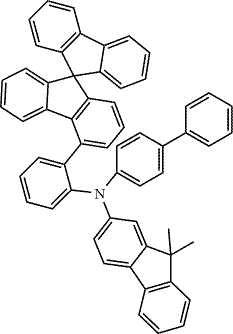 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21]

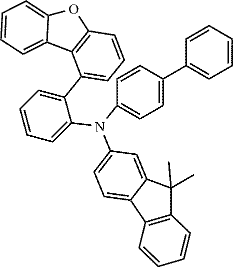 [SMILES: CC1(C)c2ccccc2-c2ccc(N(c3ccc(-c4ccccc4)cc3)c3ccccc3-c3cccc4oc5ccccc5c34)cc21]

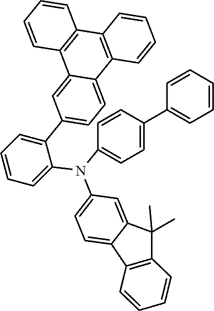 [SMILES: CC1(C)c2ccccc2-c2ccc(N(c3ccc(-c4ccccc4)cc3)c3ccccc3-c3ccc4c5ccccc5c5ccccc5c4c3)cc21]

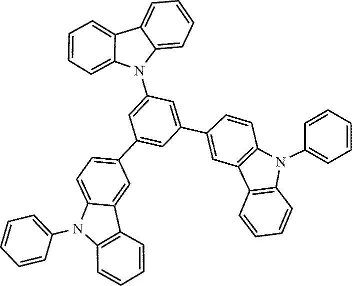 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1]

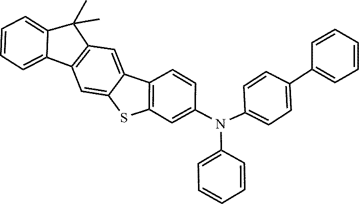 [SMILES: CC1(C)c2ccccc2-c2cc3sc4cc(N(c5ccccc5)c5ccc(-c6ccccc6)cc5)ccc4c3cc21]

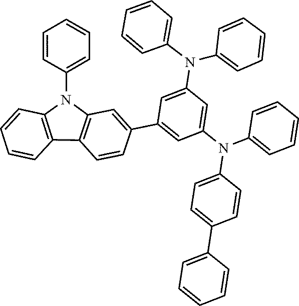 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

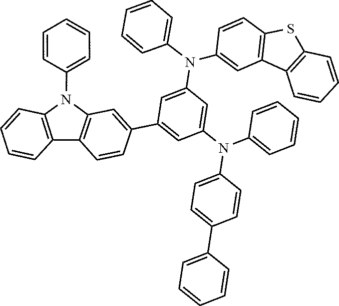 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

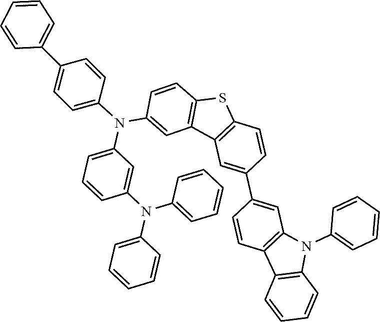 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

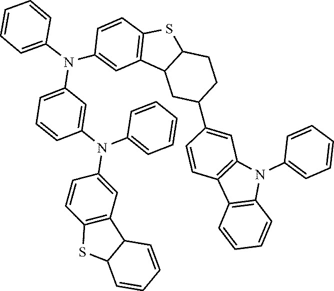 [SMILES: C1=CC2Sc3ccc(N(c4ccccc4)c4cccc(N(c5ccccc5)c5ccc6c(c5)C5CC(c7ccc8c9ccccc9n(-c9ccccc9)c8c7)CCC5S6)c4)cc3C2C=C1]

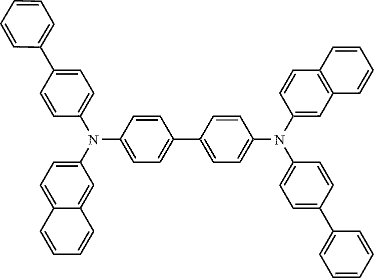 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

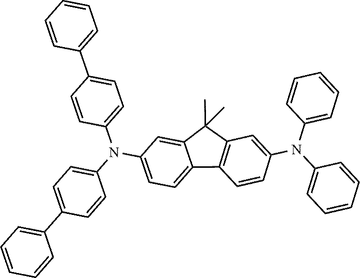 [SMILES: CC1(C)c2cc(N(c3ccccc3)c3ccccc3)ccc2-c2ccc(N(c3ccc(-c4ccccc4)cc3)c3ccc(-c4ccccc4)cc3)cc21]

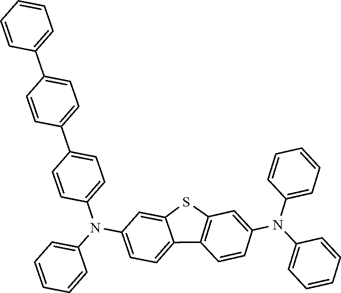 [SMILES: c1ccc(-c2ccc(-c3ccc(N(c4ccccc4)c4ccc5c(c4)sc4cc(N(c6ccccc6)c6ccccc6)ccc45)cc3)cc2)cc1]

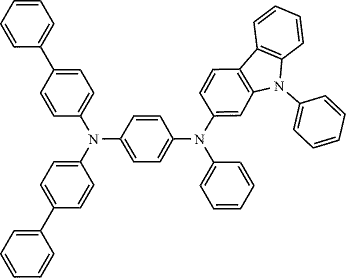 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1]

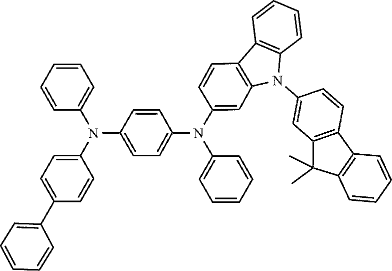 [SMILES: CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

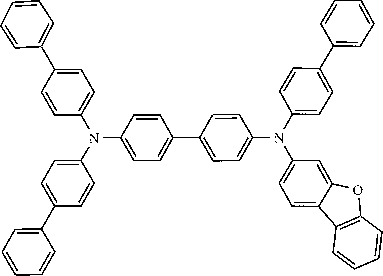 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1]

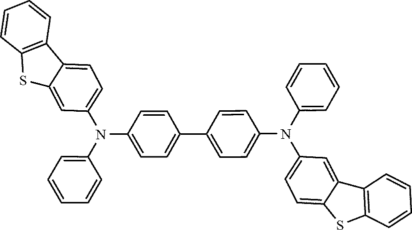 [SMILES: c1ccc(N(c2ccc(-c3ccc(N(c4ccccc4)c4ccc5sc6ccccc6c5c4)cc3)cc2)c2ccc3c(c2)sc2ccccc23)cc1]

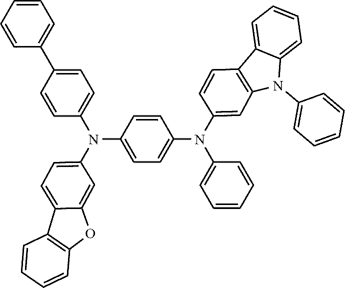 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

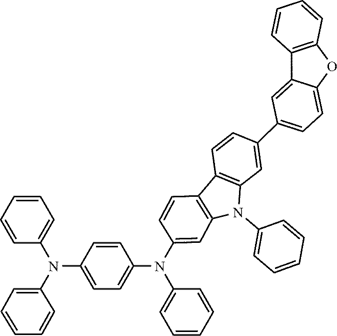 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

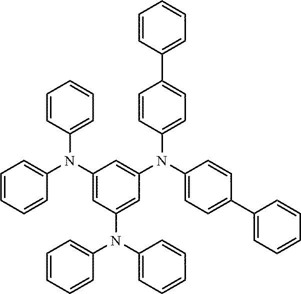 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

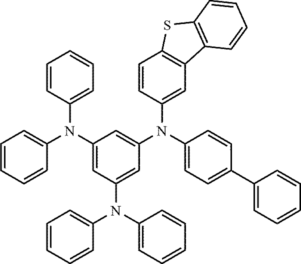 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

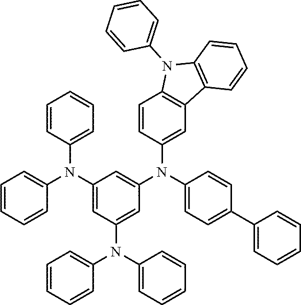 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

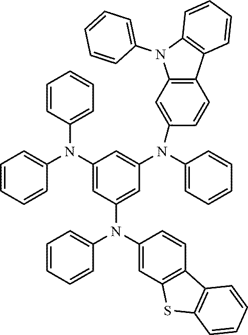 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

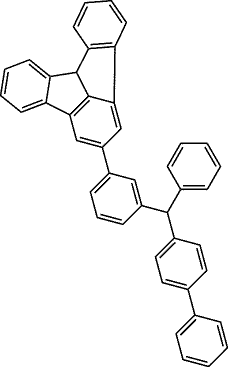 [SMILES: c1ccc(-c2ccc(C(c3ccccc3)c3cccc(-c4cc5c6c(c4)-c4ccccc4C6c4ccccc4-5)c3)cc2)cc1]

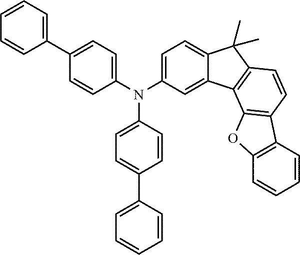 [SMILES: CC1(C)c2ccc(N(c3ccc(-c4ccccc4)cc3)c3ccc(-c4ccccc4)cc3)cc2-c2c1ccc1c2oc2ccccc21]

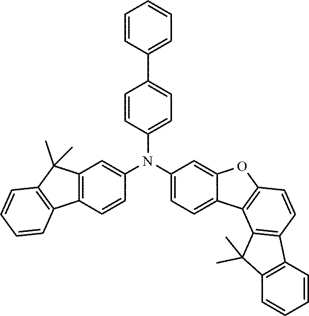 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21]

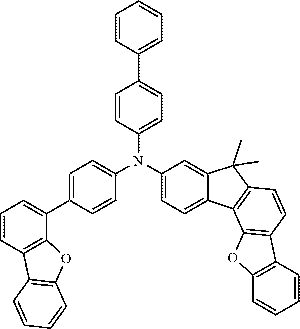 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21]

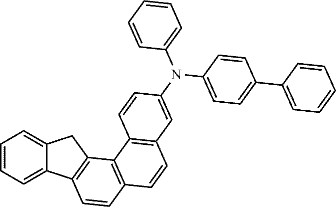 [SMILES: c1ccc(-c2ccc(N(c3ccccc3)c3ccc4c(ccc5ccc6c(c54)Cc4ccccc4-6)c3)cc2)cc1]

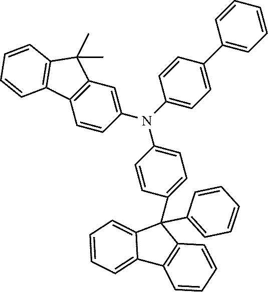 [SMILES: CC1(C)c2ccccc2-c2ccc(N(c3ccc(-c4ccccc4)cc3)c3ccc(C4(c5ccccc5)c5ccccc5-c5ccccc54)cc3)cc21]

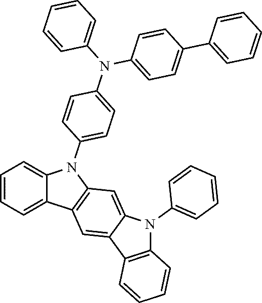 [SMILES: c1ccc(-c2ccc(N(c3ccccc3)c3ccc(-n4c5ccccc5c5cc6c7ccccc7n(-c7ccccc7)c6cc54)cc3)cc2)cc1]

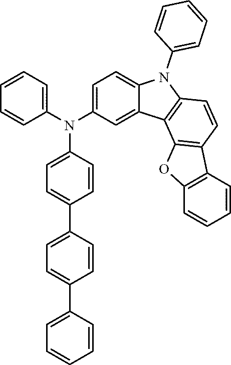 [SMILES: c1ccc(-c2ccc(-c3ccc(N(c4ccccc4)c4ccc5c(c4)c4c6oc7ccccc7c6ccc4n5-c4ccccc4)cc3)cc2)cc1]

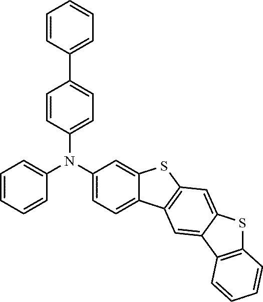 [SMILES: c1ccc(-c2ccc(N(c3ccccc3)c3ccc4c(c3)sc3cc5sc6ccccc6c5cc34)cc2)cc1]

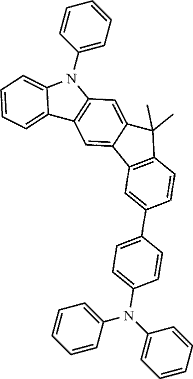 [SMILES: CC1(C)c2ccc(-c3ccc(N(c4ccccc4)c4ccccc4)cc3)cc2-c2cc3c4ccccc4n(-c4ccccc4)c3cc21]

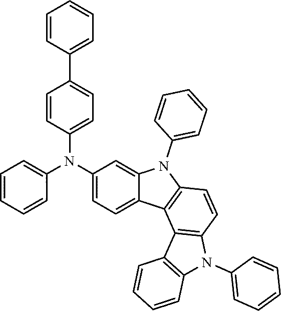 [SMILES: c1ccc(-c2ccc(N(c3ccccc3)c3ccc4c5c6c7ccccc7n(-c7ccccc7)c6ccc5n(-c5ccccc5)c4c3)cc2)cc1]

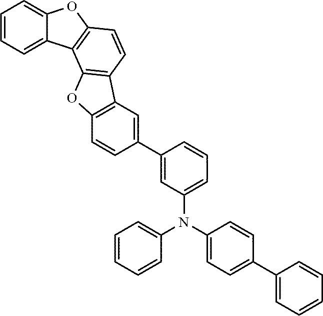 [SMILES: c1ccc(-c2ccc(N(c3ccccc3)c3cccc(-c4ccc5oc6c(ccc7oc8ccccc8c76)c5c4)c3)cc2)cc1]

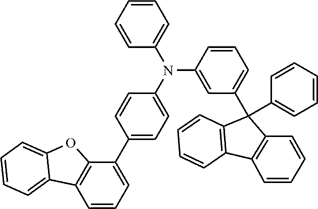 [SMILES: c1ccc(N(c2ccc(-c3cccc4c3oc3ccccc34)cc2)c2cccc(C3(c4ccccc4)c4ccccc4-c4ccccc43)c2)cc1]

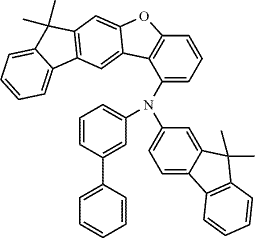 [SMILES: CC1(C)c2ccccc2-c2ccc(N(c3cccc(-c4ccccc4)c3)c3cccc4oc5cc6c(cc5c34)-c3ccccc3C6(C)C)cc21]

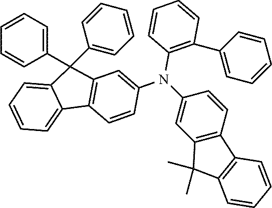 [SMILES: CC1(C)c2ccccc2-c2ccc(N(c3ccc4c(c3)C(c3ccccc3)(c3ccccc3)c3ccccc3-4)c3ccccc3-c3ccccc3)cc21]

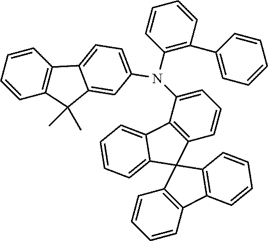 [SMILES: CC1(C)c2ccccc2-c2ccc(N(c3ccccc3-c3ccccc3)c3cccc4c3-c3ccccc3C43c4ccccc4-c4ccccc43)cc21]

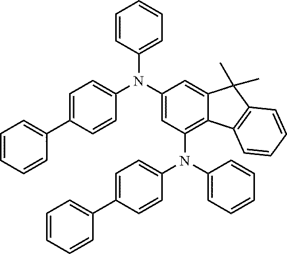 [SMILES: CC1(C)c2ccccc2-c2c(N(c3ccccc3)c3ccc(-c4ccccc4)cc3)cc(N(c3ccccc3)c3ccc(-c4ccccc4)cc3)cc21]

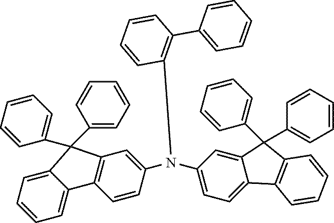 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

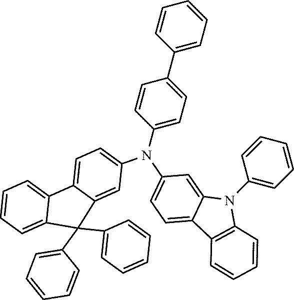 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

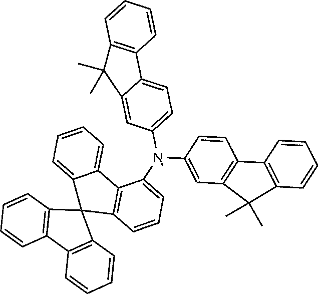

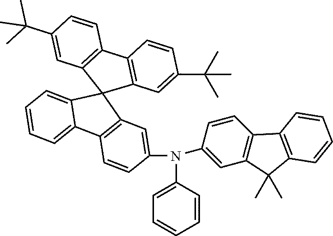 [SMILES: CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

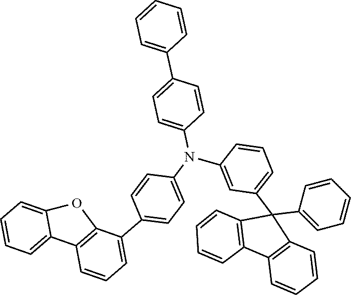 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1]

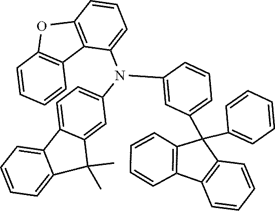 [SMILES: CC1(C)c2ccccc2-c2ccc(N(c3cccc(C4(c5ccccc5)c5ccccc5-c5ccccc54)c3)c3cccc4oc5ccccc5c34)cc21]

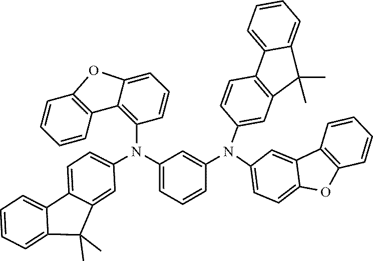 [SMILES: CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21]

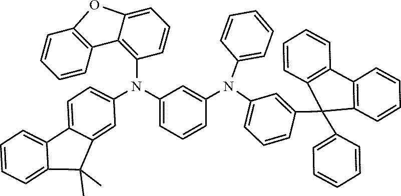 [SMILES: CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

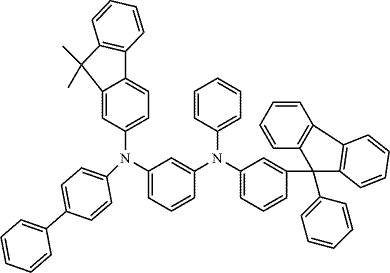 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21]

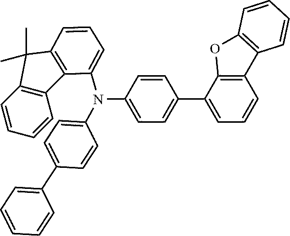 [SMILES: CC1(C)c2ccccc2-c2c(N(c3ccc(-c4ccccc4)cc3)c3ccc(-c4cccc5c4oc4ccccc45)cc3)cccc21]

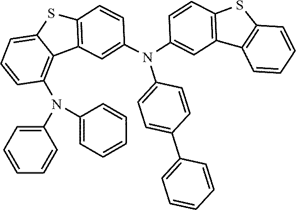 [SMILES: c1ccc(-c2ccc(N(c3ccc4sc5ccccc5c4c3)c3ccc4sc5cccc(N(c6ccccc6)c6ccccc6)c5c4c3)cc2)cc1]

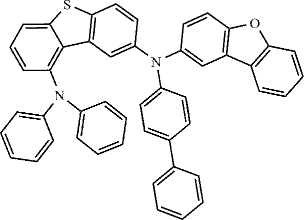 [SMILES: c1ccc(-c2ccc(N(c3ccc4oc5ccccc5c4c3)c3ccc4sc5cccc(N(c6ccccc6)c6ccccc6)c5c4c3)cc2)cc1]

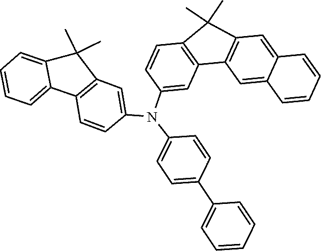 [SMILES: CC1(C)c2ccccc2-c2ccc(N(c3ccc(-c4ccccc4)cc3)c3ccc4c(c3)-c3cc5ccccc5cc3C4(C)C)cc21]

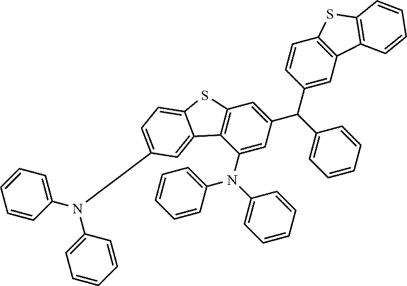 [SMILES: c1ccc(C(c2cc(N(c3ccccc3)c3ccccc3)c3c(c2)sc2ccc(N(c4ccccc4)c4ccccc4)cc23)c2ccc3sc4ccccc4c3c2)cc1]

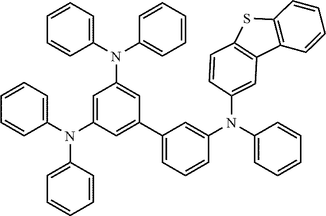 [SMILES: c1ccc(N(c2ccccc2)c2cc(-c3cccc(N(c4ccccc4)c4ccc5sc6ccccc6c5c4)c3)cc(N(c3ccccc3)c3ccccc3)c2)cc1]

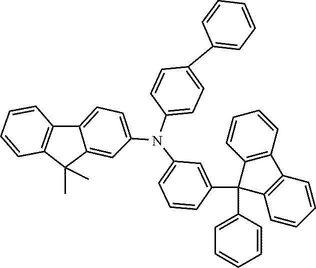 [SMILES: CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable compounds may be used in addition to the compound.

In an implementation, the charge transport region may be, e.g., an electron transport region 150.

The electron transport region 150 may help further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

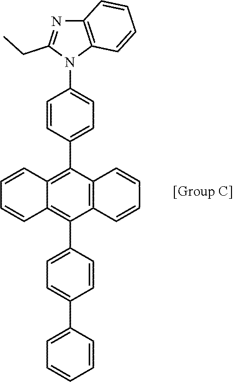 [SMILES: CCc1nc2ccccc2n1-c1ccc(-c2c3ccccc3c(-c3ccc(-c4ccccc4)cc3)c3ccccc23)cc1]

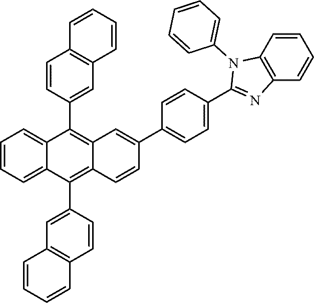 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

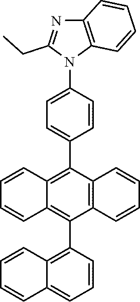 [SMILES: CCc1nc2ccccc2n1-c1ccc(-c2c3ccccc3c(-c3cccc4ccccc34)c3ccccc23)cc1]

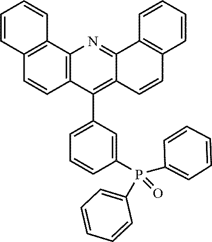 [SMILES: O=P(c1ccccc1)(c1ccccc1)c1cccc(-c2c3ccc4ccccc4c3nc3c2ccc2ccccc23)c1]

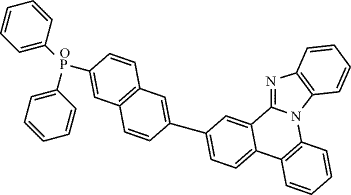

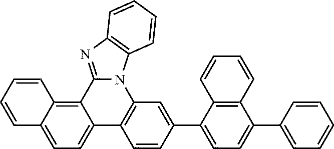 [SMILES: c1ccc(-c2ccc(-c3ccc4c5ccc6ccccc6c5c5nc6ccccc6n5c4c3)c3ccccc23)cc1]

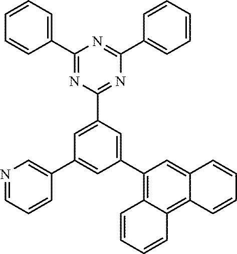 [SMILES: c1ccc(-c2nc(-c3ccccc3)nc(-c3cc(-c4cccnc4)cc(-c4cc5ccccc5c5ccccc45)c3)n2)cc1]

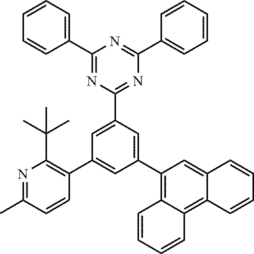 [SMILES: Cc1ccc(-c2cc(-c3nc(-c4ccccc4)nc(-c4ccccc4)n3)cc(-c3cc4ccccc4c4ccccc34)c2)c(C(C)(C)C)n1]

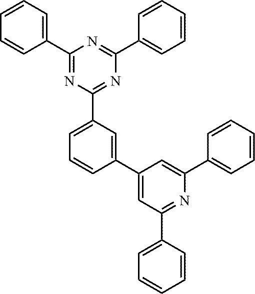 [SMILES: c1ccc(-c2cc(-c3cccc(-c4nc(-c5ccccc5)nc(-c5ccccc5)n4)c3)cc(-c3ccccc3)n2)cc1]

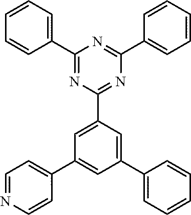 [SMILES: c1ccc(-c2cc(-c3ccncc3)cc(-c3nc(-c4ccccc4)nc(-c4ccccc4)n3)c2)cc1]

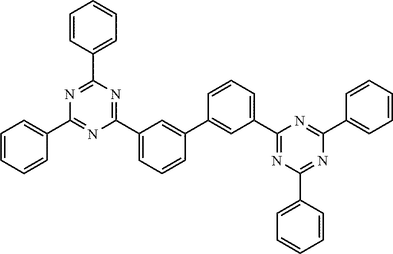 [SMILES: c1ccc(-c2nc(-c3ccccc3)nc(-c3cccc(-c4cccc(-c5nc(-c6ccccc6)nc(-c6ccccc6)n5)c4)c3)n2)cc1]

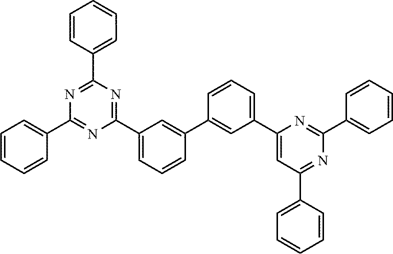 [SMILES: c1ccc(-c2cc(-c3cccc(-c4cccc(-c5nc(-c6ccccc6)nc(-c6ccccc6)n5)c4)c3)nc(-c3ccccc3)n2)cc1]

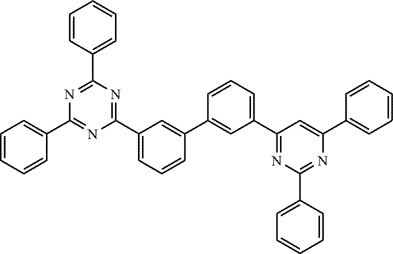 [SMILES: c1ccc(-c2cc(-c3cccc(-c4cccc(-c5nc(-c6ccccc6)nc(-c6ccccc6)n5)c4)c3)nc(-c3ccccc3)n2)cc1]

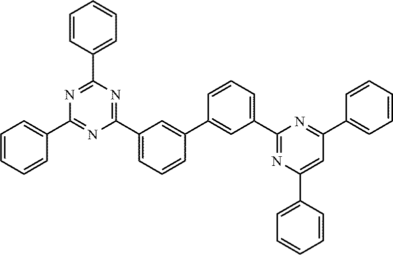 [SMILES: c1ccc(-c2cc(-c3ccccc3)nc(-c3cccc(-c4cccc(-c5nc(-c6ccccc6)nc(-c6ccccc6)n5)c4)c3)n2)cc1]

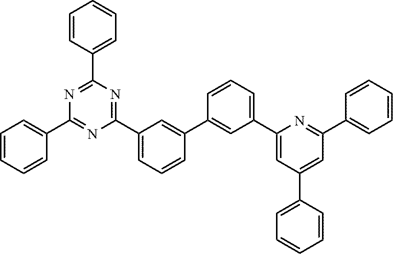 [SMILES: c1ccc(-c2cc(-c3ccccc3)nc(-c3cccc(-c4cccc(-c5nc(-c6ccccc6)nc(-c6ccccc6)n5)c4)c3)c2)cc1]

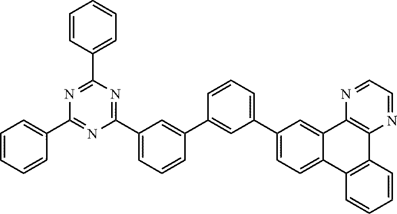 [SMILES: c1ccc(-c2nc(-c3ccccc3)nc(-c3cccc(-c4cccc(-c5ccc6c7ccccc7c7nccnc7c6c5)c4)c3)n2)cc1]

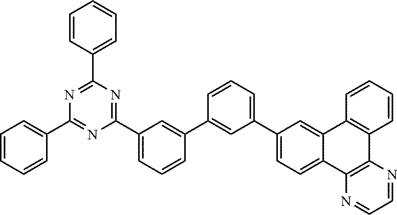 [SMILES: c1ccc(-c2nc(-c3ccccc3)nc(-c3cccc(-c4cccc(-c5ccc6c(c5)c5ccccc5c5nccnc65)c4)c3)n2)cc1]

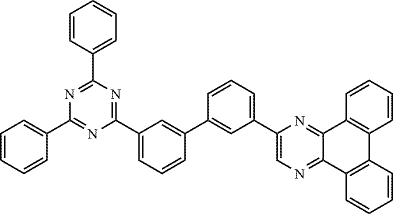 [SMILES: c1ccc(-c2nc(-c3ccccc3)nc(-c3cccc(-c4cccc(-c5cnc6c7ccccc7c7ccccc7c6n5)c4)c3)n2)cc1]

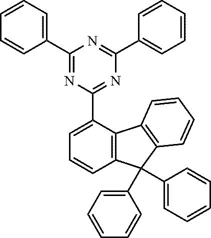 [SMILES: c1ccc(-c2nc(-c3ccccc3)nc(-c3cccc4c3-c3ccccc3C4(c3ccccc3)c3ccccc3)n2)cc1]

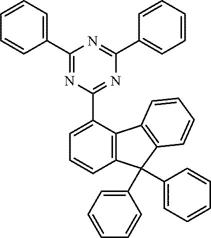 [SMILES: c1ccc(-c2nc(-c3ccccc3)nc(-c3cccc4c3-c3ccccc3C4(c3ccccc3)c3ccccc3)n2)cc1]

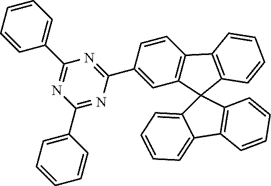 [SMILES: c1ccc(-c2nc(-c3ccccc3)nc(-c3ccc4c(c3)C3(c5ccccc5-c5ccccc53)c3ccccc3-4)n2)cc1]

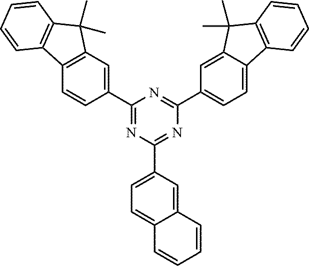 [SMILES: CC1(C)c2ccccc2-c2ccc(-c3nc(-c4ccc5c(c4)C(C)(C)c4ccccc4-5)nc(-c4ccc5ccccc5c4)n3)cc21]

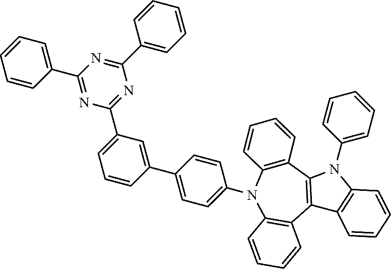 [SMILES: c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

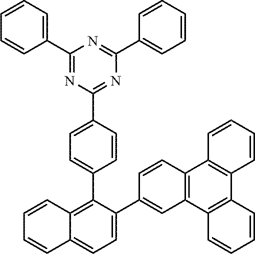 [SMILES: c1ccc(-c2nc(-c3ccccc3)nc(-c3ccc(-c4c(-c5ccc6c7ccccc7c7ccccc7c6c5)ccc5ccccc45)cc3)n2)cc1]

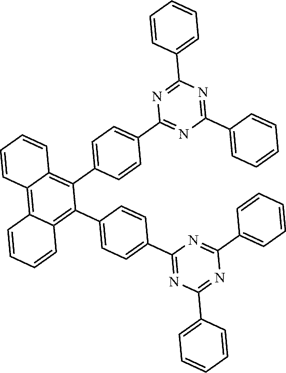 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1]

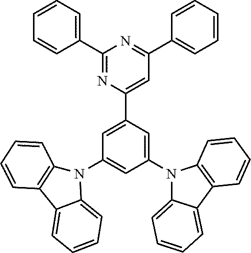 [SMILES: c1ccc(-c2cc(-c3cc(-n4c5ccccc5c5ccccc54)cc(-n4c5ccccc5c5ccccc54)c3)nc(-c3ccccc3)n2)cc1]

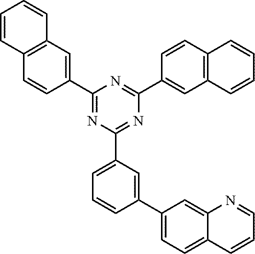 [SMILES: c1cc(-c2ccc3cccnc3c2)cc(-c2nc(-c3ccc4ccccc4c3)nc(-c3ccc4ccccc4c3)n2)c1]

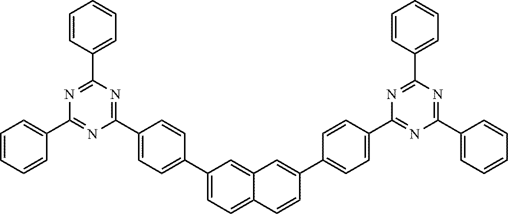 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

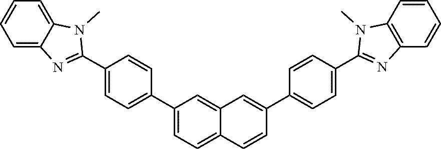 [SMILES: Cn1c(-c2ccc(-c3ccc4ccc(-c5ccc(-c6nc7ccccc7n6C)cc5)cc4c3)cc2)nc2ccccc21]

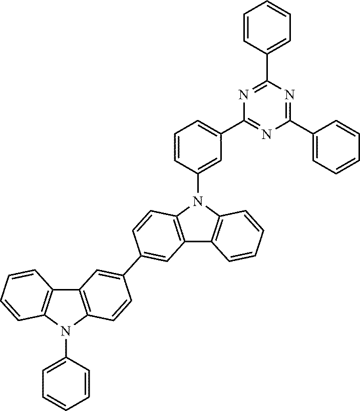 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

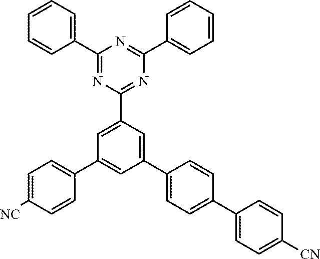

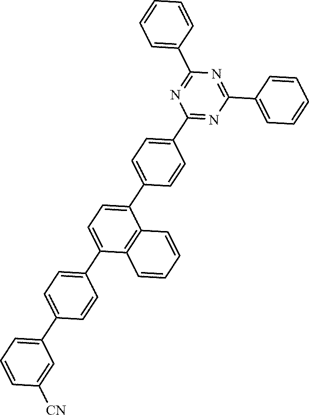 [SMILES: N#Cc1cccc(-c2ccc(-c3ccc(-c4ccc(-c5nc(-c6ccccc6)nc(-c6ccccc6)n5)cc4)c4ccccc34)cc2)c1]

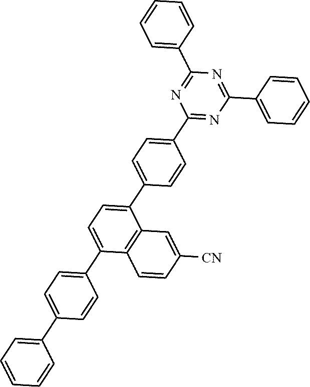 [SMILES: N#Cc1ccc2c(-c3ccc(-c4ccccc4)cc3)ccc(-c3ccc(-c4nc(-c5ccccc5)nc(-c5ccccc5)n4)cc3)c2c1]

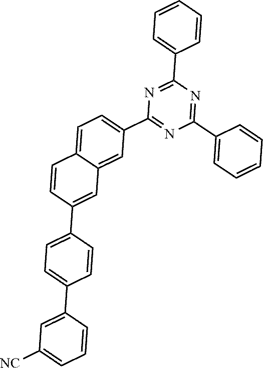

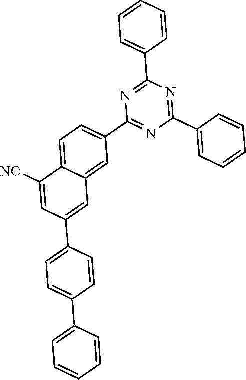

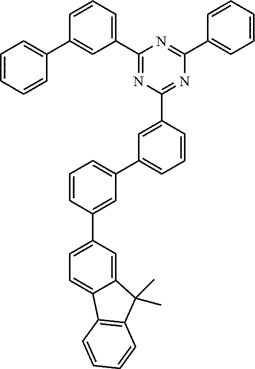 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21]

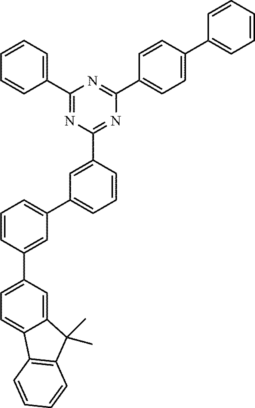 [SMILES: CC1(C)c2ccccc2-c2ccc(-c3cccc(-c4cccc(-c5nc(-c6ccccc6)nc(-c6ccc(-c7ccccc7)cc6)n5)c4)c3)cc21]

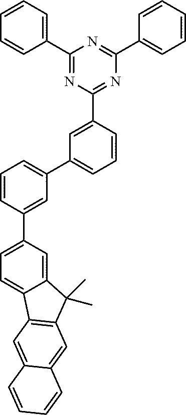 [SMILES: CC1(C)c2cc(-c3cccc(-c4cccc(-c5nc(-c6ccccc6)nc(-c6ccccc6)n5)c4)c3)ccc2-c2cc3ccccc3cc21]

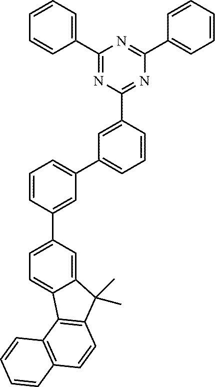 [SMILES: CC1(C)c2cc(-c3cccc(-c4cccc(-c5nc(-c6ccccc6)nc(-c6ccccc6)n5)c4)c3)ccc2-c2c1ccc1ccccc21]

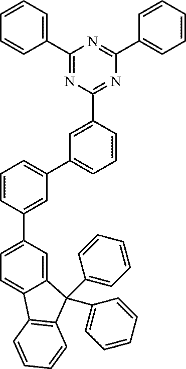 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1]

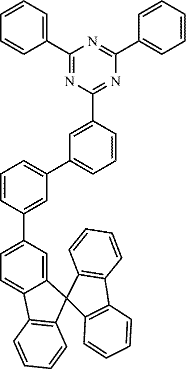 [SMILES: c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

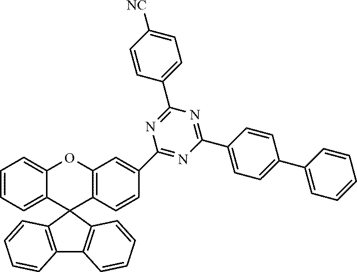 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1]

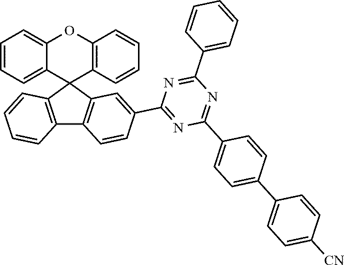 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1]

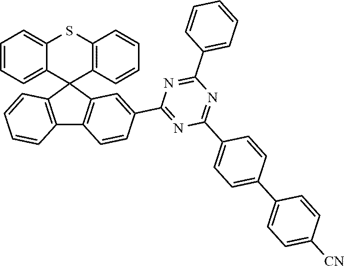 [SMILES: N#Cc1ccc(-c2ccc(-c3nc(-c4ccccc4)nc(-c4ccc5c(c4)C4(c6ccccc6Sc6ccccc64)c4ccccc4-5)n3)cc2)cc1]

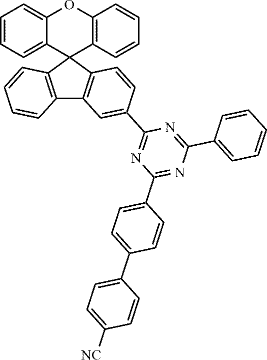 [SMILES: N#Cc1ccc(-c2ccc(-c3nc(-c4ccccc4)nc(-c4ccc5c(c4)-c4ccccc4C54c5ccccc5Oc5ccccc54)n3)cc2)cc1]

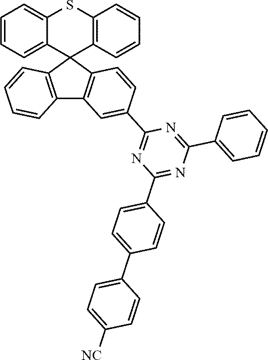 [SMILES: N#Cc1ccc(-c2ccc(-c3nc(-c4ccccc4)nc(-c4ccc5c(c4)-c4ccccc4C54c5ccccc5Sc5ccccc54)n3)cc2)cc1]

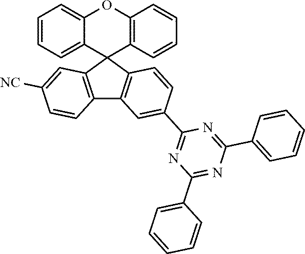 [SMILES: N#Cc1ccc2c(c1)C1(c3ccccc3Oc3ccccc31)c1ccc(-c3nc(-c4ccccc4)nc(-c4ccccc4)n3)cc1-2]

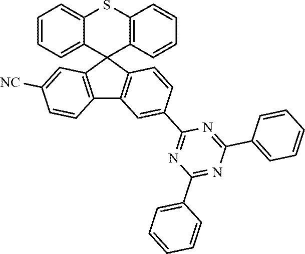 [SMILES: N#Cc1ccc2c(c1)C1(c3ccccc3Sc3ccccc31)c1ccc(-c3nc(-c4ccccc4)nc(-c4ccccc4)n3)cc1-2]

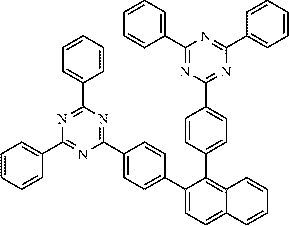 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1]

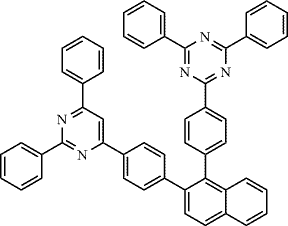 [SMILES: c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1]

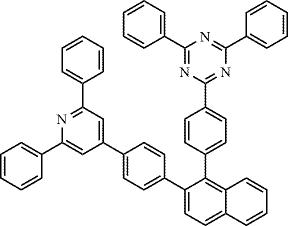 [SMILES: c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1]

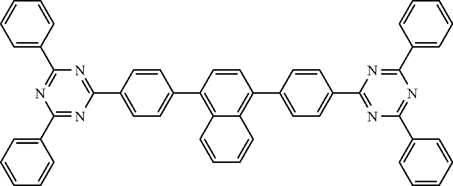 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

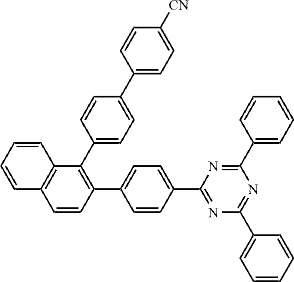 [SMILES: N#Cc1ccc(-c2ccc(-c3c(-c4ccc(-c5nc(-c6ccccc6)nc(-c6ccccc6)n5)cc4)ccc4ccccc34)cc2)cc1]

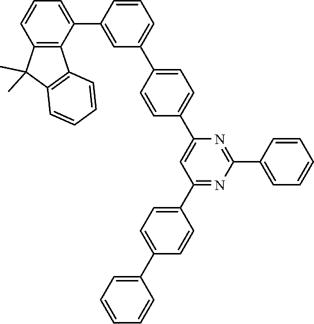 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21]

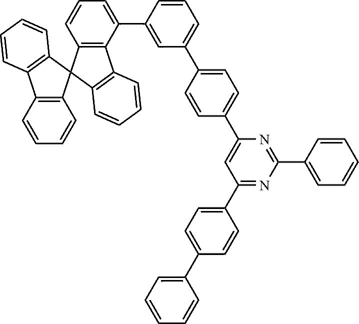 [SMILES: c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1]

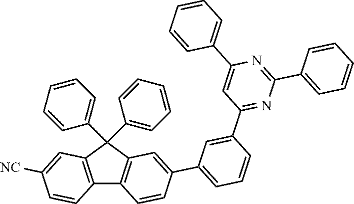 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2]

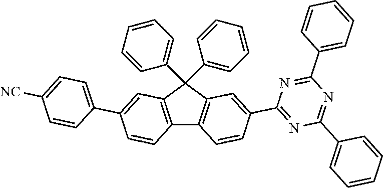

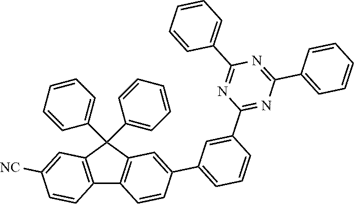

An embodiment may include an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in the FIGURE, the organic light emitting diode according to the embodiment may include a hole transport region 140 and an electron transport region 150 in addition to the light emitting layer 130 as the organic layer 105.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, or ion plating, and forming a cathode or an anode thereon.

The organic light emitting diode may be applied to an organic light emitting display device.

Hereinafter, the embodiments are illustrated in more detail with reference to examples. However, these examples are exemplary, and the scope as claimed in claims is not limited thereto.

Hereinafter, starting materials and reactants used in examples and synthesis examples were purchased from Sigma-Aldrich Co. Ltd., TCI Inc., Tokyo Chemical Industry, or P&H Tech as far as there in no particular comment, or were synthesized by suitable methods.

### Preparation of Compound for Organic Optoelectronic Device

The compound was synthesized through the following steps.

### Synthesis Example 1: Synthesis of Intermediate A

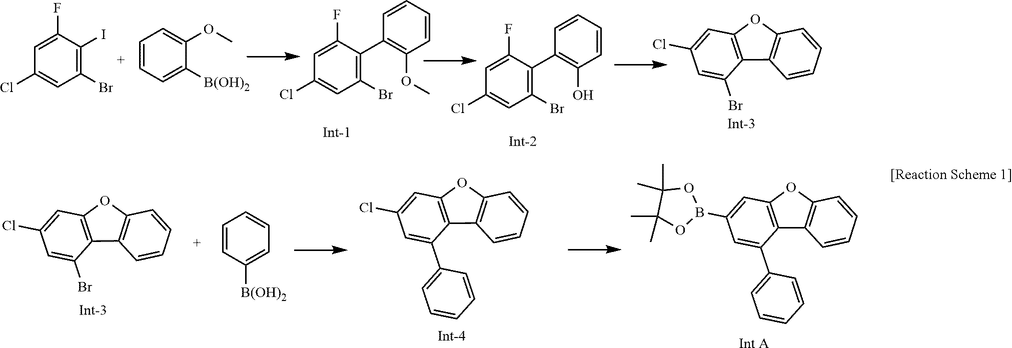

### 1St Step: Synthesis of Intermediate Int-1

105 g (313.12 mmol) of 1-bromo-5-chloro-3-fluoro-2-iodobenzene, 52.3 g (344.43 mmol) of 2-methoxyphenylboronic acid, 86.6 g (626.23 mmol) of K₂CO₃, and 18.1 g (15.7 mmol) of Pd(PPh₃)₄ were put in a round-bottomed flask and dissolved in 900 ml of 1,4-dioxane and 310 ml of distilled water and then, stirred under reflux at 90° C. for 12 hours. When a reaction was completed, after removing an aqueous layer therefrom, the residue was treated through column chromatography (hexane:DCM (dichloromethane) 25%), obtaining 68.3 g (69%) of Intermediate Int-1.

### 2Nd Step: Synthesis of Intermediate Int-2

68.3 g (216.4 mmol) of Intermediate Int-1 and 175.3 g (1517.2 mmol) of pyridine hydrochloride were put in a round-bottomed flask and stirred under reflux at 200° C. for 24 hours. When a reaction was completed, the resultant was cooled to ambient temperature and slowly poured into distilled water and then, stirred for 1 hour. A solid produced therein was filtered, obtaining 65 g (99%) of Intermediate Int-2.

### 3Rd Step: Synthesis of Intermediate Int-3

65 g (215.6 mmol) of Intermediate Int-2 and 53.6 g (388 mmol) of K₂CO₃ were put in a round-bottomed flask and dissolved in 100 ml of NMP and then, stirred under reflux at 180° C. for 12 hours. When a reaction was completed, the mixture was poured into an excess of distilled water. A solid produced therein was filtered and dissolved in ethyl acetate (EA) and then, dried with MgSO₄, and an organic layer therefrom was removed under a reduced pressure. The residue was treated through column chromatography (hexane:EA (30%)), obtaining 51 g (84%) of Intermediate Int-3.

### 4Th Step: Synthesis of Intermediate Int-4

51 g (181.2 mmol) of Intermediate Int-3, 24.3 g (199.3 mmol) of phenylboronic acid, 50.1 g (362.3 mmol) of K₂CO₃, and 10.5 g (9.1 mmol) of Pd(PPh₃)₄ were put in a round-bottomed flask and dissolved in 600 ml of 1,4-dioxane and 180 ml of distilled water and then, stirred under reflux at 90° C. for 12 hours. When a reaction was completed, after removing an aqueous layer therefrom, the residue was treated through column chromatography (hexane:DCM (25%)), obtaining 37 g (73%) of Intermediate Int-4.

### 5Th Step: Synthesis of Intermediate A

37 g (132.7 mmol) of Intermediate Int-4, 43.8 g (172.6 mmol) of bis(pinacolato)diboron, 5.4 g (6.6 mmol) of Pd(dppf)Cl₂, 8.0 g (33.2 mmol) of tricyclohexylphosphine, and 26.1 g (265.5 mmol) of potassium acetate were put in a round-bottomed flask and dissolved in 280 ml of xylene. The mixture was stirred under reflux at 120° C. for 10 hours. When a reaction was completed, the mixture was poured into an excess of distilled water and then, stirred for 1 hour. A solid was filtered therefrom and dissolved in DCM. After removing moisture with MgSO₄, an organic solvent was filtered with a silica gel pad and removed under a reduced pressure. A solid therefrom was recrystallized with ethyl acetate and hexane, obtaining 36.9 g (75%) of Intermediate A.

### Synthesis Example 2: Synthesis of Compound 15

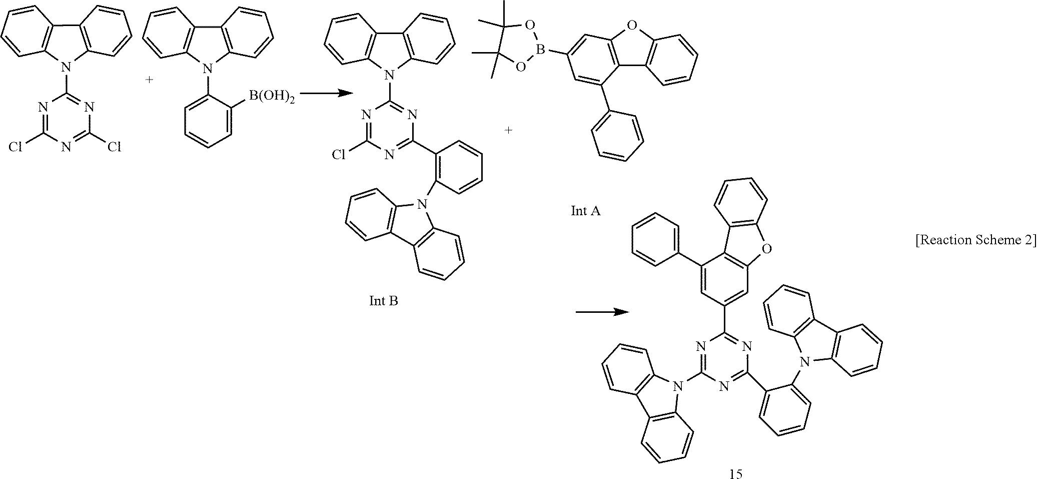

### 1St Step: Synthesis of Intermediate B

37 g (117.4 mmol) of 9-(4,6-dichloro-1,3,5-triazin-2-yl)-9H-carbazole, 33.7 g (117.4 mmol) of 2-(9H-carbazol-9-yl)phenylboronic acid, 32.5 g (234.8 mmol) of K₂CO₃, and 4.8 g (5.9 mmol) of Pd(PPh₃)₄ were put in a round-bottomed flask and dissolved in 400 ml of toluene and 120 ml of distilled water and then, stirred at 65° C. for 6 hours. When a reaction was completed, after removing an aqueous layer therefrom, the residue was treated column chromatography (hexane:DCM (20%)) and purified by sublimation, obtaining 39 g (64%) of Intermediate B.

### 2Nd Step: Synthesis of Compound 15

9.1 g (17.4 mmol) of Intermediate B, 7.1 g (19.2 mmol) of Intermediate A, 4.8 g (34.9 mmol) of K₂CO₃, and 1.1 g (0.9 mmol) of Pd(PPh₃)₄ were put in a round-bottomed flask and dissolved in 90 ml of THF and 18 ml of distilled water and then, stirred under reflux at 60° C. for 12 hours. When a reaction was completed, an excess of distilled water was added thereto and then, stirred to precipitate a solid. The solid was recrystallized with monochlorobenezene, obtaining 11 g (86%) of Compound 15.

(LC/MS: theoretical value: 729.25 g/mol, measured value: 730.45 g/mol)

### Synthesis Example 3: Synthesis of Compound 45

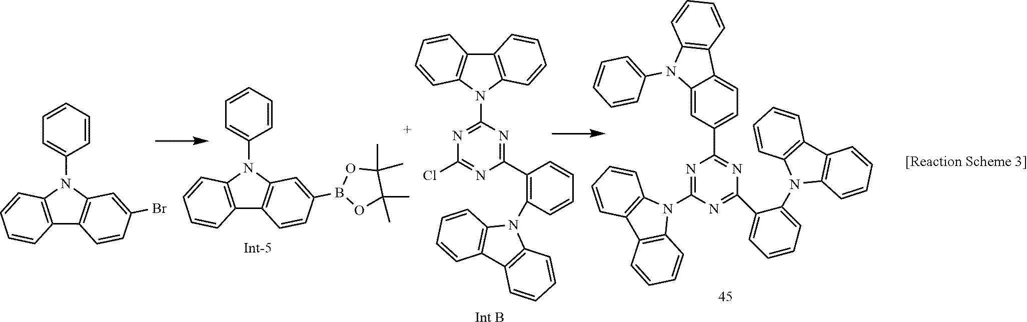

### 1St Step: Synthesis of Intermediate Int-5

25.0 g (77.6 mmol) of 2-bromo-9-phenyl-9H-carbazole, 23.6 g (93.1 mmol) of bis(pinacolato)diboron, 3.2 g (3.9 mmol) of Pd(dppf)Cl₂, and 11.4 g (116.4 mmol) of potassium acetate were put in a round-bottomed flask and dissolved in 160 ml of DMF. The mixture was stirred under reflux at 120° C. for 8 hours. When a reaction was completed, the resultant was poured into an excess of distilled water and then, stirred for 1 hour. A solid therefrom was filtered and dissolved in DCM. After removing moisture with MgSO₄, an organic solvent was filtered with a silica gel pad and removed under a reduced pressure. A solid therefrom was recrystallized with ethyl acetate and hexane, obtaining 21.8 g (76%) of Intermediate Int-5.

### 2Nd Step: Synthesis of Compound 45

11.7 g (31.7 mmol) of Intermediate Int-5, 16.5 g (31.7 mmol) of Intermediate B, 8.8 g (63.4 mmol) of K₂CO₃, and 1.8 g (1.6 mmol) of Pd(PPh₃)₄ were put in a round-bottomed flask and dissolved in 130 ml of THF and 35 ml of distilled water and then, stirred at 70° C. for 8 hours. When a reaction was completed, an excess of distilled water was added thereinto and then, stirred to precipitate a solid. The solid was recrystallized with toluene, obtaining 12.7 g (55%) of Compound 45.

(LC/MS: theoretical value: 728.27 g/mol, measured value: 729.33 g/mol)

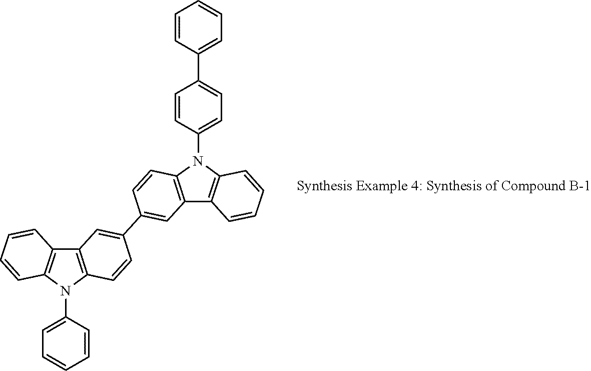 [SMILES: c1ccc(-c2ccc(-n3c4ccccc4c4cc(-c5ccc6c(c5)c5ccccc5n6-c5ccccc5)ccc43)cc2)cc1]

Compound B-1 was synthesized as described in U.S. Pat. No. 10,476,008 B2.

### Comparative Synthesis Example 1: Synthesis of Compound Y1

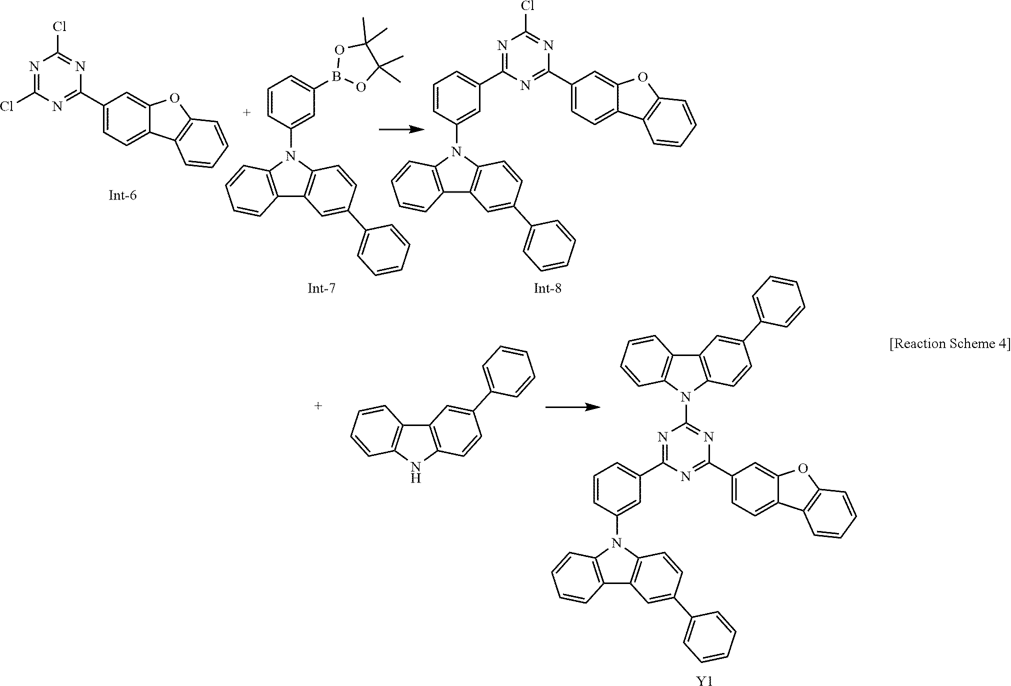

### 1St Step: Synthesis of Intermediate Int-8

20.5 g (64.8 mmol) of Intermediate Int-6, 31.8 g (71.3 mmol) of Intermediate Int-7, 17.9 g (129.7 mmol) of K₂CO₃, and 3.8 g (3.3 mmol) of Pd(PPh₃)₄ were put in a round-bottomed flask and dissolved in 260 ml of THF and 70 ml of distilled water and then, stirred under reflux at 70° C. for 8 hours. When a reaction was completed, an excess of distilled water was added thereto and then, stirred to precipitate a solid. The solid was recrystallized with toluene and purified by sublimation, obtaining 24.9 g (64%) of Intermediate Int-8.

### 2Nd Step: Synthesis of Compound Y1

15.9 g (26.5 mmol) of Intermediate Int-8, 7.8 g (31.9 mmol) of 3-phenyl-9H-carbazole, 5.1 g (53.1 mmol) of sodium tert-butoxide, 1.3 ml (2.7 mmol) of tri-tert-butyl phosphine (50% in toluene), and 1.2 g (1.3 mmol) of Pd₂(dba)₃ were put in a round-bottomed flask, and 180 ml of xylene was added thereto and then, stirred under reflux for 12 hours under a nitrogen flow. The obtained mixture was added to an excess of methanol to crystallize a solid, and the solid was filtered. After dissolving the solid in monochlorobenzene by heating, filtering the solution through silica gel pad, and removing an appropriate amount of an organic solvent therefrom, the solid was recrystallized with toluene, obtaining 10.3 g (48%) of Compound Y1.

(LC/MS: theoretical value: 805.28, measured value: 806.37 g/mol)

### Comparative Synthesis Example 2: Synthesis of Intermediate Int-12

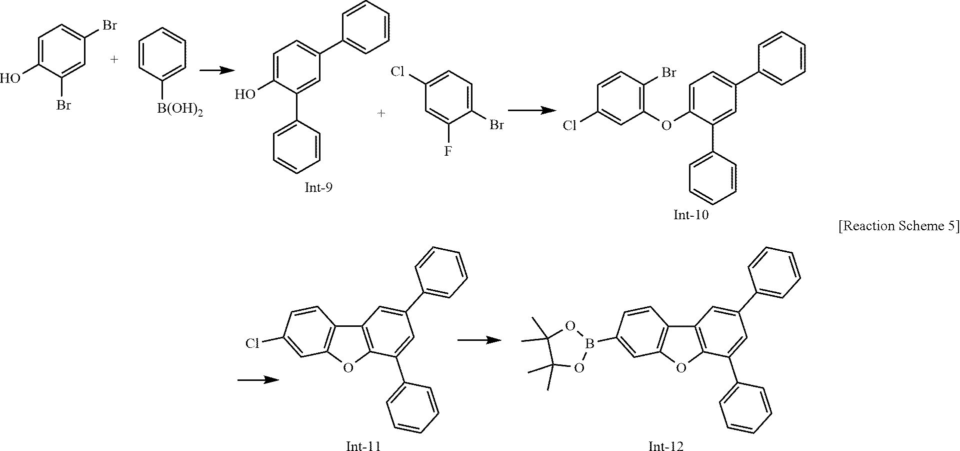

### 1St Step: Synthesis of Intermediate Int-9

50.0 g (198.5 mmol) of 2,4-dibromophenol, 53.2 g (436.7 mmol) of phenylboronic acid, 68.6 g (496.2 mmol) of K₂CO₃, and 11.5 g (9.9 mmol) of Pd(PPh₃)₄ were put in a round-bottomed flask and dissolved in 700 ml of THF and 250 ml of distilled water and then, stirred under reflux at 70° C. for 10 hours. The reactant was cooled to ambient temperature, and after removing an aqueous layer through a separatory funnel, the residue was column chromatography (hexane:DCM (35%)), obtaining 24.9 g (51%) of Intermediate Int-9.

### 2Nd Step: Synthesis of Intermediate Int-10

24.9 g (101.1 mmol) of Intermediate Int-9, 31.8 g (151.6 mmol) of 1-bromo-4-chloro-2-fluorobenzene, and 42.9 g (202.2 mmol) of K₃PO₄ were put in a round-bottomed flask and dissolved in 85 ml of NMP (N-Methyl-2-pyrrolidone) and then, stirred under reflux at 200° C. for 14 hours. The reactants were cooled to ambient temperature and filtered to remove a salt, an excess of DCM and distilled water were added thereto for extraction. The extract was treated through column chromatography (hexane:DCM (20%)), obtaining 35.2 g (80%) of Intermediate Int-10.

### 3Rd Step: Synthesis of Intermediate Int-11

35.2 g (80.8 mmol) of Intermediate Int-10, 15.9 g (161.6 mmol) of potassium acetate, and 4.7 g (4.0 mmol) of Pd(PPh₃)₄ were put in a round-bottomed flask and dissolved in 160 ml of DMAc (dimethylacetamide) and then, stirred under reflux at 160° C. for 6 hours. The reactant was cooled to ambient temperature and filtered to remove a salt, and an excess of DCM and distilled water were added thereto for extraction. The extract was treated through column chromatography (hexane:DCM (20%)), obtaining 19.8 g (69%) of Intermediate Int-11.

### 4Th Step: Synthesis of Intermediate Int-12

19.8 g (55.8 mmol) of Intermediate Int-11, 17.7 g (69.8 mmol) of bis(pinacolato)diboron, 2.6 g (2.8 mmol) of Pd₂(dba)₃, 3.4 g (14.0 mmol) of tricyclohexylphosphine, and 11.0 g (111.6 mmol) of potassium acetate were put in a round-bottomed flask and dissolved in 120 ml of xylene. The mixture was stirred under reflux at 120° C. for 8 hours. When a reaction was completed, the resultant was cooled to ambient temperature and filtered to remove a salt, and an excess of DCM and distilled water were added thereto for extraction. The extract was treated through column chromatography (hexane:DCM (30%)), obtaining 20.4 g (82%) of Intermediate Int-12.

### Comparative Synthesis Example 3: Synthesis of Compound Y2

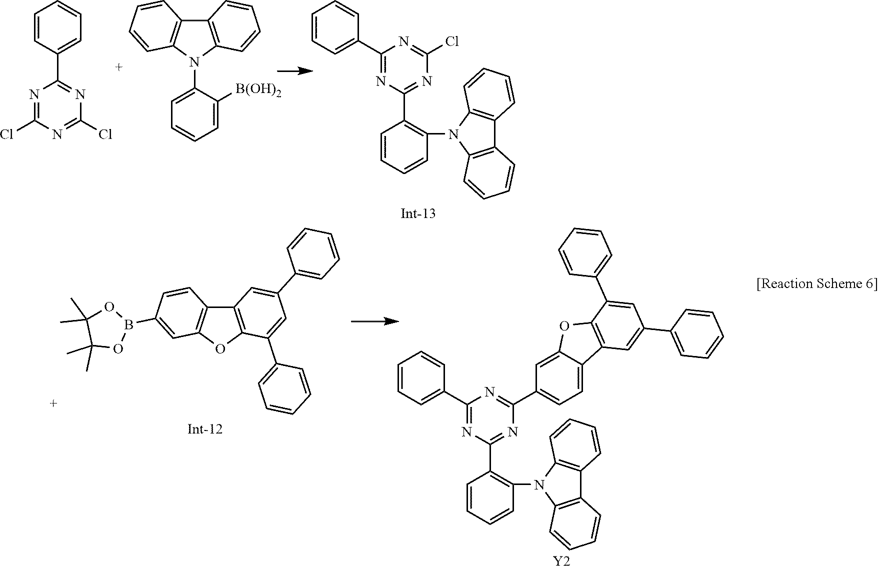

### 1St Step: Synthesis of Intermediate Int-13

13.5 g (59.7 mmol) of 2,4-dichloro-6-phenyl-1,3,5-triazine, 15.4 g (53.8 mmol) of 2-(9H-carbazol-9-yl)phenylboronic acid, 68.6 g (496.2 mmol) of K₂CO₃, and 2.4 g (3.0 mmol) of Pd(dppf)Cl₂ were put in a round-bottomed flask and dissolved in 180 ml of toluene and 60 ml of distilled water and then, stirred at 60° C. for 8 hours. When a reaction was completed, after removing an aqueous layer, the residue was treated through column chromatography (hexane:DCM (30%)) and purified sublimation, obtaining 11.2 g (48%) of Intermediate Int-13.

### 2Nd Step: Synthesis of Compound Y2

11.2 g (25.9 mmol) of Intermediate Int-13, 12.7 g (28.5 mmol) of Intermediate Int-12, 7.2 g (51.8 mmol) of K₂CO₃, and 1.5 g (1.3 mmol) of Pd(PPh₃)₄ were put in a round-bottomed flask and dissolved in 100 ml of THF and 30 ml of distilled water and then, stirred under reflux at 70° C. for 8 hours. The reactant was cooled to ambient temperature, and an excess of methanol was added thereto to extract a solid. After adding monochlorobenzene thereto to dissolve the solid, filtering the solution through silica gel pad, and removing an appropriate amount of an organic solvent therefrom, the residue was recrystallized with toluene, obtaining 9.5 g (51%) of Compound Y2.

(LC/MS: theoretical value: 716.26, measured value: 717.39 g/mol)

### Comparative Synthesis Example 4: Synthesis of Compound Y3

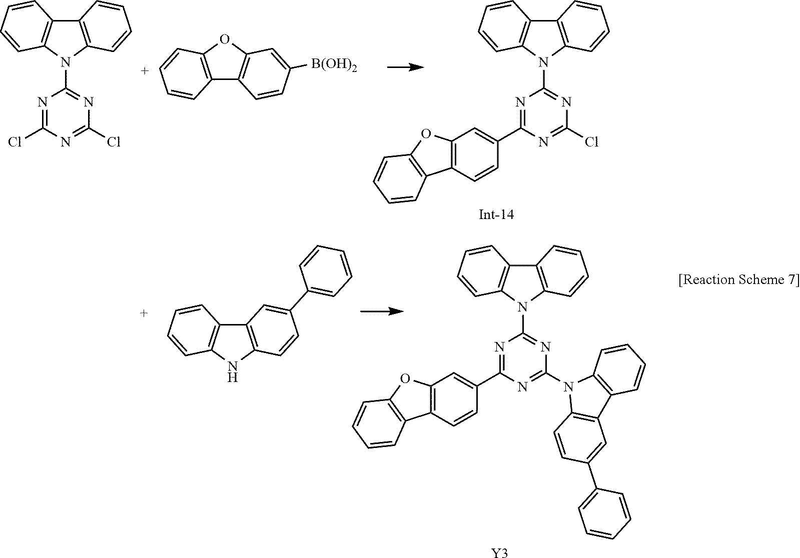

### 1St Step: Synthesis of Intermediate Int-14

20.5 g (65.1 mmol) of 9-(4,6-dichloro-1,3,5-triazin-2-yl)-9H-carbazole, 13.8 g (65.1 mmol) of dibenzo[b,d]furan-3-ylboronic acid, 18.0 g (130.1 mmol) of K₂CO₃, and 3.8 g (3.3 mmol) of Pd(PPh₃)₄ were put in a round-bottomed flask and dissolved in 150 ml of toluene and 60 ml of distilled water and then, stirred at 65° C. for 6 hours. When a reaction was completed, after removing an aqueous layer therefrom, the residue was treated through column chromatography (hexane:DCM (20%)) and purified by sublimation, obtaining 13.7 g (47%) of Intermediate Int-14.

### 2Nd Step: Synthesis of Compound Y3

13.7 g (30.7 mmol) of Intermediate Int-14, 7.5 g (30.7 mmol) of 3-phenyl-9H-carbazole, 8.5 g (61.3 mmol) of K₂CO₃, and 1.8 g (1.5 mmol) of Pd(PPh₃)₄ were put in a round-bottomed flask and dissolved in 80 ml of THF and 30 ml of distilled water and then, stirred at 65° C. for 6 hours. The reactants were cooled to ambient temperature and added to an excess of methanol to precipitate a solid. After dissolving the solid in monochlorobenzene by heating, filtering the solution through silica gel pad, and removing an appropriate amount of an organic solvent, the residue was recrystallized with monochlorobenzene, obtaining 11.8 g (59%) of Compound Y3.

### Manufacture of Organic Light Emitting Diode

### Example 1

A glass substrate coated with a thin film of indium tin oxide (ITO) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 A-thick hole injection layer, and Compound A was deposited on the hole transport layer to form a 1,350 A-thick hole transport layer. On the hole transport layer, Compound B was deposited at a thickness of 350 Å to form a hole transport auxiliary layer. On the hole transport auxiliary layer, 400 Å-thick light emitting layer was formed by using Compound 15 obtained in Synthesis Example 2 and doping 15 wt% of PhGD as a dopant by vacuum-deposition. Subsequently, on the light emitting layer, Compound C was deposited at a thickness of 50 Å to form an electron transport auxiliary layer and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The organic light emitting diode has a structure of ITO/Compound A (3% NDP-9 doping, 100 Å)/ Compound A (1,350 Å) / Compound B (350 Å) / EML[85 wt% of host (Compound 15) : 15 wt% of PhGD] (400 Å) / Compound C (50 Å) / Compound D : LiQ (300 Å) / LiQ (15 Å) / Al (1,200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-dibenzofuranyl)phenyl]-N-[4-(9-phenyl-9H-fluoren-9-yl)phenyl] [1,1 ′-biphenyl]-4-amine

Compound C: 2,4-diphenyl-6-(4′,5′,6′-triphenyl[1,1′:2′,1″:3″,1″′:3″′,1⁗-quinquephenyl]-3⁗-yl)-1,3,5-triazine

Compound D: 2-(1,1′-biphenyl-4-yl)-4-(9,9-diphenyl-9H-fluoren-4-yl)-6-phenyl-1,3,5-triazine

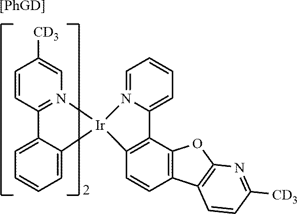

### Example 2

A glass substrate coated with a thin film of indium tin oxide (ITO) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1,350 Å-thick hole transport layer. On the hole transport layer, Compound E was deposited at a thickness of 350 Å to form a hole transport auxiliary layer. On the hole transport auxiliary layer, 400 Å-thick light emitting layer was formed by using Compound 15 obtained in Synthesis Example 2 and Compound B-1 obtained in Synthesis Example 4 simultaneously as a host and doping 10 wt% of PhGD as a dopant by vacuum-deposition. Herein, Compound 15 and Compound B-1 were used in a weight ratio of 3:7. Subsequently, Compound F was deposited on the light emitting layer at a thickness of 50 Å to form an electron transport auxiliary layer, and Compound G and LiQ were simultaneously vacuum-deposited at a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The organic light emitting diode has a structure of ITO / Compound A (3% NDP-9 doping, 100 Å) / Compound A (1,350 Å) / Compound E (350 Å)/ EML[Compound 15: Compound B-1: PhGD = 27:63:10 wt%] (400 Å) / Compound F (50 Å) / Compound G: LiQ (300 Å) / LiQ (15 Å) / Al (1,200 Å).

Compound E: N-[1,1′-Biphenyl]-4-yl-N-(9,9-dimethyl-9H-fluoren-2-yl)-7,7-dimethyl-7H-benzo[b]fluoreno[3,2-d]furan-1-amine

Compound F: 2-[3′-(9,9-dimethyl-9H-fluoren-2-yl)[1,1′-biphenyl]-3-yl]-4,6-diphenyl-1,3,5-triazine

Compound G: 2-[4-[4-(4′-cyano-1,1=′-biphenyl-4-yl)-1-naphthyl]phenyl]-4,6-diphenyl-1,3,5-triazine

### Comparative Example 1

A diode according to Comparative Example 1 was manufactured in the same method as in Example 1 except that the host was changed as shown in Table 1.

### Comparative Examples 2 and 3

Diodes according to Comparative Examples 2 and 3 were manufactured in the same method as in Example 1 except that the host was changed as shown in Table 2.

### Evaluation: Confirmation of Efficiency Increase Effect

### Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### Measurement of Luminance Change Depending on Voltage Change

Luminance was measured by using a luminance meter (Minolta Cs-1000A), while the voltage of the organic light emitting diodes was increased from 0 V to 10 V.

### Measurement of Luminous Efficiency

The luminous efficiency (cd/A) of the same current density (10 mA/cm²) was calculated using the luminance and current density measured from the (1) and (2).

The relative values based on luminous efficiency of Comparative Example 1 are shown in Table 1.

The relative values based on luminous efficiency of Comparative Example 2 are shown in Table 2.

### Measurement of Life-Span

T95 life-spans of the manufactured diodes were measured as a time when their luminance decreased down to 95% relative to the initial luminance (cd/m²) after emitting light with 24,000 cd/m² as the initial luminance (cd/m²) and measuring their luminance decreases depending on a time with a Polanonix life-span measurement system.

The relative values based on the T95 life-span of Comparative Example 2 are shown in Table 2.

### Measurement of Driving Voltage

The driving voltage of each diode was measured at 15 mA/cm² using a current-voltmeter (Keithley 2400), and the results were obtained.

The relative values based on the driving voltages of Comparative Example 1 were calculated and shown in Table 1.

TABLE 1

| | Host | Driving voltage (%) | Luminous efficiency (%) |
|---|---|---|---|
| Example 1 | Compound 15 | 92 | 112 |
| Comparative Example 1 | Compound Y1 | 100 | 100 |

TABLE 2

| | First host | Second host | Luminous efficiency (%) | Life-span T95 (%) |
|---|---|---|---|---|
| Example 2 | Compound 15 | Compound B-1 | 107 | 188 |
| Example 3 | Compound 45 | Compound B-1 | 115 | 130 |
| Comparative Example 2 | Compound Y2 | Compound B-1 | 100 | 100 |
| Comparative Example 3 | Compound Y3 | Compound B-1 | 90 | 110 |

Referring to Tables 1 and 2, the organic light emitting diodes according to the Examples exhibited significantly improved driving voltage, luminous efficiency, and life-span characteristics, compared with the organic light emitting diodes according to the Comparative Examples.

One or more embodiments may provide a compound for an organic optoelectronic device capable of implementing a low-driving and high-efficiency organic optoelectronic device.

One or more embodiments may provide a compound for an organic optoelectronic device capable of implementing high efficiency and long life-span organic optoelectronic device.

A low-driving and high-efficiency organic optoelectronic device may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 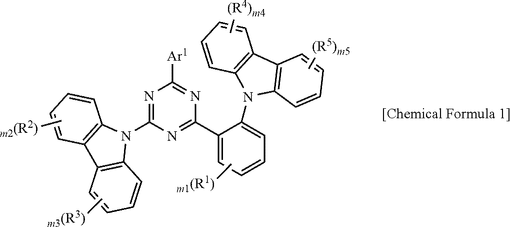
wherein, in Chemical Formula 1,
Ar¹ is a substituted or unsubstituted dibenzofuranyl group, a substituted or unsubstituted dibenzothiophenyl group, or a substituted or unsubstituted carbazolyl group,
R¹ is hydrogen, deuterium, or an unsubstituted C6 to C12 aryl group,
R² to R⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, and
m1 to m5 are each independently an integer 1 to 4.

2. The compound as claimed in claim 1, wherein:
the compound represented by Chemical Formula 1 is represented by one of Chemical Formula 1A to Chemical Formula 1D, 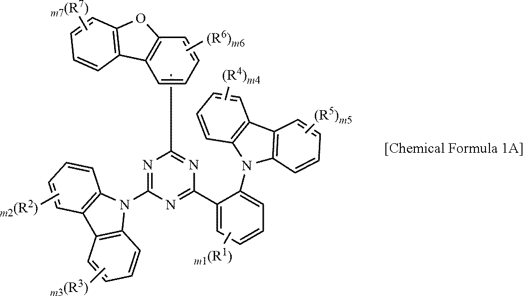 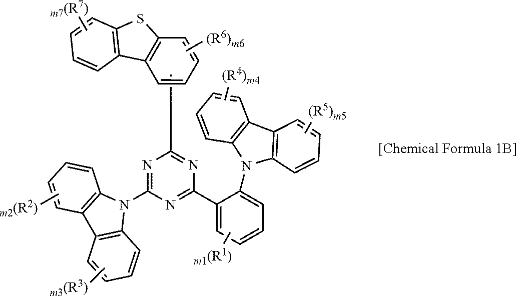 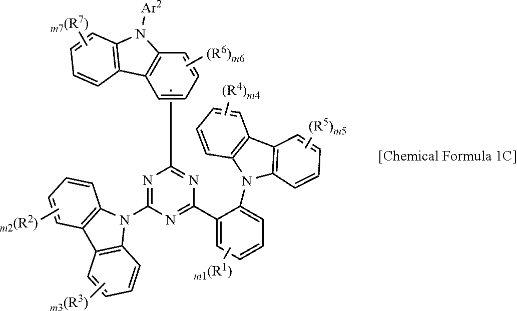 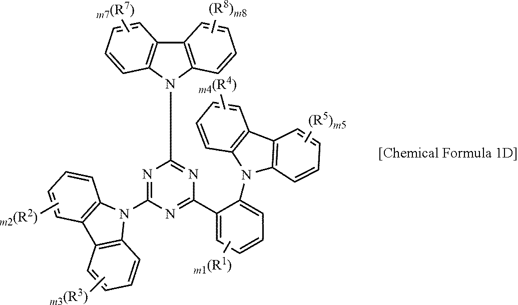
wherein, in Chemical Formula 1A to Chemical Formula 1D,
R¹ to R⁵ and m1 to m5 are defined the same as those of Chemical Formula 1,
R⁶ to R⁸ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof,
m6 is an integer of 1 to 3,
m7 and m8 are each independently an integer of 1 to 4, and
Ar² is a substituted or unsubstituted C6 to C12 aryl group.

3. The compound as claimed in claim 2, wherein:
the compound represented by Chemical Formula 1 is represented by Chemical Formula 1A,
Chemical Formula 1A is represented by one of Chemical Formula 1A-1 to Chemical Formula 1A-4, 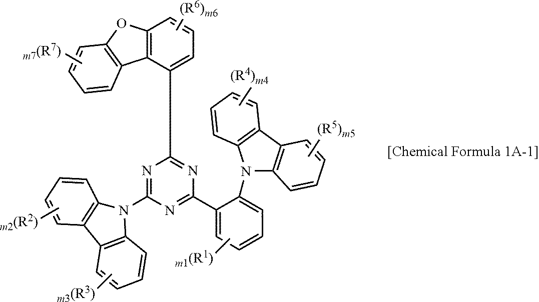 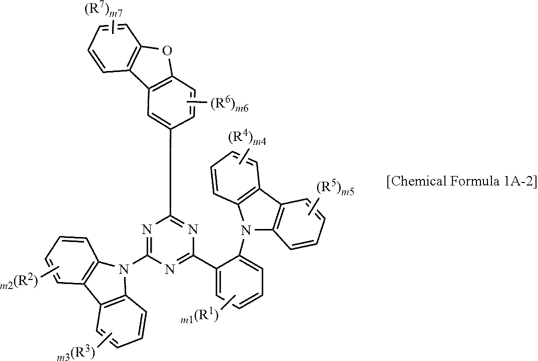 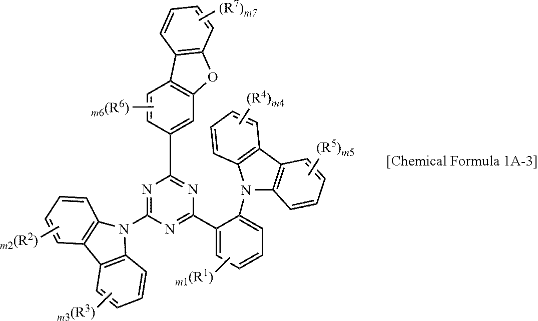 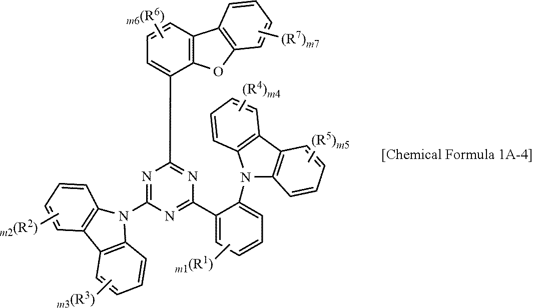
in Chemical Formula 1A-1 to Chemical Formula 1A-4, R¹ to R⁷ and m1 to m7 are defined the same as those of Chemical Formula 1 and Chemical Formula 1A.

4. The compound as claimed in claim 2, wherein:
the compound represented by Chemical Formula 1 is represented by Chemical Formula 1B,
Chemical Formula 1B is represented by one of Chemical Formula 1B-1 to Chemical Formula 1B-4, 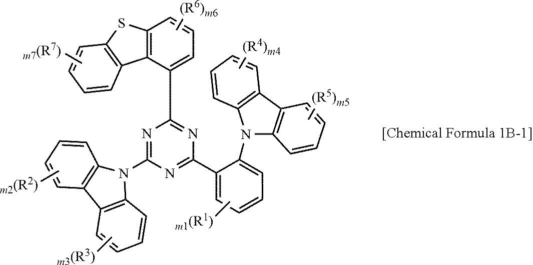 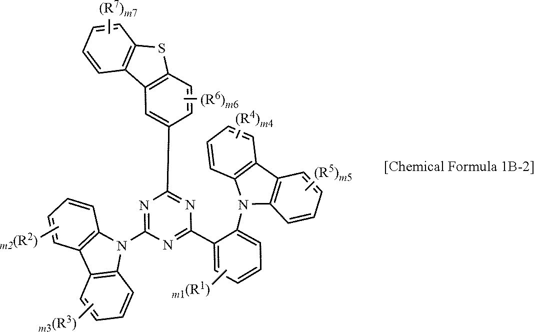 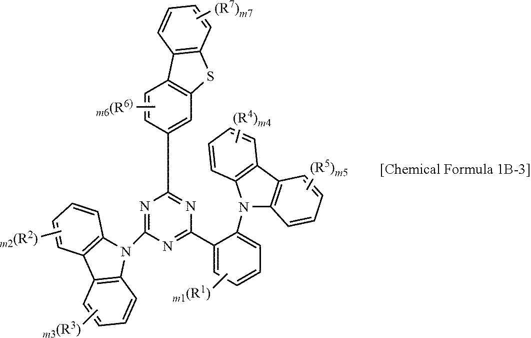 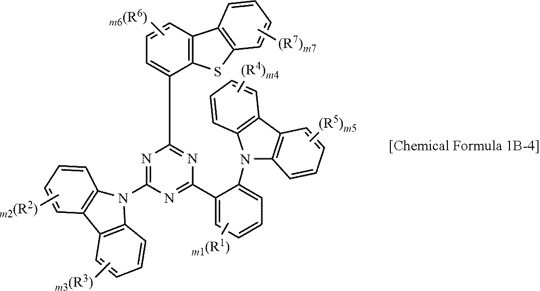
in Chemical Formula 1B-1 to Chemical Formula 1B-4, R¹ to R⁷ and m1 to m7 are defined the same as those of Chemical Formula 1 and Chemical Formula 1B.

5. The compound as claimed in claim 2, wherein:
the compound represented by Chemical Formula 1 is represented by Chemical Formula 1C,
Chemical Formula 1C is represented by one of Chemical Formula 1C-1 to Chemical Formula 1C-4: 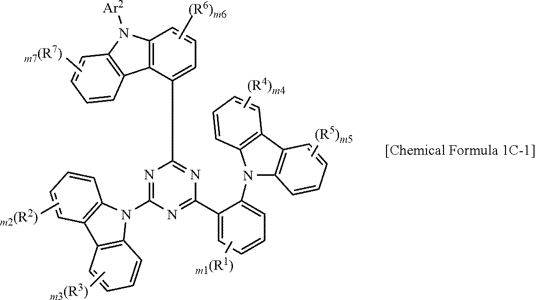 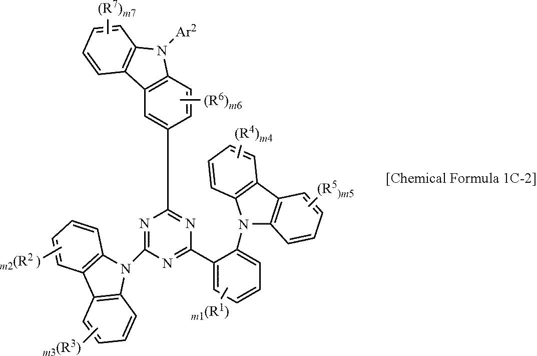 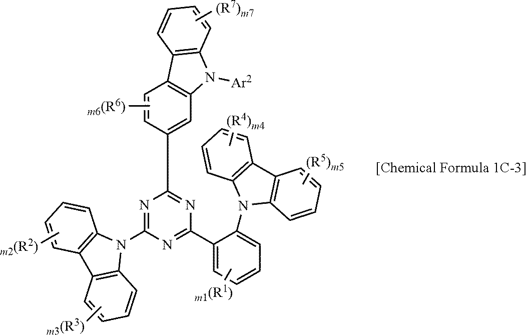 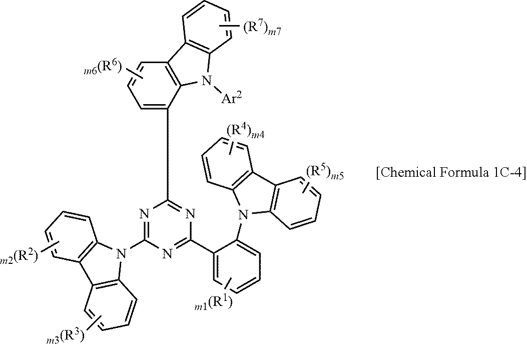
in Chemical Formula 1C-1 to Chemical Formula 1C-4, R¹ to R⁷, m1 to m7, and Ar² are defined the same as those of Chemical Formula 1 and Chemical Formula 1C.

6. The compound as claimed in claim 1, wherein:
Ar¹ is a group of Group I, 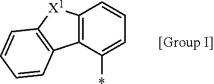 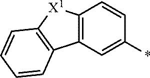 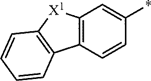 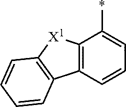 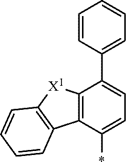 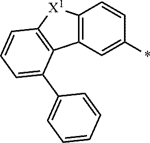 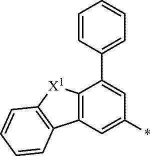 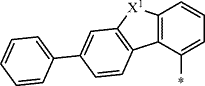 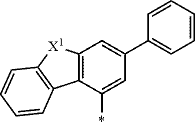 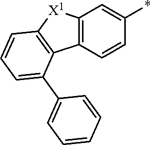 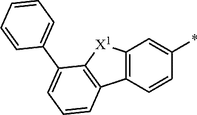 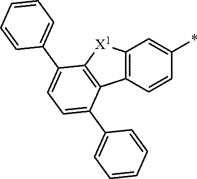 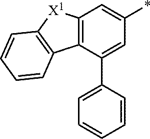 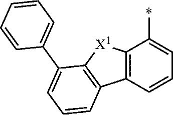 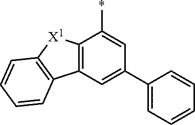 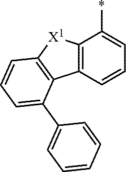 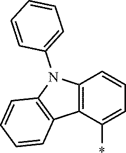 [SMILES: *c1cccc2c1c1ccccc1n2-c1ccccc1] 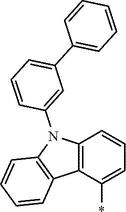 [SMILES: *c1cccc2c1c1ccccc1n2-c1cccc(-c2ccccc2)c1] 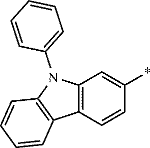 [SMILES: *c1ccc2c3ccccc3n(-c3ccccc3)c2c1] 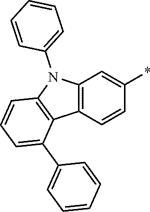 [SMILES: *c1ccc2c3c(-c4ccccc4)cccc3n(-c3ccccc3)c2c1] 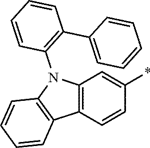 [SMILES: *c1ccc2c3ccccc3n(-c3ccccc3-c3ccccc3)c2c1] 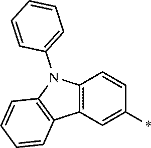 [SMILES: *c1ccc2c(c1)c1ccccc1n2-c1ccccc1] 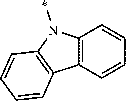 [SMILES: *n1c2ccccc2c2ccccc21] 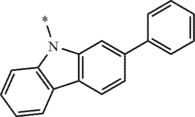 [SMILES: *n1c2ccccc2c2ccc(-c3ccccc3)cc21]
in Group I,
X¹ is O or S, and
* is a linking point.

7. The compound as claimed in claim 1, wherein the compound is a compound of Group 1:
[Group 1] 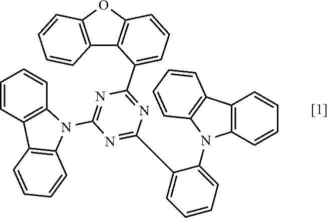 [SMILES: c1ccc(-n2c3ccccc3c3ccccc32)c(-c2nc(-c3cccc4oc5ccccc5c34)nc(-n3c4ccccc4c4ccccc43)n2)c1] 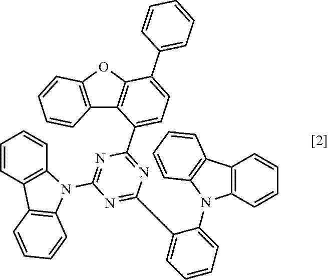 [SMILES: c1ccc(-c2ccc(-c3nc(-c4ccccc4-n4c5ccccc5c5ccccc54)nc(-n4c5ccccc5c5ccccc54)n3)c3c2oc2ccccc23)cc1] 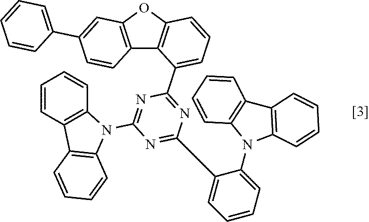 [SMILES: c1ccc(-c2ccc3c(c2)oc2cccc(-c4nc(-c5ccccc5-n5c6ccccc6c6ccccc65)nc(-n5c6ccccc6c6ccccc65)n4)c23)cc1] 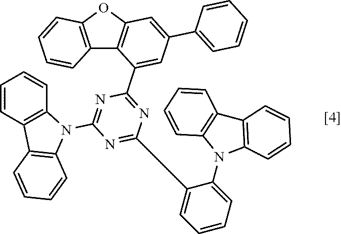 [SMILES: c1ccc(-c2cc(-c3nc(-c4ccccc4-n4c5ccccc5c5ccccc54)nc(-n4c5ccccc5c5ccccc54)n3)c3c(c2)oc2ccccc23)cc1] 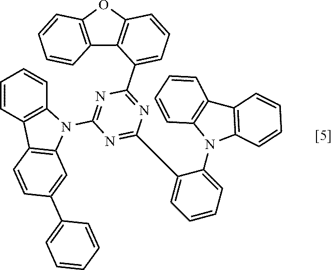 [SMILES: c1ccc(-c2ccc3c4ccccc4n(-c4nc(-c5ccccc5-n5c6ccccc6c6ccccc65)nc(-c5cccc6oc7ccccc7c56)n4)c3c2)cc1] 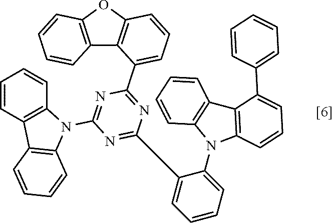 [SMILES: c1ccc(-c2cccc3c2c2ccccc2n3-c2ccccc2-c2nc(-c3cccc4oc5ccccc5c34)nc(-n3c4ccccc4c4ccccc43)n2)cc1] 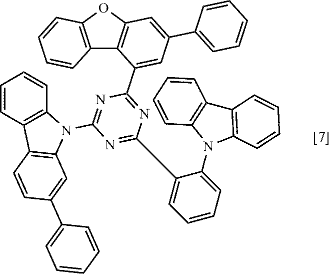 [SMILES: c1ccc(-c2cc(-c3nc(-c4ccccc4-n4c5ccccc5c5ccccc54)nc(-n4c5ccccc5c5ccc(-c6ccccc6)cc54)n3)c3c(c2)oc2ccccc23)cc1] 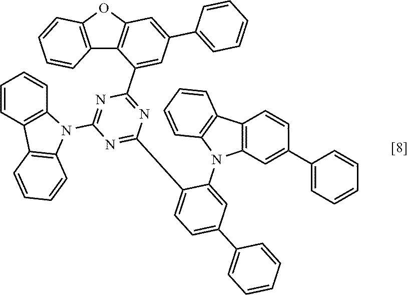 [SMILES: c1ccc(-c2ccc(-c3nc(-c4cc(-c5ccccc5)cc5oc6ccccc6c45)nc(-n4c5ccccc5c5ccccc54)n3)c(-n3c4ccccc4c4ccc(-c5ccccc5)cc43)c2)cc1] 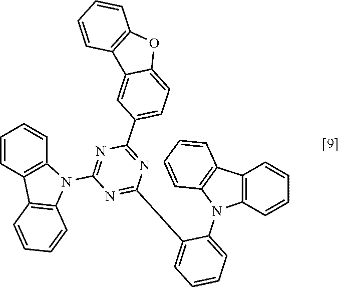 [SMILES: c1ccc(-n2c3ccccc3c3ccccc32)c(-c2nc(-c3ccc4oc5ccccc5c4c3)nc(-n3c4ccccc4c4ccccc43)n2)c1] 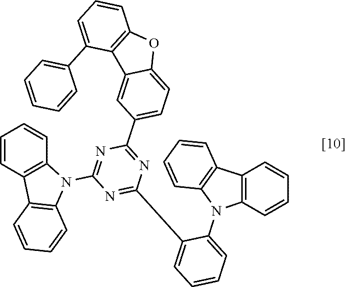 [SMILES: c1ccc(-c2cccc3oc4ccc(-c5nc(-c6ccccc6-n6c7ccccc7c7ccccc76)nc(-n6c7ccccc7c7ccccc76)n5)cc4c23)cc1] 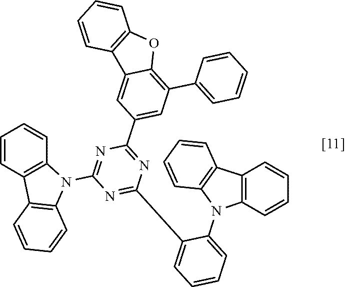 [SMILES: c1ccc(-c2cc(-c3nc(-c4ccccc4-n4c5ccccc5c5ccccc54)nc(-n4c5ccccc5c5ccccc54)n3)cc3c2oc2ccccc23)cc1] 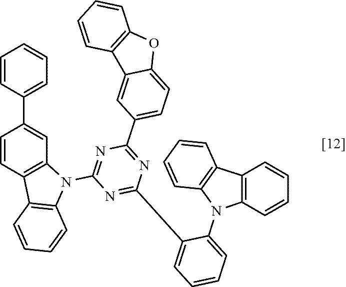 [SMILES: c1ccc(-c2ccc3c4ccccc4n(-c4nc(-c5ccc6oc7ccccc7c6c5)nc(-c5ccccc5-n5c6ccccc6c6ccccc65)n4)c3c2)cc1] 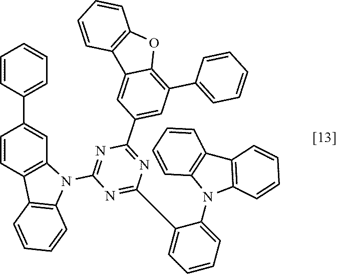 [SMILES: c1ccc(-c2ccc3c4ccccc4n(-c4nc(-c5cc(-c6ccccc6)c6oc7ccccc7c6c5)nc(-c5ccccc5-n5c6ccccc6c6ccccc65)n4)c3c2)cc1] 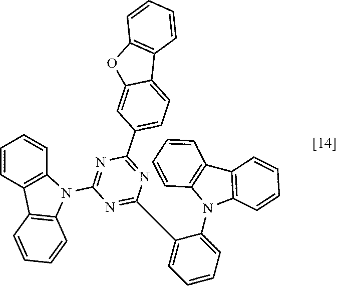 [SMILES: c1ccc(-n2c3ccccc3c3ccccc32)c(-c2nc(-c3ccc4c(c3)oc3ccccc34)nc(-n3c4ccccc4c4ccccc43)n2)c1] 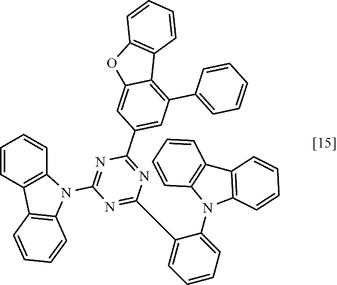 [SMILES: c1ccc(-c2cc(-c3nc(-c4ccccc4-n4c5ccccc5c5ccccc54)nc(-n4c5ccccc5c5ccccc54)n3)cc3oc4ccccc4c23)cc1] 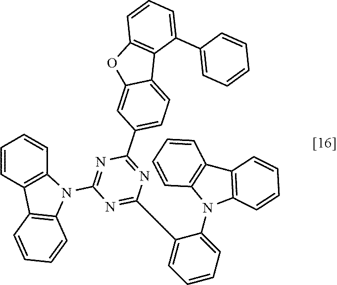 [SMILES: c1ccc(-c2cccc3oc4cc(-c5nc(-c6ccccc6-n6c7ccccc7c7ccccc76)nc(-n6c7ccccc7c7ccccc76)n5)ccc4c23)cc1] 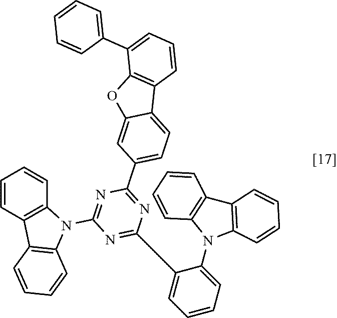 [SMILES: c1ccc(-c2cccc3c2oc2cc(-c4nc(-c5ccccc5-n5c6ccccc6c6ccccc65)nc(-n5c6ccccc6c6ccccc65)n4)ccc23)cc1] 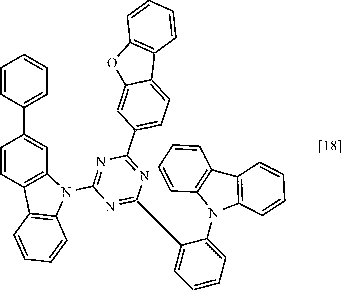 [SMILES: c1ccc(-c2ccc3c4ccccc4n(-c4nc(-c5ccc6c(c5)oc5ccccc56)nc(-c5ccccc5-n5c6ccccc6c6ccccc65)n4)c3c2)cc1] 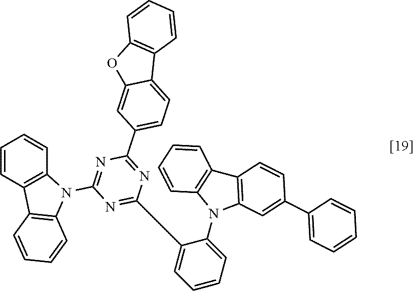 [SMILES: c1ccc(-c2ccc3c4ccccc4n(-c4ccccc4-c4nc(-c5ccc6c(c5)oc5ccccc56)nc(-n5c6ccccc6c6ccccc65)n4)c3c2)cc1] 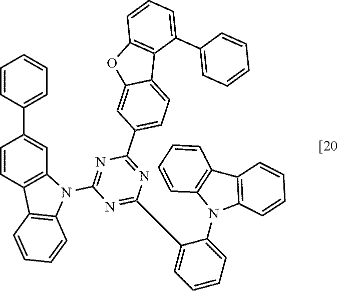 [SMILES: c1ccc(-c2ccc3c4ccccc4n(-c4nc(-c5ccc6c(c5)oc5cccc(-c7ccccc7)c56)nc(-c5ccccc5-n5c6ccccc6c6ccccc65)n4)c3c2)cc1] 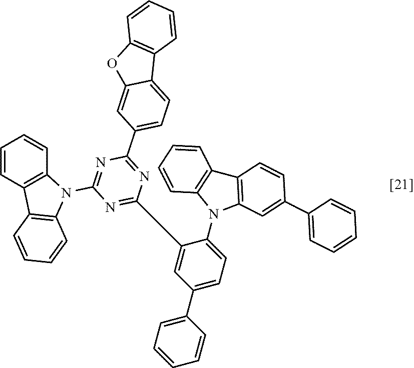 [SMILES: c1ccc(-c2ccc(-n3c4ccccc4c4ccc(-c5ccccc5)cc43)c(-c3nc(-c4ccc5c(c4)oc4ccccc45)nc(-n4c5ccccc5c5ccccc54)n3)c2)cc1] 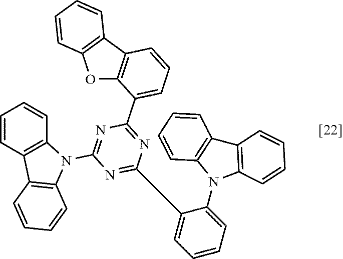 [SMILES: c1ccc(-n2c3ccccc3c3ccccc32)c(-c2nc(-c3cccc4c3oc3ccccc34)nc(-n3c4ccccc4c4ccccc43)n2)c1] 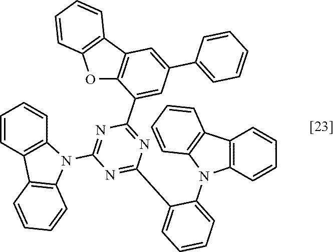 [SMILES: c1ccc(-c2cc(-c3nc(-c4ccccc4-n4c5ccccc5c5ccccc54)nc(-n4c5ccccc5c5ccccc54)n3)c3oc4ccccc4c3c2)cc1] 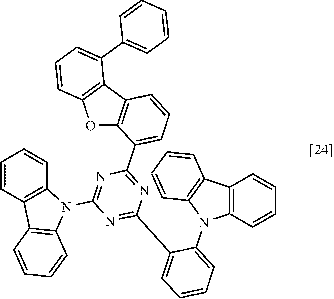 [SMILES: c1ccc(-c2cccc3oc4c(-c5nc(-c6ccccc6-n6c7ccccc7c7ccccc76)nc(-n6c7ccccc7c7ccccc76)n5)cccc4c23)cc1] 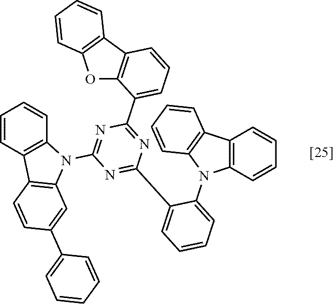 [SMILES: c1ccc(-c2ccc3c4ccccc4n(-c4nc(-c5ccccc5-n5c6ccccc6c6ccccc65)nc(-c5cccc6c5oc5ccccc56)n4)c3c2)cc1] 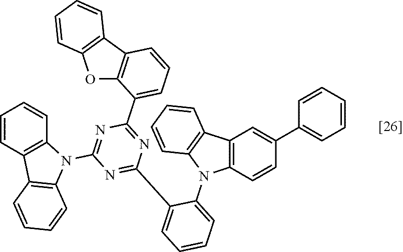 [SMILES: c1ccc(-c2ccc3c(c2)c2ccccc2n3-c2ccccc2-c2nc(-c3cccc4c3oc3ccccc34)nc(-n3c4ccccc4c4ccccc43)n2)cc1] 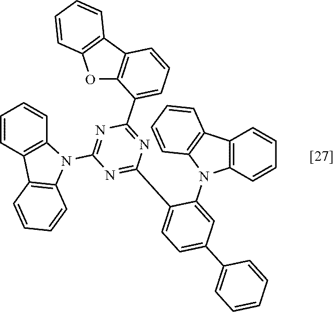 [SMILES: c1ccc(-c2ccc(-c3nc(-c4cccc5c4oc4ccccc45)nc(-n4c5ccccc5c5ccccc54)n3)c(-n3c4ccccc4c4ccccc43)c2)cc1] 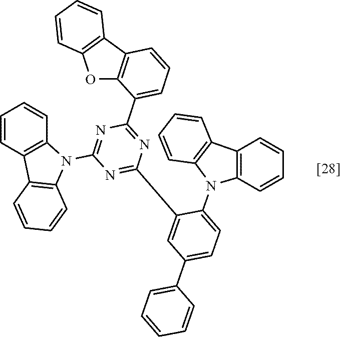 [SMILES: c1ccc(-c2ccc(-n3c4ccccc4c4ccccc43)c(-c3nc(-c4cccc5c4oc4ccccc45)nc(-n4c5ccccc5c5ccccc54)n3)c2)cc1] 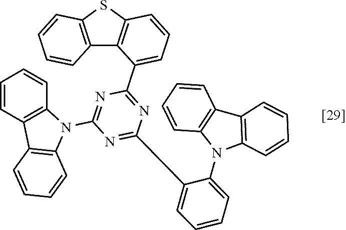 [SMILES: c1ccc(-n2c3ccccc3c3ccccc32)c(-c2nc(-c3cccc4sc5ccccc5c34)nc(-n3c4ccccc4c4ccccc43)n2)c1] 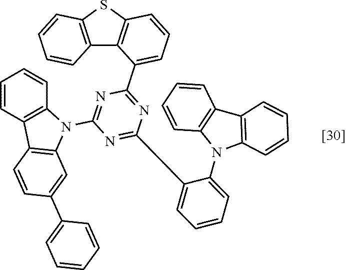 [SMILES: c1ccc(-c2ccc3c4ccccc4n(-c4nc(-c5ccccc5-n5c6ccccc6c6ccccc65)nc(-c5cccc6sc7ccccc7c56)n4)c3c2)cc1] 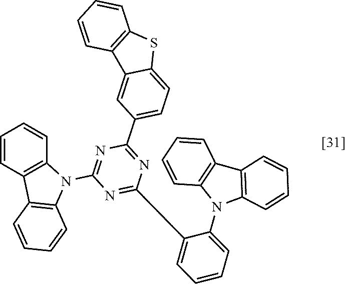 [SMILES: c1ccc(-n2c3ccccc3c3ccccc32)c(-c2nc(-c3ccc4sc5ccccc5c4c3)nc(-n3c4ccccc4c4ccccc43)n2)c1] 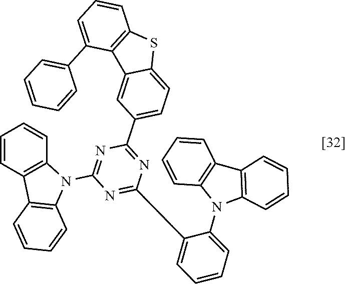 [SMILES: c1ccc(-c2cccc3sc4ccc(-c5nc(-c6ccccc6-n6c7ccccc7c7ccccc76)nc(-n6c7ccccc7c7ccccc76)n5)cc4c23)cc1] 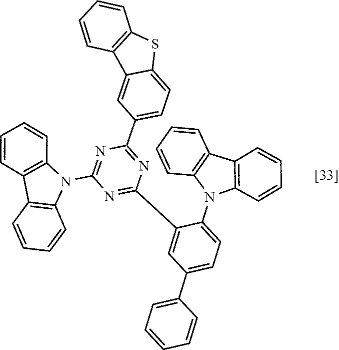 [SMILES: c1ccc(-c2ccc(-n3c4ccccc4c4ccccc43)c(-c3nc(-c4ccc5sc6ccccc6c5c4)nc(-n4c5ccccc5c5ccccc54)n3)c2)cc1] 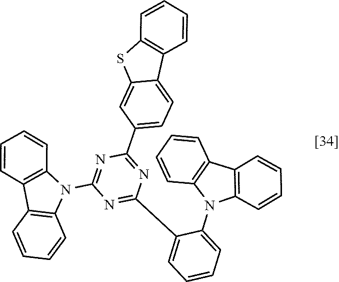 [SMILES: c1ccc(-n2c3ccccc3c3ccccc32)c(-c2nc(-c3ccc4c(c3)sc3ccccc34)nc(-n3c4ccccc4c4ccccc43)n2)c1] 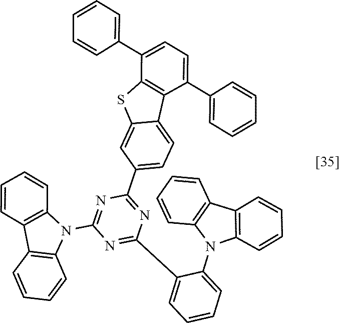 [SMILES: c1ccc(-c2ccc(-c3ccccc3)c3c2sc2cc(-c4nc(-c5ccccc5-n5c6ccccc6c6ccccc65)nc(-n5c6ccccc6c6ccccc65)n4)ccc23)cc1] 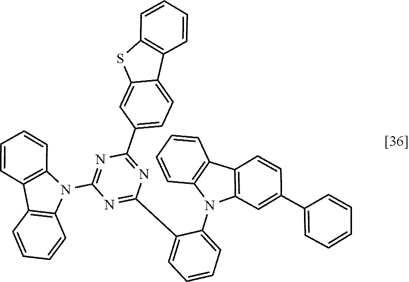 [SMILES: c1ccc(-c2ccc3c4ccccc4n(-c4ccccc4-c4nc(-c5ccc6c(c5)sc5ccccc56)nc(-n5c6ccccc6c6ccccc65)n4)c3c2)cc1] 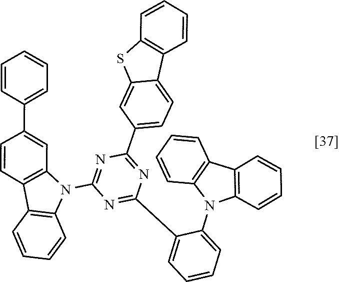 [SMILES: c1ccc(-c2ccc3c4ccccc4n(-c4nc(-c5ccc6c(c5)sc5ccccc56)nc(-c5ccccc5-n5c6ccccc6c6ccccc65)n4)c3c2)cc1] 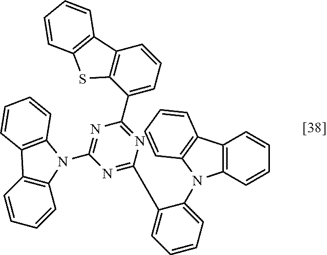 [SMILES: c1ccc(-n2c3ccccc3c3ccccc32)c(-c2nc(-c3cccc4c3sc3ccccc34)nc(-n3c4ccccc4c4ccccc43)n2)c1] 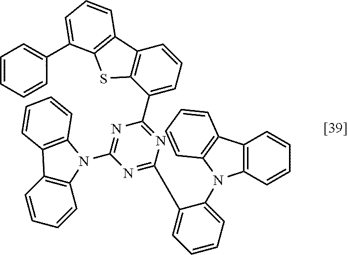 [SMILES: c1ccc(-c2cccc3c2sc2c(-c4nc(-c5ccccc5-n5c6ccccc6c6ccccc65)nc(-n5c6ccccc6c6ccccc65)n4)cccc23)cc1] 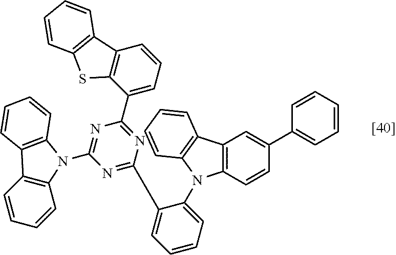 [SMILES: c1ccc(-c2ccc3c(c2)c2ccccc2n3-c2ccccc2-c2nc(-c3cccc4c3sc3ccccc34)nc(-n3c4ccccc4c4ccccc43)n2)cc1] 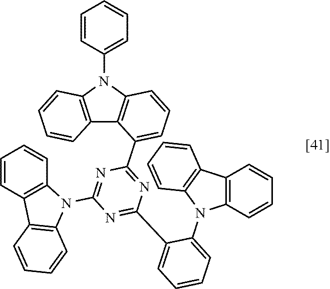 [SMILES: c1ccc(-n2c3ccccc3c3c(-c4nc(-c5ccccc5-n5c6ccccc6c6ccccc65)nc(-n5c6ccccc6c6ccccc65)n4)cccc32)cc1] 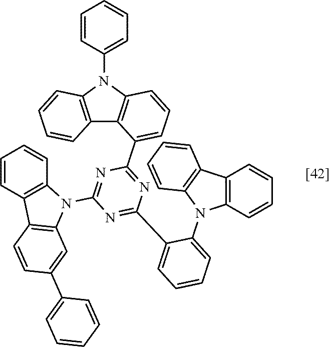 [SMILES: c1ccc(-c2ccc3c4ccccc4n(-c4nc(-c5ccccc5-n5c6ccccc6c6ccccc65)nc(-c5cccc6c5c5ccccc5n6-c5ccccc5)n4)c3c2)cc1] 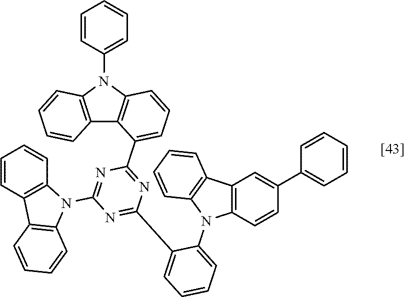 [SMILES: c1ccc(-c2ccc3c(c2)c2ccccc2n3-c2ccccc2-c2nc(-c3cccc4c3c3ccccc3n4-c3ccccc3)nc(-n3c4ccccc4c4ccccc43)n2)cc1] 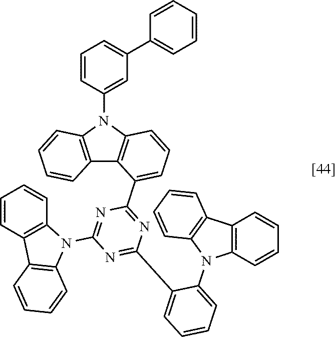 [SMILES: c1ccc(-c2cccc(-n3c4ccccc4c4c(-c5nc(-c6ccccc6-n6c7ccccc7c7ccccc76)nc(-n6c7ccccc7c7ccccc76)n5)cccc43)c2)cc1] 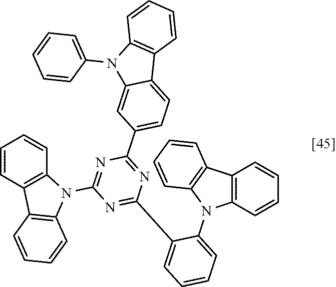 [SMILES: c1ccc(-n2c3ccccc3c3ccc(-c4nc(-c5ccccc5-n5c6ccccc6c6ccccc65)nc(-n5c6ccccc6c6ccccc65)n4)cc32)cc1] 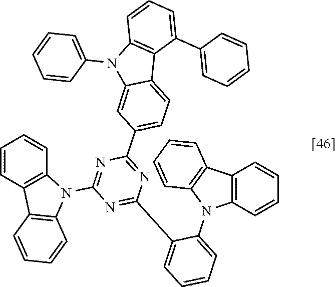 [SMILES: c1ccc(-c2cccc3c2c2ccc(-c4nc(-c5ccccc5-n5c6ccccc6c6ccccc65)nc(-n5c6ccccc6c6ccccc65)n4)cc2n3-c2ccccc2)cc1] 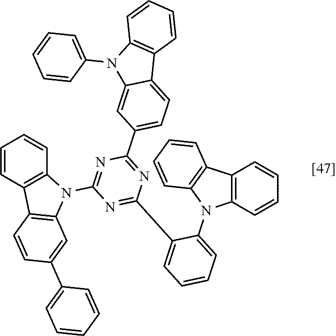 [SMILES: c1ccc(-c2ccc3c4ccccc4n(-c4nc(-c5ccc6c7ccccc7n(-c7ccccc7)c6c5)nc(-c5ccccc5-n5c6ccccc6c6ccccc65)n4)c3c2)cc1] 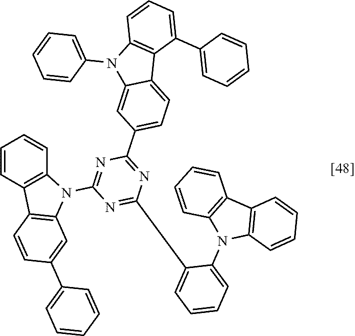 [SMILES: c1ccc(-c2ccc3c4ccccc4n(-c4nc(-c5ccc6c7c(-c8ccccc8)cccc7n(-c7ccccc7)c6c5)nc(-c5ccccc5-n5c6ccccc6c6ccccc65)n4)c3c2)cc1] 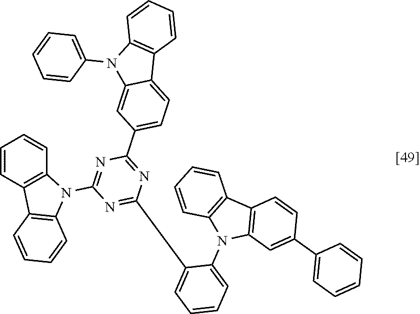 [SMILES: c1ccc(-c2ccc3c4ccccc4n(-c4ccccc4-c4nc(-c5ccc6c7ccccc7n(-c7ccccc7)c6c5)nc(-n5c6ccccc6c6ccccc65)n4)c3c2)cc1] 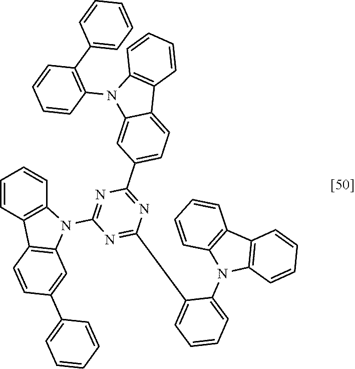 [SMILES: c1ccc(-c2ccc3c4ccccc4n(-c4nc(-c5ccc6c7ccccc7n(-c7ccccc7-c7ccccc7)c6c5)nc(-c5ccccc5-n5c6ccccc6c6ccccc65)n4)c3c2)cc1] 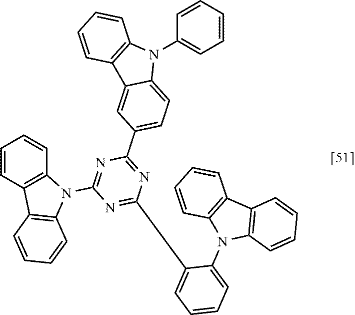 [SMILES: c1ccc(-n2c3ccccc3c3cc(-c4nc(-c5ccccc5-n5c6ccccc6c6ccccc65)nc(-n5c6ccccc6c6ccccc65)n4)ccc32)cc1] 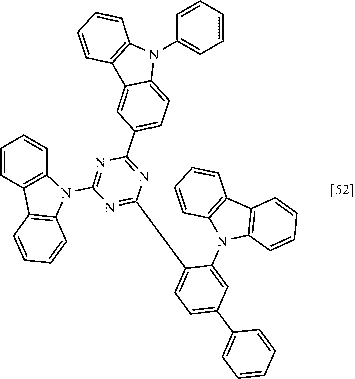 [SMILES: c1ccc(-c2ccc(-c3nc(-c4ccc5c(c4)c4ccccc4n5-c4ccccc4)nc(-n4c5ccccc5c5ccccc54)n3)c(-n3c4ccccc4c4ccccc43)c2)cc1] 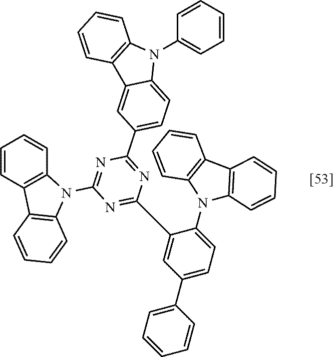 [SMILES: c1ccc(-c2ccc(-n3c4ccccc4c4ccccc43)c(-c3nc(-c4ccc5c(c4)c4ccccc4n5-c4ccccc4)nc(-n4c5ccccc5c5ccccc54)n3)c2)cc1] 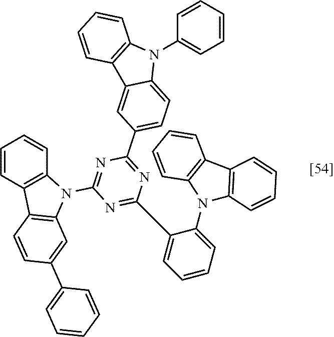 [SMILES: c1ccc(-c2ccc3c4ccccc4n(-c4nc(-c5ccc6c(c5)c5ccccc5n6-c5ccccc5)nc(-c5ccccc5-n5c6ccccc6c6ccccc65)n4)c3c2)cc1] 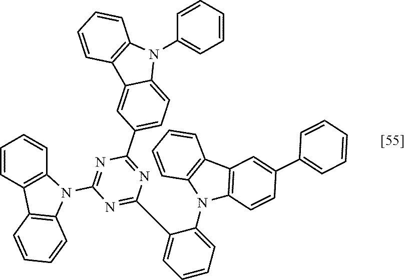 [SMILES: c1ccc(-c2ccc3c(c2)c2ccccc2n3-c2ccccc2-c2nc(-c3ccc4c(c3)c3ccccc3n4-c3ccccc3)nc(-n3c4ccccc4c4ccccc43)n2)cc1] 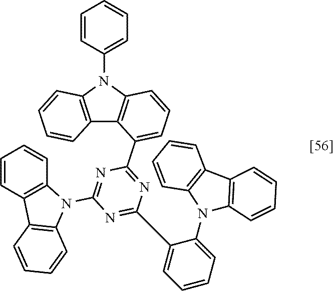 [SMILES: c1ccc(-n2c3ccccc3c3c(-c4nc(-c5ccccc5-n5c6ccccc6c6ccccc65)nc(-n5c6ccccc6c6ccccc65)n4)cccc32)cc1] 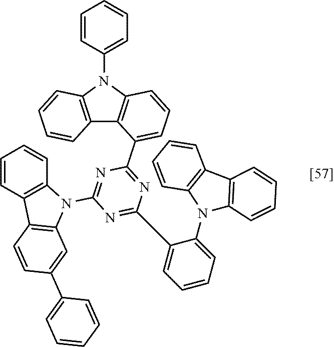 [SMILES: c1ccc(-c2ccc3c4ccccc4n(-c4nc(-c5ccccc5-n5c6ccccc6c6ccccc65)nc(-c5cccc6c5c5ccccc5n6-c5ccccc5)n4)c3c2)cc1] 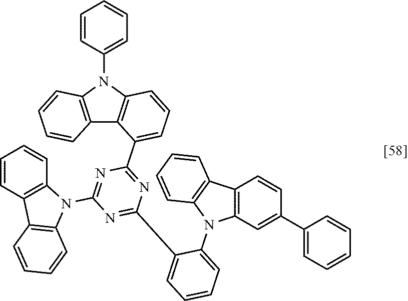 [SMILES: c1ccc(-c2ccc3c4ccccc4n(-c4ccccc4-c4nc(-c5cccc6c5c5ccccc5n6-c5ccccc5)nc(-n5c6ccccc6c6ccccc65)n4)c3c2)cc1] 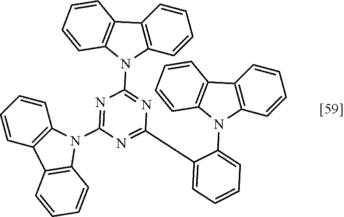 [SMILES: c1ccc(-n2c3ccccc3c3ccccc32)c(-c2nc(-n3c4ccccc4c4ccccc43)nc(-n3c4ccccc4c4ccccc43)n2)c1] 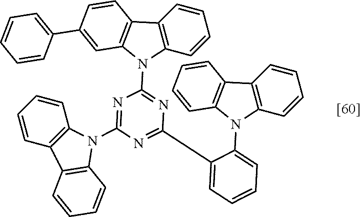 [SMILES: c1ccc(-c2ccc3c4ccccc4n(-c4nc(-c5ccccc5-n5c6ccccc6c6ccccc65)nc(-n5c6ccccc6c6ccccc65)n4)c3c2)cc1] 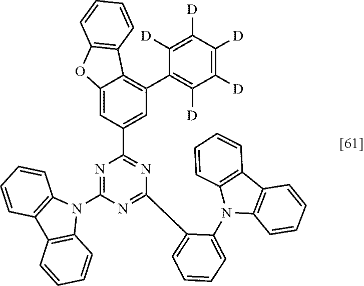 [SMILES: [2H]c1c([2H])c([2H])c(-c2cc(-c3nc(-c4ccccc4-n4c5ccccc5c5ccccc54)nc(-n4c5ccccc5c5ccccc54)n3)cc3oc4ccccc4c23)c([2H])c1[2H]] 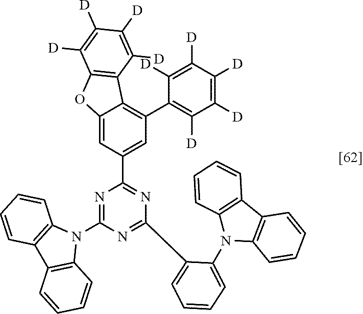 [SMILES: [2H]c1c([2H])c([2H])c(-c2cc(-c3nc(-c4ccccc4-n4c5ccccc5c5ccccc54)nc(-n4c5ccccc5c5ccccc54)n3)cc3oc4c([2H])c([2H])c([2H])c([2H])c4c23)c([2H])c1[2H]] 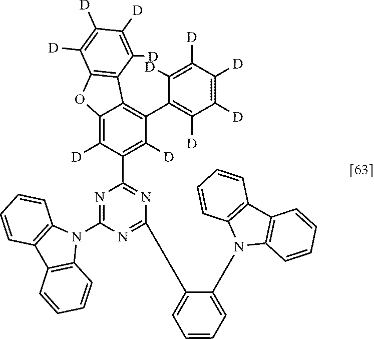 [SMILES: [2H]c1c([2H])c([2H])c(-c2c([2H])c(-c3nc(-c4ccccc4-n4c5ccccc5c5ccccc54)nc(-n4c5ccccc5c5ccccc54)n3)c([2H])c3oc4c([2H])c([2H])c([2H])c([2H])c4c23)c([2H])c1[2H]] 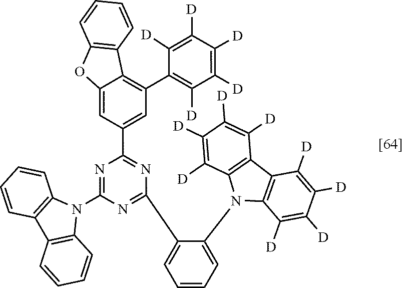 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5ccccc5c5ccccc54)n3)cc3oc4ccccc4c23)c([2H])c1[2H]] 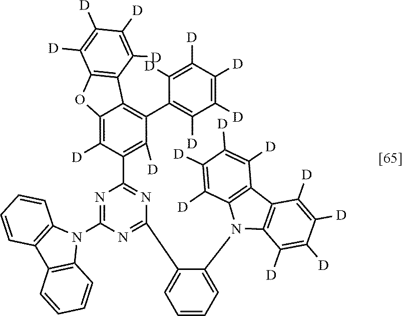 [SMILES: [2H]c1c([2H])c([2H])c(-c2c([2H])c(-c3nc(-c4ccccc4-n4c5c([2H])c([2H])c([2H])c([2H])c5c5c([2H])c([2H])c([2H])c([2H])c54)nc(-n4c5ccccc5c5ccccc54)n3)c([2H])c3oc4c([2H])c([2H])c([2H])c([2H])c4c23)c([2H])c1[2H]] 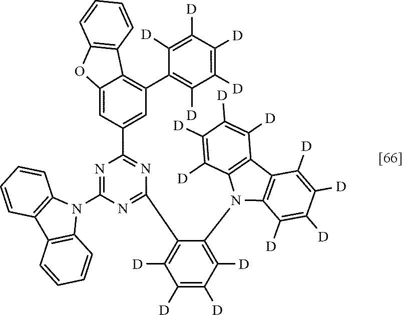 [SMILES: [2H]c1c([2H])c([2H])c(-c2cc(-c3nc(-c4c([2H])c([2H])c([2H])c([2H])c4-n4c5c([2H])c([2H])c([2H])c([2H])c5c5c([2H])c([2H])c([2H])c([2H])c54)nc(-n4c5ccccc5c5ccccc54)n3)cc3oc4ccccc4c23)c([2H])c1[2H]] 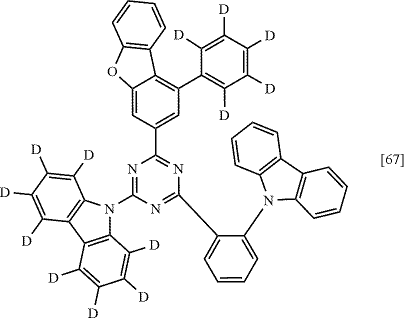 [SMILES: [2H]c1c([2H])c([2H])c(-c2cc(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)cc3oc4ccccc4c23)c([2H])c1[2H]] 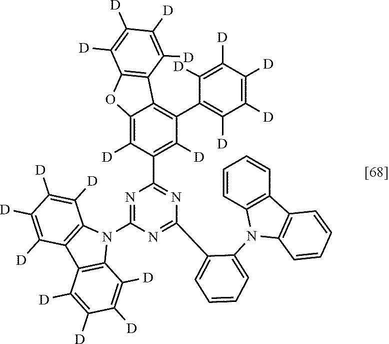 [SMILES: [2H]c1c([2H])c([2H])c(-c2c([2H])c(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]] 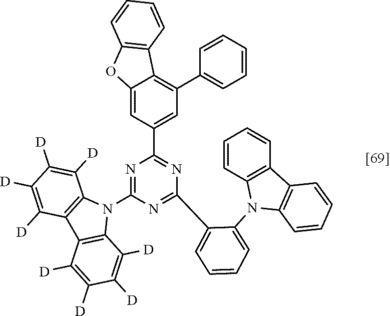 [SMILES: [2H]c1c([2H])c([2H])c2c(c1[2H])c1c([2H])c([2H])c([2H])c([2H])c1n2-c1nc(-c2cc(-c3ccccc3)c3c(c2)oc2ccccc23)nc(-c2ccccc2-n2c3ccccc3c3ccccc32)n1] 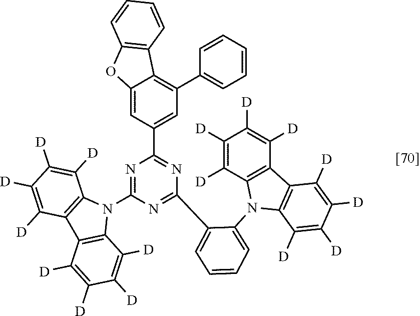 [SMILES: [2H]c1c([2H])c([2H])c2c(c1[2H])c1c([2H])c([2H])c([2H])c([2H])c1n2-c1nc(-c2cc(-c3ccccc3)c3c(c2)oc2ccccc23)nc(-c2ccccc2-n2c3c([2H])c([2H])c([2H])c([2H])c3c3c([2H])c([2H])c([2H])c([2H])c32)n1] 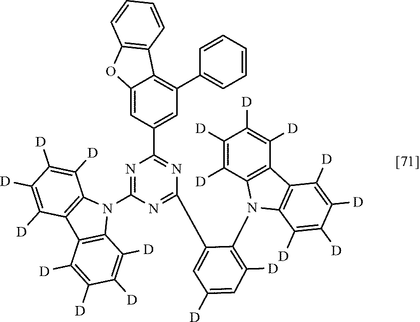 [SMILES: [2H]c1cc([2H])c(-n2c3c([2H])c([2H])c([2H])c([2H])c3c3c([2H])c([2H])c([2H])c([2H])c32)c(-c2nc(-c3cc(-c4ccccc4)c4c(c3)oc3ccccc34)nc(-n3c4c([2H])c([2H])c([2H])c([2H])c4c4c([2H])c([2H])c([2H])c([2H])c43)n2)c1] 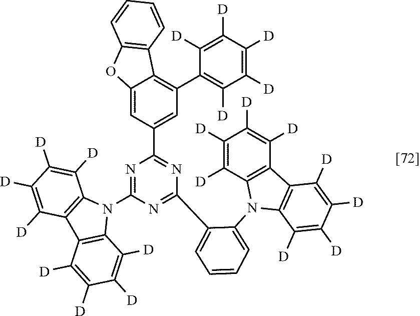 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)cc3oc4ccccc4c23)c([2H])c1[2H]] 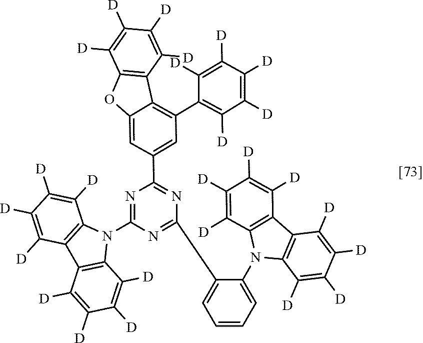 [SMILES: [2H]c1c([2H])c([2H])c(-c2cc(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)cc3oc4c([2H])c([2H])c([2H])c([2H])c4c23)c([2H])c1[2H]] 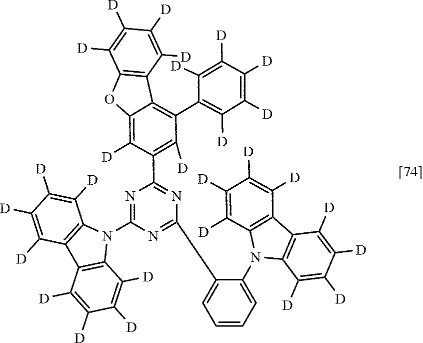 [SMILES: [2H]c1c([2H])c([2H])c(-c2c([2H])c(-c3nc(-c4ccccc4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]] 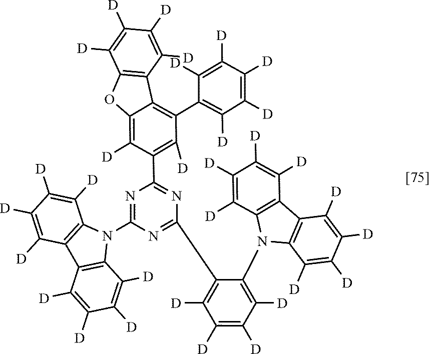 [SMILES: [2H]c1c([2H])c([2H])c(-c2c([2H])c(-c3nc(-c4c([2H])c([2H])c([2H])c([2H])c4-n4c5c([2H])c([2H])c([2H])c([2H])c5c5c([2H])c([2H])c([2H])c([2H])c54)nc(-n4c5c([2H])c([2H])c([2H])c([2H])c5c5c([2H])c([2H])c([2H])c([2H])c54)n3)c([2H])c3oc4c([2H])c([2H])c([2H])c([2H])c4c23)c([2H])c1[2H]] 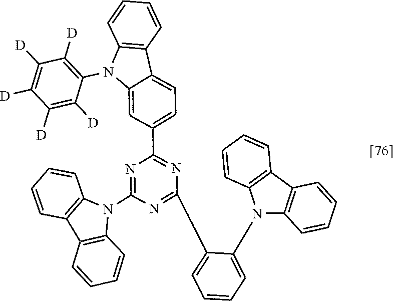 [SMILES: [2H]c1c([2H])c([2H])c(-n2c3ccccc3c3ccc(-c4nc(-c5ccccc5-n5c6ccccc6c6ccccc65)nc(-n5c6ccccc6c6ccccc65)n4)cc32)c([2H])c1[2H]] 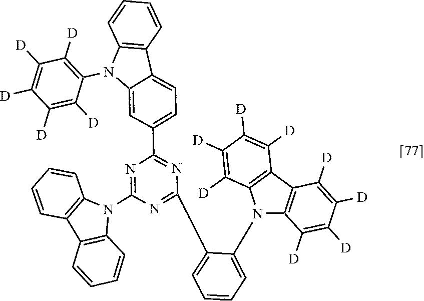 [SMILES: [2H]c1c([2H])c([2H])c(-n2c3ccccc3c3ccc(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)cc32)c([2H])c1[2H]] 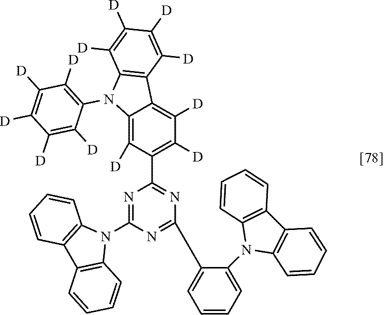 [SMILES: [2H]c1c([2H])c([2H])c(-n2c3c([2H])c([2H])c([2H])c([2H])c3c3c([2H])c([2H])c(-c4nc(-c5ccccc5-n5c6ccccc6c6ccccc65)nc(-n5c6ccccc6c6ccccc65)n4)c([2H])c32)c([2H])c1[2H]] 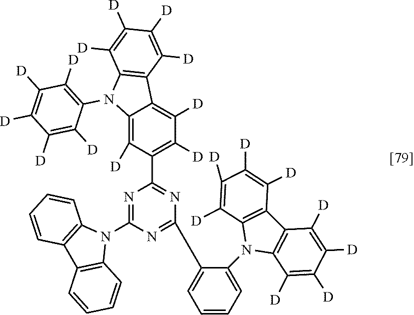 [SMILES: [2H]c1c([2H])c([2H])c(-n2c3c([2H])c([2H])c([2H])c([2H])c3c3c([2H])c([2H])c(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)c([2H])c32)c([2H])c1[2H]] 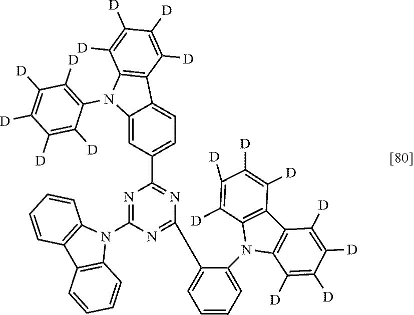 [SMILES: [2H]c1c([2H])c([2H])c(-n2c3cc(-c4nc(-c5ccccc5-n5c6c([2H])c([2H])c([2H])c([2H])c6c6c([2H])c([2H])c([2H])c([2H])c65)nc(-n5c6ccccc6c6ccccc65)n4)ccc3c3c([2H])c([2H])c([2H])c([2H])c32)c([2H])c1[2H]] 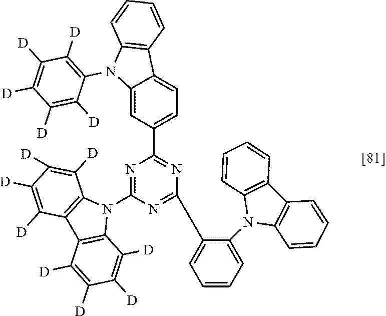 [SMILES: [2H]c1c([2H])c([2H])c(-n2c3ccccc3c3ccc(-c4nc(-c5ccccc5-n5c6ccccc6c6ccccc65)nc(-n5c6c([2H])c([2H])c([2H])c([2H])c6c6c([2H])c([2H])c([2H])c([2H])c65)n4)cc32)c([2H])c1[2H]] 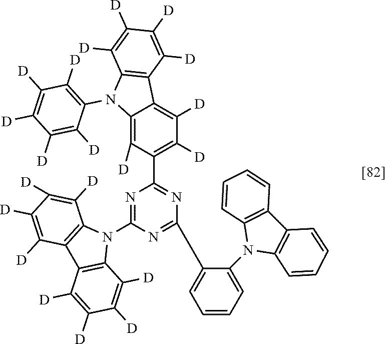 [SMILES: [2H]c1c([2H])c([2H])c(-n2c3c([2H])c([2H])c([2H])c([2H])c3c3c([2H])c([2H])c(-c4nc(-c5ccccc5-n5c6ccccc6c6ccccc65)nc(-n5c6c([2H])c([2H])c([2H])c([2H])c6c6c([2H])c([2H])c([2H])c([2H])c65)n4)c([2H])c32)c([2H])c1[2H]] 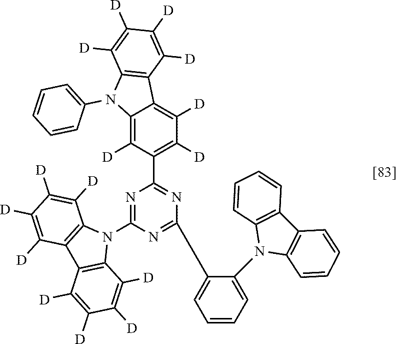 [SMILES: [2H]c1c([2H])c([2H])c2c(c1[2H])c1c([2H])c([2H])c(-c3nc(-c4ccccc4-n4c5ccccc5c5ccccc54)nc(-n4c5c([2H])c([2H])c([2H])c([2H])c5c5c([2H])c([2H])c([2H])c([2H])c54)n3)c([2H])c1n2-c1ccccc1] 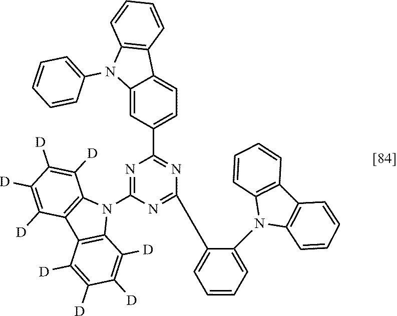 [SMILES: [2H]c1c([2H])c([2H])c2c(c1[2H])c1c([2H])c([2H])c([2H])c([2H])c1n2-c1nc(-c2ccc3c4ccccc4n(-c4ccccc4)c3c2)nc(-c2ccccc2-n2c3ccccc3c3ccccc32)n1] 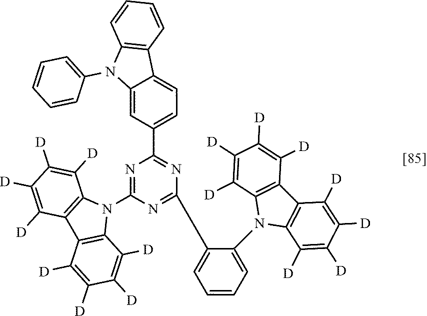 [SMILES: [2H]c1c([2H])c([2H])c2c(c1[2H])c1c([2H])c([2H])c([2H])c([2H])c1n2-c1nc(-c2ccc3c4ccccc4n(-c4ccccc4)c3c2)nc(-c2ccccc2-n2c3c([2H])c([2H])c([2H])c([2H])c3c3c([2H])c([2H])c([2H])c([2H])c32)n1] 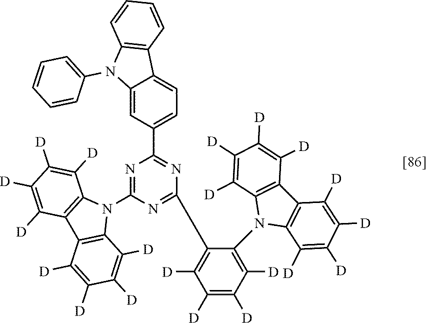 [SMILES: [2H]c1c([2H])c([2H])c(-n2c3c([2H])c([2H])c([2H])c([2H])c3c3c([2H])c([2H])c([2H])c([2H])c32)c(-c2nc(-c3ccc4c5ccccc5n(-c5ccccc5)c4c3)nc(-n3c4c([2H])c([2H])c([2H])c([2H])c4c4c([2H])c([2H])c([2H])c([2H])c43)n2)c1[2H]] 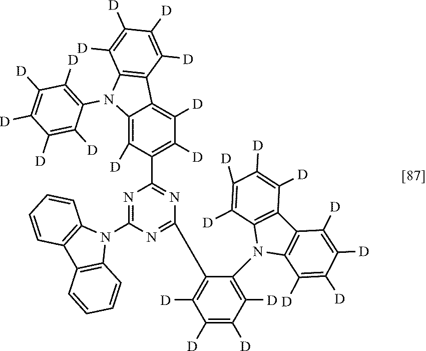 [SMILES: [2H]c1c([2H])c([2H])c(-n2c3c([2H])c([2H])c([2H])c([2H])c3c3c([2H])c([2H])c(-c4nc(-c5c([2H])c([2H])c([2H])c([2H])c5-n5c6c([2H])c([2H])c([2H])c([2H])c6c6c([2H])c([2H])c([2H])c([2H])c65)nc(-n5c6ccccc6c6ccccc65)n4)c([2H])c32)c([2H])c1[2H]] 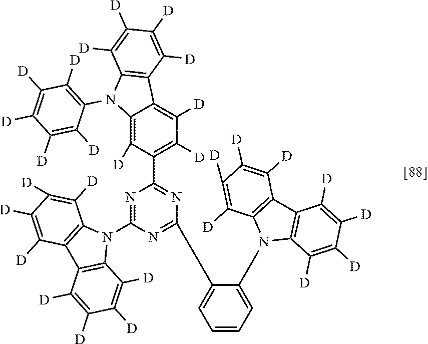 [SMILES: [2H]c1c([2H])c([2H])c(-n2c3c([2H])c([2H])c([2H])c([2H])c3c3c([2H])c([2H])c(-c4nc(-c5ccccc5-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c([2H])c32)c([2H])c1[2H]] 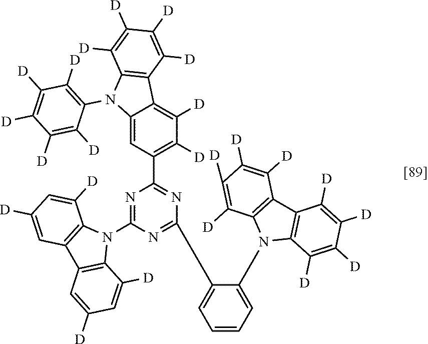 [SMILES: [2H]c1cc([2H])c2c(c1)c1cc([2H])cc([2H])c1n2-c1nc(-c2ccccc2-n2c3c([2H])c([2H])c([2H])c([2H])c3c3c([2H])c([2H])c([2H])c([2H])c32)nc(-c2cc3c(c([2H])c2[2H])c2c([2H])c([2H])c([2H])c([2H])c2n3-c2c([2H])c([2H])c([2H])c([2H])c2[2H])n1] 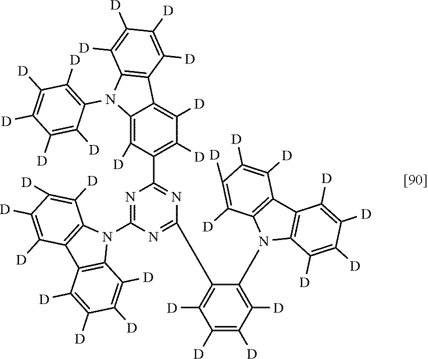 [SMILES: [2H]c1c([2H])c([2H])c(-n2c3c([2H])c([2H])c([2H])c([2H])c3c3c([2H])c([2H])c(-c4nc(-c5c([2H])c([2H])c([2H])c([2H])c5-n5c6c([2H])c([2H])c([2H])c([2H])c6c6c([2H])c([2H])c([2H])c([2H])c65)nc(-n5c6c([2H])c([2H])c([2H])c([2H])c6c6c([2H])c([2H])c([2H])c([2H])c65)n4)c([2H])c32)c([2H])c1[2H]]
.

8. A composition for an organic optoelectronic device, the composition comprising:
a first compound; and
a second compound,
wherein:
the first compound is the compound as claimed in claim 1, and
the second compound is represented by Chemical Formula 2 or is represented by a combination of Chemical Formulae 3 and 4: 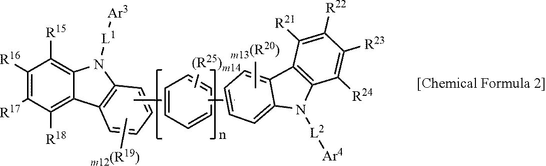
in Chemical Formula 2,
Ar³ and Ar⁴ are each independently a substituted or unsubstituted C6 to C20 aryl group or a substituted or unsubstituted C2 to C30 heterocyclic group,
L¹ and L² are each independently a single bond or a substituted or unsubstituted C6 to C20 arylene group,
R¹⁵ to R²⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m12 and m13 are each independently an integer of 1 to 3,
m14 is an integer of 1 to 4, and
n is an integer of 0 to 2; 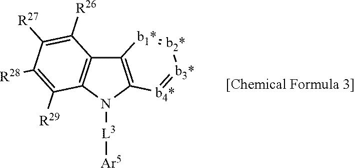 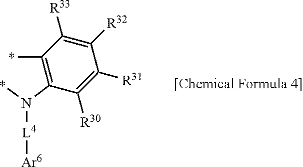
in Chemical Formulas 3 and 4,
Ar⁵ and Ar⁶ are each independently a substituted or unsubstituted C6 to C20 aryl group or a substituted or unsubstituted C2 to C30 heterocyclic group,
two adjacent ones of b₁* to b₄* of Chemical Formula 3 are linking carbons linked at * of Chemical Formula 4,
the remaining two of b₁* to b₄* of Chemical Formula 3, not linked at * of Chemical Formula 4, are each independently C—L^{a}—R^{a},
L^{a}, L³, and L⁴ are each independently a single bond or a substituted or unsubstituted C6 to C20 arylene group, and
R^{a} and R²⁶ to R³³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

9. The composition as claimed in claim 8, wherein:
the second compound is represented by Chemical Formula 2,
the compound represented by Chemical Formula 2 is represented by Chemical Formula 2-8: 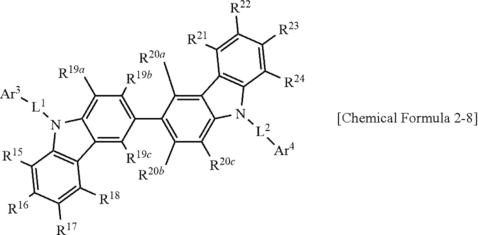
in Chemical Formula 2-8,
R¹⁵ to R¹⁸, R^{19a}, R^{19b}, R^{19c}, R^{20a}, R^{20b}, R^{20c}, and R²¹ to R²⁴ are each independently hydrogen, deuterium, or a substituted or unsubstituted C6 to C12 aryl group, and
moieties *—L¹—Ar³ and *—L²—Ar⁴ are each independently a moiety of Group II,
[Group II] 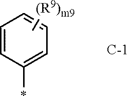 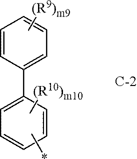 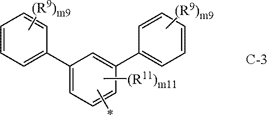 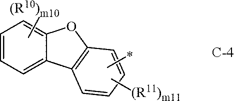 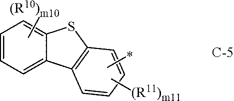 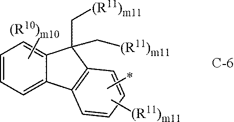 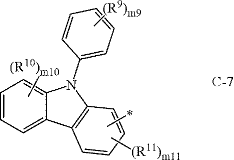 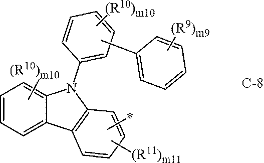 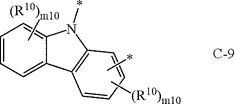 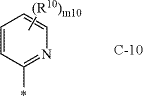 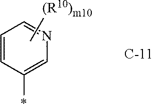 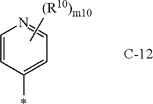 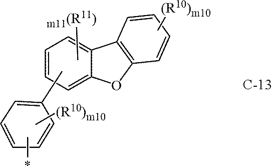 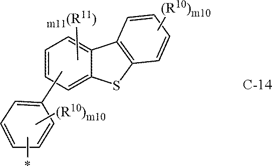 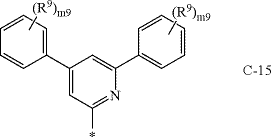 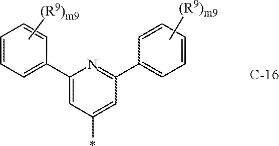 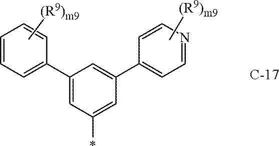 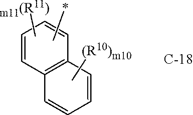 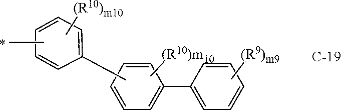 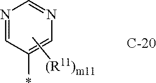 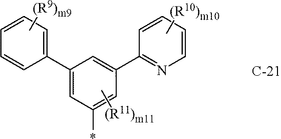 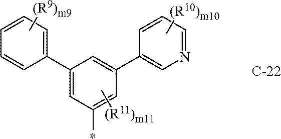 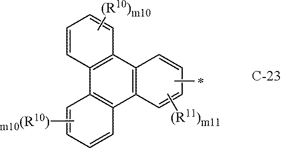 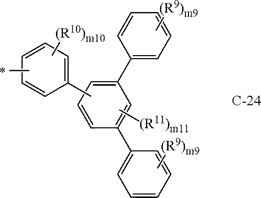 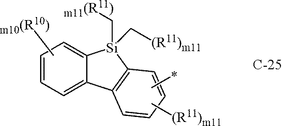 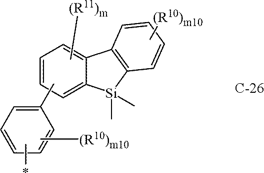
in Group II,
R⁹ to R¹¹ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m9 is one of integers from 1 to 5,
m10 is one of integers from 1 to 4,
m11 is one of integers from 1 to 3, and
* is a linking point.

10. The composition as claimed in claim 8, wherein
the second compound is represented by the combination of Chemical Formulae 3 and 4,
the combination of Chemical Formulae 3 and 4 is represented by Chemical Formula 3C: 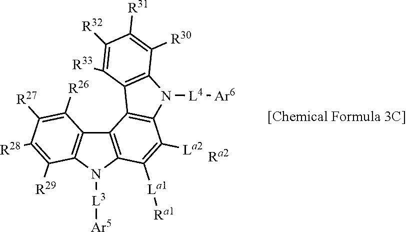
in Chemical Formula 3C,
L^{al} and L^{a2} are each a single bond,
L³ and L⁴ are each independently a single bond or a substituted or unsubstituted C6 to C12 arylene group,
R²⁶ to R³³, R^{a1} and R^{a2} are each independently hydrogen, deuterium, or a substituted or unsubstituted C6 to C12 aryl group, and
moieties *—L³—Ar⁵ and *—L⁴—Ar⁶ are each independently a moiety of Group II,
[Group II] 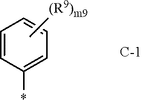 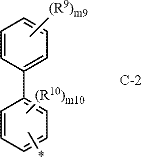 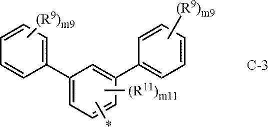 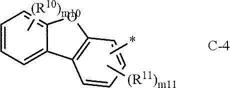 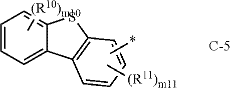 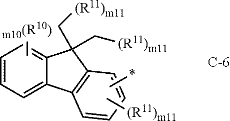 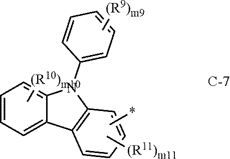 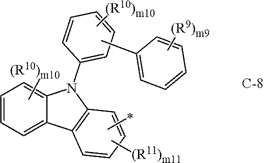 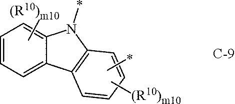 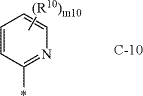 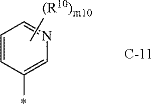 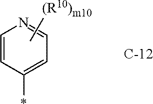 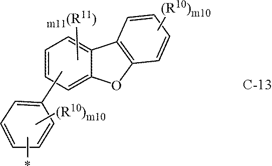 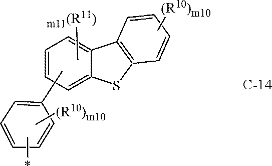 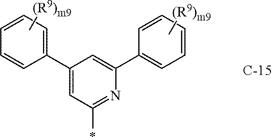 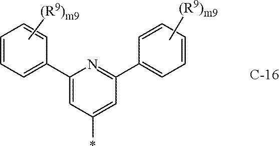 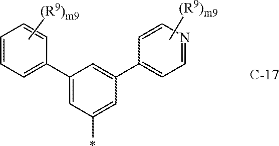 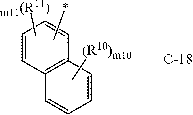 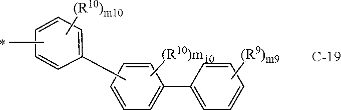 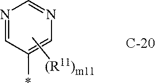 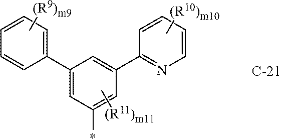 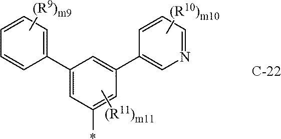 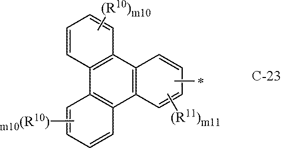 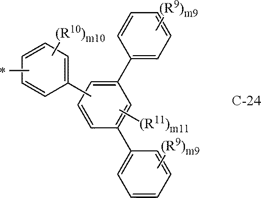 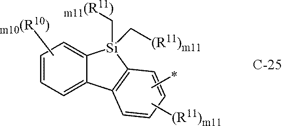 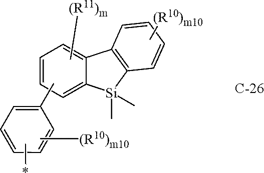
in Group II,
R⁹ to R¹¹ are each independently hydrogen, deuterium, a substituted or unsubstituted C1 to C4 alkyl group, a substituted or unsubstituted C6 to C18 aryl group, or a substituted or unsubstituted C2 to C30 heteroaryl group,
m9 is an integer of 1 to 5,
m10 is an integer of 1 to 4,
m11 is an integer of 1 to 3, and
* is a linking point.

11. An organic optoelectronic device, comprising:
an anode and a cathode facing each other, and
at least one organic layer between the anode and the cathode,
wherein the at least one organic layer includes the compound for an organic optoelectronic device as claimed in claim 1.

12. The organic optoelectronic device as claimed in claim 11, wherein:
the at least one organic layer includes a light emitting layer, and
the light emitting layer includes the compound for an organic optoelectronic device.

13. A display device comprising the organic optoelectronic device as claimed in claim 11.

14. An organic optoelectronic device, comprising:
an anode and a cathode facing each other, and
at least one organic layer between the anode and the cathode,
wherein the at least one organic layer includes the composition for an organic optoelectronic device as claimed in claim 8.

15. The organic optoelectronic device as claimed in claim 14, wherein:
the at least one organic layer includes a light emitting layer, and
the light emitting layer includes the composition for an organic optoelectronic device.

16. A display device comprising the organic optoelectronic device as claimed in claim 14.
